(19) United States
(12) Patent Application Publication
(10) Pub. No.: US 2026/0001898 A1
(43) Pub. Date: Jan. 1, 2026

(54) Heteroaryl compounds as inhibitors of TYK2/JAK1, composition and application thereof

(71) Applicant: Accro Bioscience (HK) Limited, Wan Chai (HK)

(72) Inventors: Xiaohu Zhang, Suzhou (CN); Sudan He, Suzhou (CN)

(21) Appl. No.: 18/881,281

(22) PCT Filed: Jul. 8, 2023
(86) PCT No.: PCT/US2023/027187

§ 371 Date: Jan. 4, 2025

(30) Foreign Application Priority Data
Jul. 8, 2022 (CN) 202210853468.X
Dec. 8, 2022 (CN) 202211575172.2

(51) Int. Cl. C07F 9/6558; A61K 31/675; A61K 45/06; C07F 9/58; C07F 9/6509

(52) U.S. Cl. CPC C07F 9/65583; A61K 31/675; A61K 45/06; C07F 9/58; C07F 9/650905; C07F 9/65586

(57) ABSTRACT

The present disclosure provides phosphonate-containing heterocycle compounds with TYK2/JAK1 inhibitory activities, pharmaceutical compositions comprising the same, and applications thereof. The present disclosure provides compounds of Formula (I), as inhibitors of TYK2/JAK1. These compounds can be used for preventing and/or treating TYK2/JAK1 related diseases and/or conditions without significant inhibition of JAK2 activities.

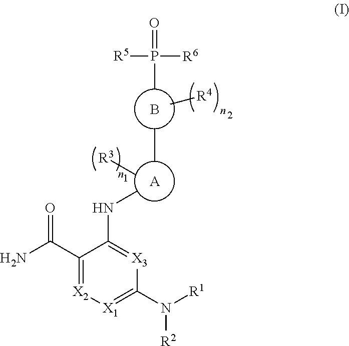

### CROSS REFERENCE TO RELATED APPLICATIONS

This application claims the benefit of Chinese Patent Applications 202210853468.X, filed on Jul. 8, 2022; and, 202211575172.2, filed on Dec. 8, 2022; all of which are hereby incorporated by reference in their entirety.

### FIELD OF INVENTION

The present invention is in the medical technology field, and relates to a compound with selective TYK2/JAK1 inhibitory activities. The present invention also relates to compositions comprising the disclosed compounds, their method of making, and their applications in therapies targeting the prevention and/or treatment of diseases associated with TYK2/JAK1, such as tumors, autoimmune diseases, neurodegenerative diseases, metabolic diseases, and genetic diseases.

### BACKGROUND OF THE INVENTION

Janus kinases or JAKs are non-receptor tyrosine kinases that bind to the intracellular portion of cell surface cytokine receptors. Currently there are four known human JAK family members: JAK1, JAK2, JAK3 and TYK2 (tyrosine kinases 2), each of which contains a kinase domain and a pseudokinase domain (Trends Pharmacol. Sci. 32 (2011), 25-34). JAK1, JAK2 and TYK2 are expressed in various human tissues, while JAK3 is mainly expressed in various hematopoietic cells. A common feature of the cell surface cytokine receptors is that the receptor itself does not have kinase activity, but the intracellular segment of the receptor has a binding site for the tyrosine kinases JAKs. Accordingly, binding of cytokines to receptors results in JAK activation and phosphorylation of JAKs and related receptors. Phosphorylation of the receptors in turn initiates the recruitment of STATs through their SH2 domains and subsequently the phosphorylation of signal transducers and activators of transcription (STAT) proteins. Phosphorylated STAT homodimers or heterodimers then translocate to the nucleus and bind to specific deoxyribonucleic acid (DNA) binding sites to regulate gene transcription, resulting in changes in cellular function (J. Med. Chem., 62 (2019), 8953-8972).

Different paired JAK family members are responsible for signal transduction between different cytokines and their respective receptors. For example, TYK2 modulates interleukin-12 (IL12) and IL23 mediated signal transductions when paired with JAK2; but modulates interferon alpha (IFN-α) mediated signal transduction when paired with JAK1. Since the JAK/STAT pathway is involved in the inflammatory response, it can be a target for the treatment of diseases related to immune disorders (J. Med. Chem., 57 (2014), 5023-5038). As a potential target for treatment of autoimmune disease, TYK2, in particular, garnered support in the research field. For example, mice deficient in TYK2 can survive and develop normally. But deficiency of JAK1 (Cell, 93 (1998), 373-383) or JAK2 (Cell, 93 (1998), 397-409) in mice is lethal. Further, JAK3-deficient mice exhibit severe B- and T-cell depletion (Science, 270 (1995), 800-802). In addition, TYK2 has been shown to be protective in multiple autoimmune deficiency disease models (multiple sclerosis, Crohn's disease, ulcerative colitis, ankylosing spondylitis, and psoriasis, among others) (Brain, 134 (2011), 693-703; Inflammation (London, U. K.) 7 (2010), 41; Nat. Rev. Rheumatol. 12 (2016), 25-36). TYK2 is also associated with some cancers, e.g., T-lineage acute lymphoblastic leukemia (Cancer Disc. 3 (2013), 564-567).

The value of inhibiting pathways involving TYK2 in the treatment of autoimmune diseases has been clinically demonstrated by a variety of antibodies. The antibody Ustekinumab targeting the p40 subunit of both IL-12 and IL-23 is currently marketed for the treatment of psoriasis, psoriatic arthritis and Crohn's disease (Drugs, 71 (2011), 1733-1753; N. Engl. J. Med., 375 (2016), 1946-1960). This antibody recently showed efficacy in patients with systemic lupus erythematosus (SLE) (Lancet, 392 (2018), 1330-1339). The antibody Guselkumab that blocks IL-23 but not IL-12 signaling by targeting the p19 subunit of IL-23 has also been shown to be an effective treatment for psoriasis (J. Am. Acad. Dermatol., 76 (2017), 405-417). Several studies have shown that type 1 interferon has a pathogenic role in systemic lupus erythematosus (SLE), which led to the success of Sifalimumab and Anifrolumab in phase II clinical trials for the treatment of SLE (Ann. Rheum. Dis., 75 (2016), 1909-1916; Arthritis Rheumatol., 69 (2017), 376-386).

Given that TYK2 may be a therapeutic target, obtaining highly selective TYK2 inhibitors may have a good therapeutic effect on the above diseases. Currently, the selective TYK2 inhibitor BMS-986165 (J. Med. Chem., 62 (2019), 8973-8995) (mainly targeting TYK2 only, no inhibitory effect on JAK1) and the JAK1/TYK2 dual inhibitor PF-06700841 (J. Med. Chem., 61 (2018), 8597-8612) (mainly targeting TYK2 and JAK1 targets, but some studies shows that it also inhibits JAK2, with the ration of JAK2 IC₅₀/TYK2 IC₅₀ about 3.3) have entered clinical trials. Accordingly, obtaining highly druggable, highly active, and highly selective TYK2 or TYK2/JAK1 inhibitors may have promising clinical applications.

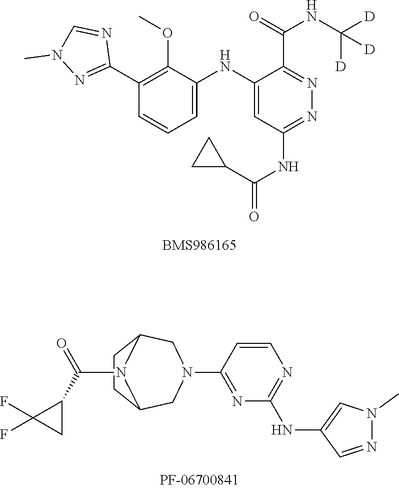 [SMILES: [2H]C([2H])([2H])NC(=O)c1nnc(NC(=O)C2CC2)cc1Nc1cccc(-c2ncn(C)n2)c1OC ; Cn1cc(Nc2nccc(N3CC4CCC(C3)N4C(=O)[C@@H]3CC3(F)F)n2)cn1]

### SUMMARY OF THE INVENTION

The present disclosure provides heterocycles as selective TYK2/JAK1 inhibitors, and compositions and applications thereof. These disclosed heterocycles, and compositions and applications thereof, may effectively and selectively inhibit TYK2/JAK1, thereby preventing or treating diseases and disorders, including, for example, autoimmune or inflammatory diseases, cancer/tumor, allergy, transplant rejection, neurodegenerative diseases, asthma and other obstructive airway diseases, etc.

One goal of the present disclosure is to provide selective TYK2/JAK1 inhibitors, and compositions and applications thereof.

An aspect of the present disclosure provides a compound of Formula (I):

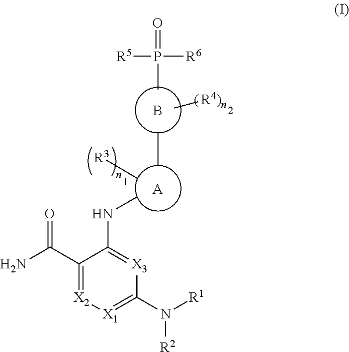

or a pharmaceutically acceptable salt, ester, solvate, prodrug, isotope-labeled derivative, or isomer thereof, wherein:
n₁ is 0, 1, 2, 3 or 4;
n₂ is 0, 1, 2, 3 or 4;
X₁ is N or CH;
X₂ is N or CH;
X₃ is Nor CR⁷;
ring A is C₆₋₁₀ aryl or 5-10 membered heteroaryl;
ring B is C₆₋₁₀ aryl or 5-10 membered heteroaryl, or ring B is absent, wherein when ring
B is absent,

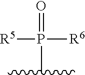

directly connects with ring A;
R¹ is hydrogen, C₁₋₆ alkyl, C₁₋₆ deuterated alkyl, C₂₋₆ alkenyl, C₂₋₆ alkynyl, C₃₋₆ cycloalkyl, 3-6 membered heterocycloalkyl, aryl, heteroaryl, —(CH₂)ₚOR^{b}, —(CH₂)ₚSR^{b}, —(CH₂)ₚC(O)R^{b}, —(CH₂)ₚC(O)OR^{b}, —(CH₂)ₚOC(O)R^{b}, —(CH₂)ₚNR^{c}R^{d}, —(CH₂)ₚC(O)NR^{c}R^{d}, —(CH₂)ₚNR^{b}C(O)R^{e}, —(CH₂)ₚNR^{b}C(O)OR^{e}, —S(O)_{q}NR^{c}R^{d} or —S(O)_{q}R^{e}, wherein C₁₋₆ alkyl, C₁₋₆ deuterated alkyl, C₂₋₆ alkenyl, C₂₋₆ alkynyl, C₃₋₆ cycloalkyl, 3-6 membered heterocycloalkyl, aryl, and heteroaryl, are unsubstituted or substituted with one or more groups independently selected from the group consisting of hydrogen, deuterium, halide, amino, —NO₂, —CN, —OH, C₁₋₆ alkyl, deuterated C₁₋₆ alky, C₁₋₆ halo-alkyl, C₁₋₆ alkoxide, C₁₋₃ halo-alkoxide, C₂₋₆ alkenyl, C₂₋₆ alkynyl, substituted or unsubstituted C₃₋₆ cycloalkyl, substituted or unsubstituted 3-6 membered heterocycloalkyl, substituted or unsubstituted aryl, and substituted or unsubstituted heteroaryl;
R² is hydrogen, C₁₋₄ alkyl, —(CH₂)ₚ-phenyl, or —(CH₂)ₚ-5-7 membered heterocycloalkyl, wherein C₁₋₄ alkyl is substituted with 0-1 R^{a}, wherein phenyl is substituted with 0-3 R^{a}, wherein 5-7 membered heterocycloalkyl comprises 1-4 hetero atoms or hetero atom groups in the ring, wherein hetero atom or hetero atom group is independently NH, N, O, S(O)_{q}, PH(O)ᵣ, or P(O)ᵣ, wherein heterocycloalkyl is substituted with 0-3 R^{a};
or R¹ and R², together with nitrogen attached thereto, form a 3-14 membered heterocycloalkyl, wherein 3-14 membered heterocycloalkyl is unsubstituted or substituted with one or more groups independently selected from the group consisting of deuterium, halide, amino, —NO₂, —CN, —OH, C₁₋₆ alkyl, deuterated C₁₋₆ alky, C₁₋₆ halo-alkyl, C₁₋₆ alkoxide, C₁₋₆ halo-alkoxide, C₂₋₆ alkenyl, and C₂₋₆ alkynyl; if present, each R³ is independently hydrogen, deuterium, halide, —OH, amino, —SH, —NO₂, —CN, —P(O)R^{c}R^{d}, C₁₋₆ alkyl, —C(O)NH₂, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₃₋₆heterocycloalkyl, C₆₋₁₀ aryl or 5-10 membered heteroaryl, wherein C₁₋₆ alkyl, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₃₋₆heterocycloalkyl, C₆₋₁₀ aryl and 5-10 membered heteroaryl are unsubstituted or substituted with one or more groups independently selected from the group consisting of deuterium, halide, amino, —NO₂, —CN, —OH, and C₁₋₃ alkyl;
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from the group consisting of deuterium, halide, amino, —NO₂, —CN and —OH;
each of R⁵ and R⁶ is independently C₁₋₆ alkyl, C₁₋₆ deuterated alkyl, C₂₋₆ alkenyl, C₂₋₆ alkynyl, or C₃₋₆ cycloalkyl, wherein C₁₋₆ alkyl, C₁₋₆ deuterated alkyl, C₂₋₆ alkenyl, C₂₋₆ alkynyl, and C₃₋₆ cycloalkyl are unsubstituted or substituted with one or more groups independently selected from the group consisting of deuterium, halide, amino, —NO₂, —CN and —OH;
or R⁵ and R⁶, together with phosphorus attached thereto, form a 5-6 membered heterocycloalkyl, wherein 5-6 membered heterocycloalkyl is unsubstituted or substituted with one or more groups independently selected from the group consisting of deuterium, halide, amino, —NO₂, —CN and —OH;
if present, R⁷ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from the group consisting of deuterium, halide, amino, —NO₂, —CN and —OH;
if present, each of R^{a}, R^{b}, R^{c}, R^{d} and R^{e} is independently hydrogen, deuterium, halide, amino, —NO₂, —CN, —OH, alkyl, deuterated alkyl, halo-alkyl, alkoxy, halo-alkoxy, alkenyl, alkynyl, cycloalkyl, heterocycloalkyl, aryl, or heteroaryl, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from the group consisting of deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted cycloalkyl, substituted and unsubstituted heterocycloalkyl, substituted or unsubstituted aryl, and substituted or unsubstituted heteroaryl; or any two of adjacent or non-adjacent R^{a}, R^{b}, R^{c}, R^{d} and R^{e} form a cycloalkyl, heterocycloalkyl, aryl or heteroaryl, wherein cycloalkyl, heterocycloalkyl, aryl and heteroaryl are unsubstituted or substituted with one or more groups independently selected from the group consisting of deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted 6-10 membered aryl, and substituted or unsubstituted 5-10 membered heteroaryl;
if present, each p is independently 0, 1 or 2;
if present, each q is independently 1 or 2; and
if present, each r is 0 or 1;
with the proviso that when B is absent and

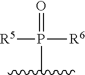

is directly connects with ring A, the compound is not

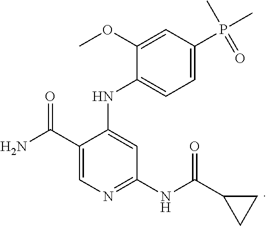 [SMILES: COc1cc(P(C)(C)=O)ccc1Nc1cc(NC(=O)C2CC2)ncc1C(N)=O]

In some embodiments of the compounds according to Formula (I) or the pharmaceutically acceptable salt, ester, solvate, prodrug, isotope-labeled derivative, or isomer thereof, wherein:
R¹ is hydrogen, C₁₋₆ alkyl, C₁₋₆ deuterated alkyl, C₂₋₆ alkenyl, C₂₋₆ alkynyl, C₃₋₆ cycloalkyl, 3-6 membered heterocycloalkyl, C₆₋₁₀ aryl, 5-10 membered heteroaryl, —C(O)R^{b}, —C(O)OR^{b}, —C(O)NR^{c}R^{d}, —S(O)_{q}NR^{c}R^{d} or —S(O)_{q}R^{e}, wherein C₁₋₆ alkyl, C₁₋₆ deuterated alkyl, C₂₋₆ alkenyl, C₂₋₆ alkynyl, C₃₋₆ cycloalkyl, 3-6 membered heterocycloalkyl, C₆₋₁₀ aryl, and 5-10 membered heteroaryl, are unsubstituted or substituted with one or more groups independently selected from the group consisting of deuterium, halide, amino, —NO₂, —CN, —OH, C₁₋₆ alkyl, deuterated C₁₋₆ alky, C₁₋₆ halo-alkyl, C₁₋₆ alkoxide, C₁₋₆ halo-alkoxide, C₂₋₆ alkenyl, C₂₋₆ alkynyl, substituted or unsubstituted C₃₋₆ cycloalkyl, substituted or unsubstituted 3-6 membered heterocycloalkyl, substituted or unsubstituted C₆₋₁₀ aryl, and substituted or unsubstituted 5-10 membered heteroaryl;
if present, each of R^{a}, R^{b}, R^{c}, R^{d} and R^{e} is independently hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, and 5-10 membered heteroaryl are unsubstituted or substituted with one or more groups independently selected from the group consisting of deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl; or any two of adjacent or non-adjacent R^{a}, R^{b}, R^{c}, R^{d} and R^{e} form a C₃₋₆ cycloalkyl, C₃₋₆heterocycloalkyl, C₆₋₁₀ aryl or 5-10 membered heteroaryl, wherein C₃₋₆ cycloalkyl, C₃₋₆heterocycloalkyl, C₆₋₁₀ aryl and 5-10 membered heteroaryl are unsubstituted or substituted with one or more groups independently selected from the group consisting of deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted or unsubstituted 3-6 membered heterocycloalkyl, substituted or unsubstituted C₆₋₁₀ aryl, and substituted or unsubstituted 5-10 membered heteroaryl; and
if present, q is independently 1 or 2.

In some embodiments of the compounds according to Formula (I) or the pharmaceutically acceptable salt, ester, solvate, prodrug, isotope-labeled derivative, or isomer thereof, wherein:
ring A is phenyl or 5-6 membered heteroaryl;
ring B is C₆₋₁₀ aryl or 5-10 membered heteroaryl, preferably phenyl or 5-6 membered heteroaryl;
if present, each R³ is independently halide, —CN, C₁₋₆ alkyl, C₁₋₆ deuterated alkyl, —O(C₁₋₆ alkyl), —O(C₁₋₆ deuterated alkyl), C₃₋₆ cycloalkyl, or —C(O)NH₂, preferably halide or —O(C₁₋₆ alkyl);
R⁵ is C₁₋₆ alkyl or C₃₋₆ cycloalkyl; preferably C₁₋₃ alkyl or cyclopropyl; and
R⁶ is C₁₋₆ alkyl or C₃₋₆ cycloalkyl; preferably C₁₋₃ alkyl or cyclopropyl.

In some embodiments, the compound is of Formula (II):

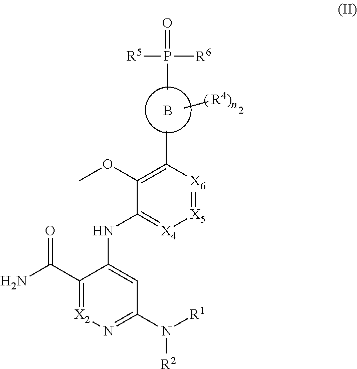

or a pharmaceutically acceptable salt, ester, solvate, prodrug, isotope-labeled derivative, or isomer thereof, wherein:
X₄ is N or CR⁸;
X₅ is N or CRR;
X₆ is N or CRR;
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₄ cycloalkyl, C₂₋₆ alkenyl and C₂₋₆ alkynyl are unsubstituted or substituted with one or more groups independently selected from the group consisting of deuterium, halide, amino, —NO₂, —CN and —OH; and
n₂, X₂, ring B, R¹, R², R⁴, R⁵ and R⁶ are defined as those in Formula (I), respectively.

In some embodiments of the compounds according to Formula (I) or Formula (II), or the pharmaceutically acceptable salt, ester, solvate, prodrug, isotope-labeled derivative, or isomer thereof, wherein:
R² is defined as that in Formula (T);
R¹ is independently selected from the group consisting of:

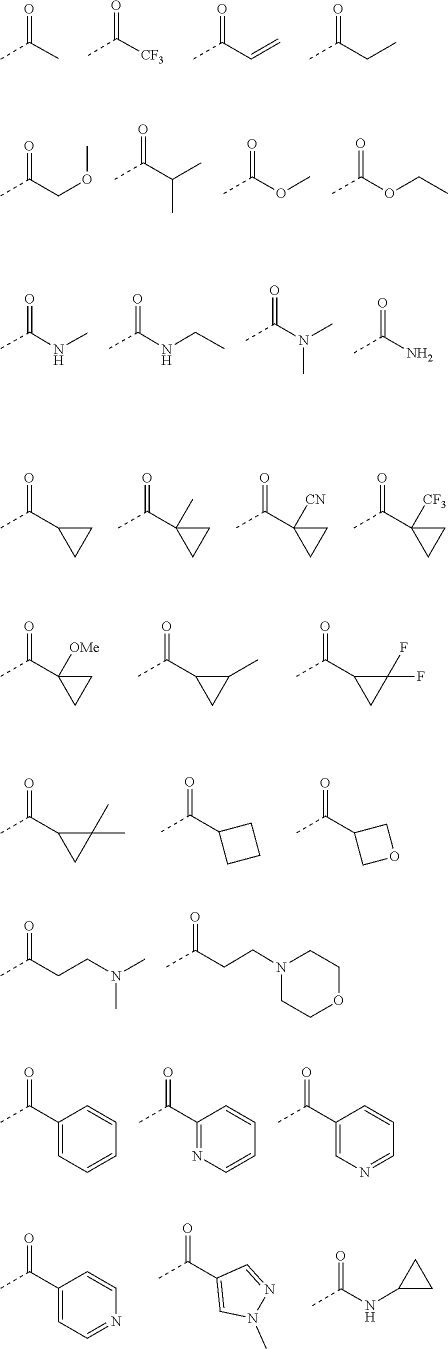

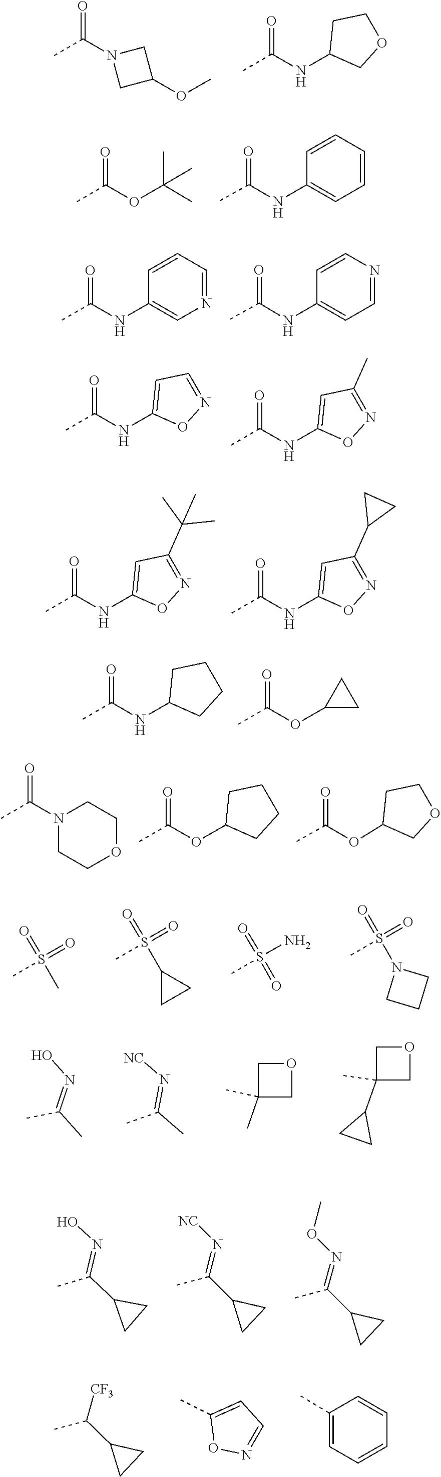

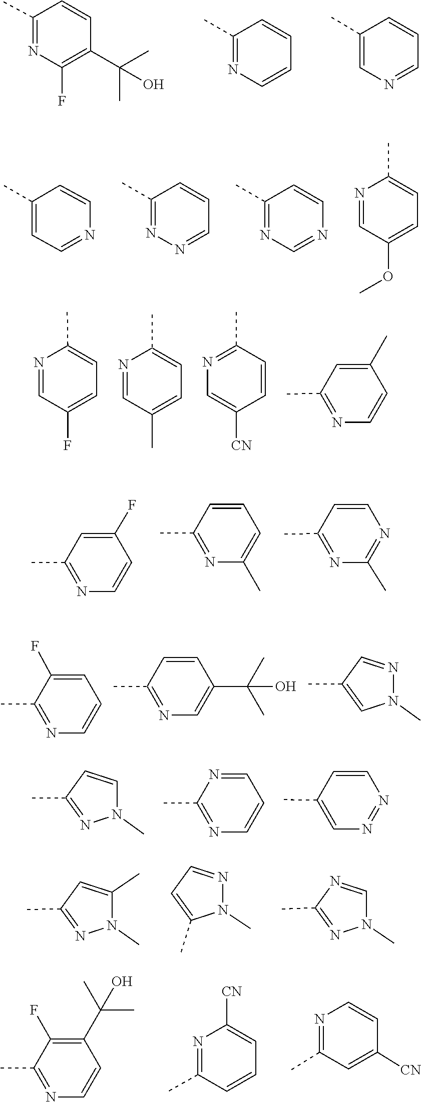

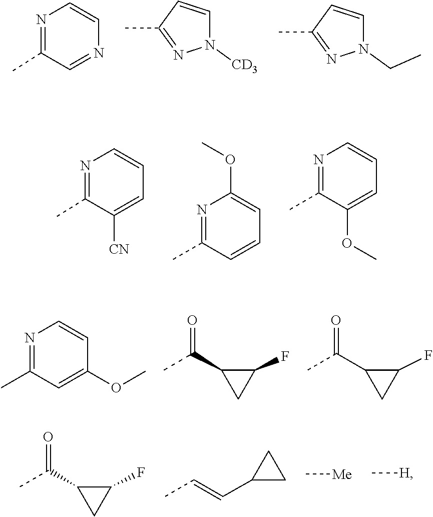

each substituted independently with 0-3 R⁹;
preferably, R¹ is independently selected from the group consisting of:

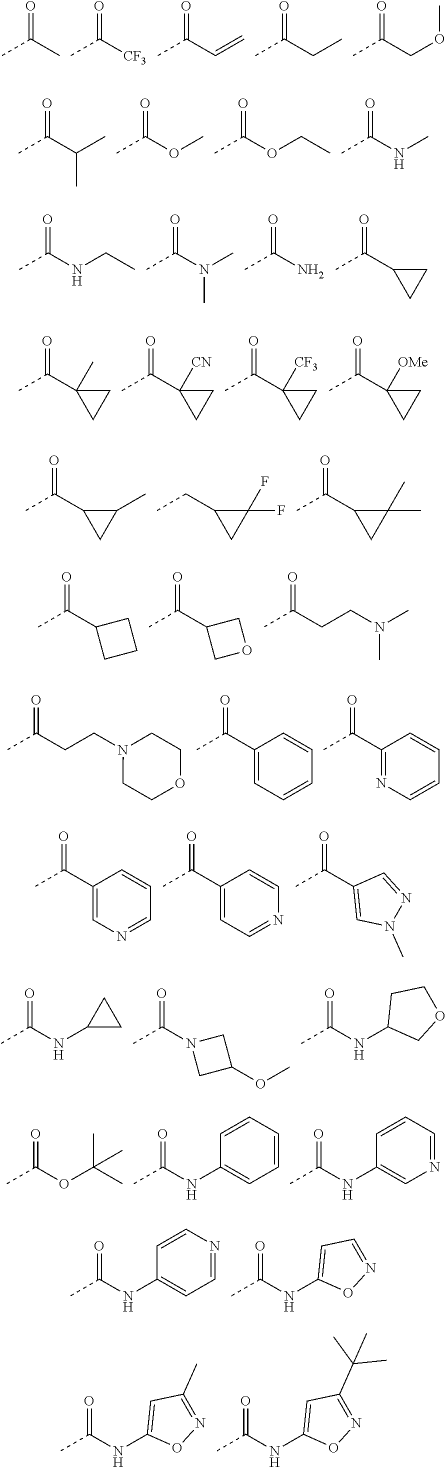

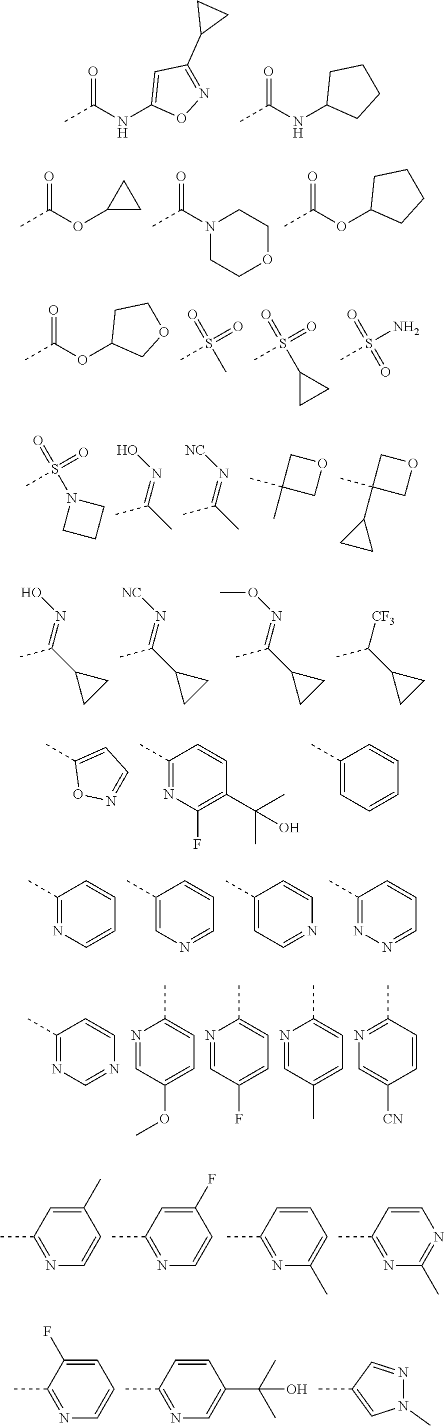

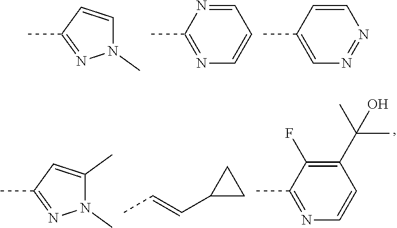

each substituted independently with 0-3 R⁹; and
if present, each R⁹ is independently deuterium, halide, amino, —NO₂, —CN, —OH, C₁₋₃ alkyl, or C₁₋₃ alkoxide.

In some embodiments of the compounds according to Formula (I) or the pharmaceutically acceptable salt, ester, solvate, prodrug, isotope-labeled derivative, or isomer thereof, wherein:
R¹ and R², together with nitrogen connected thereto, form a heterocycloalkyl selected from the group consisting of:

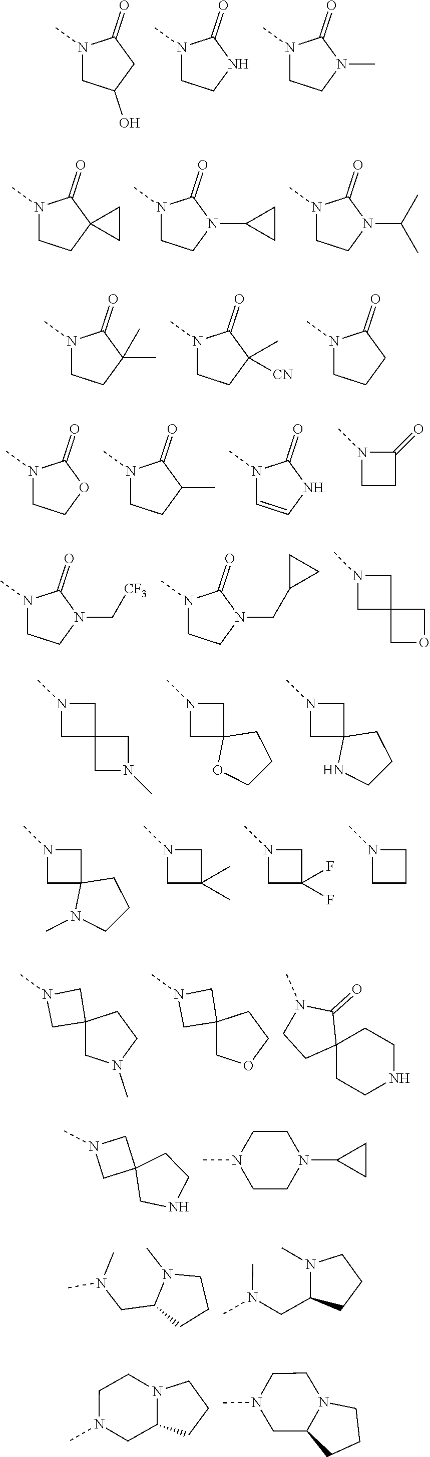

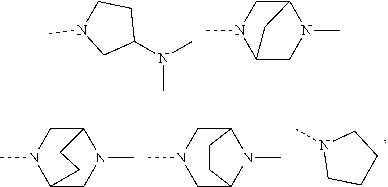

each substituted independently with 0-3 R¹⁰;
preferably, R¹ and R², together with nitrogen connected thereto, form a heterocycloalkyl selected from the group consisting of:

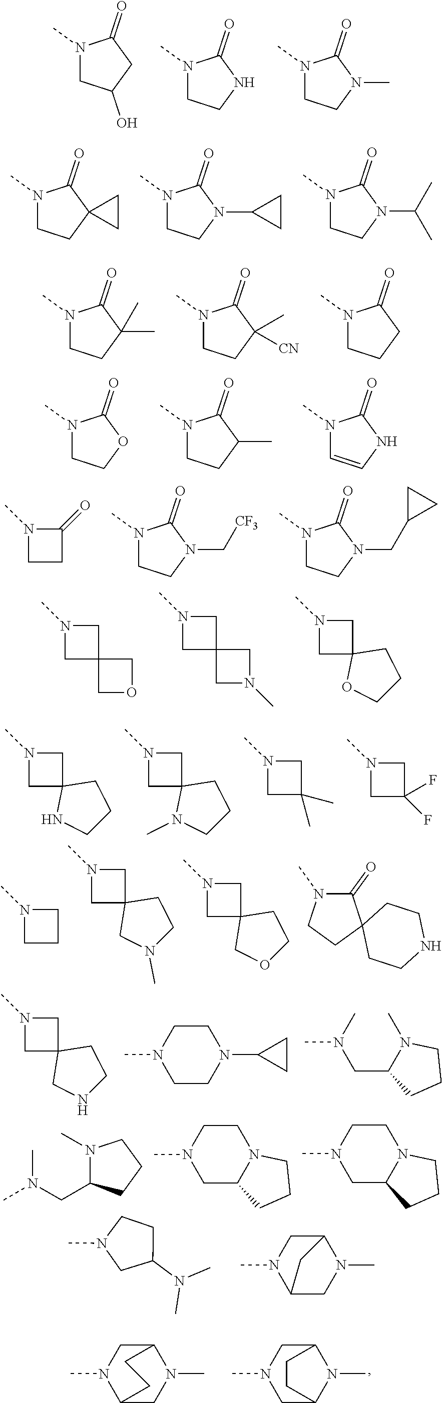

each substituted with 0-3 R¹⁰;
if present, R¹⁰ is independently deuterium, halide, amino, —NO₂, —CN, —OH, C₁₋₃ alkyl, or C₁₋₃ alkoxide.

In some embodiments, the compound is of Formula (III):

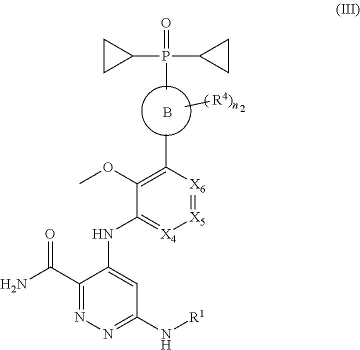

or a pharmaceutically acceptable salt, ester, solvate, prodrug, isotope-labeled derivative, or isomer thereof, wherein n₂, ring B, R¹, R⁴, X₄, X₅ and X₆ are defined as those in Formula (II), respectively.

In some embodiments, the compounds according to Formula (III) or the pharmaceutically acceptable salt, ester, solvate, prodrug, isotope-labeled derivative, or isomer thereof, wherein:

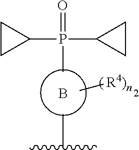

is selected from the group consisting of:

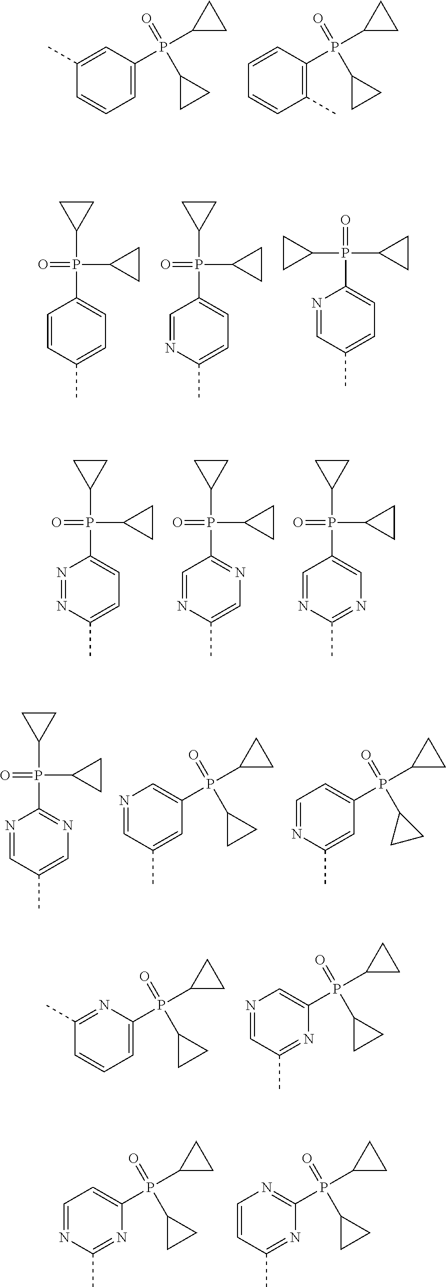

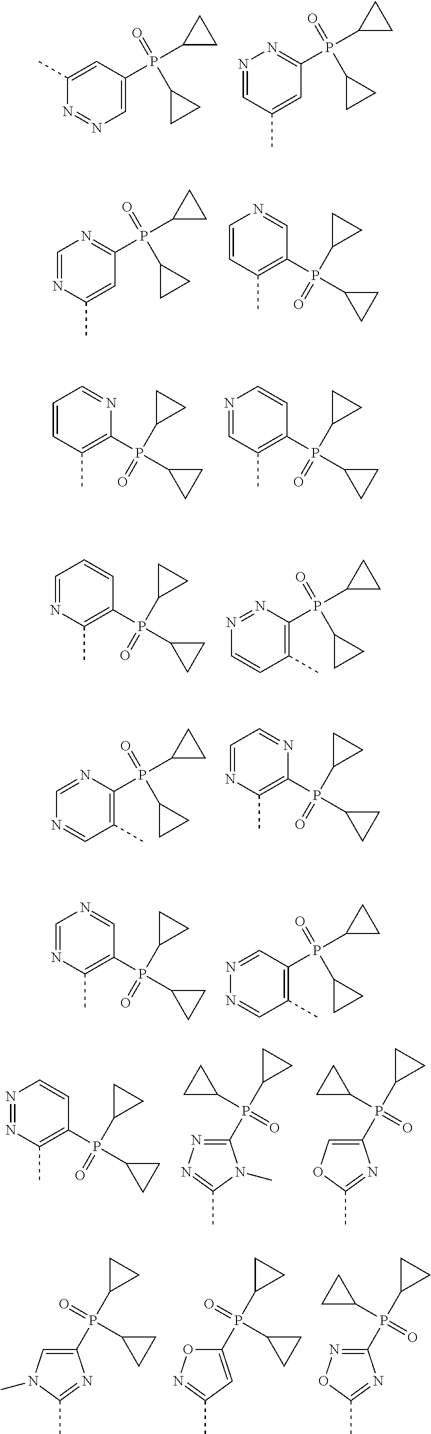

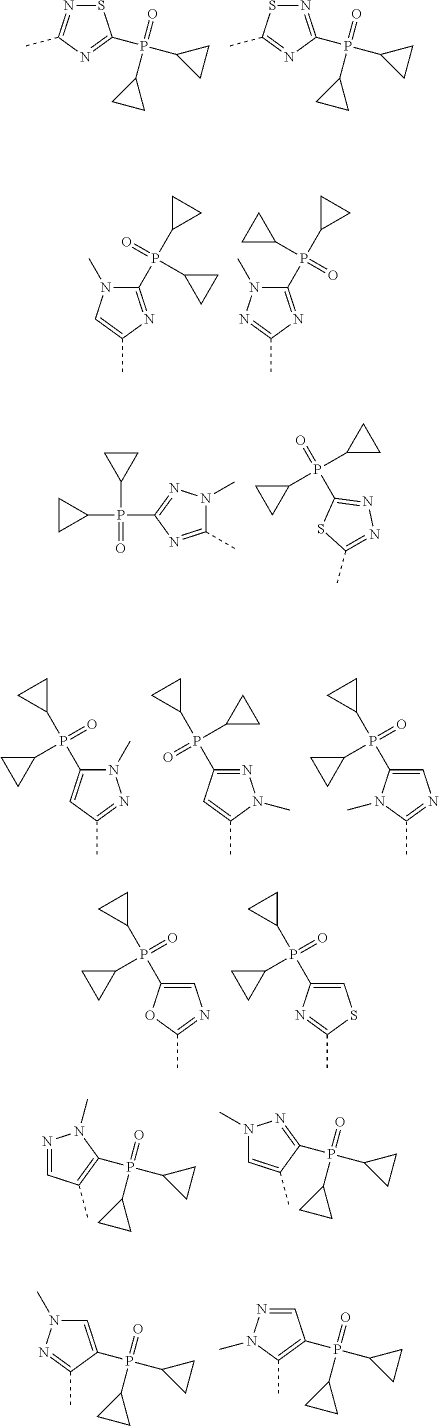

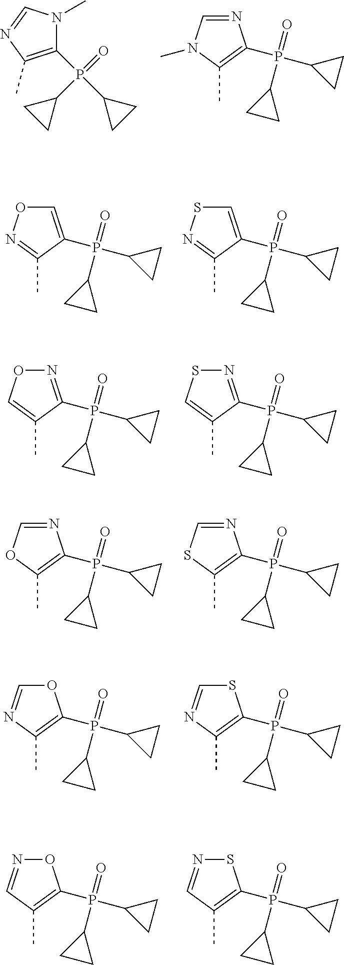

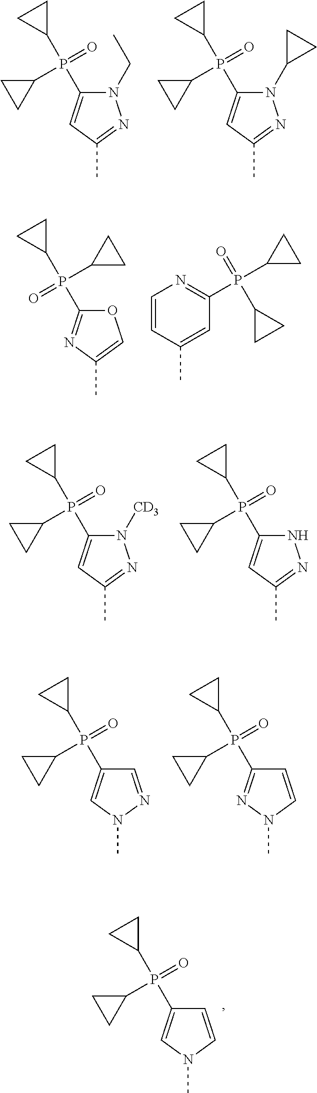

each substituted with 0 to 3 R¹¹;
preferably,

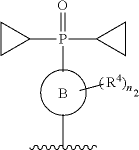

is selected from the group consisting of:

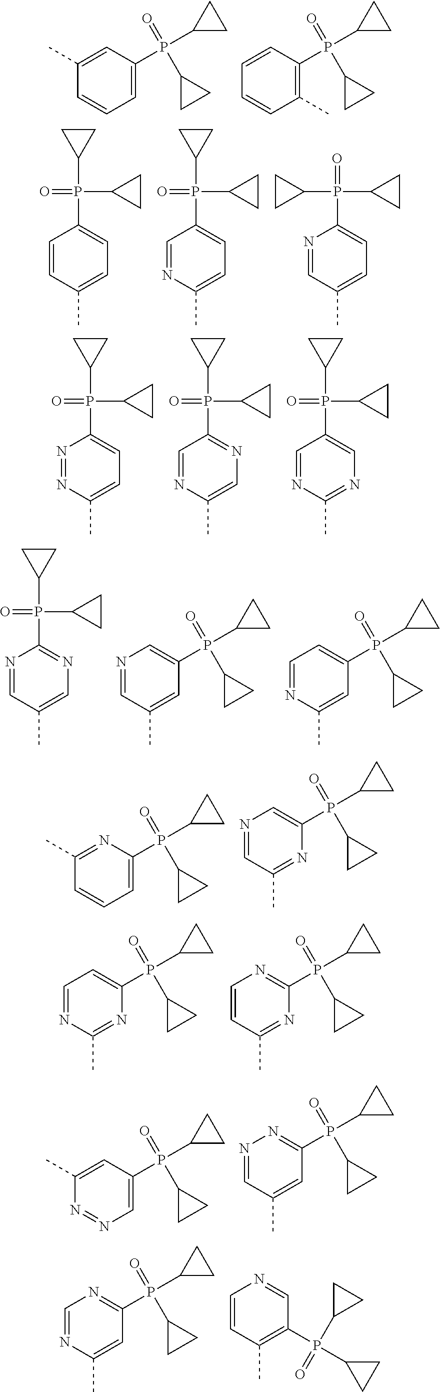

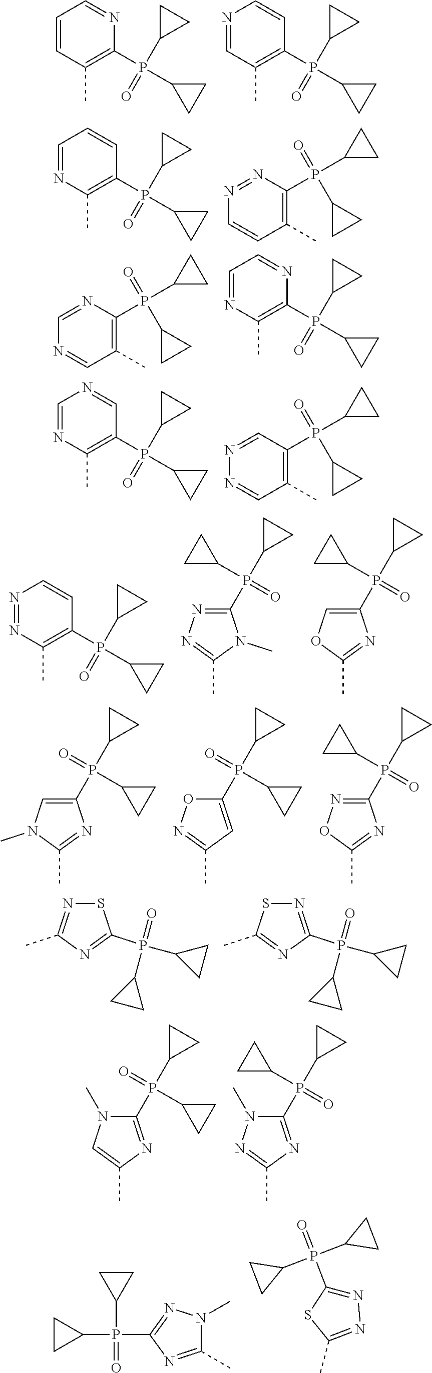

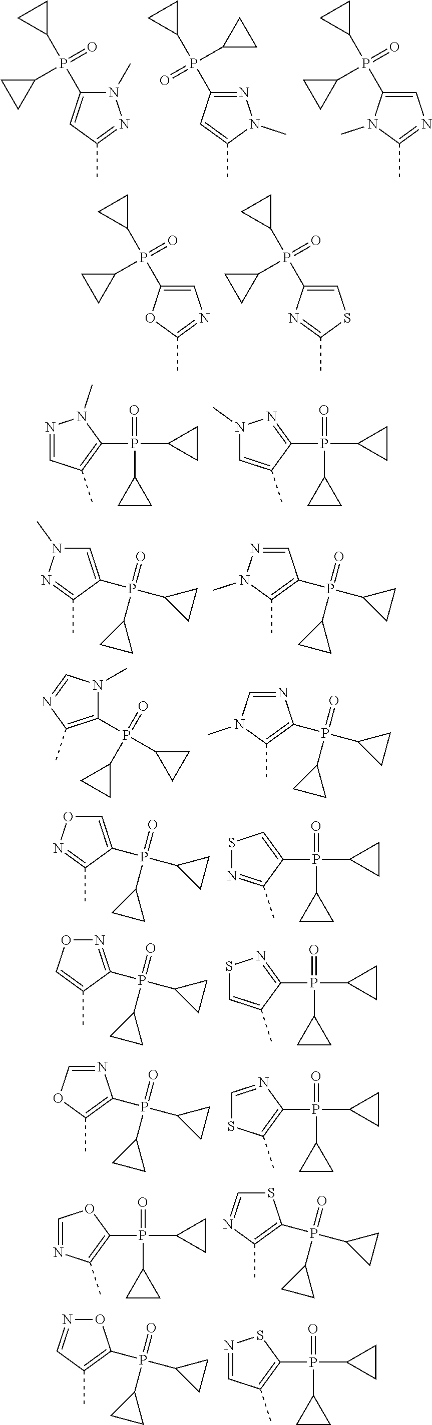

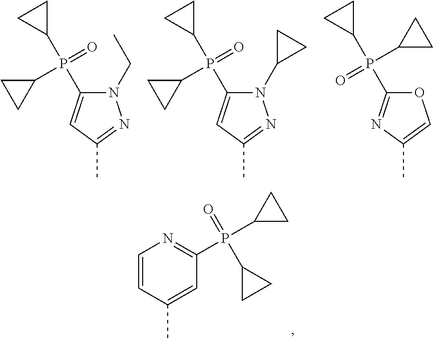

each substituted with 0 to 3 R¹¹; and
if present, each R¹¹ is independently deuterium, halide, amino, —CN, —OH, C₁₋₃ alkyl or C₁₋₃ alkoxy.

In some embodiments of the compounds according to Formula (III), or the pharmaceutically acceptable salt, ester, solvate, prodrug, isotope-labeled derivative, or isomer thereof, wherein:
R¹ is independently selected from the group consisting of:

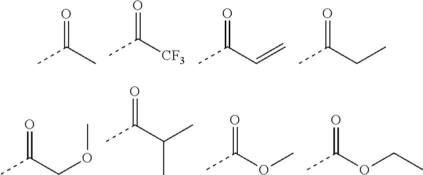

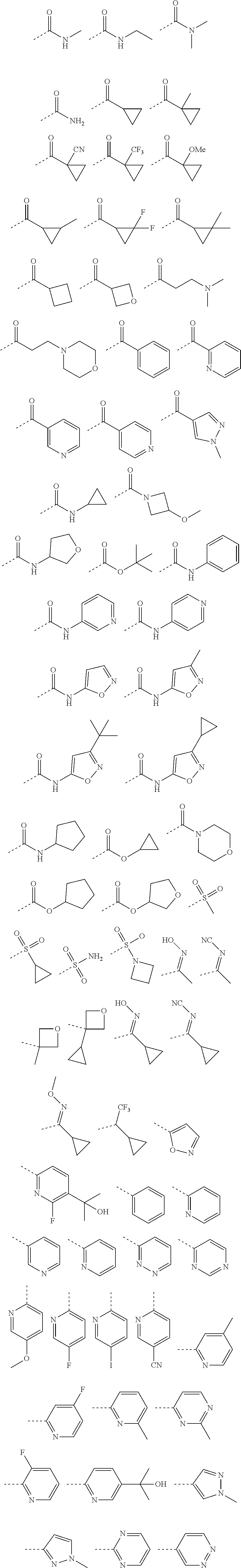

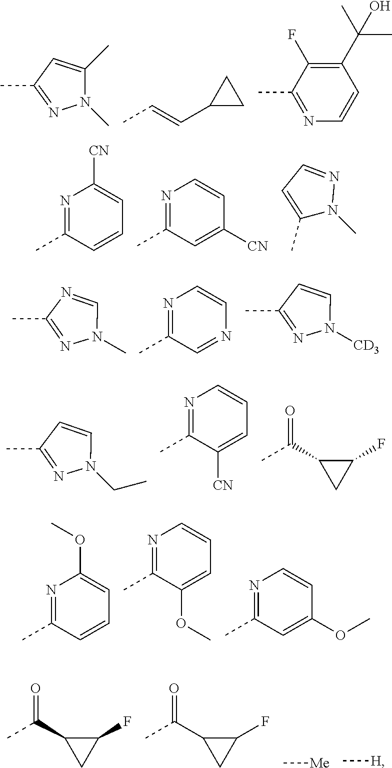

each substituted with 0 to 3 R⁹, with the proviso that when R¹ is H, R¹ is unsubstituted;
preferably, R¹ is independently selected from the group consisting of:

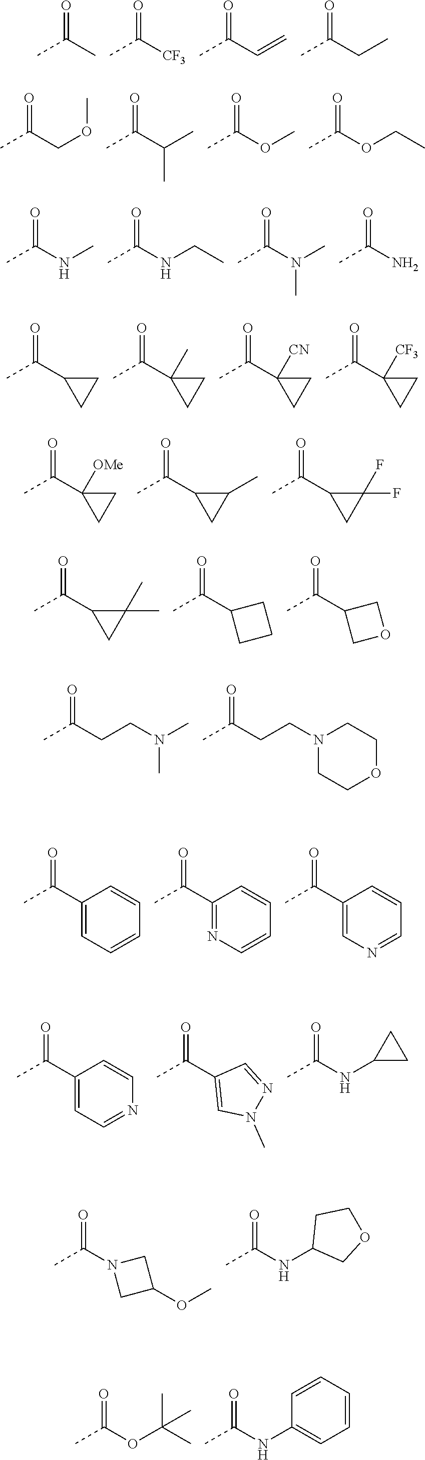

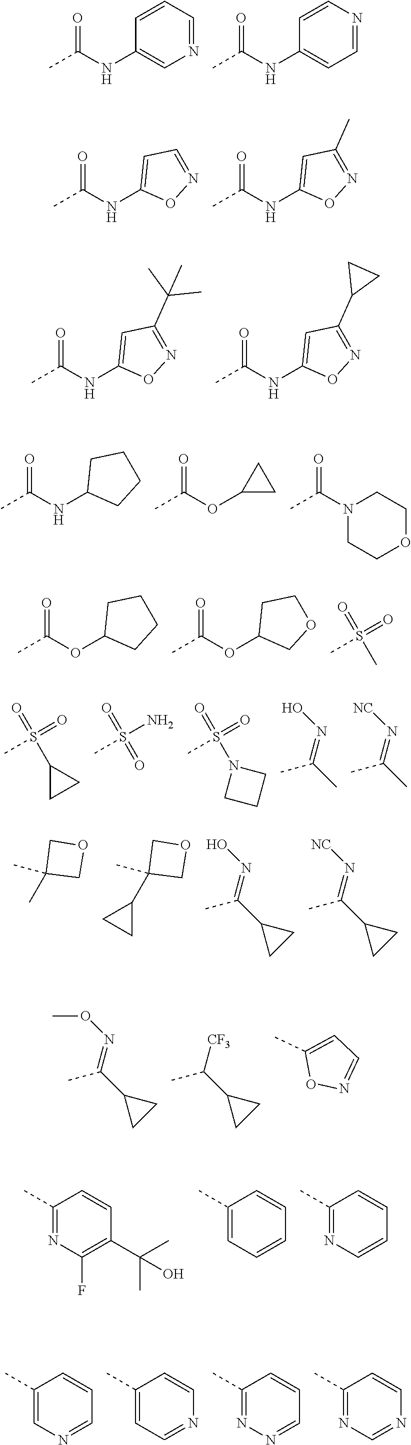

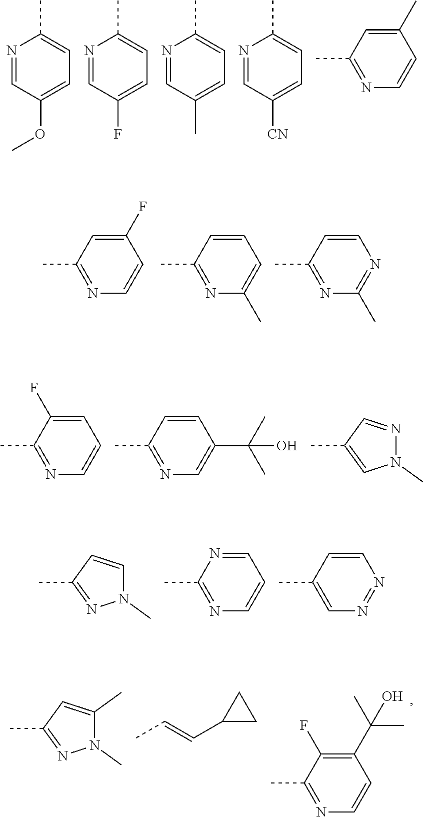

each substituted with 0 to 3 R⁹;
if present, R⁹ is independently deuterium, halide, amino, —NO₂, —CN, —OH, C₁₋₃ alkyl, or C₁₋₃ alkoxide.

In some embodiments, the compound is according to Formula (IV):

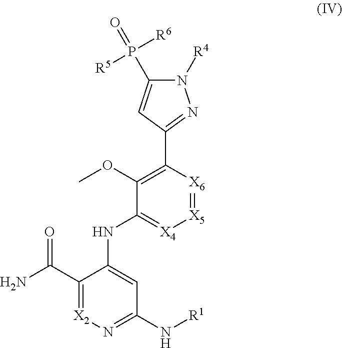

or a pharmaceutically acceptable salt, ester, solvate, prodrug, isotope-labeled derivative, or isomer thereof, wherein:
X₂ is N or CH;
X₄ is N or CR⁸;
X₅ is N or CR⁸;
X₆ is N or CR⁸;
if present, each R⁸ is independently hydrogen, deuterium, halide, —OH, amino, or —CN;
R¹ is hydrogen, C₁₋₆ alkyl, C₃₋₆ cycloalkyl, 3-6 membered heterocycloalkyl, C₆₋₁₀ aryl, 5-10 membered heteroaryl, —C(O)R^{b}, —C(O)OR^{b}, —C(O)NR^{c}R^{d} or —S(O)_{q}R^{e}, wherein C₁₋₆ alkyl, C₃₋₆ cycloalkyl, 3-6 membered heterocycloalkyl, C₆₋₁₀ aryl, and 5-10 membered heteroaryl, are unsubstituted or substituted with one or more groups independently selected from the group consisting of deuterium, halide, amino, —CN, —OH, C₁₋₆ alkyl, deuterated C₁₋₆ alkyl and C₁₋₆ alkoxide;
R⁴ is hydrogen, C₁₋₆ alkyl, deuterated C₁₋₆ alkyl, and C₃₋₆ cycloalkyl, wherein C₁₋₆ alkyl and C₃₋₆ cycloalkyl are unsubstituted or substituted with one or more groups independently selected from the group consisting of halide, amino, —CN, and —OH;
each of R⁵ and R⁶ is independently C₁₋₆ alkyl or C₃₋₆ cycloalkyl;
if present, each of R^{b}, R^{c}, R^{d} and R^{e} is independently hydrogen, halide, amino, —CN, —OH, C₁₋₆ alkyl or C₃₋₆ cycloalkyl, wherein C₁₋₆ alkyl and C₃₋₆ cycloalkyl are unsubstituted or substituted with one or more groups independently selected from the group consisting of halide, amino, —CN and —OH; and
if present, q is 1 or 2.

In some embodiments, the compound of Formula (IV) or the pharmaceutically acceptable salt, ester, solvate, prodrug, isotope-labeled derivative, or isomer thereof, wherein:
X₂ is N;
X₄ is N or CR⁸;
X₅ is N or CR⁸;
X₆ is Nor CR⁸;
if present, each R⁸ is independently hydrogen or halide, preferably hydrogen;
R¹ is hydrogen, C₁₋₆ alkyl, 5-10 membered heteroaryl, —C(O)R^{b}, —C(O)OR^{b}, —C(O)NR^{c}R^{d} or —S(O)_{q}R^{e}, wherein C₁₋₆ alkyl and 5-10 membered heteroaryl, are unsubstituted or substituted with one or more groups independently selected from the group consisting of halide, —CN, C₁₋₆ alkyl, deuterated C₁₋₆ alkyl and C₁₋₆ alkoxide;
R⁴ is hydrogen, C₁₋₆ alkyl, deuterated C₁₋₆ alkyl, or C₃₋₆ cycloalkyl;
each of R⁵ and R⁶ is independently C₁₋₆ alkyl or C₃₋₆ cycloalkyl;
if present, each of R^{b}, R^{c}, R^{d} and R^{e} is independently hydrogen, C₁₋₆ alkyl or C₃₋₆ cycloalkyl, wherein C₁₋₆ alkyl and C₃₋₆ cycloalkyl are unsubstituted or substituted with one or more halides; and
if present, q is 2.

In some embodiments, the compound is according to Formula (V):

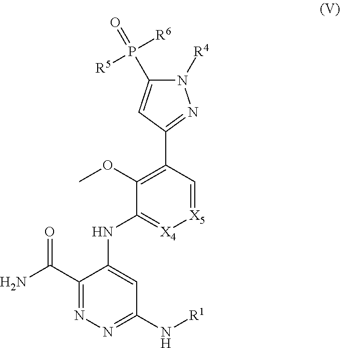

or a pharmaceutically acceptable salt, ester, solvate, prodrug, isotope-labeled derivative, or isomer thereof, wherein:
X₄ is N or CH;
X₅ is N or CR⁸;
if present, each R⁸ is independently hydrogen or fluorine, preferable hydrogen;
R¹ is 5-10 membered heteroaryl or —C(O)R^{b}, wherein 5-10 membered heteroaryl is unsubstituted or substituted with one or more groups independently selected from the group consisting of halide, —CN, C₁₋₃ alkyl, deuterated C₁₋₃ alkyl and C₁₋₃ alkoxide;
R⁴ is independently hydrogen, C₁₋₃ alkyl, deuterated C₁₋₃ alkyl and C₃₋₆ cycloalkyl;
each of R⁵ and R⁶ is independently C₁₋₃ alkyl or C₃₋₆ cycloalkyl; and
if present, R^{b} is independently C₃₋₆ cycloalkyl, wherein C₃₋₆ cycloalkyl is unsubstituted or substituted with one or more fluorine.

Another aspect of the present disclosure provides a compound selected from the group consisting of:

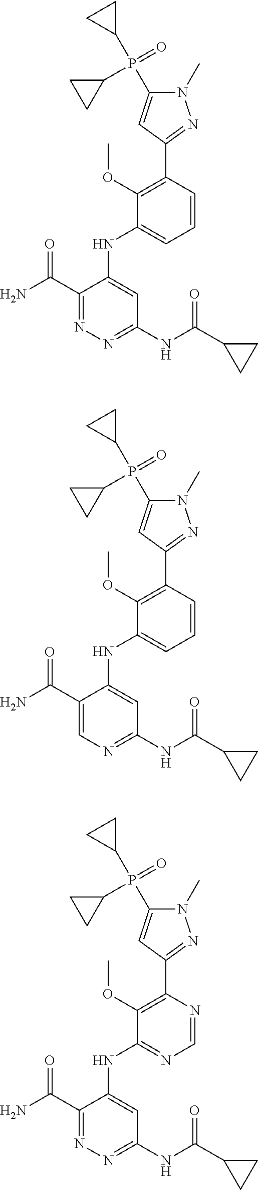 [SMILES: COc1c(Nc2cc(NC(=O)C3CC3)nnc2C(N)=O)cccc1-c1cc(P(=O)(C2CC2)C2CC2)n(C)n1 ; COc1c(Nc2cc(NC(=O)C3CC3)ncc2C(N)=O)cccc1-c1cc(P(=O)(C2CC2)C2CC2)n(C)n1 ; COc1c(Nc2cc(NC(=O)C3CC3)nnc2C(N)=O)ncnc1-c1cc(P(=O)(C2CC2)C2CC2)n(C)n1]

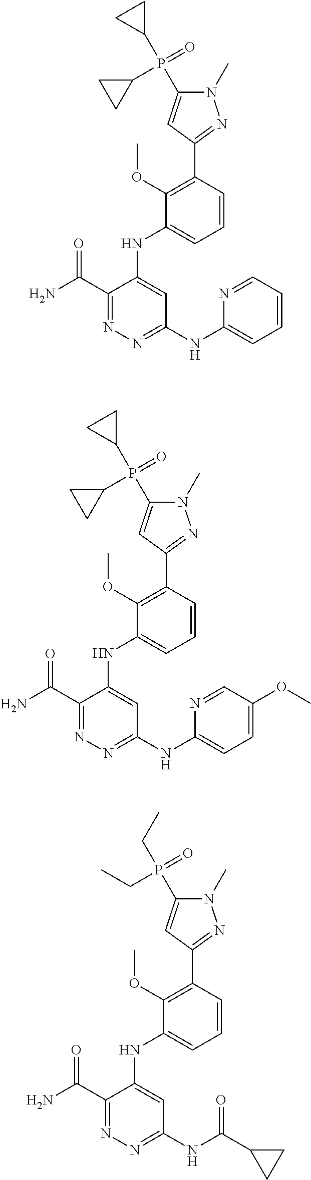 [SMILES: COc1c(Nc2cc(Nc3ccccn3)nnc2C(N)=O)cccc1-c1cc(P(=O)(C2CC2)C2CC2)n(C)n1 ; COc1ccc(Nc2cc(Nc3cccc(-c4cc(P(=O)(C5CC5)C5CC5)n(C)n4)c3OC)c(C(N)=O)nn2)nc1 ; CCP(=O)(CC)c1cc(-c2cccc(Nc3cc(NC(=O)C4CC4)nnc3C(N)=O)c2OC)nn1C]

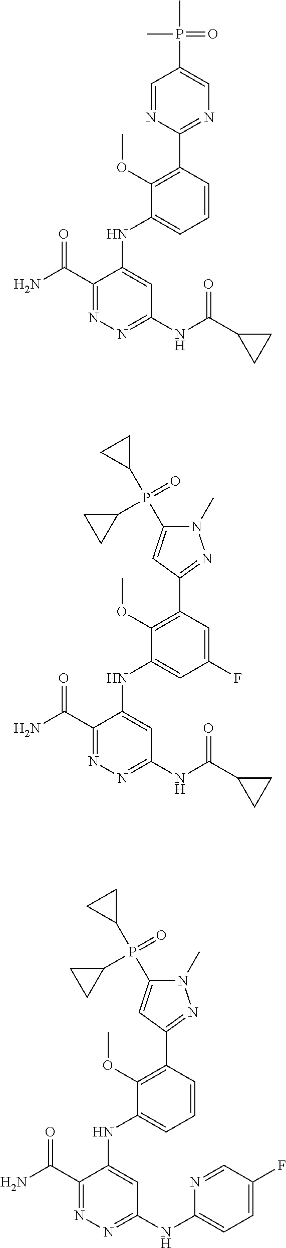 [SMILES: COc1c(Nc2cc(NC(=O)C3CC3)nnc2C(N)=O)cccc1-c1ncc(P(C)(C)=O)cn1 ; COc1c(Nc2cc(NC(=O)C3CC3)nnc2C(N)=O)cc(F)cc1-c1cc(P(=O)(C2CC2)C2CC2)n(C)n1 ; COc1c(Nc2cc(Nc3ccc(F)cn3)nnc2C(N)=O)cccc1-c1cc(P(=O)(C2CC2)C2CC2)n(C)n1]

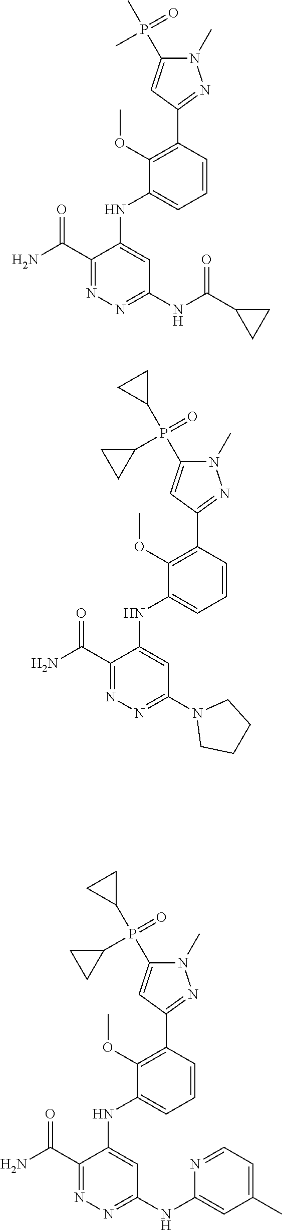 [SMILES: COc1c(Nc2cc(NC(=O)C3CC3)nnc2C(N)=O)cccc1-c1cc(P(C)(C)=O)n(C)n1 ; COc1c(Nc2cc(N3CCCC3)nnc2C(N)=O)cccc1-c1cc(P(=O)(C2CC2)C2CC2)n(C)n1 ; COc1c(Nc2cc(Nc3cc(C)ccn3)nnc2C(N)=O)cccc1-c1cc(P(=O)(C2CC2)C2CC2)n(C)n1]

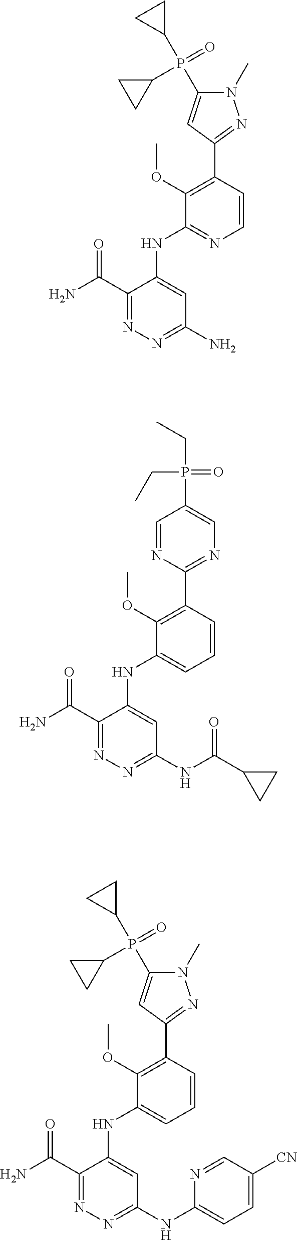 [SMILES: COc1c(-c2cc(P(=O)(C3CC3)C3CC3)n(C)n2)ccnc1Nc1cc(N)nnc1C(N)=O ; CCP(=O)(CC)c1cnc(-c2cccc(Nc3cc(NC(=O)C4CC4)nnc3C(N)=O)c2OC)nc1 ; COc1c(Nc2cc(Nc3ccc(C#N)cn3)nnc2C(N)=O)cccc1-c1cc(P(=O)(C2CC2)C2CC2)n(C)n1]

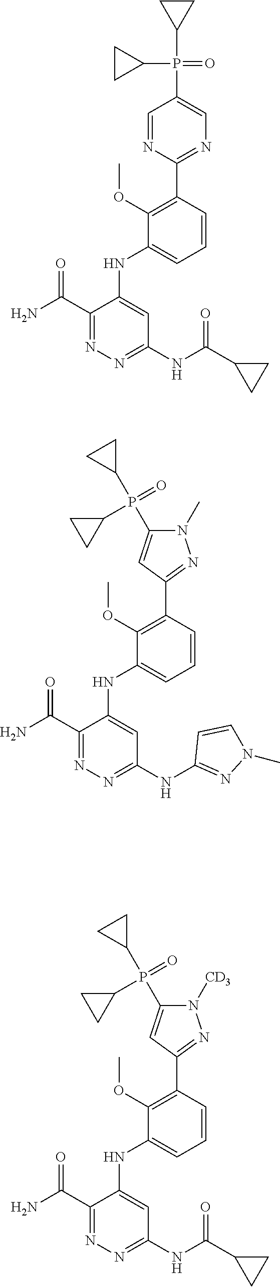 [SMILES: COc1c(Nc2cc(NC(=O)C3CC3)nnc2C(N)=O)cccc1-c1ncc(P(=O)(C2CC2)C2CC2)cn1 ; COc1c(Nc2cc(Nc3ccn(C)n3)nnc2C(N)=O)cccc1-c1cc(P(=O)(C2CC2)C2CC2)n(C)n1 ; [2H]C([2H])([2H])n1nc(-c2cccc(Nc3cc(NC(=O)C4CC4)nnc3C(N)=O)c2OC)cc1P(=O)(C1CC1)C1CC1]

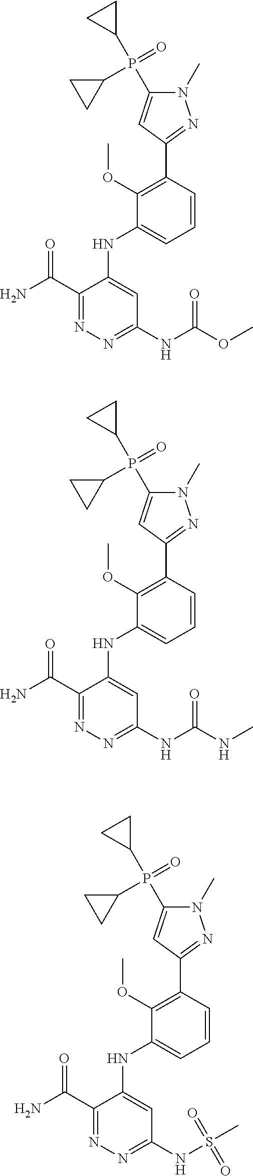 [SMILES: COC(=O)Nc1cc(Nc2cccc(-c3cc(P(=O)(C4CC4)C4CC4)n(C)n3)c2OC)c(C(N)=O)nn1 ; CNC(=O)Nc1cc(Nc2cccc(-c3cc(P(=O)(C4CC4)C4CC4)n(C)n3)c2OC)c(C(N)=O)nn1 ; COc1c(Nc2cc(NS(C)(=O)=O)nnc2C(N)=O)cccc1-c1cc(P(=O)(C2CC2)C2CC2)n(C)n1]

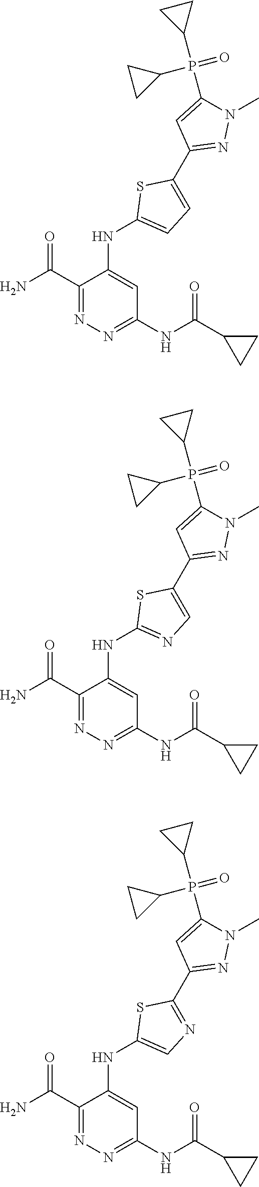 [SMILES: Cn1nc(-c2ccc(Nc3cc(NC(=O)C4CC4)nnc3C(N)=O)s2)cc1P(=O)(C1CC1)C1CC1 ; Cn1nc(-c2cnc(Nc3cc(NC(=O)C4CC4)nnc3C(N)=O)s2)cc1P(=O)(C1CC1)C1CC1 ; Cn1nc(-c2ncc(Nc3cc(NC(=O)C4CC4)nnc3C(N)=O)s2)cc1P(=O)(C1CC1)C1CC1]

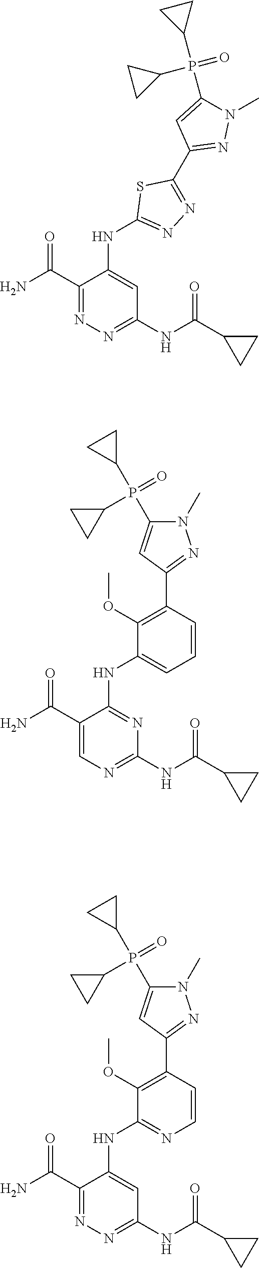 [SMILES: Cn1nc(-c2nnc(Nc3cc(NC(=O)C4CC4)nnc3C(N)=O)s2)cc1P(=O)(C1CC1)C1CC1 ; COc1c(Nc2nc(NC(=O)C3CC3)ncc2C(N)=O)cccc1-c1cc(P(=O)(C2CC2)C2CC2)n(C)n1 ; COc1c(-c2cc(P(=O)(C3CC3)C3CC3)n(C)n2)ccnc1Nc1cc(NC(=O)C2CC2)nnc1C(N)=O]

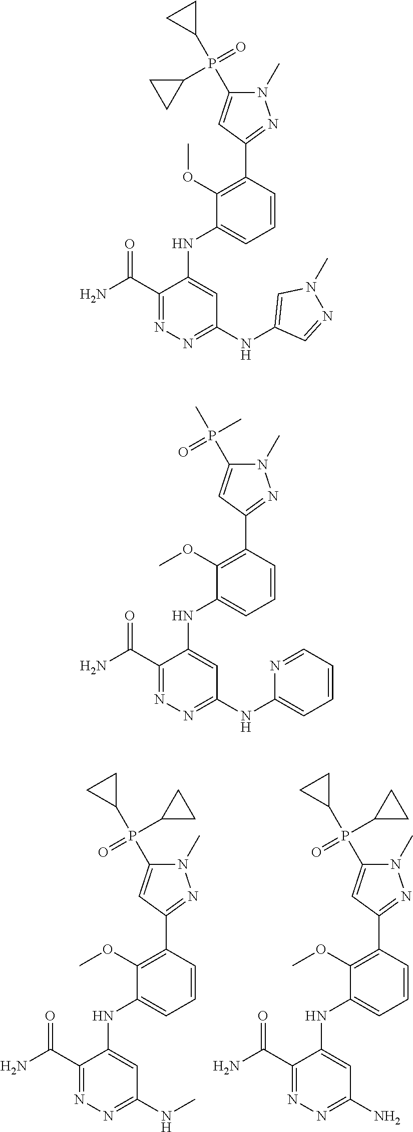 [SMILES: COc1c(Nc2cc(Nc3cnn(C)c3)nnc2C(N)=O)cccc1-c1cc(P(=O)(C2CC2)C2CC2)n(C)n1 ; COc1c(Nc2cc(Nc3ccccn3)nnc2C(N)=O)cccc1-c1cc(P(C)(C)=O)n(C)n1 ; CNc1cc(Nc2cccc(-c3cc(P(=O)(C4CC4)C4CC4)n(C)n3)c2OC)c(C(N)=O)nn1 ; COc1c(Nc2cc(N)nnc2C(N)=O)cccc1-c1cc(P(=O)(C2CC2)C2CC2)n(C)n1]

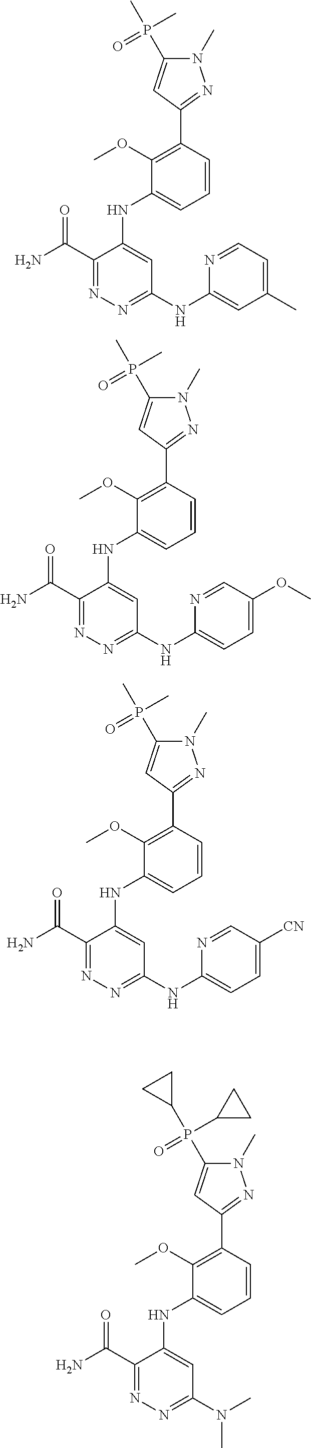

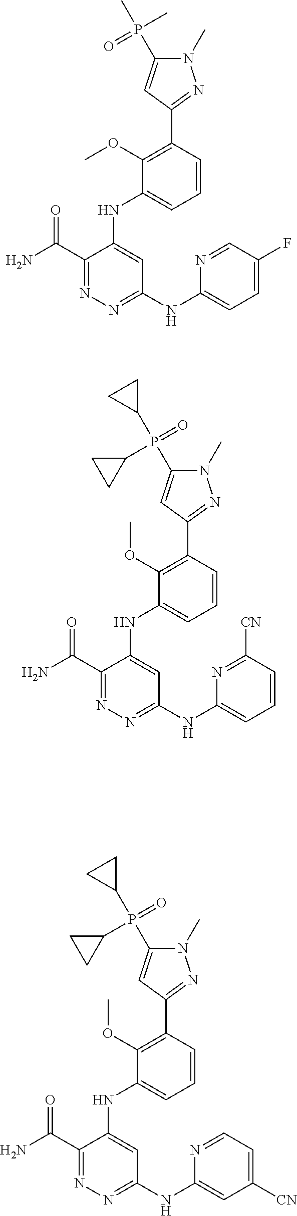 [SMILES: COc1c(Nc2cc(Nc3ccc(F)cn3)nnc2C(N)=O)cccc1-c1cc(P(C)(C)=O)n(C)n1 ; COc1c(Nc2cc(Nc3cccc(C#N)n3)nnc2C(N)=O)cccc1-c1cc(P(=O)(C2CC2)C2CC2)n(C)n1 ; COc1c(Nc2cc(Nc3cc(C#N)ccn3)nnc2C(N)=O)cccc1-c1cc(P(=O)(C2CC2)C2CC2)n(C)n1]

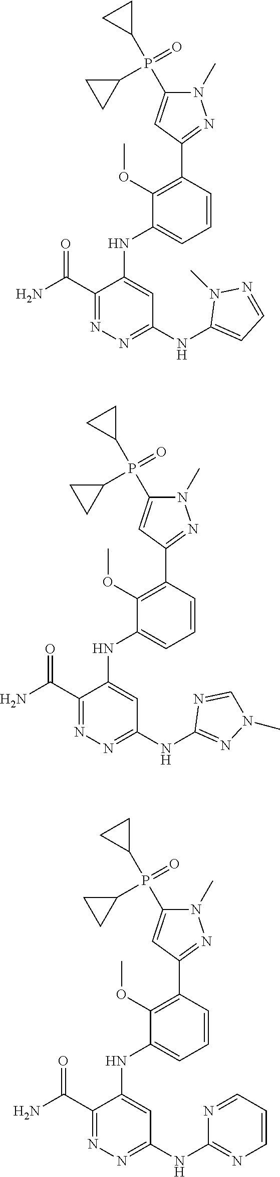 [SMILES: COc1c(Nc2cc(Nc3ccnn3C)nnc2C(N)=O)cccc1-c1cc(P(=O)(C2CC2)C2CC2)n(C)n1 ; COc1c(Nc2cc(Nc3ncn(C)n3)nnc2C(N)=O)cccc1-c1cc(P(=O)(C2CC2)C2CC2)n(C)n1 ; COc1c(Nc2cc(Nc3ncccn3)nnc2C(N)=O)cccc1-c1cc(P(=O)(C2CC2)C2CC2)n(C)n1]

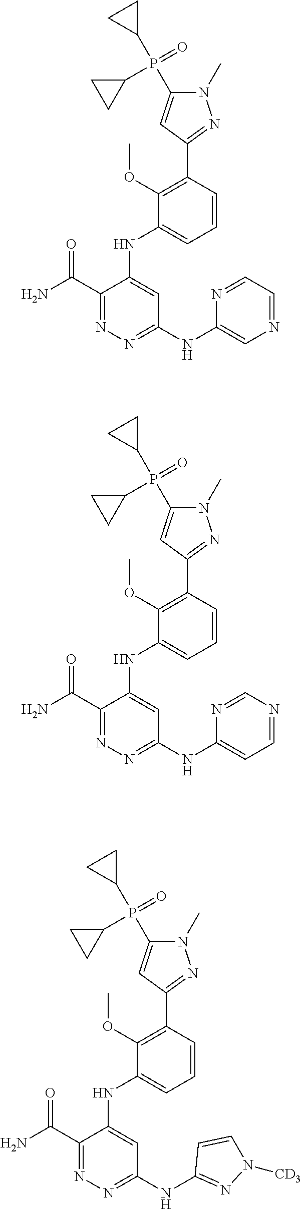 [SMILES: COc1c(Nc2cc(Nc3cnccn3)nnc2C(N)=O)cccc1-c1cc(P(=O)(C2CC2)C2CC2)n(C)n1 ; COc1c(Nc2cc(Nc3ccncn3)nnc2C(N)=O)cccc1-c1cc(P(=O)(C2CC2)C2CC2)n(C)n1 ; [2H]C([2H])([2H])n1ccc(Nc2cc(Nc3cccc(-c4cc(P(=O)(C5CC5)C5CC5)n(C)n4)c3OC)c(C(N)=O)nn2)n1]

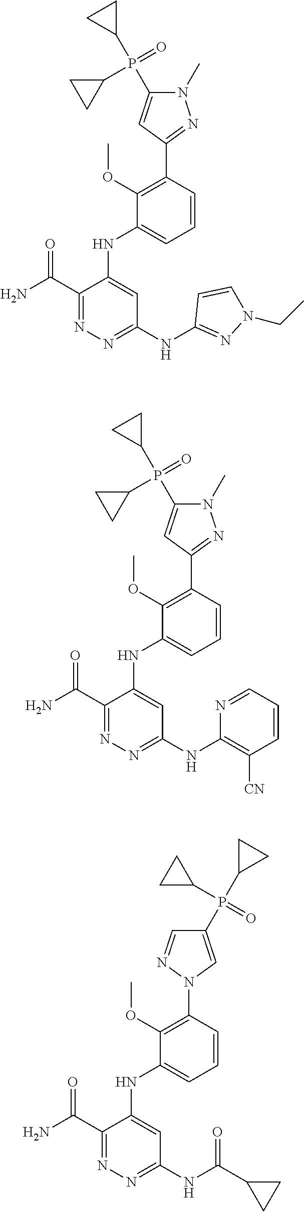 [SMILES: CCn1ccc(Nc2cc(Nc3cccc(-c4cc(P(=O)(C5CC5)C5CC5)n(C)n4)c3OC)c(C(N)=O)nn2)n1 ; COc1c(Nc2cc(Nc3ncccc3C#N)nnc2C(N)=O)cccc1-c1cc(P(=O)(C2CC2)C2CC2)n(C)n1 ; COc1c(Nc2cc(NC(=O)C3CC3)nnc2C(N)=O)cccc1-n1cc(P(=O)(C2CC2)C2CC2)cn1]

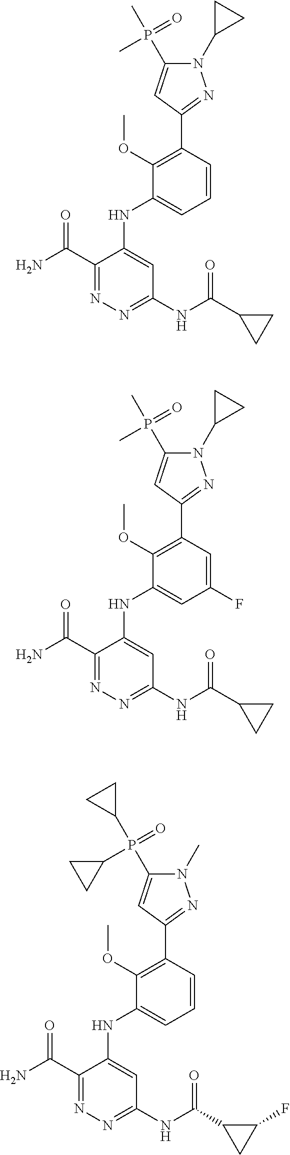

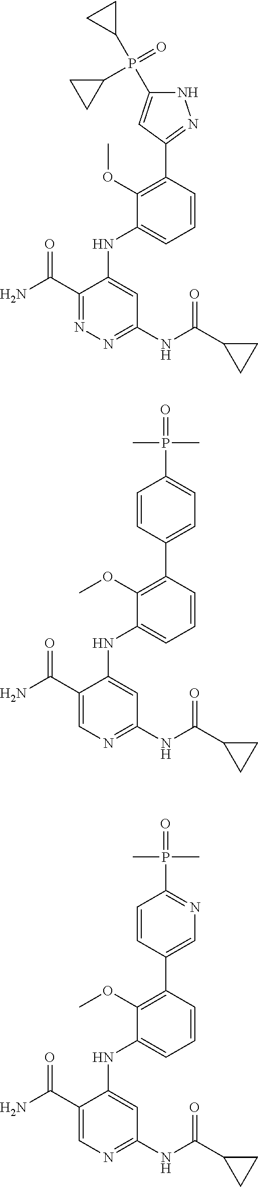 [SMILES: COc1c(Nc2cc(NC(=O)C3CC3)nnc2C(N)=O)cccc1-c1cc(P(=O)(C2CC2)C2CC2)[nH]n1 ; COc1c(Nc2cc(NC(=O)C3CC3)ncc2C(N)=O)cccc1-c1ccc(P(C)(C)=O)cc1 ; COc1c(Nc2cc(NC(=O)C3CC3)ncc2C(N)=O)cccc1-c1ccc(P(C)(C)=O)nc1]

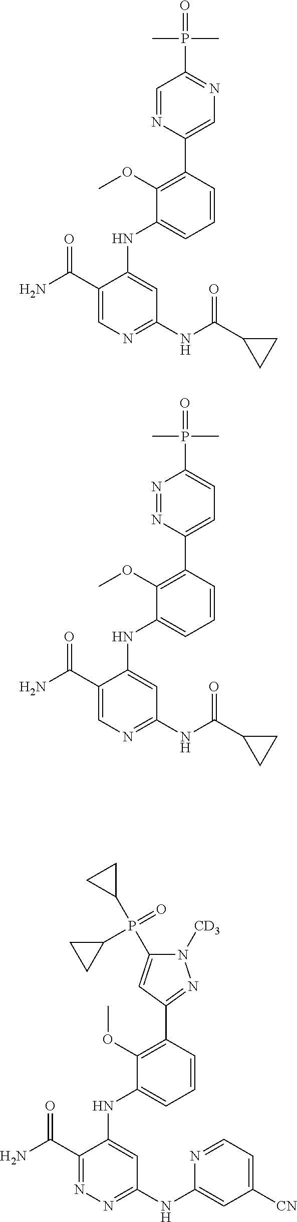 [SMILES: COc1c(Nc2cc(NC(=O)C3CC3)ncc2C(N)=O)cccc1-c1cnc(P(C)(C)=O)cn1 ; COc1c(Nc2cc(NC(=O)C3CC3)ncc2C(N)=O)cccc1-c1ccc(P(C)(C)=O)nn1 ; [2H]C([2H])([2H])n1nc(-c2cccc(Nc3cc(Nc4cc(C#N)ccn4)nnc3C(N)=O)c2OC)cc1P(=O)(C1CC1)C1CC1]

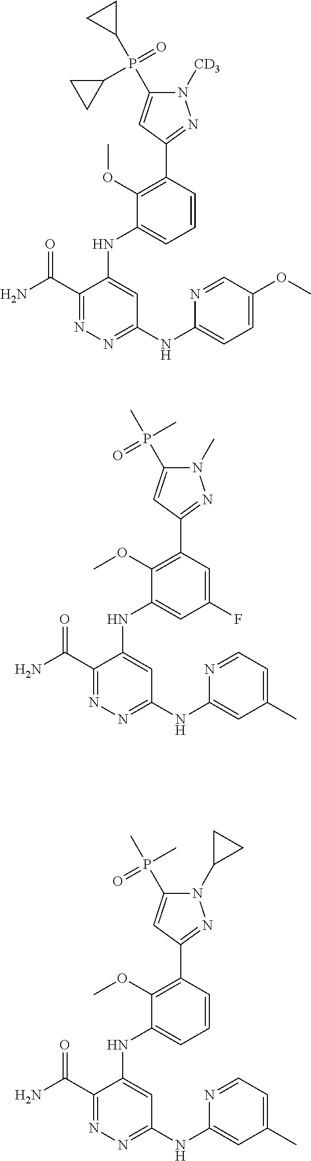 [SMILES: [2H]C([2H])([2H])n1nc(-c2cccc(Nc3cc(Nc4ccc(OC)cn4)nnc3C(N)=O)c2OC)cc1P(=O)(C1CC1)C1CC1 ; COc1c(Nc2cc(Nc3cc(C)ccn3)nnc2C(N)=O)cc(F)cc1-c1cc(P(C)(C)=O)n(C)n1 ; COc1c(Nc2cc(Nc3cc(C)ccn3)nnc2C(N)=O)cccc1-c1cc(P(C)(C)=O)n(C2CC2)n1]

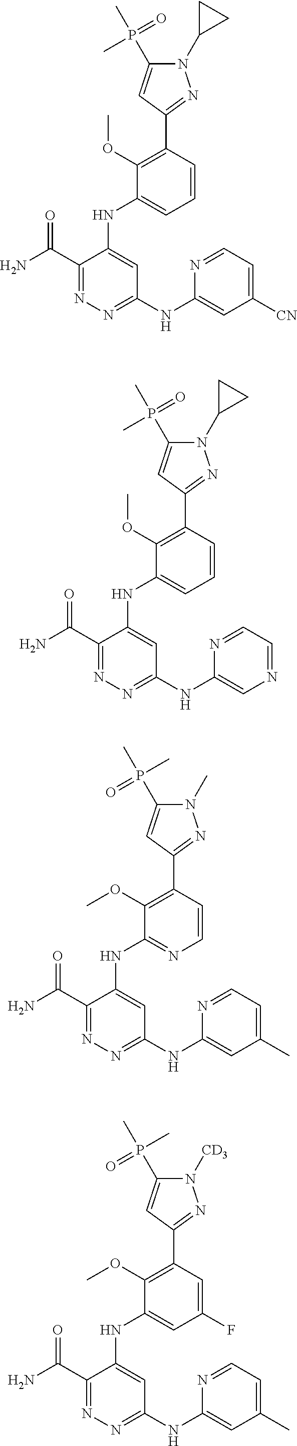 [SMILES: COc1c(Nc2cc(Nc3cc(C#N)ccn3)nnc2C(N)=O)cccc1-c1cc(P(C)(C)=O)n(C2CC2)n1 ; COc1c(Nc2cc(Nc3cnccn3)nnc2C(N)=O)cccc1-c1cc(P(C)(C)=O)n(C2CC2)n1 ; COc1c(-c2cc(P(C)(C)=O)n(C)n2)ccnc1Nc1cc(Nc2cc(C)ccn2)nnc1C(N)=O ; [2H]C([2H])([2H])n1nc(-c2cc(F)cc(Nc3cc(Nc4cc(C)ccn4)nnc3C(N)=O)c2OC)cc1P(C)(C)=O]

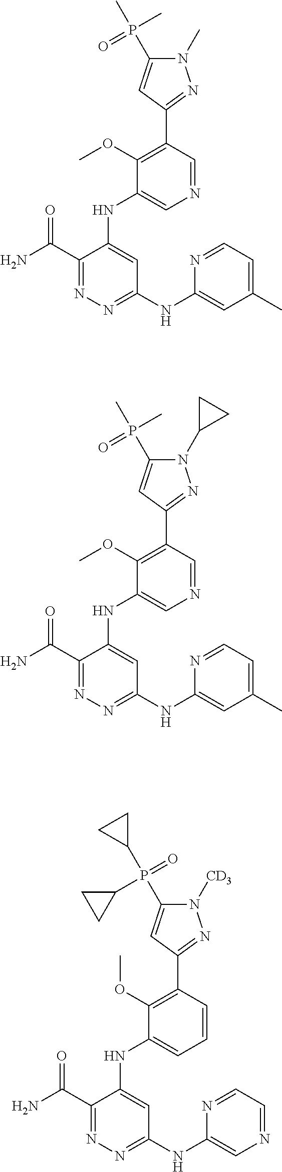 [SMILES: COc1c(Nc2cc(Nc3cc(C)ccn3)nnc2C(N)=O)cncc1-c1cc(P(C)(C)=O)n(C)n1 ; COc1c(Nc2cc(Nc3cc(C)ccn3)nnc2C(N)=O)cncc1-c1cc(P(C)(C)=O)n(C2CC2)n1 ; [2H]C([2H])([2H])n1nc(-c2cccc(Nc3cc(Nc4cnccn4)nnc3C(N)=O)c2OC)cc1P(=O)(C1CC1)C1CC1]

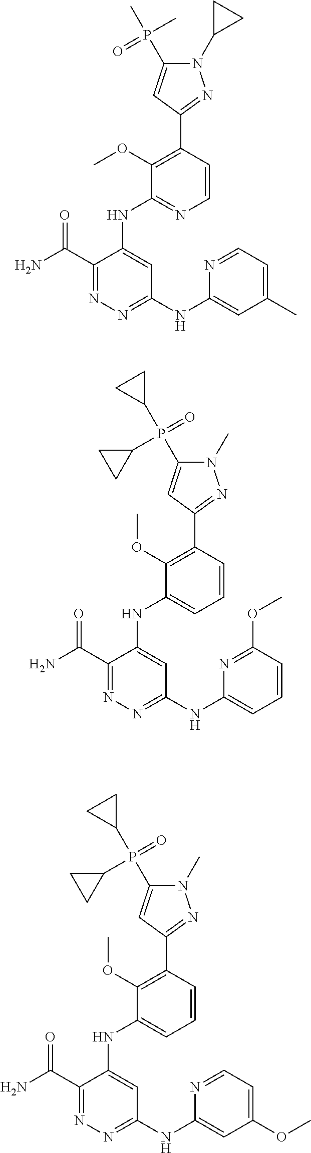 [SMILES: COc1c(-c2cc(P(C)(C)=O)n(C3CC3)n2)ccnc1Nc1cc(Nc2cc(C)ccn2)nnc1C(N)=O ; COc1cccc(Nc2cc(Nc3cccc(-c4cc(P(=O)(C5CC5)C5CC5)n(C)n4)c3OC)c(C(N)=O)nn2)n1 ; COc1ccnc(Nc2cc(Nc3cccc(-c4cc(P(=O)(C5CC5)C5CC5)n(C)n4)c3OC)c(C(N)=O)nn2)c1]

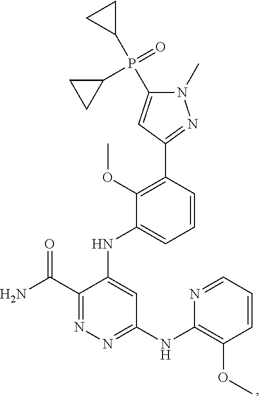 [SMILES: COc1cccnc1Nc1cc(Nc2cccc(-c3cc(P(=O)(C4CC4)C4CC4)n(C)n3)c2OC)c(C(N)=O)nn1 ; ","]

or a pharmaceutically acceptable salt, ester, solvate, prodrug, isotope-labelled derivative, or isomer thereof.

Another aspect of the present disclosure provides a pharmaceutical composition comprising a therapeutically effective amount of the compound of any one of the hitherto described embodiments, or a pharmaceutically acceptable salt, ester, solvate, prodrug, isotope-labeled derivative, or isomer thereof, and a pharmaceutically acceptable carrier.

Another aspect of the present disclosure provides a composition comprising the compound of any one of the hitherto described embodiments, or a pharmaceutically acceptable salt, ester, solvate, prodrug, isotope-labeled derivative or isomer thereof, or the pharmaceutical composition of any one of the hitherto described embodiments, and one or more additional therapeutic agent selected from the group consisting of anti-autoimmune/anti-inflammatory agent, anti-tumor/anti-cancer agent, anti-allergic agent, anti-transplant rejection agent, anti-neurodegenerative agent, anti-asthma agent and other anti-obstructive airway disease agent.

Another aspect of the present disclosure provides a method for treating a disease or disorder by inhibiting TYK2 and/or JAK1 mediated signal transduction in a subject suffering therefrom, comprising administering to the subject a therapeutically effective amount of the compound of any one of the hitherto described embodiments, or a pharmaceutically acceptable salt, ester, solvate, prodrug, isotope-labeled derivative or isomer thereof, or the pharmaceutical composition of any one of the hitherto described embodiments, or the composition of any one of the hitherto described embodiments.

In some embodiments of the above method, the disease or disorder is autoimmune disease or inflammation disease, cancer or tumor, allergy, transplant rejection, neurodegenerative disease, asthma or other obstructive airway diseases.

In some embodiments, the autoimmune disease or inflammation disease is enteritis, skin disease, eye disease, arthritis, Hashimoto's thyroiditis, autoimmune hemolytic anemia, autoimmune atrophic gastritis, autoimmune encephalomyelitis, Goodpasture syndrome, autoimmune thrombocytopenia, sympathetic ophthalmitis, myositis, primary biliary cirrhosis, hepatitis, primary sclerosing cholangitis, chronic invasive hepatitis, nonalcoholic fatty liver disease, nonalcoholic steatohepatitis, ulcerative colitis, membranous glomerulopathy, systemic lupus erythematosus, rheumatoid arthritis, psoriatic arthritis, polyarthritis dermatomyositis, type I interferonopathies (including Aicardi-Goutières syndrome) and other systemic sclerosis caused by over-expression of type I interferon, Mendelian disease, multiple arteritis nodosa, multiple sclerosis, relapsing multiple sclerosis, primary progressive multiple sclerosis, secondary progressive multiple sclerosis and bullous pemphigus, Cogan's syndrome, ankylosing spondylitis, Wegener's granulomatosis, autoimmune alopecia, diabetes, or thyroid inflammation.

In some embodiments, the enteritis is Crohn's disease, ulcerative colitis, inflammatory bowel disease, celiac disease, proctitis, eosinophilic gastroenteritis, or mastocytosis.

In some embodiments, the skin disease is atopic dermatitis, eczema, psoriasis, scleroderma, pruritus or other symptoms of itching, vitiligo, or alopecia.

In some embodiments, the eye disease is keratoconjunctivitis, uveitis (including uveitis associated with Behçet's disease and uveitis caused by the lens), keratitis, herpetic keratitis, keratoconus, muscular dystrophic epithelial keratitis inflammation, corneal leukopenia, anterior uveitis, scleritis, Mooren's ulcer, Graves ophthalmopathy, Vogt-Koyanagi-Harada syndrome, keratoconjunctivitis sicca, vesicular, iridocyclitis iridosarcoidosis, endocrine ophthalmopathy, sympathetic ophthalmitis, allergic conjunctivitis, or ocular neovascularization.

In some embodiments, the diabetes is type 1 diabetes or diabetic complications.

In some embodiments, the cancer or tumor is digestive/gastrointestinal cancers, colon cancers, liver cancers, skin cancers (including mast cell and squamous cell carcinomas), breast cancers, ovarian cancers, prostate cancers, lymphomas, leukemia (including acute myeloid leukemia and chronic myeloid leukemia), kidney cancer, lung cancer, muscle cancer, bone cancer, bladder cancer, brain cancer, melanoma (including oral and metastatic melanoma), Kaposi's sarcoma (including multiple myeloma), myeloproliferative disorders, proliferative diabetic retinopathy, or diseases/tumors associated with vascular hyperplasia.

In some embodiments, the neurodegenerative disease is motor neuron disease, Alzheimer's disease, Parkinson's disease, amyotrophic lateral sclerosis, Huntington's disease, cerebral ischemia, neurodegenerative diseases caused by trauma, injury, glutamate neurotoxicity or hypoxia, stroke, myocardial ischemia, renal ischemia, heart disease, cardiac hypertrophy, atherosclerosis, arteriosclerosis, ischemia/reperfusion injury of organ hypoxia or platelet aggregation.

In some embodiments, the allergy is allergic dermatitis in subjects (including allergic diseases in horses, such as allergy to bites), summer eczema, itchy horseshoes, cramps, airway inflammation, recurrent airway obstruction, airway hyperresponsiveness, and chronic obstructive pulmonary disease.

In some embodiments, the asthma or other obstructive airway diseases are chronic or excessive asthma, delayed asthma, bronchitis, bronchial asthma, allergic asthma, intrinsic asthma, extrinsic asthma or dusty asthma.

In some embodiments, the transplant rejection is islet transplant rejection, bone marrow transplant rejection, graft-versus-host disease, organ and cell transplant rejection (the organ and cell are bone marrow, cartilage, cornea, heart, intervertebral disc, islet, kidney, extremity, liver, lung, muscle, myoblasts, nerves, pancreas, skin, small intestine or trachea), or xenograft rejection.

The present disclosure also provides a formulation of the compound disclosed herein including all embodiments, the pharmaceutical composition disclosed herein including all embodiments, or the composition disclosed herein including all embodiments, wherein the formulation is tablet, capsule, injection agent, granule, powder, suppository, pill, gel, powder, oral solution, inhalation agent, suspension, or dry suspension.

In some embodiments, including any one of the hitherto described embodiments, the disclosed compound selectively inhibits TYK2/JAK1, can be used as effective inhibitors of TYK2/JAK1, and can be used to prevent or treat diseases and/or symptoms caused by TYK2/JAK1. In some embodiments, the disclosed compounds unexpectedly show no or substantially no inhibition on JAK2.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1 depicts the Western Blot experimental results of the inhibition of JAK2/JAK2 pathway by Compounds A1-A7 according to Example 27;

FIG. 2 depicts the Western Blot experimental results of the inhibition of JAK2/JAK2 pathway by Compound A8 according to Example 27;

FIG. 3 depicts the Western Blot experimental results of the inhibition of JAK2/JAK2 pathway by Compound A9 according to Example 27;

FIG. 4 depicts the Western Blot experimental results of the inhibition of JAK2/JAK2 pathway by Compound A12 according to Example 27;

FIG. 5 depicts the Western Blot experimental results of the inhibition of JAK2/JAK2 pathway by Compound A17 according to Example 27;

FIG. 6 depicts the Western Blot experimental results of the inhibition of JAK2/JAK2 pathway by Compound A29 according to Example 27;

FIG. 7 depicts the Western Blot experimental results of the inhibition of JAK2/JAK2 pathway by Compounds A35, A38-A42, and A49-A50 according to Example 27;

FIG. 8 depicts the Western Blot experimental results of the inhibition of TYK2/JAK2 pathway by Compounds A1-A5 according to Example 27;

FIG. 9 depicts the Western Blot experimental results of the inhibition of TYK2/JAK2 pathway by Compound A8 according to Example 27;

FIG. 10 depicts the Western Blot experimental results of the inhibition of TYK2/JAK2 pathway by Compounds A4, A5, A9, and A12 according to Example 27;

FIG. 11 depicts the Western Blot experimental results of the inhibition of TYK2/JAK2 pathway by Compounds A12, A17, A29, A35, and A38 according to Example 27;

FIG. 12 depicts the Western Blot experimental results of the inhibition of TYK2/JAK2 pathway by Compounds A39-A42 and A49-A50 according to Example 27;

FIG. 13 depicts the Western Blot experimental results of the inhibition of JAK1/JAK2 pathway by Compounds A1, A5, and A6 according to Example 27;

FIG. 14 depicts the Western Blot experimental results of the inhibition of JAK1/JAK2 pathway by Compounds A4 and A5 according to Example 27;

FIG. 15 depicts the Western Blot experimental results of the inhibition of JAK1/JAK2 pathway by Compounds A2, A8-A9, A12, A17, and A29 according to Example 27; and

FIG. 16 depicts the Western Blot experimental results of the inhibition of JAK1/JAK2 pathway by Compounds A35, A38-A42, and A49-A50 according to Example 27.

Before proceeding with the detailed description, it is to be appreciated that the following detailed description is merely exemplary in nature and is not intended to limit the invention or the application and uses thereof. Hence, although the present disclosure is, for convenience of explanation, depicted and described as shown in certain illustrative embodiments, it will be appreciated that it can be implemented in various other types of embodiments and equivalents, and in various other systems and environments. Furthermore, there is no intention to be bound by any theory presented in the preceding background or the following detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms.

As used herein, the singular forms “a,” “an” and “the” include plural referents unless the context clearly dictates otherwise. Thus, for example, reference to “a molecule” includes a plurality of such molecules, and the like.

The term “about” or “nearly” as used herein generally refers to within +/−15%, 10%, 9%, 8%, 7%, 6%, 5%, 4%, 3%, 2%, or 1% of the designated amount.

The term “halogen” or “halide” as used herein generally refers to fluorine, chlorine, bromine, and iodine. The term “haloalkyl” or “halo-alkyl” as used herein generally refers to an alkyl group that is substituted with one or more independently chosen halogens (e.g., “C₁-C₆ haloalkyl” groups have from 1 to 6 carbon atoms and at least one halogen). Examples of haloalkyl groups include, but are not limited to, mono-, di- or tri-fluoromethyl; mono-, di- or tri-chloromethyl; mono-, di-, tri-, tetra- or penta-fluoroethyl; mono-, di-, tri-, tetra- or penta-chloroethyl; and 1,2,2,2-tetrafluoro-1-trifluoromethyl-ethyl. The term “haloalkoxy” or “halo-alkoxy” as used herein generally refers to an alkoxy group that is substituted with one or more independently chosen halogens (e.g., “C₁-C₆ haloalkoxy” or “C₁-C₆ halo-alkoxy” groups have from 1 to 6 carbon atoms and at least one halogen attached to one of the carbon atoms). Examples of haloalkoxy groups include, but are not limited to, mono- or di-fluoromethoxy; mono- or di-chloromethoxy; mono-, di-, tri-, or tetra-fluoroethoxy; and mono-, di-, tri-, or tetra-chloroethoxy.

The term “alkyl” as used herein generally refers to a straight or branched chain saturated aliphatic hydrocarbon. Alkyl groups include groups having from 1 to 8 carbon atoms (C₁₋₈ alkyl), from 1 to 6 carbon atoms (C₁₋₆ alkyl) and from 1 to 4 carbon atoms (C₁-C₄ alkyl), including, for example, methyl, ethyl, propyl, isopropyl, n-butyl, ser-butyl, isobutyl, tert-butyl, pentyl, 2-pentyl, 3-pentyl, 2-methyl-2-butyl, 3-methyl-2-butyl, 3-methyl-1-butyl, 2-methyl-1-butyl, n-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. In some embodiments, “C₁₋₆ alkyl” is, preferably, methyl, ethyl, n-propyl, isopropyl or tert-butyl. Similarly, C₁₋₃ alkyl refers to an alkyl group having from 1 to 3 carbon atoms in a straight or branched chain, including, for example, methyl, ethyl, propyl, and isopropyl. In some instances, a substituent of an alkyl group is specifically indicated. For example, “cyanoalkyl” refers to an alkyl group substituted with at least one cyano substituent.

The term “alkenyl” as used herein generally refers to straight or branched chain alkene groups, which comprise at least one unsaturated carbon-carbon double bond. Alkenyl groups include C₂₋₈ alkenyl, C₂₋₆ alkenyl and C₂₋₄ alkenyl groups, which have from 2 to 8, 2 to 6, or 2 to 4 carbon atoms, respectively, including, for example, ethenyl, allyl and isopropenyl.

The term “alkynyl” as used herein generally refers to straight or branched chain alkyne groups, which have one or more unsaturated carbon-carbon bonds, at least one of which is a triple bond. Alkynyl groups include C₂₋₈ alkynyl, C₂₋₆ alkynyl and C₂₋₄ alkynyl groups, which have from 2 to 8, 2 to 6 or 2 to 4 carbon atoms, respectively, including, for example, ethynyl and propargyl.

The term “alkoxy” as used herein generally refers to an alkyl group as described above attached via an oxygen bridge to another chemical moiety. Alkoxy groups include different length of the alkyl groups, such as, for example, C₁₋₆ alkoxy and C₁₋₄ alkoxy groups, which have from 1 to 6 or from 1 to 4 carbon atoms, respectively. The term “OC₁₋₆ alkyl” as used herein generally refers to alkoxy groups include an alkyl group (with 1 to 6 carbon atoms) attached to an oxygen atom. Methoxy, ethoxy, n-propoxy, isopropoxy, n-butoxy, sec-butoxy, isobutoxy, tert-butoxy, n-pentoxy, 2-pentoxy, 3-pentoxy, isopentoxy, neopentoxy, hexoxy, 2-hexoxy, 3-hexoxy, and 3-methylpentoxy are representative alkoxy groups.

The term “cycloalkyl” as used herein generally refers to a group that comprises one or more saturated rings in which all ring members are carbon. For example, certain cycloalkyl groups are C₃₋₈ cycloalkyl, in which the cycloalkyl group contains one or more rings having from 3 to 8 ring members, all of which are carbon, including, for example, cyclopropyl, cyclobutyl, cyclopentyl, cyclohexyl, cycloheptyl, and cyclooctyl. Other example of cycloalkyl group includes adamantyl. Cycloalkyl groups do not comprise an aromatic ring or a heterocyclic ring. The term “cycloalkenyl” as used herein generally refers to a group that comprises one or more unsaturated rings in which all ring members are carbon.

The terms “heterocyclic” or “heterocycle” or “heterocyclyl” or “cycloheteroalkyl” as used herein generally refer to a ring structure (monocycle or polycycle) containing 3-12 ring atoms (3-12 membered heterocycle), 3-8 ring atoms (3-8 membered heterocycle or 3-8 membered cycloheteroalkyl), 3-6 ring atoms (3-6 membered heterocycle or 3-6 membered cycloheteroalkyl), or 5-6 ring atoms (5-6 membered heterocycle or 5-6 membered cycloheteroalkyl), in which at least one ring atom is carbon, and at least one ring atom is heteroatom selected from N, O, and S or at least one heteroatom group is selected from C(═O), S(═O), and S(═O)₂. A heterocyclic group may be aromatic or non-aromatic. Piperidine and oxetane are non-limiting examples of non-aromatic heterocycles. Thiazole and pyridine are non-limiting examples of aromatic heterocycles. Other examples of heterocycle include: aziridinyl, azetidinyl, oxetanyl, pyrrolidinyl, tetrahydrofuranyl, tetrahydrothienyl, piperidinyl, morpholinyl, piperazinyl, thiomorpholinyl, tetrahydropyranyl, 1,1-dioxothiomorpholinyl, butyrolactam, valerolactam, caprolactam, butyrolactone, valerolactone and caprolactone. Similarly, the term “cycloheteroalkenyl” refers to a monocycle or polycycle ring structure comprising carbon atom(s) and heteroatom(s)/heteroatom group(s), wherein the cycloheteroalkenyl comprises at least one C═C double bond, at least one ring atom that is carbon and at least one ring atom that is heteroatom selected from N, O, and S or a heteroatom group selected from C(═O), S(═O), and S(═O)₂.

The term “aryl” as used herein generally refers to an all-carbon monocyclic or fused-ring polycyclic groups of 6 to 12 (C₆₋₁₂ aryl) or 6 to 10 carbon atoms (C₆₋₁₀ aryl) having a completely conjugated pi-electron system. Examples, without limitation, of aryl groups are phenyl, naphthalenyl, tetrahydronaphthyl, indanyl, biphenyl, and anthracenyl. The aryl group may be substituted or unsubstituted. Typical substituents include halo, trihalomethyl, alkyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, C-amido, N-amido, sulfinyl, sulfonyl, amino and —NR^{X}R^{Y}, wherein R^{X} and R^{Y} are independently selected from the group consisting of hydrogen, alkyl, cycloalkyl, aryl, carbonyl, acetyl, sulfonyl, trifluoromethanesulfonyl and, combined, a five- or six-membered heteroalicyclic ring. Illustrative substituted alkyl group include, but are not limited to, fluoromethyl, difluoromethyl, trifluoromethyl, aminomethyl, aminoethyl, hydoxymethyl, methoxymethyl, 2-fluoroethyl, and 2-methoxyethyl, etc.

The term “heteroaryl” as used herein generally refers to an aromatic group in which at least one aromatic ring comprises at least one heteroatom selected from N, O and S. Heteroaryls include, for example, 5-12 membered heteroaryls, 5-10 membered heteroaryls, 5-7 membered monocyclic structures or 7-12 membered bicyclic structures. The number of heteroatoms in a heteroaryl can be 1, 2, 3, 4, or more. Examples included but are not limited to thienyl, pyridyl, pyrimidinyl, pyrazinyl, pyrazolyl, pyridazinyl, pyridine-2(1H)-keto, pyridine-4(1H)-keto, pyrrolyl, pyrazolyl, thiazolyl, 1,2,3-triazolyl, 1,2,4-triazolyl, 1,2,5-oxadiazolyl, imidazolyl, furanyl, tetrazolyl, isothiazolyl, oxazolyl, isoxazolyl, thiadiazolyl, oxadiazolyl, naphthyl, benzothienyl, indolyl, benzimidazolyl, benzothiazolyl, benzofuranyl, quinolinyl, isoquinolinyl, and quinazolinyl. The heteroaryl group may be substituted or unsubstituted. Typical substituents include halo, trihalomethyl, alkyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, C-amido, N-amido, sulfinyl, sulfonyl, amino and —NR^{X}R^{Y}, with R^{X} and R^{Y} as defined above.

The term “amino” as used herein generally refers to primary amino group (—NH₂), secondary amino group (—NH—), and tertiary amino group

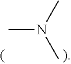

The term “alkylamino” as used herein generally refers to a secondary or tertiary amine that has the general structure —NH—R¹ or —N(R¹)(R²), respectively, wherein R¹ and R² are selected independently from alkyl, cycloalkyl and (cycloalkyl)alkyl groups. Such groups include, but are not limited to, for example, “—NH(C₁₋₆ alkyl)” and “—N(C₁₋₆ alkyl)₂” comprising mono- and di-(C₁₋₆ alkyl) groups, respectively, in which each C₁₋₆ alkyl may be the same or different. It will be apparent that the definition of “alkyl” as used in the term “alkylamino” differs from the definition of “alkyl” used for all other alkyl-containing groups, in the inclusion of cycloalkyl and (cycloalkyl)alkyl groups.

The term “alkylthio” as used herein generally refers to an alkyl-substituted thio group, wherein the term alkyl is as defined above.

The terms “substituent” and “substituted”, as used herein, generally denote that a molecular moiety is covalently bonded to an atom within a molecule of interest. The atom attached to can be carbon or nitrogen. The molecule can be alkyl, cycloalkyl, heterocycloalkyl, aryl, or heteroaryl. For example, a ring substituent may be a moiety such as a halogen, an alkyl group, a haloalkyl group or other group that is covalently bonded to an atom (preferably a carbon or nitrogen atom) that is a ring member. Substituents of aromatic groups are generally covalently bonded to a ring carbon atom or a ring nitrogen atom. A straight chain substituent may be a moiety such as a halogen, an alkyl group, a haloalkyl group or other group that is covalently bonded to an atom (preferably a carbon or nitrogen atom) that is a member of a straight chain.

The term “pharmaceutically acceptable” as used herein generally refers to a form of the compound that is safe for administration to a subject. For example, a free base, a salt form, a solvate, a hydrate, a prodrug or derivative form of a compound described herein, which has been approved for mammalian use, via oral ingestion or any other route of administration, by a governing authority or regulatory agency, such as the Food and Drug Administration (FDA) of the United States, is pharmaceutically acceptable.

Included in the compounds of Formulas (I), (II), (III), (IV), and (V) are the pharmaceutically acceptable salt forms of the free-base compounds. The term “pharmaceutically-acceptable salt” as used herein generally refers to salts, commonly used to form alkali metal salts and to form addition salts of free acids or free bases, which have been approved by a regulatory agency. Salts are formed from ionic associations, charge-charge interactions, covalent bonding, complexation, coordination, etc. The nature of the salt is not critical, provided that it is pharmaceutically acceptable. For example, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. For example, Berge et al. describes pharmaceutically acceptable salts in detail in Pharmaceutical Sciences (1977) 66: 1-19. Pharmaceutically acceptable salts of the compounds provided herein include those derived from suitable inorganic and organic acids and bases. Inorganic acids from which salts can be derived include, but are not limited to, hydrochloric acid, hydrobromic acid, sulfuric acid, nitric acid, phosphoric acid, and the like. Organic acids from which salts can be derived include, but are not limited to, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and other amine salt. Inorganic bases from which salts can be derived include, but are not limited to, sodium, potassium, lithium, ammonium, calcium, magnesium, iron, zinc, copper, manganese, aluminum, and the like. Organic bases from which salts can be derived include, but are not limited to, primary, secondary, and tertiary amines, substituted amines, including naturally occurring substituted amines, cyclic amines, basic ion exchange resins, and the like, examples include, but are not limited to, isopropylamine, trimethylamine, diethylamine, triethylamine, tripropylamine, and ethanolamine. In some embodiments, the pharmaceutically acceptable base addition salt is ammonium, potassium, sodium, calcium, or magnesium salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. Bis salts (i.e., two counterions) and higher salts (e.g., three or more counterions) are encompassed within the meaning of pharmaceutically acceptable salts.

As used herein, the term “ester” refers to organic compounds comprising an ester bond, including monoester, diester, trimester, and polyester.

As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Other solvates include, but are not limited to, methanol, ethanol, isopropanol, ethyl acetate, tetrahydrofuran, dimethyl sulfoxide, and N,N-dimethylformamide. Pharmaceutically acceptable solvates and hydrates are complexes that, for example, can include 1 to about 100, or 1 to about 10, or one to about 2, 3 or 4, solvent or water molecules.

As used herein, and unless otherwise specified, “prodrug” refers to a compound that can be converted under physiological conditions or by solvolysis to a biologically active compound described herein. Thus, the term “prodrug” refers to a precursor of a biologically active compound that is pharmaceutically acceptable. A prodrug can be inactive when administered to a subject, but is converted in vivo to an active compound, for example, by hydrolysis. A discussion of prodrugs is provided in Higuchi, T., et al, “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compounds according to Formulas (I), (II), (III), (IV), or (V) in vivo when such prodrug is administered to a mammalian subject. Prodrugs of an active compound, as described herein, can be prepared by modifying functional groups present in the active compounds according to Formulas (I), (II), (III), (IV), or (V) in such a way that the modifications are cleaved, either in routine manipulation or in vivo, to the parent active compound. Prodrugs include compounds wherein a hydroxy, amino or mercapto group is bonded to any group that, when the prodrug of the active compounds according to Formulas (I), (II), (III), (IV), or (V) is administered to a mammalian subject, cleaves to form a free hydroxy, free amino or free mercapto group, respectively.

The terms “isotope-labeled”, “isotope label”, “isotope-labeled derivative” and “isotopically labeled” refer to unnatural proportions of atomic isotopes at one or more of atoms that constitute such compounds. For example, the compounds can be radio labeled with radioactive isotopes, such as, for example, tritium (³H), iodine-125 (¹²⁵I), carbon-14 (¹⁴C). The compounds can also be isotope-labeled with ²H, ¹¹C, ¹³C, ¹⁵N, ¹⁷O, ¹⁸O, ¹⁸F, ³²P, ³⁵S, and ³⁶Cl. Certain isotope-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of compounds of the present disclosure, whether radioactive or not, are encompassed within the scope of the present disclosure.

The term “isomers” as used herein generally refers to different compounds that have the same molecular formula, including any and all geometric isomers, tautomers and stereoisomers. “Stereoisomers” are isomers that differ only in the way the atoms are arranged in space. For example, “isomers” include geometric double bond cis- and trans-isomers, also termed E- and Z-isomers; R- and S-enantiomers; diastereomers, (d)-isomers and (l)-isomers, racemic mixtures thereof; and other mixtures thereof, as falling within the scope of this disclosure, unless specified otherwise. As used herein, the term “tautomer” is a type of isomer that includes two or more interconvertible compounds resulting from at least one formal migration of a hydrogen atom and at least one change in valency (e.g., a single bond to a double bond, a triple bond to a single bond, or vice versa).

The term “each independently”, as used herein, means that at least two groups (or ring systems) present in a structure with the same or similar value ranges may have the same or different meanings under certain circumstances. For example, if substituent X and substituent Y are each independently hydrogen, halogen, hydroxyl, cyano, alkyl or aryl, then when substituent X is hydrogen, substituent Y can be hydrogen, halogen, hydroxyl, cyano, alkyl or aryl. Similarly, when the substituent Y is hydrogen, the substituent X can be hydrogen, halogen, hydroxyl, cyano, alkyl or aryl.

The terms “optional” or “optionally”, as used herein, mean that the subsequently described event or circumstance may or may not occur, and that the description includes both the occurrence and the non-occurrence of the subsequent event or circumstance.

In some embodiments, the compound(s) of Formulas (I), (II), (III), (IV), or (V) is used to treat a subject by administering the compound(s) as a pharmaceutical composition. To this end, the compound(s), in one embodiment, is combined with one or more pharmaceutically acceptable excipients, including carriers, diluents or adjuvants, to form a suitable composition, which is described in more detail herein.

The term “excipient” as used herein generally refers to any pharmaceutically acceptable additive, carrier, adjuvant, or other suitable ingredient, other than the active pharmaceutical ingredient (API), which is typically included for formulation and/or administration purposes.

The term “diluent” as used herein generally refers to an agent used as filler in order to achieve the desired composition volume or weight. The diluent may be present in the pharmaceutical composition within granules in the form of a single compound or in the form of a mixture of compounds. Non-limiting examples of diluent include lactose, starch, pregelatinized starch, microcrystalline cellulose, silicified microcrystalline cellulose, cellulose acetate, dextrose, mannitol, sodium phosphate, potassium phosphate, calcium phosphate, fructose, maltose, sorbitol, or sucrose.

The term “adjuvant,” as used herein generally refers to any substance or mixture of substances that increases the efficacy or potency of a compound disclosed herein on a target where the adjuvant is used together with the compound disclosed herein. However, when the adjuvant is used alone, no pharmacological effect is observed on the same target.

The terms “treat”, “treating,” “treatment,” and “therapy” as used herein generally refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals. Treatment includes the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder, preventative treatment, that is, treatment directed to minimizing or partially or completely inhibiting the development of the associated disease, pathological condition, or disorder, and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “effective amount” or “therapeutically effective amount”, as used herein, refers to a sufficient amount of an agent or a compound being administered which will relieve one or more of the symptoms of the disease or condition being treated to some extent; achieve the goal of improvement in disorder severity and the frequency of incidence over treatment of each agent by itself, the result thereof can be reduction and/or alleviation of the signs, symptoms, or causes of a disease, or any other desired alteration of a biological system; while avoiding adverse side effects typically associated with alternative therapies. For example, an “effective amount” for therapeutic uses is the amount of the composition as disclosed herein required to provide a clinically significant decrease in disease symptoms. An appropriate “effective” amount in any individual case may be determined using techniques, such as a dose escalation study. The effective amount, in one embodiment, is administered in a single dosage form or in multiple dosage forms.

Regardless of the route of administration selected, the compounds of the present invention, which may be used in a suitable hydrated form, and/or the pharmaceutical compositions of the present invention, are formulated into pharmaceutically acceptable dosage forms or by other conventional methods known to those of skill in the art.

Actual dosage levels of the active ingredients in the pharmaceutical compositions of the present invention may be varied so as to obtain an effective amount of the active ingredient to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular hedgehog inhibitor employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day. The mode of administration can have a large effect on dosage. Higher doses may be used for localized routes of delivery.

If desired, the effective daily dose of the active compound may be administered as two, three, four, five, six or more sub-doses administered separately at appropriate intervals throughout the day, optionally, in unit dosage forms. Those of skill in the art will readily appreciate that dose levels can vary as a function of the specific compound, the severity of the symptoms and the susceptibility of the subject to side effects. Dosages for a given compound disclosed herein are readily determinable by those of skill in the art by a variety of means.

### Pharmaceutical Compositions/Formulations

One embodiment provides a pharmaceutical composition comprising a compound of Formulas (I), (II), (III), (IV), or (V), or a stereoisomer, tautomer, hydrate, solvate or pharmaceutically acceptable salt thereof, and at least one pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed., Easton, Pa.: Mack Publishing Company (1995); Hoover, John E., Remington's Pharmaceutical Sciences, Mack Publishing Co., Easton, Pennsylvania (1975); Liberman, H. A. and Lachman, L., Eds., Pharmaceutical Dosage Forms, Marcel Decker, New York, N.Y. (1980); and Pharmaceutical Dosage Forms and Drug Delivery Systems, Seventh Ed., Lippincott Williams & Wilkins (1999), herein incorporated by reference for such disclosure.

A pharmaceutical composition, as used herein, refers to a mixture of a compound of Formulas (I), (II), (III), (IV), or (V) with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

All formulations for oral administration are in dosages suitable for such administration. Examples of such dosage units are tablets or capsules. In some embodiments, these contain an amount of active ingredient from about 1 to 2000 mg, advantageously from about 1 to 500 mg, and typically from about 5 to 150 mg. A suitable daily dose for a human or other mammal vary widely depending on the condition of the patient and other factors, but, once again, can be determined using routine methods and practices.

Conventional formulation techniques include, e.g., one or a combination of methods: (1) dry mixing, (2) direct compression. (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

### Synthetic Methods

The examples and preparations provided below illustrated and exemplify the compounds described herein and methods of preparing such compounds. In general, the compounds described herein may be prepared by processes known in the general chemical arts.

The compounds of the present invention can be prepared using various synthetic routes, including those described below, starting from commercially available materials. Starting materials of the invention, are either known, commercially available, or can be synthesized in analogy to or according to methods that are known in the art. Many starting materials may be prepared according to known processes and, in particular, can be prepared using processes described in the examples. In synthesizing starting materials, functional groups in some cases are protected with suitable protecting groups when necessary. Functional groups may be removed according to known procedures in the art.

The protection of functional groups by protecting groups, the protecting groups themselves, and their removal reactions (commonly referred to as “deprotection”) are described, for example, in standard reference works, such as J. F. W. McOmie, Protective Groups in Organic Chemistry, Plenum Press, London and New York (1973), in T. W. Greene, Protective Groups in Organic Synthesis, Wiley, New York (1981), in The Peptides, Volume 3, E. Gross and J. Meienhofer editors, Academic Press, London and New York (1981).

All synthetic procedures described herein can be carried out under known reaction conditions, advantageously under those described herein, either in the absence or in the presence (usually) of solvents or diluents.

The invention further encompasses “intermediate” compounds, including structures produced from the synthetic procedures described, whether isolated or not, prior to obtaining the finally desired compound. Structures resulting from carrying out steps from a transient starting material, structures resulting from divergence from the described method(s) at any stage, and structures forming starting materials under the reaction conditions are all “intermediates” included in the invention. Further, structures produced by using starting materials in the form of a reactive derivative or salt, or produced by a compound obtainable by means of the process according to the invention and structures resulting from processing the compounds of the invention in situ are also within the scope of the invention.

New starting materials and/or intermediates, as well as processes for the preparation thereof, are likewise the subject of this invention. In select embodiments, such starting materials are used and reaction conditions so selected as to obtain the desired compound(s).

Starting materials of the invention, are either known, commercially available, or can be synthesized in analogy to or according to methods that are known in the art. Many starting materials may be prepared according to known processes and, in particular, can be prepared using processes described in the examples. In synthesizing starting materials, functional groups in some cases are protected with suitable protecting groups when necessary. Protecting groups, their introduction and removal are described above.

All reagents and solvents were obtained commercially unless stated otherwise. All commercial reagents and solvent were used without purification unless stated otherwise. When required, some reagents and solvents were purified by standard techniques. For example, tetrahydrofuran may be purified by distillation from sodium. All thin-layer chromatography (TLC, GF254) analyses and column purification (100-200 mesh) were performed on silica gel (Qingdao Haiyang Chemical Co. Ltd. or Yantai Chemical Co. Ltd.), using petroleum ether (b.p. 60-90° C.)/ethyl acetate (v/v) as eluent; and spots revealed by UV visualization at 254 nm and I₂ vapor or phosphomolybdic acid. All organic layers after extraction were dried over anhydrous Na₂SO₄ unless stated otherwise. All nuclear magnetic resonance spectra (¹H NMR) were recorded using a Varian-400 spectrometer at 400 MHz using TMS as an internal standard. LC-MS was run using an Agilent 1100 system with LC-MSDTrap recorder, diode array detector (DAD) with detecting wavelength at 214 nm and 254 nm, and ESI source. The HPCL column is an AgelaDurashell C18 3.5 μm 4.6×50 mm column. Gradients were run using 0.1 NH₄HCO₃ aqueous solution and acetonitrile with gradient 5/95 to 95/5 in the run time indicated (for example, 5 min), flow rate at 1.8 mL/min.

The size and scale of the synthetic methods will vary depending on the desired amount of end product. It is understood that while specific reactants and amounts are provided in the Examples, one of skill in the art knows other alternative and equally feasible sets of reactants that will also yield the same compounds. Thus, where general oxidizers, reducers, solvents of various nature (aprotic, apolar, polar, etc.) are utilized, equivalents will be known in the art and are herein contemplated for use in the present methods.

Many of the steps below indicate various work-ups following termination of the reaction. A work-up involves generally quenching of a reaction to terminate any remaining catalytic activity and starting reagents. This is generally followed by addition of an organic solvent and separation of the aqueous layer from the organic layer. The product is typically obtained from the organic layer and unused reactants and other spurious side products and unwanted chemicals are generally trapped in the aqueous layer and discarded. The work-up in standard organic synthetic procedures found throughout the literature is generally followed by drying the product by exposure to a drying agent, such as anhydrous Na₂SO₄, to remove any excess water or aqueous byproducts remaining partially dissolved in the organic layer and concentration of the remaining organic layer. Concentration of product dissolved in solvent may be achieved by any known means, such as evaporation under pressure, evaporation under increased temperature and pressure, and the like. Such concentrating may be achieved by use of standard laboratory equipment such as rotary-evaporator distillation, and the like. This is optionally followed by one or more purification steps which may include, but is not limited to, flash column chromatography, filtration through various media and/or other preparative methods known in the art and/or crystallization/recrystallization. (See, for instance, Addison Ault, “Techniques and Experiments for Organic Chemistry,” 6th Ed., University Science Books, Sausalito, Calif., 1998, Ann B. McGuire, Ed., pp. 45-59).

### Abbreviations

DBU means 1,8-diazabicyclo[5.4.0]undec-7-ene.
DMF means N,N-dimethylformamide.
EDCI is 1-ethyl-3-(3-dimethylaminopropyl)carbodiimide.
HOBT is hydroxybenzotriazole.
IPA is isopropyl alcohol.
NMI is 1-methylimidazole or N-methylimidazole.
NMP is N-methyl-2-pyrrolidone.
Pd₂(dba)₃ is tris(dibenzylideneacetone)dipalladium(0).
Pd(dppf)Cl₂ is 1,1′-bis(diphenylphosphino)ferrocene]dichloropalladium(I).
Pd(OAc)₂ is palladium(II) acetate.
TEA or Et₃N is triethylamine.
THF is tetrahydrofuran.
DCM means dichloromethane.
Xantphos is (9,9-Dimethyl-9H-xanthene-4,5-diyl)bis(diphenylphosphane).
MTBE is methyl tert-butyl ether.
NaOAc is sodium acetate.
LiHMDS is lithium bis(trimethylsilyl)amide.
DPPA is diphenylphosphorylazide.
t-BuOH is tert-butyl alcohol.
(BPIN)₂ is bis(pinacolato)diboron.
EtOAc or EA means ethyl acetate.
DCE means 1,2-dichloroethane.
NaH is sodium hydride.
PE is petroleum ether.
TLC means thin layer chromatography.
HPLC means high-performance liquid chromatography.
LC-MS means liquid chromatography-mass spectrometry.
NMR means nuclear magnetic resonance.

### General Synthetic Routes

The following Methods AA-AR are embodiments for some general synthetic routes leading to compounds of Formulas (I), (II), (III), (IV), or (V). Detailed reaction conditions for each Method can be found in the examples shown vide infra.

### Method AA:

### Method AA

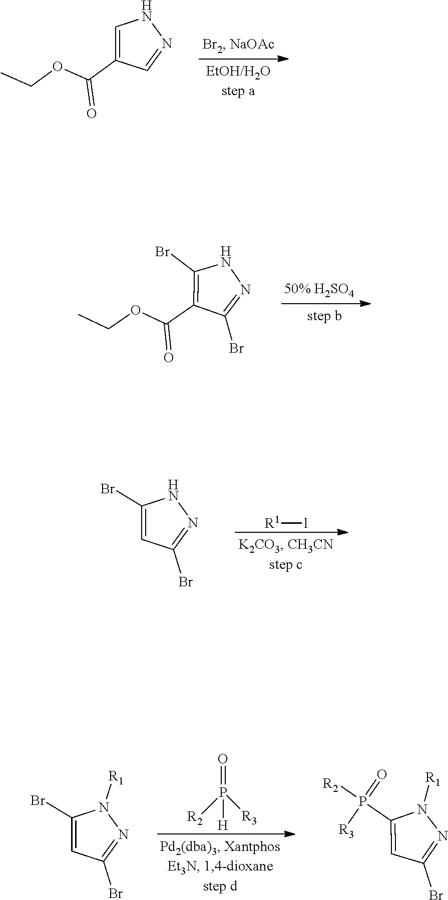

Bromination of ethyl 1H-pyrazole-4-carboxylate with bromine followed by decarboxylation with 50% H₂SO₄ yielded 3,5-dibromo-1H-pyrazole (steps a, b). After alkylation under basic conditions (step c), the intermediates were coupled with alkyl phosphorus oxide to give the desired products (step d).

### Method AB

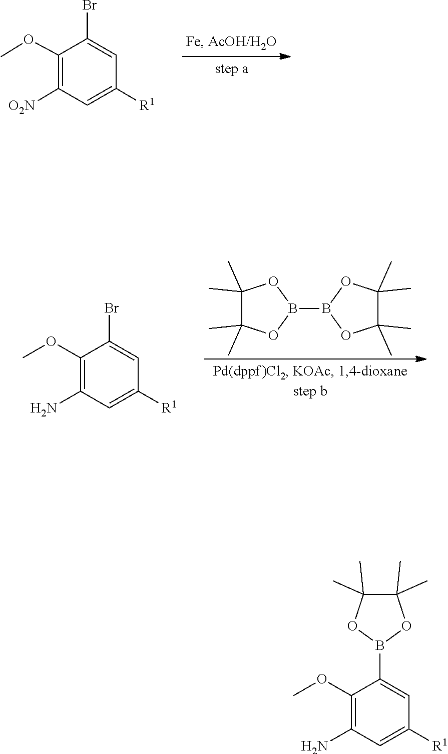

Reduction of a nitro group with iron powder followed by a cross-coupling reaction gave the corresponding compounds (steps a, b).

### Method AC

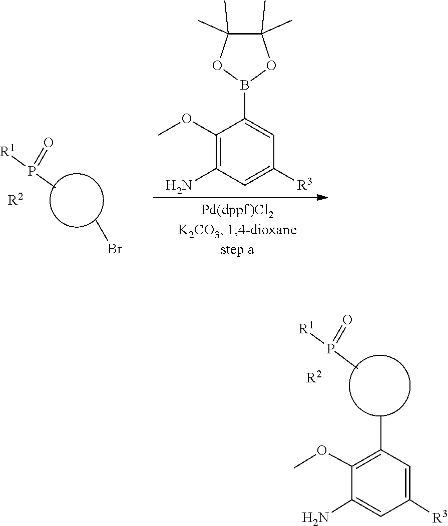

The desired products were obtained through Suzuki coupling reaction (step a).

### Method AD

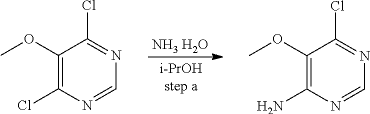

The final compound was obtained through S_{N}Ar reaction (step a).

### Method AE

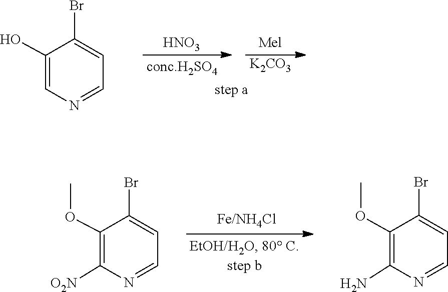

Nitration of 4-bromopyridin-3-ol with HNO₃ and methylation of hydroxyl group with iodomethane gave the corresponding intermediate (step a). Treatment with iron powder yielded pyridin-2-amine derivative (step b).

### Method AF

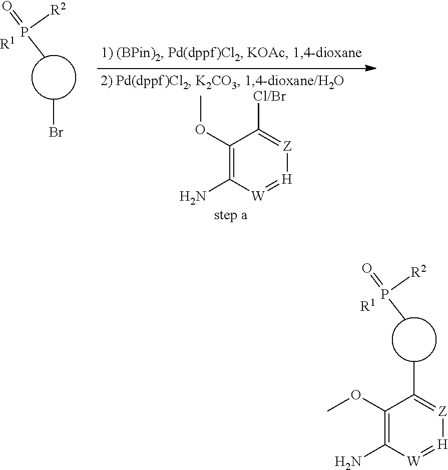

The final compounds were obtained through Suzuki coupling reaction (step a).

### Method AG

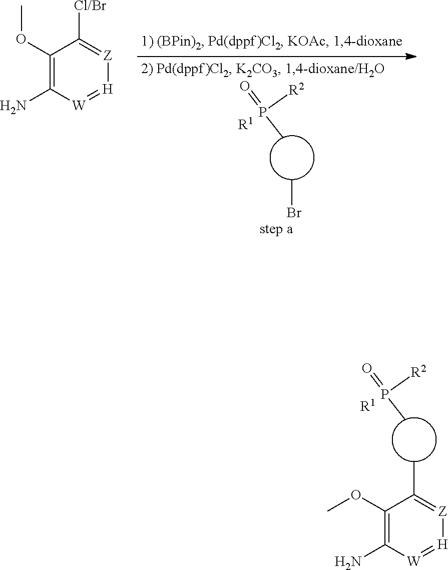

The final compounds were obtained through Suzuki coupling reaction (step a).

### Method AH

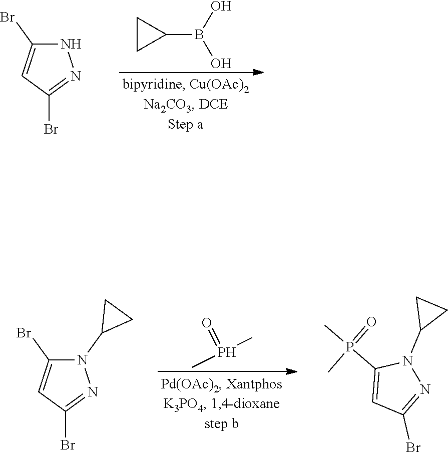

The desired compound was obtained through copper-mediated coupling followed by Buchwald-Hartwig reaction (steps a, b).

### Method AI

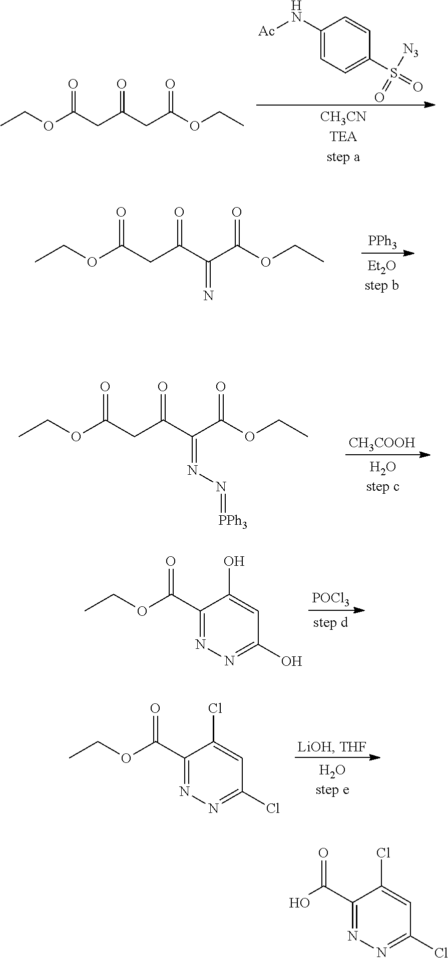

Regitz diazo transfer reaction of diethyl 3-oxopentanedioate was carried out with arylsulfonylazides (step a). Treatment with triphenylphosphine followed by a cyclization reaction yielded pyridazine derivative (steps b, c). Chlorination of the hydroxyl group with phosphorus oxychloride followed by ester hydrolysis provided the final compound (steps d, e).

### Method AJ

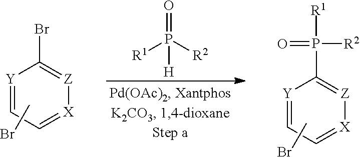

The various benzene or heterocyclic dibromide derivatives were coupled with alkyl phosphorus oxide to give the desired products (step a).

### Method AK

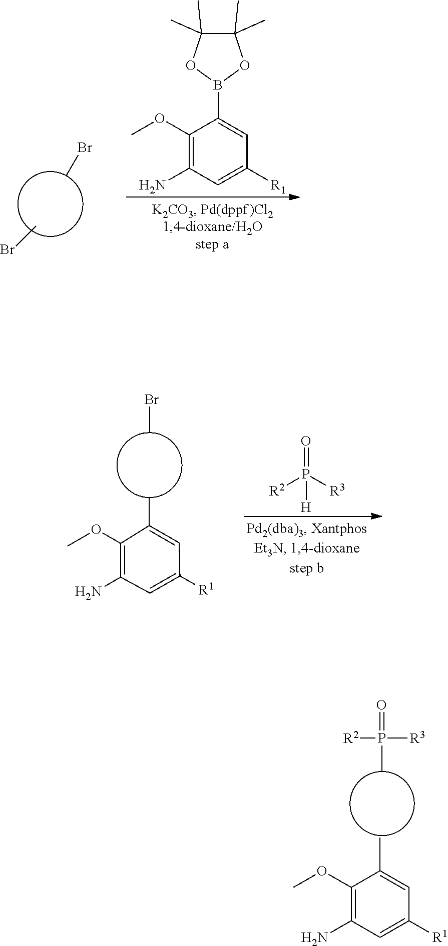

The desired compounds were obtained through Suzuki reaction and Buchwald-Hartwig reaction (steps a, b)

### Method AL

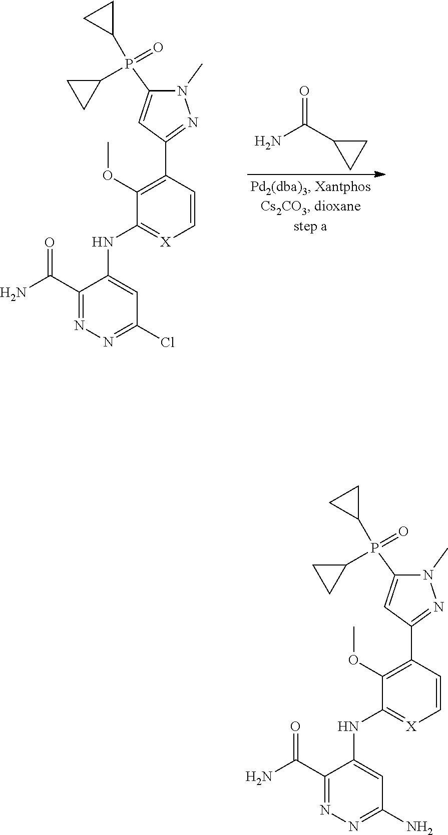

The final compounds were obtained through Buchwald-Hartwig reaction while cyclopropanecarbonyl was hydrolyzed during this process.

### Method AM

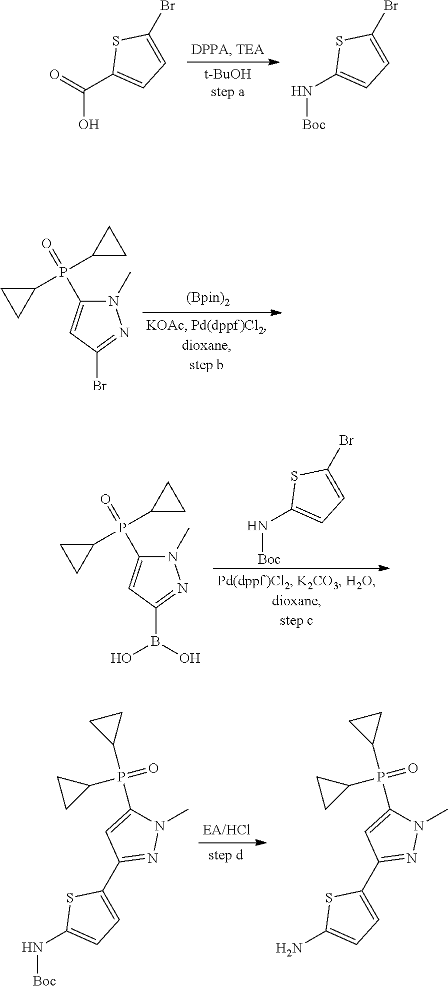

Curtius rearrangement reaction of 5-bromothiophene-2-carboxylic acid with DPPA and t-BuOH gave tert-butyl (5-bromothiophen-2-yl)carbamate (step a), which underwent Miyaura-Suzuki coupling between pyrazole and thiophen afforded the corresponding intermediate (steps b, c). De-protection with EA/HCl led to the desired compound (step d).

### Method AN

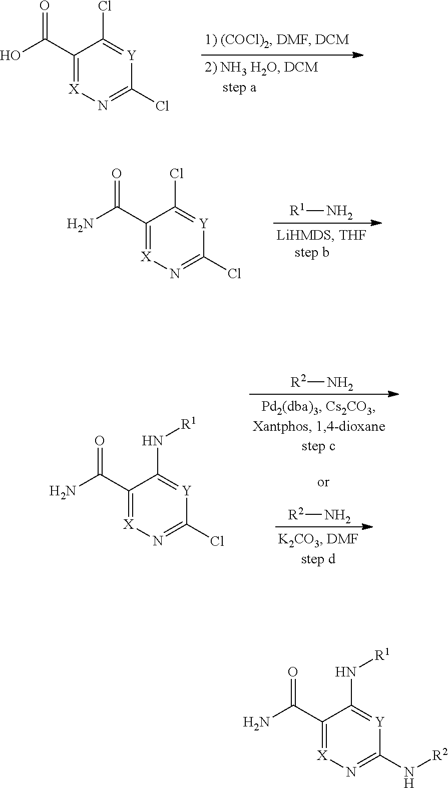

Activation of the carboxylic acid using oxalyl chloride facilitated the addition-elimination process to form the corresponding amides (step a). S_{N}Ar reaction was carried out under basic conditions (step b). Palladium-catalyzed Buchwald-Hartwig (step c) or S_{N}Ar reaction yielded the desired compounds (step d).

### Method AO

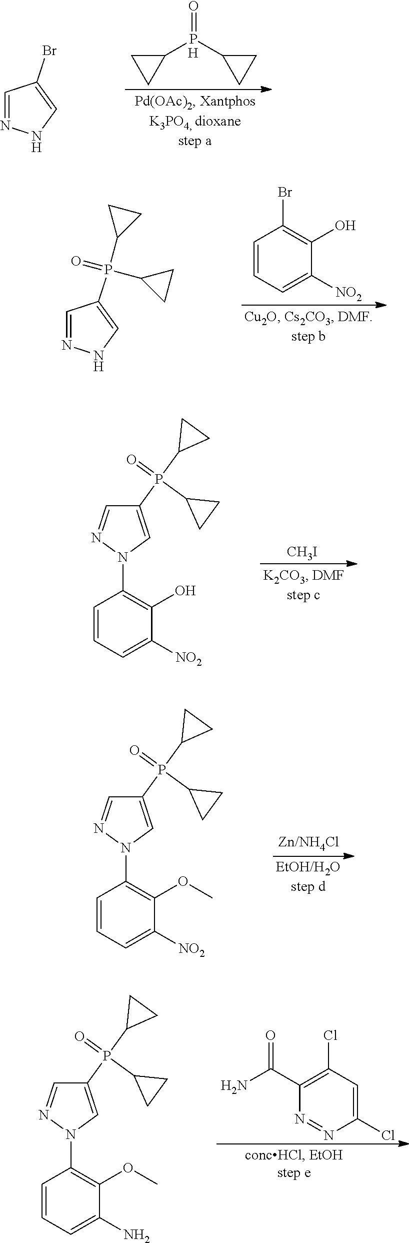

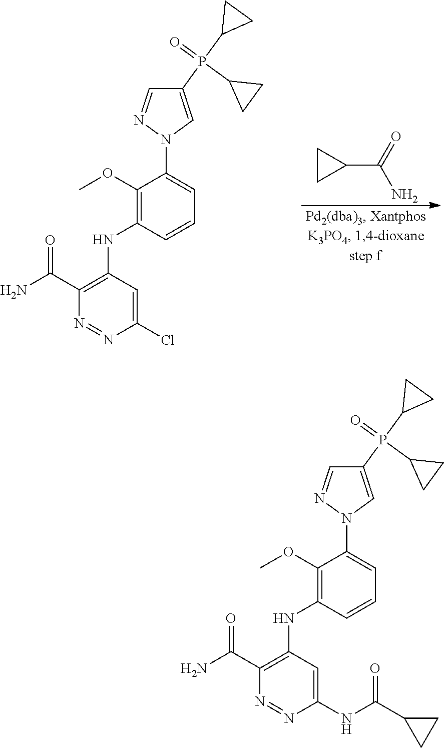 [SMILES: COc1c(Nc2cc(Cl)nnc2C(N)=O)cccc1-n1cc(P(=O)(C2CC2)C2CC2)cn1 ; NC(=O)C1CC1 ; COc1c(Nc2cc(NC(=O)C3CC3)nnc2C(N)=O)cccc1-n1cc(P(=O)(C2CC2)C2CC2)cn1]

Palladium-catalyzed coupling of 4-bromo-1H-pyrazole and dicyclopropylphosphine oxide gave dicyclopropyl(1H-pyrazol-4-yl)phosphine oxide (step a), which was coupled with 2-bromo-6-nitrophenol to give the corresponding intermediate (step b). Methylation with CH₃I was followed by nitro reduction with zinc powder to give (1-(3-amino-2-methoxyphenyl)-1H-pyrazol-4-yl)dicyclopropylphosphine oxide (step d). S_{N}Ar reaction was carried out under acidic conditions followed by Buchwald-Hartwig reaction to give the desired compound (steps e, f).

### Method AP

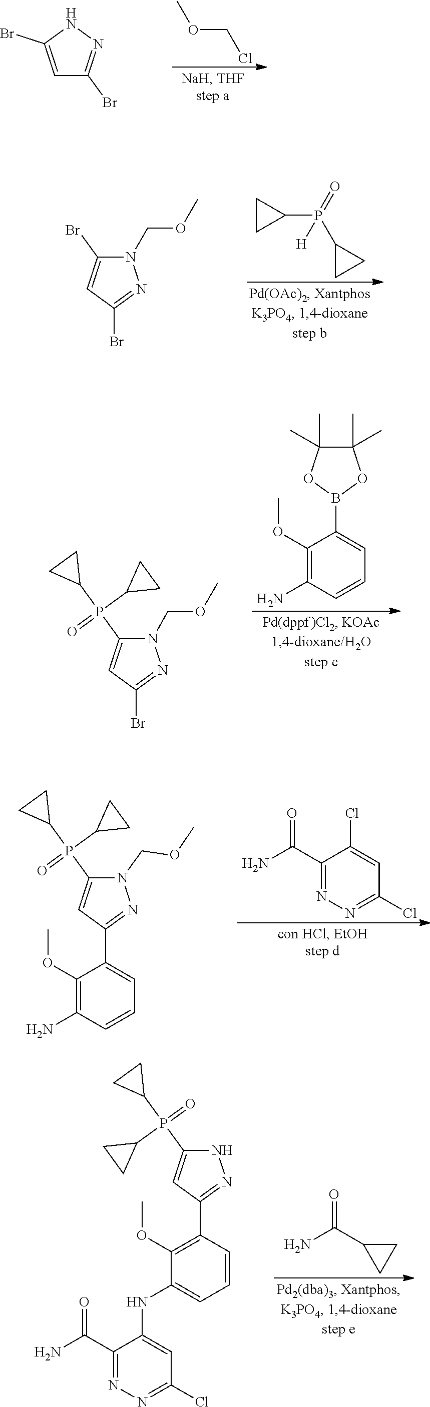

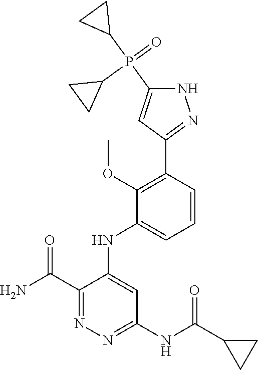 [SMILES: COc1c(Nc2cc(NC(=O)C3CC3)nnc2C(N)=O)cccc1-c1cc(P(=O)(C2CC2)C2CC2)[nH]n1]

Protection of N-pyrazol with MOMCl (step a) followed by coupling reaction between 3,5-dibromo-1-(methoxymethyl)-1H-pyrazole and dicyclopropylphosphine oxide gave the corresponding intermediate (step b), which reacted with 2-methoxy-3-(4,4,5,5-tetramethyl-1,3,2-dioxaborolan-2-yl)aniline through Suzuki reaction (step c). S_{N}Ar reaction was carried out under acidic conditions followed by Buchwald-Hartwig reaction to give the desired compound (steps d, e).

### Method AQ

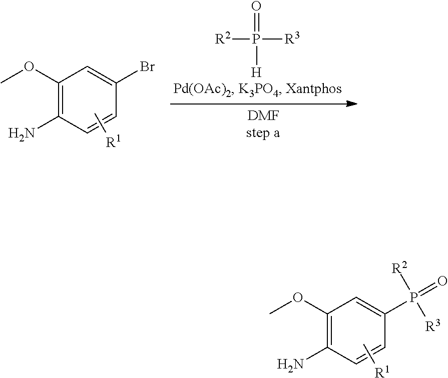

Coupling of the starting materials with alkyl phosphorus oxide gave the desired compounds (step a).

### Method AR

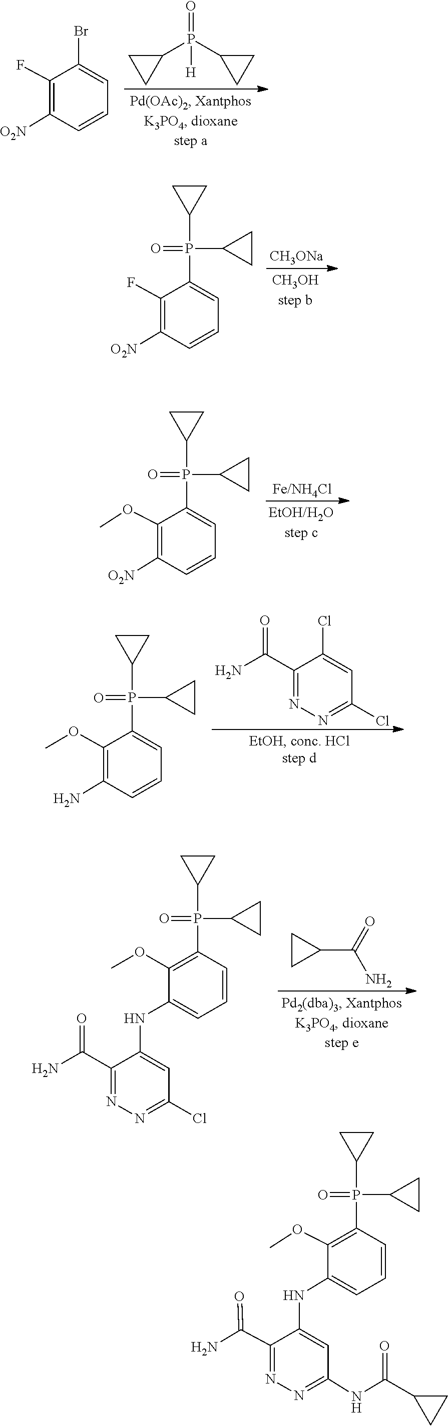

Coupling reaction of the starting material with 1-bromo-2-fluoro-3-nitrobenzene (step a) followed by substitution of fluorine on C2 of the aryl ring with sodium methoxide gave the corresponding intermediate (step b). Reduction of nitro with iron powder followed by S_{N}Ar and Buchwald-Hartwig reaction gave the desired product (steps c, d, e).

### EXAMPLES

General reaction progress was monitored by analytical thin layer chromatography performed on silica gel HSGF254 pre-coated plates. Organic solutions were dried over anhydrous Na₂SO₄, and the solvents were removed under reduced pressure. Final compounds were purified with silica gel 100-200 mesh for column chromatography. ¹H NMR were obtained on 300 MHz (Varian) spectrometer, and ¹³C NMR were obtained on 151 MHz or 101 MHz (Varian) spectrometer. Chemical shifts were given in ppm using tetramethylsilane as internal standard. Mass spectra were obtained using an Agilent 1100 LC/MSD Trap SL version Mass Spectrometer. HRMS analysis was recorded on an Agilent 6540 UHD Accurate-Mass Q-TOF LC/MS.

### Example 1, Methods AA, AB, AC, AI, AN

### Preparation of 6-(cyclopropanecarboxamido)-4-((3-(5-(dicyclopropylphosphoryl)-1-methyl-1H-pyrazol-3-yl)-2-methoxyphenyl)amino)pyridazine-3-carboxamide (A1)

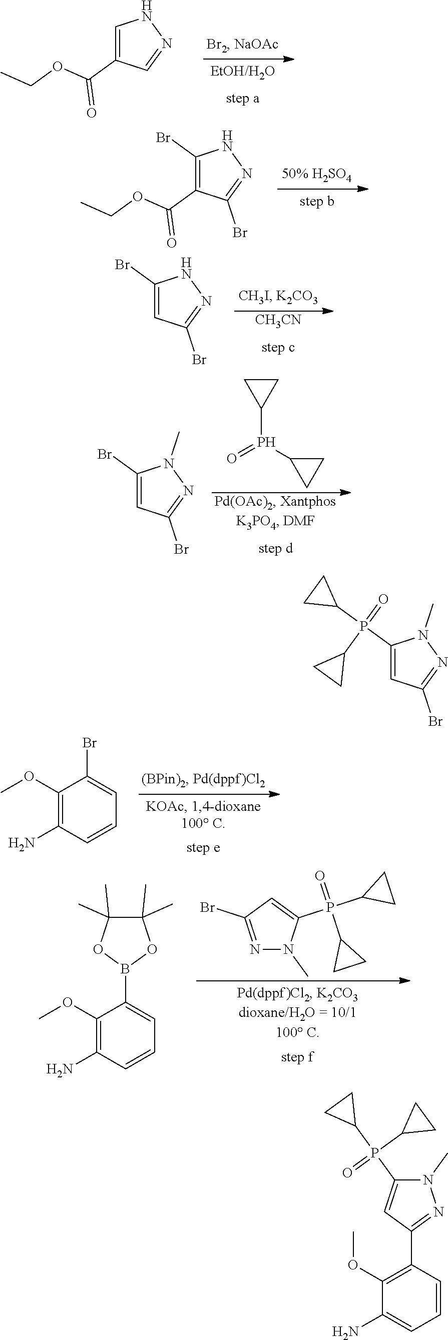

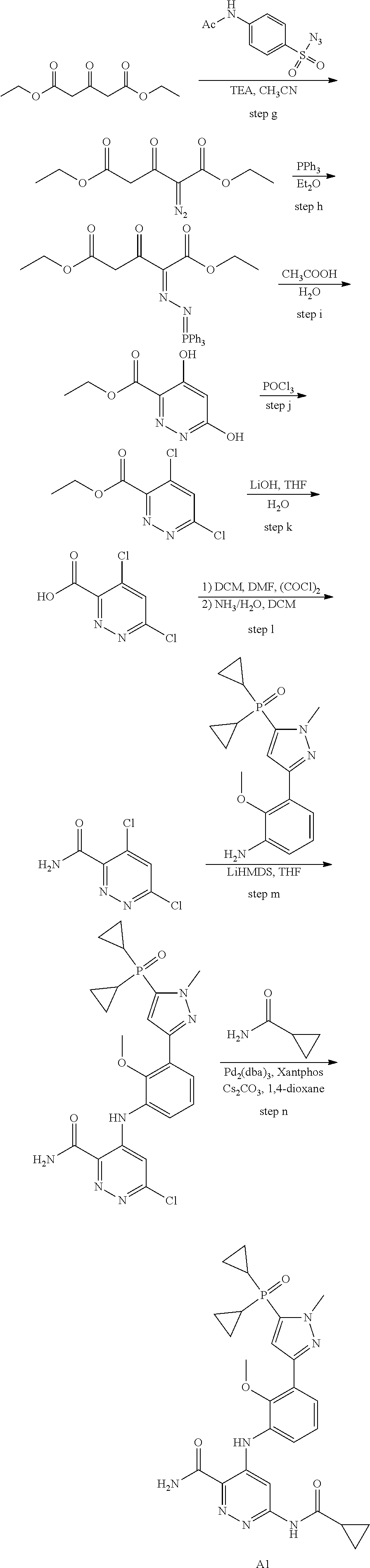

Step a. Ethyl 3,5-dibromo-1H-pyrazole-4-carboxylate: To a solution of ethyl 1H-pyrazole-4-carboxylate (3.0 g, 21.3 mmol) in ethanol/water (18 mL/27 mL) was added NaOAc (6.9 g, 95.0 mmol) and bromide (8.4 g, 53.0 mmol) dropwise. The mixture was stirred at room temperature for 4 hours. Saturated Na₂SO₃ aqueous solution (60 mL) was added to quench the reaction. The aqueous layer was extracted with dichloromethane (60 mL×3). The organic layers were combined, dried by Na₂SO₄ and concentrated to give the desired compound (6.1 g, 96%) as a white solid. ¹H NMR (400 MHz, CDCl₃): δ 4.37-4.19 (m, 2H), 1.42-1.23 (m, 3H). LC-MS: 296.9 [M+H]⁺.

Step b. 3,5-Dibromo-1H-pyrazole: Ethyl 3,5-dibromo-1H-pyrazole-4-carboxylate (3.0 g, 10.0 mmol) was dissolved in 50% H₂SO₄ (30 mL). The solution was stirred at 160° C. for 2 hours. After cooling to room temperature, saturated NaHCO₃ aqueous solution (100 mL) was added to neutralize the acid. The aqueous layer was extracted with ethyl acetate (20 mL×4). The organic layers were combined, dried by Na₂SO₄ and concentrated. The residue was purified by silica gel chromatography column (PE/EA=10/1) to give the final compound (1.4 g, 62%) as a white solid. ¹H NMR (300 MHz, DMSO-d₆): δ 13.92 (s, 1H), 6.58 (s, 1H). LC-MS: 225.0 [M+H]⁺.

Step c. 3,5-Dibromo-1-methyl-1H-pyrazole: To a solution of 3,5-dibromo-1H-pyrazole (500 mg, 2.2 mmol) in acetonitrile (10 mL) was added K₂CO₃ (607 mg, 4.4 mmol) and iodomethane (369 mg, 2.6 mmol). The mixture was stirred at 80° C. overnight. The solvent was removed and the residue was purified by silica gel chromatography column (PE/EA=10/1) to give the final compound (350 mg, 66%) as a colorless oil. ¹H NMR (400 MHz, CDCl₃): δ 6.29 (s, 1H), 3.84 (s, 3H). LC-MS: 239.0 [M+H]⁺.

Step d. (3-Bromo-1-methyl-1H-pyrazol-5-yl)dicyclopropylphosphine oxide: To a solution of 3,5-dibromo-1-methyl-1H-pyrazole (1.5 g, 6.3 mmol) in DMF (5 mL) was added K₃PO₄ (1.7 g, 12.6 mmol), dicyclopropylphosphine oxide (1.5 g, 11.5 mmol), Pd(OAc)₂ (135 mg, 0.6 mmol) and Xantphos (347 mg, 0.6 mmol). The mixture was stirred at 110° C. under N₂ atmosphere for 2 hours. The solvent was concentrated and the residue was purified by silica gel chromatography column (PE/EA=10/1) to give the final compound (470 mg, 26%) as a light yellow oil. ¹H-NMR (300 MHz, CDCl₃): δ 6.59 (d, J=1.2 Hz, 1H), 4.14 (s, 3H), 1.04-0.93 (m, 10H). LC-MS: 289.0 [M+H]⁺.

Step e. 2-Methoxy-3-(4,4,5,5-tetramethyl-1,3,2-dioxaborolan-2-yl)aniline: To a solution of 3-bromo-2-methoxyaniline (6.1 g, 30 mmol) in 1,4-dioxane (60 mL) was added (Bpin)₂ (11.4 g, 45 mmol), KOAc (8.8 g, 90 mmol) and Pd(dppf)Cl₂ (1.3 g, 1.8 mmol). The mixture was stirred at 100° C. under N₂ atmosphere overnight. The solvent was removed and the residue was purified by silica gel chromatography column (PE/EA=4/1) to give the final compound (7.0 g, 93%) as a yellow oil. ¹H NMR (300 MHz, CDCl₃): δ 7.11 (d, J=6.9 Hz, 1H), 6.93 (t, J=7.5 Hz, 1H), 6.85 (d, J=7.2 Hz, 1H), 4.86 (s, 2H), 3.81 (s, 3H), 1.36 (s, 12H). LC-MS: 250.1 [M+H]⁺.

Step f. (3-(3-Amino-2-methoxyphenyl)-1-methyl-1H-pyrazol-5-yl)dicyclopropylphosphine oxide: To a solution of 2-methoxy-3-(4,4,5,5-tetramethyl-1,3,2-dioxaborolan-2-yl)aniline (10.4 g, 41.7 mmol) and (3-bromo-1-methyl-1H-pyrazol-5-yl)dicyclopropylphosphine oxide (8.0 g, 27.8 mmol) in 1,4-dioxane/water (30 mL/3 mL) was added Pd(dppf)Cl₂ (2.0 g, 2.8 mmol) and K₂CO₃ (7.7 g, 55.6 mmol). The mixture was stirred at 110° C. overnight under N₂ atmosphere. The solvent was removed and the residue was purified by silica gel chromatography column (PE/EA=5/1) to give the final compound (8.1 g, 59%) as a white solid. ¹H-NMR (300 MHz, CDCl₃): δ 7.20-7.30 (m, 1H), 7.15-7.08 (m, 1H), 6.97 (t, J=7.5 Hz, 1H), 6.74 (d, J=8.4 Hz, 1H), 4.23 (s, 3H), 3.88 (s, 2H), 3.64 (s, 3H), 1.15-0.75 (m, 10H). LC-MS: 331.9 [M+H]⁺.

Step g. Diethyl 2-diazo-3-oxopentanedioate: To a solution of diethyl 3-oxopentanedioate (7.0 g, 34.6 mmol) in acetonitrile (100 mL) was added Et₃N (3.8 g, 38.1 mmol) and 4-acetamidobenzenesulfonyl azide (8.7 g, 36.9 mmol) at 0° C. The mixture was stirred at room temperature for 1 hour. The solvent was filtered and the filtrate was concentrated. The residue was dissolved in ethyl ether (200 mL) and the solution was filtered again. The filtrate was concentrated to give the crude product (7.4 g, 94%) as a yellow solid.

Step h. Diethyl 3-oxo-2-((triphenyl-15-phosphanylidene)hydrazono)pentanedioate: To a solution of diethyl 2-diazo-3-oxopentanedioate (7.4 g, 32.5 mmol) in ethyl ether (250 mL) was added PPh₃ (9.6 g, 36.5 mmol). The mixture was stirred at room temperature for 48 hours. The solvent was concentrated to give the crude product (17.0 g, 94%) as a yellow oil.

Step i. Ethyl 4,6-dihydroxypyridazine-3-carboxylate: diethyl 3-oxo-2-((triphenyl-15-phosphanylidene)hydrazono)pentanedioate (17.0 g, 34.7 mmol) was dissolved in acetic acid/water (80 mL/8 mL). The mixture was refluxed for 12 hours. The solvent was removed and the residue was rinsed by ethyl acetate (50 mL) to give the desired product (3.0 g, 47%) as a white solid. ¹H NMR (300 MHz, CDCl₃): δ 12.30 (s, 1H), 10.62 (s, 1H), 6.33 (s, 1H), 4.53 (q, J=7.2 Hz, 2H), 1.49 (t, J=7.2 Hz, 3H). LC-MS: 185.1 [M+H]⁺.

Step j. Ethyl 4,6-dichloropyridazine-3-carboxylate: ethyl 4,6-dihydroxypyridazine-3-carboxylate (21 g, 114.1 mmol) was dissolved in POCl₃ (250 mL). The mixture was stirred at 115° C. overnight. The solvent was removed and saturated NaCl aqueous solution (200 mL) was added. The aqueous layer was extracted with ethyl acetate (200 mL×3). The organic layers were combined and concentrated. The residue was purified by silica gel chromatography column (PE/EA=10/1) to give the desired product (10.6 g, 42%) as a brown oil.

Step k. 4,6-Dichloropyridazine-3-carboxylic acid: To a solution of ethyl 4,6-dichloropyridazine-3-carboxylate in tetrahydrofuran (100 mL) was added 1N LiOH (92 mL, 92 mmol). The mixture was stirred at room temperature for 1 hour. The solvent was removed and 1.5 N HCl was added to adjust pH 2. The aqueous layer was extracted with ethyl acetate (200 mL). The organic layer was dried over Na₂SO₄ and concentrated to give the crude product (10.6 g, 90%) as a yellow solid.

Step 1. 4,6-Dichloropyridazine-3-carboxamide: To a solution of 4,6-dichloropyridazine-3-carboxylic acid (2.0 g, 10.4 mmol) in dichloromethane (20 mL) was added oxalyl dichloride (1.3 mL, 15.6 mmol) and DMF (one drop) at 0° C. The mixture was stirred at room temperature for 2 hours. The solvent was removed and the residue was dissolved in dichloromethane (20 mL). Ammonium hydroxide (2 mL) was added to the solution. The mixture was stirred at room temperature overnight. The solvent was removed and the residue was purified by silica gel chromatography column (DCM) to give the final compound (1.8 g, 90%) as a yellow solid. ¹H NMR (300 MHz, DMSO-d₆) δ 8.48 (s, 1H), 8.36 (s, 1H), 8.13 (s, 1H).

Step m. 6-Chloro-4-((3-(5-(dicyclopropylphosphoryl)-1-methyl-1H-pyrazol-3-yl)-2-methoxyphenyl)amino)pyridazine-3-carboxamide: To a solution of 4,6-dichloropyridazine-3-carboxamide (69.6 mg, 0.4 mmol) and (3-(3-amino-2-methoxyphenyl)-1-methyl-1H-pyrazol-5-yl)dicyclopropylphosphine oxide (100 mg, 0.3 mmol) in anhydrous tetrahydrofuran (8 mL) was added 1N LiHMDS (0.8 mL, 0.8 mmol) under N₂ atmosphere. The mixture was stirred at room temperature for 3 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=20/1) to give the final compound (50 mg, 29%) as a yellow solid. ¹H NMR (300 MHz, DMSO-d₆) δ 11.17 (s, 1H), 8.78 (s, 1H), 8.11 (s, 1H), 7.75 (d, J=7.5 Hz, 1H), 7.53 (d, J=7.5 Hz, 1H), 7.31-7.24 (m, 1H), 7.23 (s, 2H), 4.13 (s, 3H), 3.63 (s, 3H), 1.42-1.28 (m, 2H), 0.99-0.87 (m, 4H), 0.84-0.64 (m, 4H).

Step n. 6-(Cyclopropanecarboxamido)-4-((3-(5-(dicyclopropylphosphoryl)-1-methyl-1H-pyrazol-3-yl)-2-methoxyphenyl)amino)pyridazine-3-carboxamide (A1): To a solution of 6-chloro-4-((3-(5-(dicyclopropylphosphoryl)-1-methyl-1H-pyrazol-3-yl)-2-methoxyphenyl)amino)pyridazine-3-carboxamide (16 mg, 0.03 mmol) in 1,4-dioxane (2 mL) was added cyclopropanecarboxamide (8.33 mg, 0.09 mmol), Pd₂(dba)₃ (9.16 mg, 0.01 mmol), Xantphos (5.79 mg, 0.01 mmol) and K₃PO₄ (3.9 g, 12.0 mmol). The mixture was stirred at 120° C. under N₂ atmosphere in a microwave apparatus for 2 hours. The solvent was filtered and the filtrate was concentrated. The residue was purified by silica gel chromatography column (DCM/MeOH=20/1) to give the crude product, which was rinsed by Et₂O to give the desired product (7 mg, 40%) as a white solid.

### Example 2, Methods AA, AB, AC, AN

### Preparation of 6-(cyclopropanecarboxamido)-4-((3-(5-(dicyclopropylphosphoryl)-1-methyl-1H-pyrazol-3-yl)-2-methoxyphenyl)amino)nicotinamide (A2)

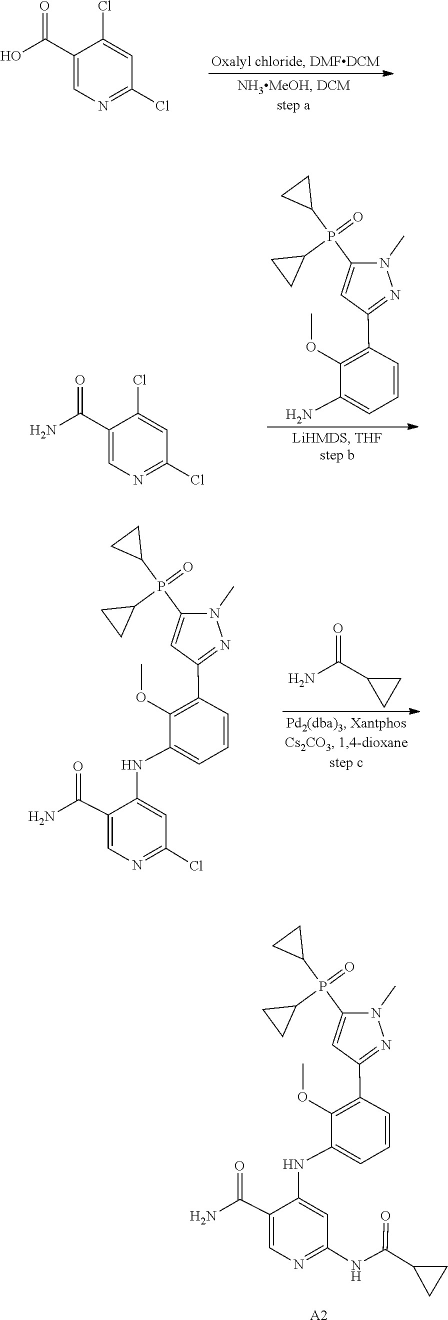

Step a. 4,6-Dichloronicotinamide: To a solution of 4,6-dichloronicotinic acid (500 mg, 2.6 mmol) in dichloromethane (20 mL) was added oxalyl dichloride (434 mg, 3.4 mmol) and DMF (one drop) at 0° C. The mixture was stirred at room temperature for 2 hours. The solvent was removed and the residue was dissolved in dichloromethane (20 mL). Ammonia 7M in Methanol (1.1 mL, 7.7 mmol) was added to the solution. The mixture was stirred at room temperature overnight. The solvent was removed and the residue was purified by silica gel chromatography column (DCM) to give the final compound (240 mg, 48%) as a white solid. ¹H-NMR (400 MHz, DMSO-d₆): δ 8.49 (s, 1H), 8.11 (s, 1H), 7.88 (s, 2H).

Step b. 6-Chloro-4-((3-(5-(dicyclopropylphosphoryl)-1-methyl-1H-pyrazol-3-yl)-2-methoxyphenyl)amino)nicotinamide: To a solution of 4,6-dichloronicotinamide (37 mg, 0.19 mmol) and (3-(3-amino-2-methoxyphenyl)-1-methyl-1H-pyrazol-5-yl)dicyclopropylphosphine oxide (66 mg, 0.20 mmol) in anhydrous tetrahydrofuran (8 mL) was added 1N LiHMDS (0.44 mL, 0.44 mmol) under N₂ atmosphere. The mixture was stirred at room temperature for 3 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=20/1) to give the final compound (53 mg, 57%) as a yellow solid. ¹H-NMR (300 MHz, CDCl₃): δ 10.86 (s, 1H), 8.60 (s, 1H), 8.35 (s, 1H), 7.39 (s, 1H), 7.71 (d, J=7.8 Hz, 1H), 7.45 (d, J=7.2 Hz, 1H), 7.27 (d, J=7.8 Hz, 1H), 7.22 (s, 1H), 6.93 (s, 1H), 4.12 (s, 3H), 3.61 (s, 3H), 1.45-1.26 (m, 2H), 0.99-0.75 (m, 8H).

Step c. 6-(Cyclopropanecarboxamido)-4-((3-(5-(dicyclopropylphosphoryl)-1-methyl-1H-pyrazol-3-yl)-2-methoxyphenyl)amino)nicotinamide (A2): To a solution of 6-chloro-4-((3-(5-(dicyclopropylphosphoryl)-1-methyl-1H-pyrazol-3-yl)-2-methoxyphenyl)amino)nicotinamide (53 mg, 0.11 mmol) in 1,4-dioxane (25 mL) was added cyclopropanecarboxamide (28 mg, 0.33 mmol), Pd₂(dba)₃ (9 mg, 0.01 mmol), Xantphos (5 mg, 0.01 mmol) and Cs₂CO₃ (72 mg, 0.22 mmol). The mixture was stirred at 130° C. under N₂ atmosphere in a microwave apparatus for 2 hours. The solvent was filtered and the filtrate was concentrated. The residue was purified by silica gel chromatography column (DCM/MeOH=30/1) to give the crude product, which was rinsed by Et₂O to give the desired product (10 mg, 17%) as a yellow solid.

### Example 3, Methods AA, AD, AF, AI, AN

### Preparation of 6-(cyclopropanecarboxamido)-4-((6-(5-(dicyclopropylphosphoryl)-1-methyl-1H-pyrazol-3-yl)-5-methoxypyrimidin-4-yl)amino)pyridazine-3-carboxamide (A3)

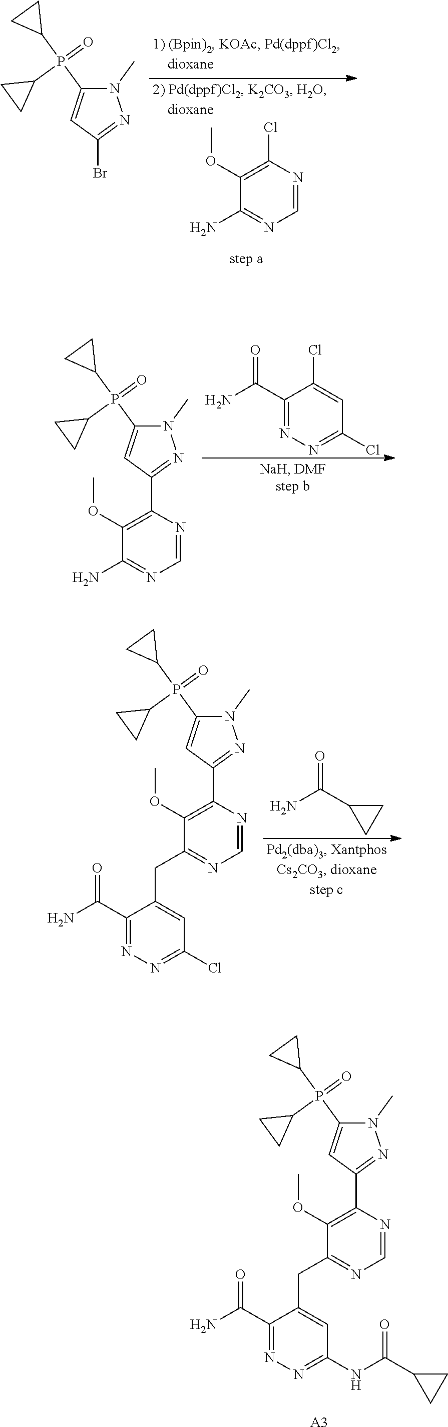

Step a. (3-(6-Amino-5-methoxypyrimidin-4-yl)-1-methyl-1H-pyrazol-5-yl)dicyclopropylphosphine oxide: To a solution of (3-bromo-1-methyl-1H-pyrazol-5-yl)dicyclopropylphosphine oxide (500 mg, 1.7 mmol) in 1,4-dioxane (20 mL) was added (Bpin)₂ (635 mg, 1.5 mmol), KOAc (500 mg, 5.1 mmol), Pd(dppf)Cl₂ (124 mg, 0.17 mmol). The mixture was stirred at 100° C. under N₂ atmosphere overnight. To above mixture was added water (1 mL), K₂CO₃ (469 mg, 3.4 mmol), Pd(dppf)Cl₂ (124 mg, 0.17 mmol) and 6-chloro-5-methoxypyrimidin-4-amine (270 mg, 1.7 mmol). The mixture was stirred at 100° C. for 10 hours. The solvent was removed and the residue was purified by silica gel chromatography column (DCM/MeOH/NH₃·H₂O=50/2/1) to give the desired product (530 mg, 93%) as a black solid.

Step b. 6-Chloro-4-((6-(5-(dicyclopropylphosphoryl)-1-methyl-1H-pyrazol-3-yl)-5-methoxypyrimidin-4-yl)amino)pyridazine-3-carboxamide: To a solution of 4,6-dichloropyridazine-3-carboxamide (128 mg, 0.67 mmol) and (3-(6-amino-5-methoxypyrimidin-4-yl)-1-methyl-1H-pyrazol-5-yl)dicyclopropylphosphine oxide (150 mg, 3 0.45 mmol) in anhydrous tetrahydrofuran (8 mL) was added NaH (108 mg, 2.25 mmol) under N₂ atmosphere. The mixture was stirred at room temperature for 24 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=20/1) to give the final compound (50 mg, 23%) as a yellow oil. ¹H-NMR (300 MHz, DMSO-d₆): δ 12.94 (s, 1H), 9.25 (s, 1H), 8.99 (s, 1H), 8.75 (s, 1H), 8.35 (s, 1H), 7.45 (s, 1H), 4.20 (s, 3H), 3.92 (s, 3H), 1.53-1.30 (m, 2H), 0.97-0.73 (m, 8H). LC-MS: m/z 488.7 [M+H]⁺.

Step c. 6-(Cyclopropanecarboxamido)-4-((6-(5-(dicyclopropylphosphoryl)-1-methyl-1H-pyrazol-3-yl)-5-methoxypyrimidin-4-yl)amino)pyridazine-3-carboxamide (A3): To a solution of 6-chloro-4-((6-(5-(dicyclopropylphosphoryl)-1-methyl-1H-pyrazol-3-yl)-5-methoxypyrimidin-4-yl)amino)pyridazine-3-carboxamide (50 mg, 0.10 mmol) in 1,4-dioxane (2 mL) was added cyclopropanecarboxamide (25 mg, 0.30 mmol), Pd₂(dba)₃ (9 mg, 0.01 mmol), Xantphos (5 mg, 0.01 mmol) and Cs₂CO₃ (65 mg, 0.20 mmol). The mixture was stirred at 130° C. under N₂ atmosphere in a microwave apparatus for 2 hours. The solvent was filtered and the filtrate was concentrated. The residue was purified by silica gel chromatography column (DCM/MeOH=30/1) to give the crude product, which was rinsed by Et₂O to give the desired product (4 mg, 17%) as a yellow solid.

### Example 4, Methods AA, AB, AC, AI, AN

### Preparation of 4-((3-(5-(dicyclopropylphosphoryl)-1-methyl-1H-pyrazol-3-yl)-2-methoxyphenyl)amino)-6-(pyridin-2-ylamino)pyridazine-3-carboxamide (A4)

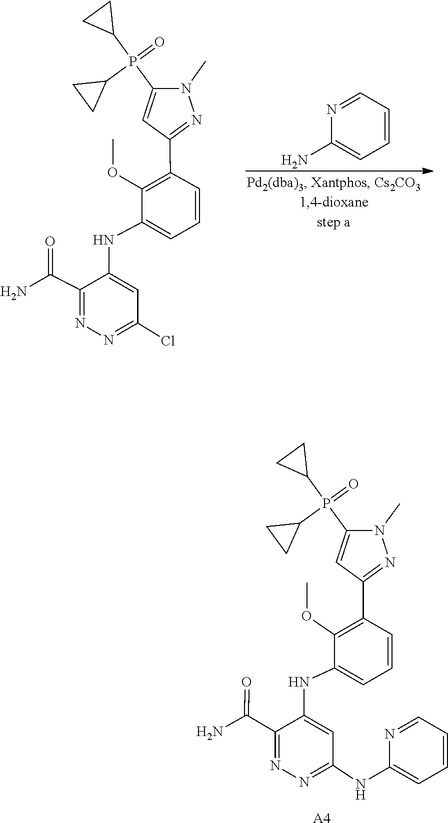

Step a. 4-((3-(5-(Dicyclopropylphosphoryl)-1-methyl-1H-pyrazol-3-yl)-2-methoxyphenyl)amino)-6-(pyridin-2-ylamino)pyridazine-3-carboxamide (A4): To a solution of 6-chloro-4-((3-(5-(dicyclopropylphosphoryl)-1-methyl-1H-pyrazol-3-yl)-2-methoxyphenyl)amino)pyridazine-3-carboxamide (100 mg, 0.2 mmol) in 1,4-dioxane (2 mL) was added pyridin-2-amine (60 mg, 0.3 mmol), Pd₂(dba)₃ (18 mg, 0.02 mmol), Xantphos (12 mg, 0.02 mmol) and Cs₂CO₃ (128 mg, 0.4 mmol). The mixture was stirred at 130° C. under N₂ atmosphere in a microwave apparatus for 2 hours. The solvent was filtered and the filtrate was concentrated. The residue was purified by silica gel chromatography column (DCM/MeOH=30/1) to give the crude product, which was rinsed by Et₂O to give the desired product (7 mg, 6%) as a yellow solid.

The similar procedure described in Example 4 was carried out to obtain Compounds A5, A9, A12, A15, A17, A20, A21, A22, A23, A34, A35, A36, A37, A38, A39, A40, A41, A42, A43, and A47.

### Example 5, Methods AA, AB, AC, AI, AN

### Preparation of 6-(cyclopropanecarboxamido)-4-((3-(5-(diethylphosphoryl)-1-methyl-1H-pyrazol-3-yl)-2-methoxyphenyl)amino)pyridazine-3-carboxamide (A6)

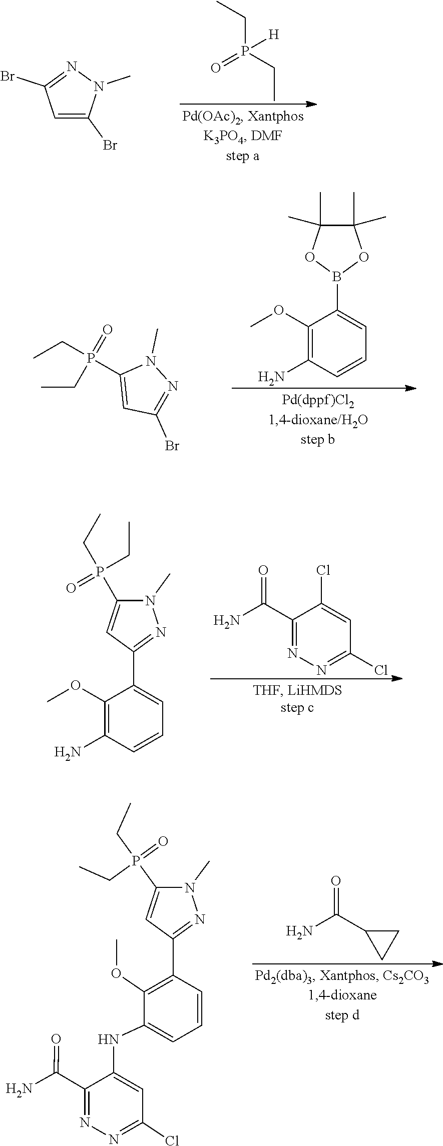

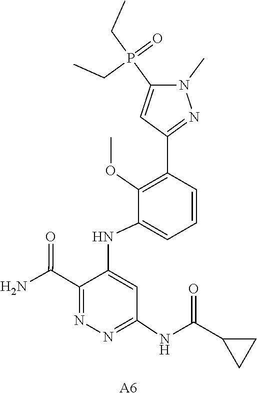 [SMILES: CCP(=O)(CC)c1cc(-c2cccc(Nc3cc(NC(=O)C4CC4)nnc3C(N)=O)c2OC)nn1C ; "A6"]

Step a. (3-Bromo-1-methyl-1H-pyrazol-5-yl)diethylphosphine oxide: To a solution of 3,5-dibromo-1-methyl-1H-pyrazole (500 mg, 2.1 mmol) in DMF (10 mL) was added K₃PO₄ (1.30 g, 6.3 mmol), diethylphosphine oxide (339 mg, 3.2 mmol), Pd(OAc)₂ (135 mg, 0.6 mmol) and Xantphos (116 mg, 0.2 mmol). The mixture was stirred at 130° C. under N₂ atmosphere for 2 hours. The solvent was concentrated and the residue was purified by silica gel chromatography column (DCM/MeOH=100/3) to give the final compound (240 mg, 43%) as a light yellow oil. LC-MS: m/z 265.0 [M+H]⁺.

Step b. (3-(3-Amino-2-methoxyphenyl)-1-methyl-1H-pyrazol-5-yl)diethylphosphine oxide: To a solution of 2-methoxy-3-(4,4,5,5-tetramethyl-1,3,2-dioxaborolan-2-yl)aniline (311 mg, 1.25 mmol) and (3-bromo-1-methyl-1H-pyrazol-5-yl)diethylphosphine oxide (220 mg, 0.83 mmol) in 1,4-dioxane/water (30 mL/3 mL) was added Pd(dppf)Cl₂ (59 mg, 0.08 mmol) and K₂CO₃ (229 mg, 1.6 mmol). The mixture was stirred at 110° C. overnight under N₂ atmosphere. The solvent was removed and the residue was purified by silica gel chromatography column (DCM/MeOH=100/3) to give the compound (240 mg, 94%) as a yellow oil. ¹H-NMR (300 MHz, CDCl₃): δ 7.22 (d, J=6.9 Hz, 1H), 7.02-6.93 (m, 1H), 6.88 (s, 1H), 6.74 (d, J=7.2 Hz, 1H), 4.28 (s, 3H), 3.93 (s, 2H), 3.62 (s, 3H), 2.11-1.95 (m, 4H), 1.30-1.20 (m, 6H).

Step c. 6-Chloro-4-((3-(5-(diethylphosphoryl)-1-methyl-1H-pyrazol-3-yl)-2-methoxyphenyl)amino)pyridazine-3-carboxamide: To a solution of 4,6-dichloropyridazine-3-carboxamide (155 mg, 0.8 mmol) and (3-(3-amino-2-methoxyphenyl)-1-methyl-1H-pyrazol-5-yl)diethylphosphine oxide (165 mg, 0.5 mmol) in anhydrous tetrahydrofuran (8 mL) was added 1N LiHMDS (1.4 mL, 1.4 mmol) under N₂ atmosphere. The mixture was stirred at room temperature for 3 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=20/1) to give the final compound (120 mg, 32%) as a yellow solid. ¹H-NMR (300 MHz, CDCl₃): δ 10.76 (s, 1H), 8.10 (s, 1H), 7.84 (s, 1H), 7.29-7.21 (m, 2H), 7.07-6.97 (m, 1H), 6.95-6.86 (m, 1H), 5.80 (s, 1H), 4.29 (s, 3H), 3.60 (s, 3H), 2.11-1.93 (m, 4H), 1.25-1.12 (m, 6H). LC-MS: m/z 462.7 [M+H]⁺.

Step d. 6-(Cyclopropanecarboxamido)-4-((3-(5-(diethylphosphoryl)-1-methyl-1H-pyrazol-3-yl)-2-methoxyphenyl)amino)pyridazine-3-carboxamide (A6): To a solution of 6-chloro-4-((3-(5-(diethylphosphoryl)-1-methyl-1H-pyrazol-3-yl)-2-methoxyphenyl)amino)pyridazine-3-carboxamide (130 mg, 0.3 mmol) in 1,4-dioxane (2 mL) was added cyclopropanecarboxamide (72 mg, 0.8 mmol), Pd₂(dba)₃ (27 mg, 0.03 mmol), Xantphos (17 mg, 0.03 mmol) and Cs₂CO₃ (65 mg, 0.20 mmol). The mixture was stirred at 130° C. under N₂ atmosphere in a microwave apparatus for 1.5 hours. The solvent was filtered and the filtrate was concentrated. The residue was purified by silica gel chromatography column (DCM/MeOH=30/1) to give the crude product, which was rinsed by Et₂O to give the desired product (14 mg, 10%) as a yellow solid.

The similar procedure described in Example 5 was carried out to get the Compounds A8, A10, A26, A29, A30, A31, and A33.

### Example 6, Methods AB, AK, AI, AN

### Preparation of 6-(cyclopropanecarboxamido)-4-((3-(5-(dimethylphosphoryl)pyrimidin-2-yl)-2-methoxyphenyl)amino)pyridazine-3-carboxamide (A7)

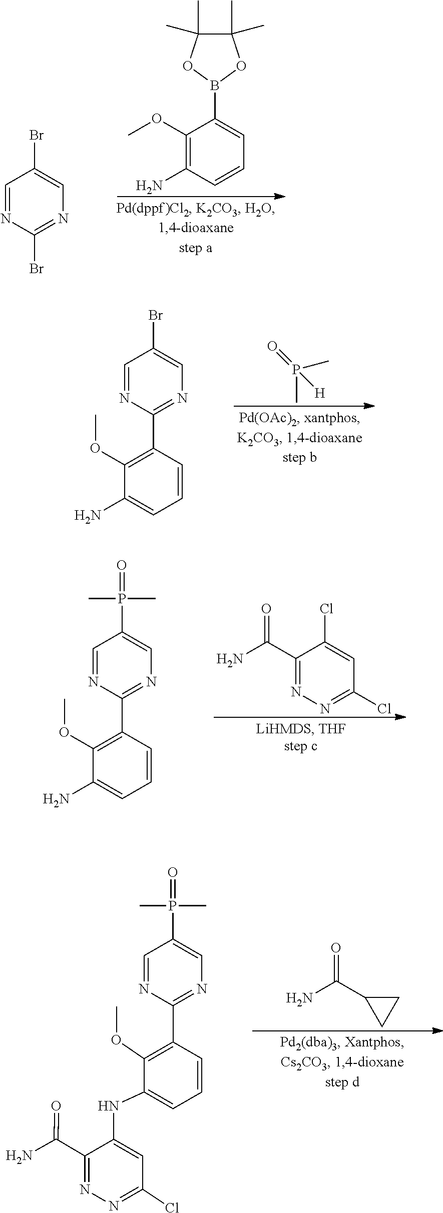

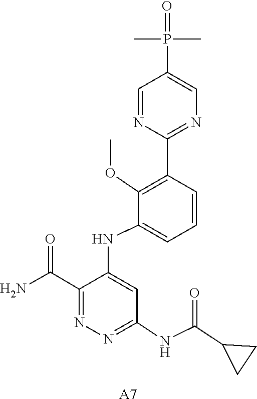 [SMILES: COc1c(Nc2cc(NC(=O)C3CC3)nnc2C(N)=O)cccc1-c1ncc(P(C)(C)=O)cn1 ; "A7"]

Step a. 3-(5-Bromopyrimidin-2-yl)-2-methoxyaniline: To a solution of 2-methoxy-3-(4,4,5,5-tetramethyl-1,3,2-dioxaborolan-2-yl)aniline (1.3 g, 5.1 mmol) and 2,5-dibromopyrimidine (800 mg, 3.4 mmol) in 1,4-dioxane/water (30 mL/3 mL) was added Pd(dppf)Cl₂ (250 mg, 0.34 mmol) and K₂CO₃ (938 mg, 6.8 mmol). The mixture was stirred at 95° C. overnight under N₂ atmosphere. The solvent was removed and the residue was purified by silica gel chromatography column (PE/EA=2/1) to give the compound (645 mg, 68%) as a yellow oil. ¹H-NMR (300 MHz, CDCl₃): δ 8.90 (s, 2H), 7.22-7.16 (m, 1H), 7.08-6.97 (m, 1H), 6.91-6.85 (m, 1H), 3.96 (s, 2H), 3.68 (s, 3H). LC-MS: m/z 280.0 [M+H]⁺.

Step b. (2-(3-Amino-2-methoxyphenyl)pyrimidin-5-yl)dimethylphosphine oxide: To a solution of 3-(5-bromopyrimidin-2-yl)-2-methoxyaniline (645 mg, 2.3 mmol) in 1,4-dioxane (10 mL) was added K₂CO₃ (635 mg, 4.6 mmol), dimethylphosphine oxide (269 mg, 3.5 mmol), Pd(OAc)₂ (52 mg, 0.23 mmol) and Xantphos (133 mg, 0.23 mmol). The mixture was stirred at 120° C. under N₂ atmosphere for 2 hours. The solvent was concentrated and the residue was purified by silica gel chromatography column (DCM/MeOH=100/3) to give the final compound (200 mg, 31%) as a yellow oil. ¹H-NMR (300 MHz, DMSO-d₆): δ 9.15 (s, 1H), 9.13 (s, 1H), 7.28 (d, J=4.8 Hz, 1H), 7.11-7.02 (m, 1H), 6.93 (d, J=7.8 Hz, 1H), 4.01 (s, 2H), 3.74 (s, 3H), 1.87 (d, J=13.2 Hz, 6H). LC-MS: m/z 278.1 [M+H]⁺.

Step c. 6-Chloro-4-((3-(5-(dimethylphosphoryl)pyrimidin-2-yl)-2-methoxyphenyl)amino)pyridazine-3-carboxamide: To a solution of 4,6-dichloropyridazine-3-carboxamide (95 mg, 0.5 mmol) and (2-(3-amino-2-methoxyphenyl)pyrimidin-5-yl)dimethylphosphine oxide (70 mg, 0.25 mmol) in anhydrous tetrahydrofuran (3 mL) was added 1N LiHMDS (0.75 mL, 0.75 mmol) under N₂ atmosphere. The mixture was stirred at room temperature for 2 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=15/1) to give the final compound (25 mg, 23%) as a yellow solid. ¹H-NMR (300 MHz, CDCl₃): δ 10.75 (s, 1H), 9.16 (s, 2H), 8.09 (s, 1H), 7.84 (s, 1H), 7.48 (s, 1H), 7.42-7.26 (m, 2H), 7.00 (s, 1H), 3.78 (s, 3H), 1.89 (d, J=10.2 Hz, 6H).

Step d. 6-(Cyclopropanecarboxamido)-4-((3-(5-(dimethylphosphoryl)pyrimidin-2-yl)-2-methoxyphenyl)amino)pyridazine-3-carboxamide (A7): To a solution of 6-chloro-4-((3-(5-(dimethylphosphoryl)pyrimidin-2-yl)-2-methoxyphenyl)amino)pyridazine-3-carboxamide (24 mg, 0.05 mmol) in 1,4-dioxane (2 mL) was added cyclopropanecarboxamide (14 mg, 0.16 mmol), Pd₂(dba)₃ (5 mg, 0.005 mmol), Xantphos (3 mg, 0.005 mmol) and Cs₂CO₃ (32 mg, 0.10 mmol). The mixture was stirred at 130° C. under N₂ atmosphere in a microwave apparatus for 1.5 hours. The solvent was filtered and the filtrate was concentrated. The residue was purified by silica gel chromatography column (DCM/MeOH=30/1) to give the crude product, which was rinsed by Et₂O to give the desired product (12 mg, 50%) as a yellow solid.

The similar procedure described in Example 6 was carried out to get the Compounds A14 and A16.

### Example 7, Methods AA, AB, AC, AI, AN

### Preparation of 4-((3-(5-(dicyclopropylphosphoryl)-1-methyl-1H-pyrazol-3-yl)-2-methoxyphenyl)amino)-6-(pyrrolidin-1-yl)pyridazine-3-carboxamide (A11)

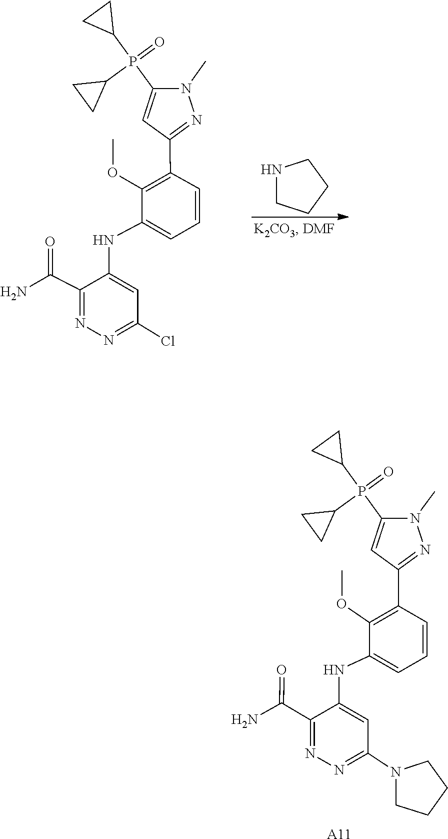 [SMILES: COc1c(Nc2cc(Cl)nnc2C(N)=O)cccc1-c1cc(P(=O)(C2CC2)C2CC2)n(C)n1 ; C1CCNC1 ; COc1c(Nc2cc(N3CCCC3)nnc2C(N)=O)cccc1-c1cc(P(=O)(C2CC2)C2CC2)n(C)n1 ; "A11"]

Step a. 4-((3-(5-(Dicyclopropylphosphoryl)-1-methyl-1H-pyrazol-3-yl)-2-methoxyphenyl)amino)-6-(pyrrolidin-1-yl)pyridazine-3-carboxamide (A11): To a solution of 6-chloro-4-((3-(5-(dicyclopropylphosphoryl)-1-methyl-1H-pyrazol-3-yl)-2-methoxyphenyl)amino)pyridazine-3-carboxamide (50 mg, 0.1 mmol) in DMF (2 mL) was added pyrrolidine (28 mg, 0.4 mmol) and K₂CO₃ (41 mg, 0.3 mmol). The mixture was stirred at 110° C. in a microwave apparatus for 2.5 hours. The reaction mixture was diluted with water (10 mL) and extracted with EA (30 mL×3). The combined organic layer was dried by Na₂SO₄ and concentrated. The residue was purified by silica gel chromatography column (DCM/MeOH=20/1) to give the final compound (22 mg, 17%) as a white solid.

The similar procedure described in Example 7 was carried out to get the Compounds A27, A28, and A32.

### Example 8, Methods AA, AE, AG, AI, AL

### Preparation of 6-amino-4-((4-(5-(dicyclopropylphosphoryl)-1-methyl-H-pyrazol-3-yl)-3-methoxypyridin-2-yl)amino)pyridazine-3-carboxamide (A13)

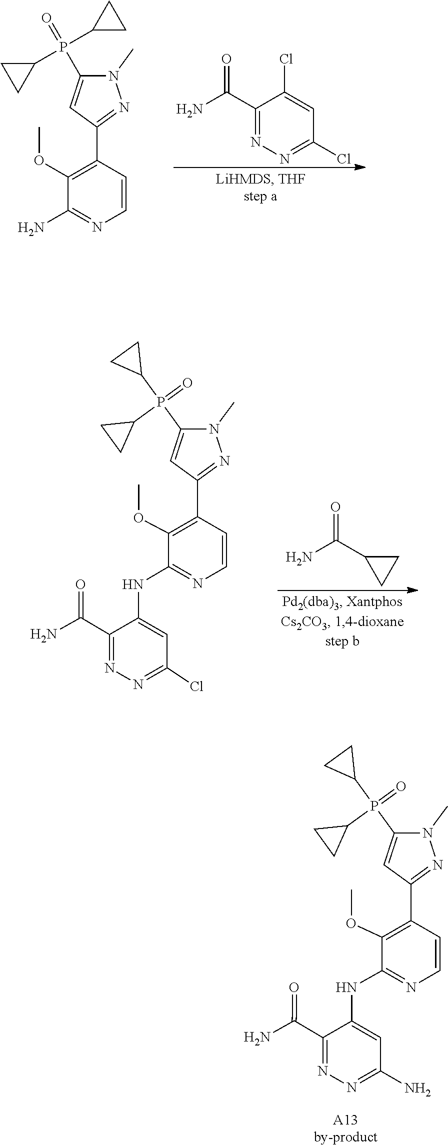 [SMILES: COc1c(-c2cc(P(=O)(C3CC3)C3CC3)n(C)n2)ccnc1N ; NC(=O)c1nnc(Cl)cc1Cl ; COc1c(-c2cc(P(=O)(C3CC3)C3CC3)n(C)n2)ccnc1Nc1cc(Cl)nnc1C(N)=O ; NC(=O)C1CC1 ; COc1c(-c2cc(P(=O)(C3CC3)C3CC3)n(C)n2)ccnc1Nc1cc(N)nnc1C(N)=O]

Step a. 6-Chloro-4-((4-(5-(dicyclopropylphosphoryl)-1-methyl-1H-pyrazol-3-yl)-3-methoxypyridin-2-yl)amino)pyridazine-3-carboxamide: To a solution of 4,6-dichloropyridazine-3-carboxamide (276 mg, 1.4 mmol) and (3-(2-amino-3-methoxypyridin-4-yl)-1-methyl-1H-pyrazol-5-yl)dicyclopropylphosphine oxide (320 mg, 0.96 mmol) in anhydrous tetrahydrofuran (10 mL) was added 1N LiHMDS (2.9 mL, 2.9 mmol) under N₂ atmosphere. The mixture was stirred at room temperature for 24 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=15/1) to give the final compound (116 mg, 25%) as a yellow solid. ¹H-NMR (300 MHz, DMSO-d₆): δ 12.62 (s, 1H), 9.24 (s, 1H), 8.89 (s, 1H), 8.33-8.04 (m, 2H), 7.55 (d, J=4.8 Hz, 1H), 7.39 (s, 1H), 4.17 (s, 3H), 3.79 (s, 3H), 1.52-1.22 (m, 2H), 1.06-0.72 (m, 8H).

Step b. 6-Amino-4-((4-(5-(dicyclopropylphosphoryl)-1-methyl-1H-pyrazol-3-yl)-3-methoxypyridin-2-yl)amino)pyridazine-3-carboxamide (A13): To a solution of 6-chloro-4-((4-(5-(dicyclopropylphosphoryl)-1-methyl-1H-pyrazol-3-yl)-3-methoxypyridin-2-yl)amino)pyridazine-3-carboxamide (30 mg, 0.06 mmol) in 1,4-dioxane (2 mL) was added cyclopropanecarboxamide (15 mg, 0.18 mmol), Pd₂(dba)₃ (5 mg, 0.006 mmol), Xantphos (4 mg, 0.006 mmol) and Cs₂CO₃ (40 mg, 0.12 mmol). The mixture was stirred at 130° C. under N₂ atmosphere in a microwave apparatus for 1.5 hours. The solvent was filtered and the filtrate was concentrated. The residue was purified by silica gel chromatography column (DCM/MeOH=30/1) to give the by-product (4 mg, 14%) as a yellow solid.

### Example 9, Methods AA, AE, AG, AI, AN

### Preparation of 6-(cyclopropanecarboxamido)-4-((4-(5-(dicyclopropylphosphoryl)-1-methyl-1H-pyrazol-3-yl)-3-methoxypyridin-2-yl)amino)pyridazine-3-carboxamide (A18)

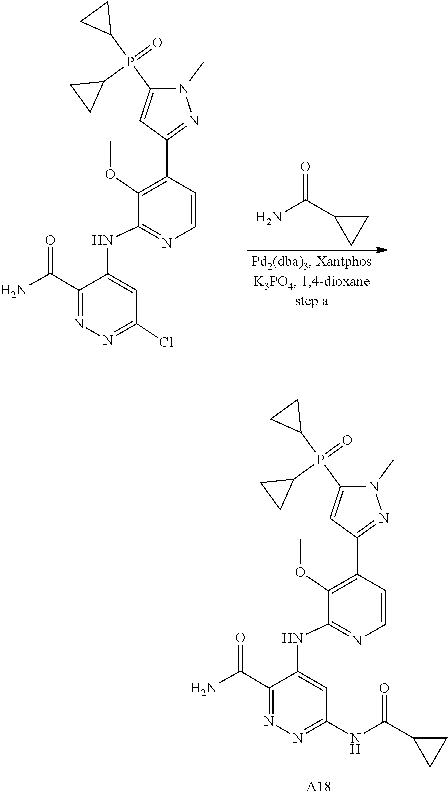 [SMILES: COc1c(-c2cc(P(=O)(C3CC3)C3CC3)n(C)n2)ccnc1Nc1cc(Cl)nnc1C(N)=O ; NC(=O)C1CC1 ; COc1c(-c2cc(P(=O)(C3CC3)C3CC3)n(C)n2)ccnc1Nc1cc(NC(=O)C2CC2)nnc1C(N)=O ; "A18"]

Step a. 6-(Cyclopropanecarboxamido)-4-((4-(5-(dicyclopropylphosphoryl)-1-methyl-1H-pyrazol-3-yl)-3-methoxypyridin-2-yl)amino)pyridazine-3-carboxamide (A18): To a solution of 6-chloro-4-((4-(5-(dicyclopropylphosphoryl)-1-methyl-1H-pyrazol-3-yl)-3-methoxypyridin-2-yl)amino)pyridazine-3-carboxamide (20 mg, 0.04 mmol) in 1,4-dioxane (2 mL) was added cyclopropanecarboxamide (10 mg, 0.12 mmol), Pd₂(dba)₃ (4 mg, 0.004 mmol), Xantphos (4 mg, 0.006 mmol) and K₃PO₄ (17 mg, 0.08 mmol). The mixture was stirred at 120° C. under N₂ atmosphere in a microwave apparatus for 1 hour. The solvent was filtered and the filtrate was concentrated. The residue was purified by silica gel chromatography column (DCM/MeOH=15/1) to give the desired product (8 mg, 14%) as a yellow solid.

### Example 10, Methods AA, AB, AC, AN

### Preparation of 2-(cyclopropanecarboxamido)-4-((3-(5-(dicyclopropylphosphoryl)-1-methyl-1H-pyrazol-3-yl)-2-methoxyphenyl)amino)pyrimidine-5-carboxamide (A19)

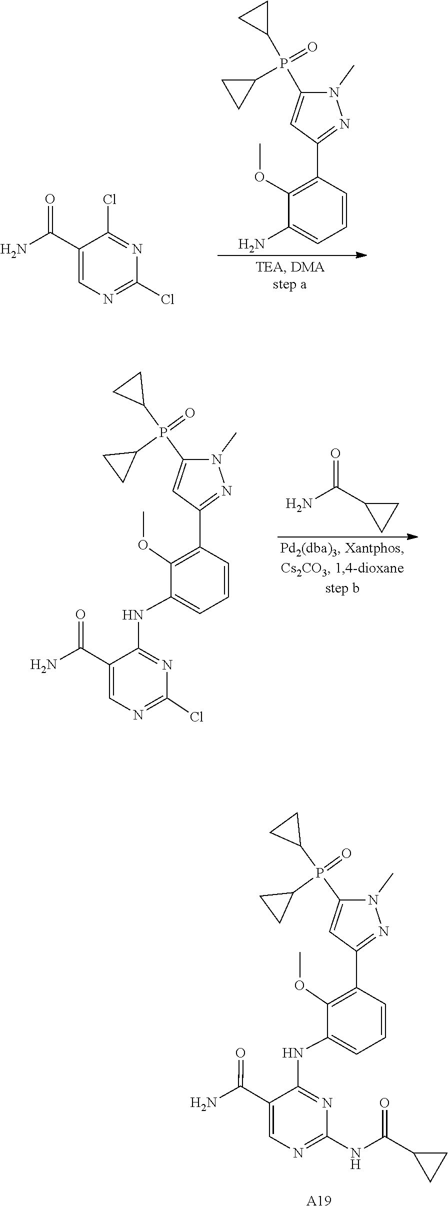 [SMILES: NC(=O)c1cnc(Cl)nc1Cl ; COc1c(N)cccc1-c1cc(P(=O)(C2CC2)C2CC2)n(C)n1 ; COc1c(Nc2nc(Cl)ncc2C(N)=O)cccc1-c1cc(P(=O)(C2CC2)C2CC2)n(C)n1 ; NC(=O)C1CC1 ; COc1c(Nc2nc(NC(=O)C3CC3)ncc2C(N)=O)cccc1-c1cc(P(=O)(C2CC2)C2CC2)n(C)n1 ; "A19"]

Step a. 2-Chloro-4-((3-(5-(dicyclopropylphosphoryl)-1-methyl-1H-pyrazol-3-yl)-2-methoxyphenyl)amino)pyrimidine-5-carboxamide: To a solution of 2,4-dichloropyrimidine-5-carboxamide (122 mg, 0.3 mmol) and (3-(2-amino-3-methoxypyridin-4-yl)-1-methyl-1H-pyrazol-5-yl)dicyclopropylphosphine oxide (210 mg, 0.3 mmol) in DMA (8 mL) was added TEA (128 mg, 1.2 mmol). The mixture was stirred at room temperature overnight. The solvent was concentrated and the residue was purified by silica gel chromatography column (DCM/MeOH=50/1) to give the final compound (130 mg, 42%) as a yellow solid. ¹H-NMR (300 MHz, DMSO-d₆): δ 12.08 (s, 1H), 8.82 (s, 1H), 8.45 (s, 1H), 8.39-8.34 (m, 1H), 7.95 (s, 1H), 7.64-7.52 (m, 1H), 7.30-7.18 (m, 2H), 4.13 (s, 3H), 3.67 (s, 3H), 1.44-1.30 (m, 2H), 0.97-0.71 (m, 8H). LC-MS: m/z 486.7 [M+H]⁺.

Step b. 2-(Cyclopropanecarboxamido)-4-((3-(5-(dicyclopropylphosphoryl)-1-methyl-1H-pyrazol-3-yl)-2-methoxyphenyl)amino)pyrimidine-5-carboxamide (A19): To a solution of 6-chloro-4-((4-(5-(dicyclopropylphosphoryl)-1-methyl-1H-pyrazol-3-yl)-3-methoxypyridin-2-yl)amino)pyridazine-3-carboxamide (125 mg, 0.30 mmol) in 1,4-dioxane (2 mL) was added cyclopropanecarboxamide (65 mg, 0.80 mmol), Pd₂(dba)₃ (27 mg, 0.030 mmol), Xantphos (20 mg, 0.030 mmol) and Cs₂CO₃ (195 mg, 0.60 mmol). The mixture was stirred at 130° C. under N₂ atmosphere in a microwave apparatus for 1.5 hours. The solvent was filtered and the filtrate was concentrated. The residue was purified by silica gel chromatography column (DCM/MeOH=30/1) to give the crude product, which was rinsed by Et₂O to give the desired product (5 mg, 4%) as a yellow solid.

### Example 11, Methods AA, AB, AC, AI, AN

### Preparation of 6-(cyclopropanecarboxamido)-4-((3-(5-(dicyclopropylphosphoryl)-1-(methyl-d3)-1H-pyrazol-3-yl)-2-methoxyphenyl)amino)pyridazine-3-carboxamide (A24)

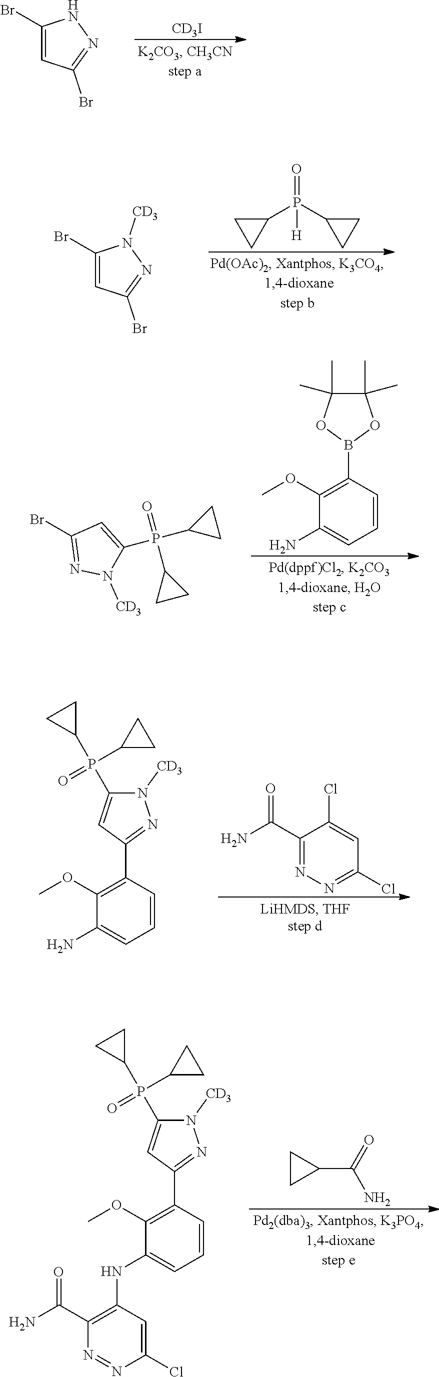

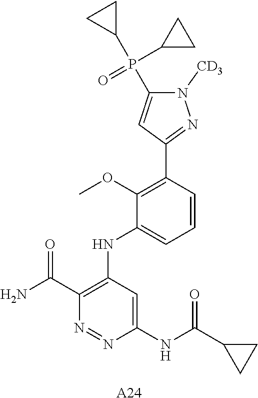 [SMILES: [2H]C([2H])([2H])n1nc(-c2cccc(Nc3cc(NC(=O)C4CC4)nnc3C(N)=O)c2OC)cc1P(=O)(C1CC1)C1CC1 ; "A24"]

Step a. 3,5-Dibromo-1-(methyl-d₃)-1H-pyrazole: To a solution of 3,5-dibromo-1H-pyrazole (500 mg, 2.2 mmol) in acetonitrile (10 mL) was added K₂CO₃ (613 mg, 4.4 mmol) and iodomethane-d₃ (0.17 mL, 2.7 mmol). The mixture was stirred at 80° C. for 2 hours. The solvent was removed and the residue was purified by silica gel chromatography column (PE/EA=10/1) to give the final compound (443 mg, 82%). ¹H-NMR (300 MHz, CDCl₃): δ 6.28 (s, 1H).

Step b. (3-Bromo-1-(methyl-d₃)-1H-pyrazol-5-yl)dicyclopropylphosphine oxide: To a solution of 3,5-dibromo-1-(methyl-d₃)-1H-pyrazole (3.2 g, 13 mmol) in 1,4-dioxane (5 mL) was added K₃PO₄ (5.6 g, 26 mmol), dicyclopropylphosphine oxide (3.4 g, 26 mmol), Pd(OAc)₂ (295 mg, 1.3 mmol) and Xantphos (761 mg, 1.3 mmol). The mixture was stirred at 110° C. under N₂ atmosphere for 2 hours. The solvent was concentrated and the residue was purified by silica gel chromatography column (DCM/MeOH=100/3) to give the final compound (1.8 g, 47%) as a light yellow solid. ¹H-NMR (300 MHz, CDCl₃): δ 6.59 (d, J=1.2 Hz, 1H), 4.14 (s, 3H), 1.04-0.93 (m, 10H). ¹H-NMR (300 MHz, CDCl₃): δ 6.59 (s, 1H), 1.09-0.86 (m, 10H). LC-MS: m/z 291.8 [M+H]⁺.

Step c. (3-(3-Amino-2-methoxyphenyl)-1-(methyl-d₃)-1H-pyrazol-5-yl)dicyclopropylphosphine oxide: To a solution of 2-methoxy-3-(4,4,5,5-tetramethyl-1,3,2-dioxaborolan-2-yl)aniline (1.8 g, 7.2 mmol) and (3-bromo-1-(methyl-d₃)-1H-pyrazol-5-yl)dicyclopropylphosphine oxide (1.4 g, 4.8 mmol) in 1,4-dioxane/water (30 mL/3 mL) was added Pd(dppf)Cl₂ (351 mg, 0.50 mmol) and K₂CO₃ (1.3 g, 9.6 mmol). The mixture was stirred at 110° C. overnight under N₂ atmosphere. The solvent was removed and the residue was purified by silica gel chromatography column (DCM/MeOH=100/3) to give the compound (1.4 g, 87%) as a yellow oil. ¹H-NMR (300 MHz, CDCl₃): δ 7.22 (d, J=8.1 Hz, 1H), 7.12 (s, 1H), 6.97 (t, J=7.8 Hz, 1H), 6.74 (d, J=7.5 Hz, 1H), 3.89 (s, 2H), 3.64 (s, 3H), 1.16-0.88 (m, 10H). LC-MS: m/z 334.9 [M+H]⁺.

Step d. 6-Chloro-4-((3-(5-(dicyclopropylphosphoryl)-1-(methyl-d₃)-1H-pyrazol-3-yl)-2-methoxyphenyl)amino)pyridazine-3-carboxamide: To a solution of 4,6-dichloropyridazine-3-carboxamide (776 mg, 4.0 mmol) and (3-(3-amino-2-methoxyphenyl)-1-(methyl-d₃)-1H-pyrazol-5-yl)dicyclopropylphosphine oxide (900 mg, 2.7 mmol) in anhydrous tetrahydrofuran (8 mL) was added 1N LiHMDS (11 mL, 11 mmol) under N₂ atmosphere. The mixture was stirred at room temperature overnight. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=20/1) to give the final compound (800 mg, 61%) as a yellow solid. ¹H-NMR (300 MHz, DMSO-d₆): δ 11.15 (s, 1H), 8.75 (s, 1H), 8.09 (s, 1H), 7.75 (d, J=7.5 Hz, 1H), 7.53 (d, J=8.1 Hz, 1H), 7.33-7.14 (m, 3H), 3.62 (s, 3H), 1.43-1.28 (m, 2H), 1.02-0.69 (m, 8H). LC-MS: m/z 489.7 [M+H]⁺.

Step e. 6-(Cyclopropanecarboxamido)-4-((3-(5-(dicyclopropylphosphoryl)-1-(methyl-d₃)-1H-pyrazol-3-yl)-2-methoxyphenyl)amino)pyridazine-3-carboxamide (A24): To a solution of 6-chloro-4-((3-(5-(dicyclopropylphosphoryl)-1-(methyl-d₃)-1H-pyrazol-3-yl)-2-methoxyphenyl)amino)pyridazine-3-carboxamide (80 mg, 0.16 mmol) in 1,4-dioxane (2 mL) was added cyclopropanecarboxamide (42 mg, 0.49 mmol), Pd₂(dba)₃ (15 mg, 0.020 mmol), Xantphos (10 mg, 0.020 mmol) and K₃PO₄ (69 mg, 0.33 mmol). The mixture was stirred at 120° C. under N₂ atmosphere in a microwave apparatus for 1.5 hours. The solvent was filtered and the filtrate was concentrated. The residue was purified by silica gel chromatography column (DCM/MeOH=30/1) to give the crude product, which was rinsed by Et₂O to give the desired product (10 mg, 11%) as a brown solid.

The similar procedure described in Example 11 was carried out to get the Compounds A49, A50, A59, and A65-A67.

### Example 12, Methods AA, AM, AI, AN

### Preparation of 6-(cyclopropanecarboxamido)-4-((5-(5-(dicyclopropylphosphoryl)-1-methyl-1H-pyrazol-3-yl)thiophen-2-yl)amino)pyridazine-3-carboxamide (A25)

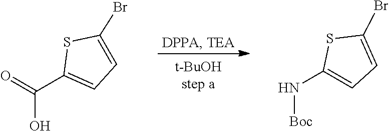 [SMILES: O=C(O)c1ccc(Br)s1 ; CC(C)(C)OC(=O)Nc1ccc(Br)s1]

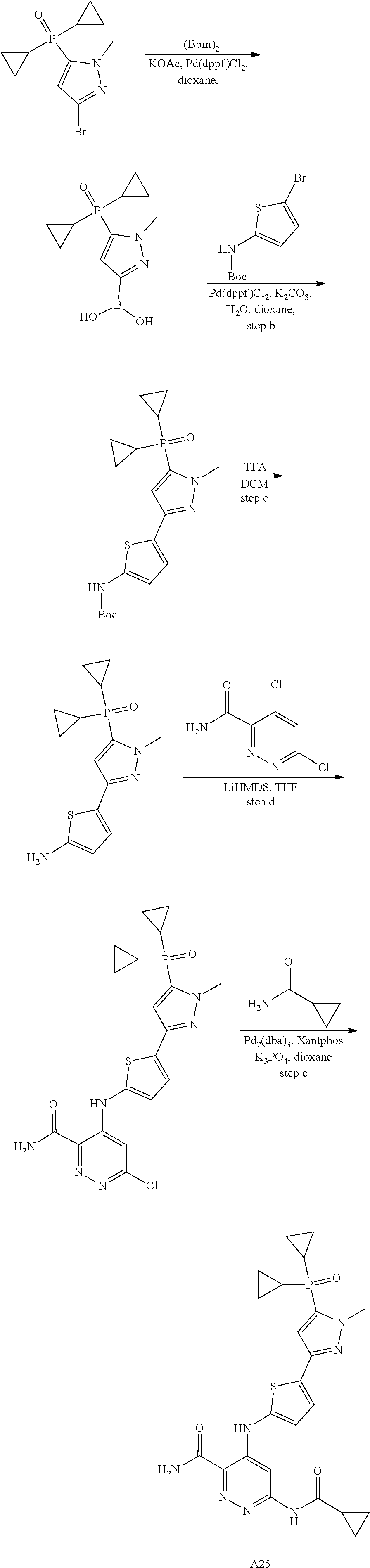

Step a. tert-Butyl (5-bromothiophen-2-yl)carbamate: To a solution of 5-bromothiophene-2-carboxylic acid (300 mg, 1.5 mmol) in anhydrous t-BuOH (15 mL) was added TEA (303 mg, 3.0 mmol) and DPPA (467 mg, 1.7 mmol) under N₂ atmosphere. The mixture was stirred at 70° C. for 24 hours. The mixture was added water (10 mL) and extracted with EA (10 mL×3). The combined organic layer was dried by Na₂SO₄ and concentrated. The residue was purified by silica gel chromatography column (PE/EA=10/1) to give the final compound (280 mg, 67%) as a white solid. ¹H-NMR (300 MHz, CDCl₃): δ 7.39 (s, 1H), 6.75 (d, J=3.9 Hz, 1H), 6.23 (d, J=3.9 Hz, 1H), 2.17 (s, 9H). LC-MS: m/z 299.7 [M+Na]+.

Step b. tert-Butyl (5-(5-(dicyclopropylphosphoryl)-1-methyl-1H-pyrazol-3-yl)thiophen-2-yl)carbamate: To a solution of (3-bromo-1-methyl-1H-pyrazol-5-yl)dicyclopropylphosphine oxide (500 mg, 1.7 mmol) in 1,4-dioxane (20 mL) was added (Bpin)₂ (660 mg, 1.5 mmol), KOAc (500 mg, 5.1 mmol), Pd(dppf)Cl₂ (124 mg, 0.17 mmol). The mixture was stirred at 100° C. under N₂ atmosphere overnight. To above mixture was added water (2 mL), K₂CO₃ (469 mg, 3.4 mmol), Pd(dppf)Cl₂ (124 mg, 0.17 mmol) and tert-butyl (5-bromothiophen-2-yl)carbamate (692 mg, 2.5 mmol). The mixture was stirred at 100° C. for 12 hours. The solvent was removed and the residue was purified by silica gel chromatography column (DCM/MeOH=30/1) to give the desired product (450 mg, 65%) as a black solid. ¹H-NMR (300 MHz, CDCl₃): δ 7.51 (s, 1H), 7.06 (d, J=3.0 Hz, 1H), 6.69 (s, 1H), 6.47 (d, J=3.9 Hz, 1H), 4.14 (s, 3H), 1.23 (s, 9H), 1.06-0.93 (m, 10H). LC-MS: m/z 407.8 [M+H]⁺.

Step c. (3-(5-Aminothiophen-2-yl)-1-methyl-1H-pyrazol-5-yl)dicyclopropylphosphine oxide: To a solution of tert-butyl (5-(5-(dicyclopropylphosphoryl)-1-methyl-1H-pyrazol-3-yl)thiophen-2-yl)carbamate (100 mg, 0.24 mmol) in DCE (5 mL) was added TFA (136 mg, 1.2 mmol). The mixture was stirred at room temperature for 4 hours. The solvent was concentrated in vacuum and diluted with saturated NaHCO₃ aqueous solution (10 mL). The mixture was extracted with DCM (10 mL×3) and the combined organic layer was dried by Na₂SO₄. The organic layer was concentrated and the residue was purified by silica gel chromatography column (DCM/MeOH=30/1) to give the desired product (30 mg, 41%) as a brown oil. ¹H-NMR (300 MHz, CDCl₃): δ 7.52 (s, 1H), 6.92-6.87 (m, 1H), 6.15-6.11 (m, 1H), 4.14 (s, 3H), 3.87 (s, 2H), 1.12-0.85 (m, 10H). LC-MS: m/z 307.8 [M+H]⁺.

Step d. 6-Chloro-4-((5-(5-(dicyclopropylphosphoryl)-1-methyl-1H-pyrazol-3-yl)thiophen-2-yl)amino)pyridazine-3-carboxamide: To a solution of 4,6-dichloropyridazine-3-carboxamide (126 mg, 0.66 mmol) and (3-(5-aminothiophen-2-yl)-1-methyl-1H-pyrazol-5-yl)dicyclopropylphosphine oxide (136 mg, 0.44 mmol) in anhydrous tetrahydrofuran (8 mL) was added 1N LiHMDS (1.3 mL, 1.3 mmol) under N₂ atmosphere. The mixture was stirred at room temperature for 3 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=20/1) to give the final compound (95 mg, 47%) as a yellow solid. ¹H-NMR (300 MHz, CDCl₃): δ 10.48 (s, 1H), 8.07 (s, 1H), 7.22-7.17 (m. 1H), 7.10 (s, 1H), 6.86-6.83 (m. 1H), 6.78 (s, 1H), 5.73 (s, 1H), 4.19 (s, 3H), 1.33-1.23 (m. 2H), 1.12-0.96 (m. 8H). LC-MS: m/z 462.7 [M+H]⁺.

Step e. 6-(Cyclopropanecarboxamido)-4-((5-(5-(dicyclopropylphosphoryl)-1-methyl-1H-pyrazol-3-yl)thiophen-2-yl)amino)pyridazine-3-carboxamide (A25): To a solution of 6-chloro-4-((5-(5-(dicyclopropylphosphoryl)-1-methyl-1H-pyrazol-3-yl)thiophen-2-yl)amino)pyridazine-3-carboxamide (50 mg, 0.10 mmol) in 1,4-dioxane (2 mL) was added cyclopropanecarboxamide (27 mg, 0.30 mmol), Pd₂(dba)₃ (9 mg, 0.010 mmol), Xantphos (5 mg, 0.010 mmol 1) and K₃PO₄ (42 mg, 0.20 mmol). The mixture was stirred at 120° C. under N₂ atmosphere in a microwave apparatus for 2 hours. The solvent was filtered and the filtrate was concentrated. The residue was purified by silica gel chromatography column (DCM/MeOH=30/1) to give the crude product, which was rinsed by Et₂O to give the desired product (15 mg, 29%) as a yellow solid.

### Example 13, Method AO

### Preparation of 6-(cyclopropanecarboxamido)-4-((3-(4-(dicyclopropylphosphoryl)-1H-pyrazol-1-yl)-2-methoxyphenyl)amino)pyridazine-3-carboxamide (A44)

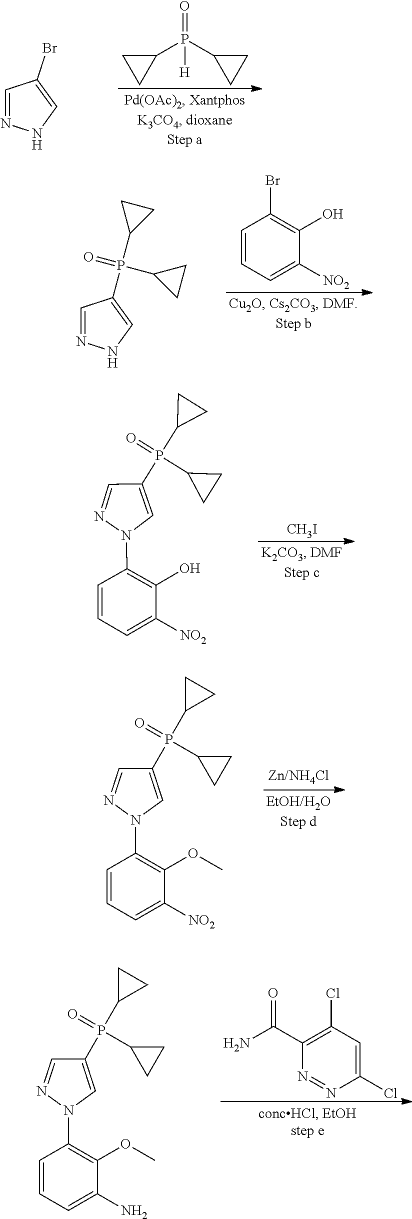

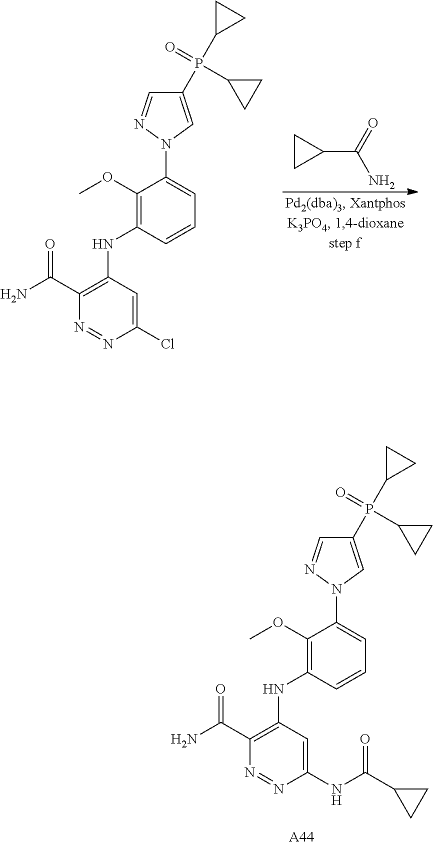 [SMILES: COc1c(Nc2cc(Cl)nnc2C(N)=O)cccc1-n1cc(P(=O)(C2CC2)C2CC2)cn1 ; NC(=O)C1CC1 ; COc1c(Nc2cc(NC(=O)C3CC3)nnc2C(N)=O)cccc1-n1cc(P(=O)(C2CC2)C2CC2)cn1 ; "A44"]

Step a. Dicyclopropyl(1H-pyrazol-4-yl)phosphine oxide: To a solution of 4-bromo-1H-pyrazole (1.5 g, 10 mmol) in 1,4-dioxane (5 mL) was added K₃PO₄ (3.2 g, 15 mmol), dicyclopropylphosphine oxide (2.7 g, 20 mmol), Pd(OAc)₂ (229 mg, 1.0 mmol) and Xantphos (590 mg, 1.0 mmol). The mixture was stirred at 110° C. under N₂ atmosphere for 0.5 hour. The solvent was concentrated and the residue was purified by silica gel chromatography column (DCM/MeOH=100/3) to give the final compound (465 mg, 24%) as a light yellow solid. ¹H-NMR (300 MHz, CDCl₃): δ 7.82 (s, 2H), 1.01-0.85 (m, 10H). LC-MS: m/z 197.0 [M+H]⁺.

Step b. Dicyclopropyl(1-(2-hydroxy-3-nitrophenyl)-1H-pyrazol-4-yl)phosphine oxide: To a solution of dicyclopropyl(1H-pyrazol-4-yl)phosphine oxide (515 mg, 2.6 mmol), 2-bromo-6-nitrophenol (382 mg, 1.8 mmol) in DMF (5 mL) was added Cu₂O (250 mg, 1.8 mmol) and Cs₂CO₃ (1.1 g, 3.5 mmol). The mixture was stirred at 135° C. in microwave reactor for 2 hours. The mixture was filtered and concentrated in vacuum. The residue was used for the next step without further purification. LC-MS: m/z 333.8 [M+H]⁺.

Step c. Dicyclopropyl(1-(2-methoxy-3-nitrophenyl)-1H-pyrazol-4-yl)phosphine oxide: To a solution of dicyclopropyl(1-(2-hydroxy-3-nitrophenyl)-1H-pyrazol-4-yl)phosphine oxide in DMF (10 mL) was added K₂CO₃ (363 mg, 2.6 mmol) and iodomethane (373 mg, 2.6 mmol). The mixture was stirred at room temperature for 2 hours. The solvent was removed and the residue was purified by silica gel chromatography column (DCM/MeOH=20/1) to give the final compound as a yellow oil. (655 mg, 72%). ¹H-NMR (300 MHz, CDCl₃): δ 8.41 (s, 1H), 8.01 (s, 1H), 7.97 (d, J=8.1 Hz, 1H), 7.83 (d, J=8.1 Hz, 1H), 7.37 (t, J=8.1 Hz, 1H), 3.67 (s, 3H), 1.08-0.87 (m, 10H). LC-MS: m/z 347.8 [M+H]⁺.

Step d. (1-(3-Amino-2-methoxyphenyl)-1H-pyrazol-4-yl)dicyclopropylphosphine oxide: To a solution of dicyclopropyl(1-(2-methoxy-3-nitrophenyl)-1H-pyrazol-4-yl)phosphine oxide (650 mg, 1.9 mmol) in a mixed solvent of EtOH and water (30 mL/10 mL) was added zinc powder (487 mg, 7.5 mmol) and NH₄Cl (202 mg, 3.7 mmol). The mixture was stirred at 50° C. for 2 hours, and then filtered. The filtrate was concentrated and the residue was purified by silica gel chromatography column (DCM/MeOH=20/1) to give the desired product (400 mg, 67%) as a red oil. ¹H-NMR (300 MHz, CDCl₃): δ 8.33 (s, 1H), 7.94 (s, 1H), 7.03-6.94 (m, 2H), 6.76 (t, J=4.2 Hz, 1H), 4.01 (s, 2H), 3.47 (s, 3H), 1.05-0.82 (m, 10H). LC-MS: m/z 317.9 [M+H]⁺.

Step e. 6-Chloro-4-((3-(4-(dicyclopropylphosphoryl)-1H-pyrazol-1-yl)-2-methoxyphenyl)amino)pyridazine-3-carboxamide: To a solution of (1-(3-amino-2-methoxyphenyl)-1H-pyrazol-4-yl)dicyclopropylphosphine oxide (300 mg, 0.95 mmol) and 4,6-dichloropyridazine-3-carboxamide (218 mg, 1.1 mmol) in EtOH (3 mL) was added a catalytic amount of conc. HCl (one drop). The mixture was stirred at 120° C. for 2.5 hours in a microwave reactor. The solvent was concentrated in vacuum and the residue was purified by silica gel chromatography column (DCM/MeOH=20/1) to give the desired product (180 mg, 40%) as a yellow solid. ¹H-NMR (300 MHz, DMSO-d₆): δ 11.22 (s, 1H), 8.78 (s, 1H), 8.52 (s, 1H), 8.13 (s, 1H), 8.05 (s, 1H), 7.64 (d, J=8.1 Hz, 1H), 7.54 (d, J=7.5 Hz, 1H), 7.37 (t, J=8.1 Hz, 1H), 7.31 (s, 1H), 3.48 (s, 3H), 1.20-1.14 (m, 2H), 0.91-0.63 (m, 8H). LC-MS: m/z 472.7 [M+H]⁺.

Step f. 6-(Cyclopropanecarboxamido)-4-((3-(4-(dicyclopropylphosphoryl)-1H-pyrazol-1-yl)-2-methoxyphenyl)amino)pyridazine-3-carboxamide (A44): To a solution of 6-chloro-4-((3-(4-(dicyclopropylphosphoryl)-1H-pyrazol-1-yl)-2-methoxyphenyl)amino)pyridazine-3-carboxamide (120 mg, 0.25 mmol) in 1,4-dioxane (2 mL) was added cyclopropanecarboxamide (65 mg, 0.76 mmol), Pd₂(dba)₃ (23 mg, 0.030 mmol), Xantphos (15 mg, 0.030 mmol) and K₃PO₄ (108 mg, 0.51 mmol). The mixture was stirred at 110° C. under N₂ atmosphere in a microwave apparatus for 2 hours. The solvent was filtered and the filtrate was concentrated. The residue was purified by silica gel chromatography column (DCM/MeOH=20/1) to give the crude product, which was rinsed by Et₂O to give the desired product (25 mg, 19%) as a yellow solid.

### Example 14, Method AB, AC, AH, AI, AN

### Preparation of 6-(cyclopropanecarboxamido)-4-((3-(1-cyclopropyl-5-(dimethylphosphoryl)-1H-pyrazol-3-yl)-2-methoxyphenyl)amino)pyridazine-3-carboxamide (A45)

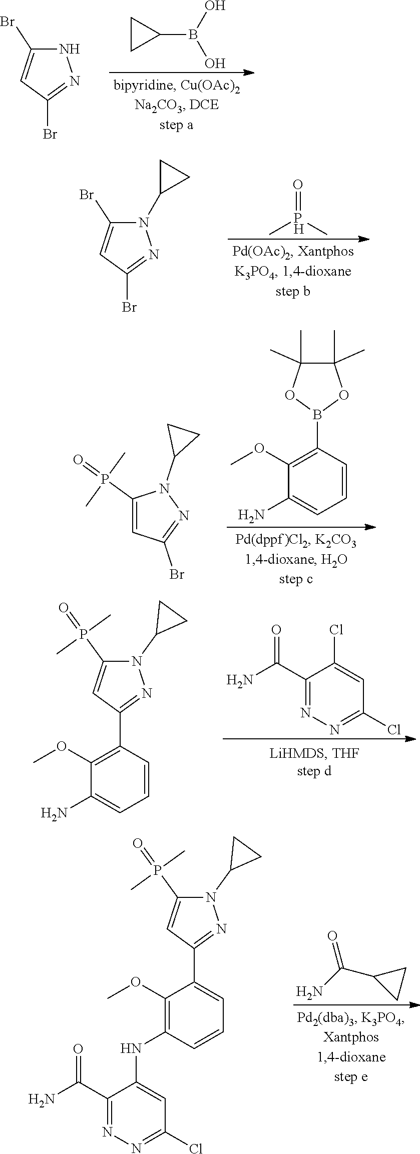

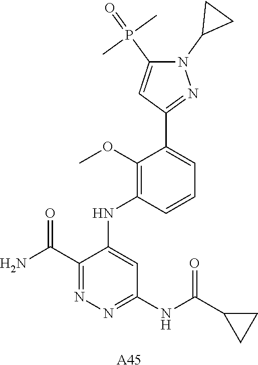 [SMILES: COc1c(Nc2cc(NC(=O)C3CC3)nnc2C(N)=O)cccc1-c1cc(P(C)(C)=O)n(C2CC2)n1 ; "A45"]

Step a. 3,5-Dibromo-1-cyclopropyl-1H-pyrazole: To a solution of 3,5-dibromo-1H-pyrazole (1.7 g, 7.6 mmol) in DCE (30 mL) was added cyclopropylboronic acid (1.3 g, 15.1 mmol), bipyridine (1.2 g, 7.6 mmol), Cu(OAc)₂ and Na₂CO₃. The mixture was stirred at 75° C. overnight in the open air. The mixture was diluted with saturated NH₄Cl aqueous solution (30 mL) and extracted with DCM (20 mL×3). The combined organic layer was washed with saturated NaCl aqueous solution (30 mL) and dried by Na₂SO₄. The combined organic layer was concentrated in vacuum and the residue was purified by silica gel chromatography column (PE/EA=20/1) to give the desired product (756 mg, 38%) as a yellow solid. ¹H-NMR (300 MHz, CDCl₃): δ 6.29 (s, 1H), 3.50-3.36 (m, 1H), 1.27-1.17 (m, 2H), 1.12-1.03 (m, 2H). LC-MS: m/z 264.8 [M+H]⁺

Step b. (3-Bromo-1-cyclopropyl-1H-pyrazol-5-yl)dimethylphosphine oxide: To a solution of 3,5-dibromo-1-cyclopropyl-1H-pyrazole (756 mg, 2.8 mmol) in 1,4-dioxane (5 mL) was added K₃PO₄ (720 mg, 3.4 mmol), dimethylphosphine oxide (334 mg, 4.3 mmol), Pd(OAc)₂ (51 mg, 0.23 mmol) and Xantphos (133 mg, 0.23 mmol). The mixture was stirred at 130° C. under N₂ atmosphere for 1 hour. The solvent was concentrated and the residue was purified by silica gel chromatography column (DCM/MeOH=75/1) to give the final compound (175 mg, 23%) as a light yellow solid. ¹H-NMR (300 MHz, CDCl₃): δ 6.43 (s, 1H), 4.31-4.13 (m, 1H), 1.86 (d, J=13.5 Hz, 6H), 1.43-1.34 (m, 2H), 1.15-1.01 (m, 2H). LC-MS: m/z 263.0 [M+H]⁺.

Step c. (3-(3-Amino-2-methoxyphenyl)-1-cyclopropyl-1H-pyrazol-5-yl)dimethylphosphine oxide: To a solution of 2-methoxy-3-(4,4,5,5-tetramethyl-1,3,2-dioxaborolan-2-yl)aniline (249 mg, 1.0 mmol) and (3-bromo-1-cyclopropyl-1H-pyrazol-5-yl)dimethylphosphine oxide (176 mg, 0.67 mmol) in 1,4-dioxane/water (10 mL/1 mL) was added Pd(dppf)Cl₂ (49 mg, 0.070 mmol) and K₂CO₃ (185 mg, 1.3 mmol). The mixture was stirred at 100° C. overnight under N₂ atmosphere. The solvent was removed and the residue was purified by silica gel chromatography column (DCM/MeOH=100/3) to give the compound (140 mg, 68%) as a yellow oil. ¹H-NMR (300 MHz, CDCl₃): δ 7.18 (d, J=7.8 Hz, 1H), 6.99-6.90 (m, 2H), 6.72 (d, J=7.5 Hz, 1H), 4.36-4.18 (m, 1H), 3.87 (s, 2H), 3.63 (s, 3H), 1.89 (d, J=13.5 Hz, 6H), 1.53-1.43 (m, 2H), 1.15-1.03 (m, 2H). LC-MS: m/z 306.1 [M+H]⁺

Step d. 6-Chloro-4-((3-(1-cyclopropyl-5-(dimethylphosphoryl)-1H-pyrazol-3-yl)-2-methoxyphenyl)amino)pyridazine-3-carboxamide: To a solution of 4,6-dichloropyridazine-3-carboxamide (255 mg, 1.3 mmol) and (3-(3-amino-2-methoxyphenyl)-1-cyclopropyl-1H-pyrazol-5-yl)dimethylphosphine oxide (270 mg, 0.90 mmol) in anhydrous tetrahydrofuran (8 mL) was added 1N LiHMDS (3.6 mL, 3.6 mmol) under N₂ atmosphere. The mixture was stirred at room temperature for 8 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=20/1) to give the final compound (280 mg, 68%) as a yellow solid. ¹H-NMR (300 MHz, DMSO-d₆): δ 11.13 (s, 1H), 8.76 (s, 1H), 8.11 (s, 1H), 7.73-7.67 (m, 1H), 7.53-7.49 (m, 1H), 7.29-7.23 (m, 1H), 7.17 (s, 1H), 7.07 (s, 1H), 4.34-4.26 (m, 1H), 3.62 (s, 3H), 1.86 (d, J=13.8 Hz, 6H), 1.32-1.28 (m, 2H), 1.10-1.06 (m, 2H). LC-MS: m/z 460.7 [M+H]⁺.

Step e. 6-(Cyclopropanecarboxamido)-4-((3-(1-cyclopropyl-5-(dimethylphosphoryl)-1H-pyrazol-3-yl)-2-methoxyphenyl)amino)pyridazine-3-carboxamide (A45): To a solution of 6-chloro-4-((3-(1-cyclopropyl-5-(dimethylphosphoryl)-1H-pyrazol-3-yl)-2-methoxyphenyl)amino)pyridazine-3-carboxamide (120 mg, 0.26 mmol) in 1,4-dioxane (2 mL) was added cyclopropanecarboxamide (68 mg, 0.80 mmol), Pd₂(dba)₃ (28 mg, 0.030 mmol), Xantphos (32 mg, 0.056 mmol) and K₃PO₄ (112 mg, 0.52 mmol). The mixture was stirred at 120° C. under N₂ atmosphere in a microwave apparatus for 2 hours. The solvent was filtered and the filtrate was concentrated. The residue was purified by silica gel chromatography column (DCM/MeOH=12/1) to give the crude product, which was rinsed by Et₂O to give the desired product (31 mg, 24%) as a white solid.

The similar procedure described in Example 14 was carried out to get the Compounds A46, A52, A53, and A54.

### Example 15, Method AB, AP

### Preparation of 6-(cyclopropanecarboxamido)-4-((3-(5-(dicyclopropylphosphoryl)-1H-pyrazol-3-yl)-2-methoxyphenyl)amino)pyridazine-3-carboxamide (A48)

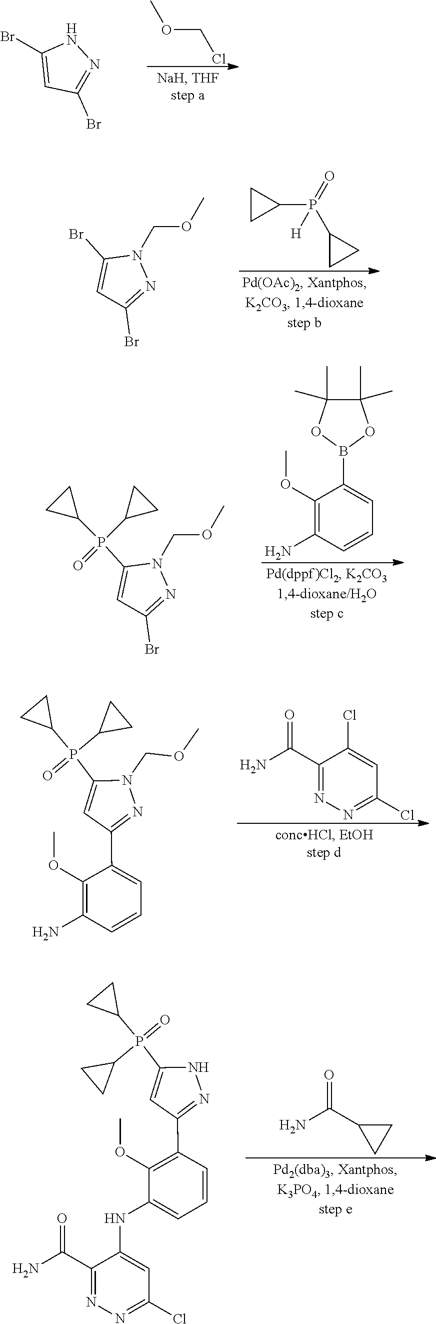

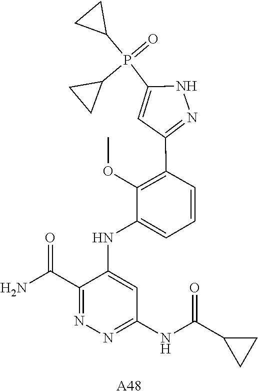 [SMILES: COc1c(Nc2cc(NC(=O)C3CC3)nnc2C(N)=O)cccc1-c1cc(P(=O)(C2CC2)C2CC2)[nH]n1 ; "A48"]

Step a. 3,5-Dibromo-1-(methoxymethyl)-1H-pyrazole: To a solution of 3,5-dibromo-1H-pyrazole (500 mg, 2.2 mmol) in anhydrous THF (15 mL) was added 60% NaH (195 mg, 4.9 mmol) and the mixture was stirred at 0° C. for 1 hour. After adding MOMCl (267 mg, 3.3 mmol), the mixture was stirred at 0° C. for 2 hours followed by stirring at room temperature overnight. The reaction was quenched with ice water (20 mL) and the mixture was extracted with EA (20 mL×3). The combined organic layer was dried by Na₂SO₄ and concentrated in vacuum. The residue was purified by silica gel chromatography column (DCM/MeOH=20/1) to give the final compound (550 mg, 93%) as a colorless oil. ¹H-NMR (300 MHz, CDCl₃): δ 6.37 (s, 1H), 5.39 (s, 2H), 3.37 (s, 3H). LC-MS: m/z 268.7 [M+H]⁺.

Step b. (3-Bromo-1-(methoxymethyl)-1H-pyrazol-5-yl)dicyclopropylphosphine oxide: To a solution of 3,5-dibromo-1-(methoxymethyl)-1H-pyrazole (550 mg, 2.0 mmol) in 1,4-dioxane (10 mL) was added K₂CO₃ (424 mg, 3.1 mmol), dicyclopropylphosphine oxide (400 mg, 3.1 mmol), Pd(OAc)₂ (44.8 mg, 0.20 mmol) and Xantphos (116 mg, 0.20 mmol). The mixture was stirred at 110° C. under N₂ atmosphere for 5 hours. The solvent was concentrated and the residue was purified by silica gel chromatography column (DCM/MeOH=30/1) to give the crude product as a black solid. LC-MS: m/z 318.8 [M+H]⁺.

Step c. (3-(3-Amino-2-methoxyphenyl)-1-(methoxymethyl)-1H-pyrazol-5-yl)dicyclopropylphosphine oxide: To a solution of 2-methoxy-3-(4,4,5,5-tetramethyl-1,3,2-dioxaborolan-2-yl)aniline (350 mg, 1.4 mmol) and (3-bromo-1-(methoxymethyl)-1H-pyrazol-5-yl)dicyclopropylphosphine oxide (300 mg, 0.94 mmol) in 1,4-dioxane/water (10 mL/1 mL) was added Pd(dppf)Cl₂ (66 mg, 0.090 mmol) and K₂CO₃ (248 mg, 1.8 mmol). The mixture was stirred at 100° C. for 6 hours under N₂ atmosphere. The solvent was removed and the residue was purified by silica gel chromatography column (DCM/MeOH=50/1) to give the crude product as a black oil. LC-MS: m/z 361.9 [M+H]⁺

Step d. 6-Chloro-4-((3-(5-(dicyclopropylphosphoryl)-1H-pyrazol-3-yl)-2-methoxyphenyl)amino)pyridazine-3-carboxamide: To a solution of (3-(3-amino-2-methoxyphenyl)-1-(methoxymethyl)-1H-pyrazol-5-yl)dicyclopropylphosphine oxide (140 mg, 0.39 mmol) and 4,6-dichloropyridazine-3-carboxamide (89.4 mg, 0.46 mmol) in EtOH (8 mL) was added a catalytic amount of conc. HCl (one drop). The mixture was stirred at 120° C. for 1 hour in a microwave reactor. The solvent was concentrated in vacuum and the residue was purified by silica gel chromatography column (DCM/MeOH=20/1) to give the desired product (68 mg, 37%) as a yellow solid. ¹H-NMR (300 MHz, DMSO-d₆): δ 13.80 (s, 1H), 11.17 (s, 1H), 8.77 (s, 1H), 8.12 (s, 1H), 7.68-7.47 (m, 2H), 7.41-7.25 (m, 1H), 7.28-7.20 (m, 1H), 7.18-7.05 (m, 1H), 3.60 (s, 3H), 1.03-0.64 (m, 10H). LC-MS: m/z 472.7 [M+H]⁺.

Step e. 6-(Cyclopropanecarboxamido)-4-((3-(5-(dicyclopropylphosphoryl)-1H-pyrazol-3-yl)-2-methoxyphenyl)amino)pyridazine-3-carboxamide (A48): To a solution of 6-chloro-4-((3-(5-(dicyclopropylphosphoryl)-1H-pyrazol-3-yl)-2-methoxyphenyl)amino)pyridazine-3-carboxamide (60 mg, 0.13 mmol) in 1,4-dioxane (2 mL) was added cyclopropanecarboxamide (33 mg, 0.38 mmol), Pd₂(dba)₃ (12 mg, 0.013 mmol), Xantphos (8.0 mg, 0.013 mmol) and K₃PO₄ (55 mg, 0.26 mmol). The mixture was stirred at 120° C. under N₂ atmosphere in a microwave apparatus for 2 hours. The solvent was filtered and the filtrate was concentrated. The residue was purified by silica gel chromatography column (DCM/MeOH=15/1) to give the crude product, which was rinsed by Et₂O to give the desired product (10 mg, 15%) as a yellow solid.

### Example 16, Methods AA, AB, AC, AI, AN

### Preparation of 4-((3-(5-(dimethylphosphoryl)-1-methyl-1H-pyrazol-3-yl)-5-fluoro-2-methoxyphenyl)amino)-6-((4-methylpyridin-2-yl)amino)pyridazine-3-carboxamide (A51)

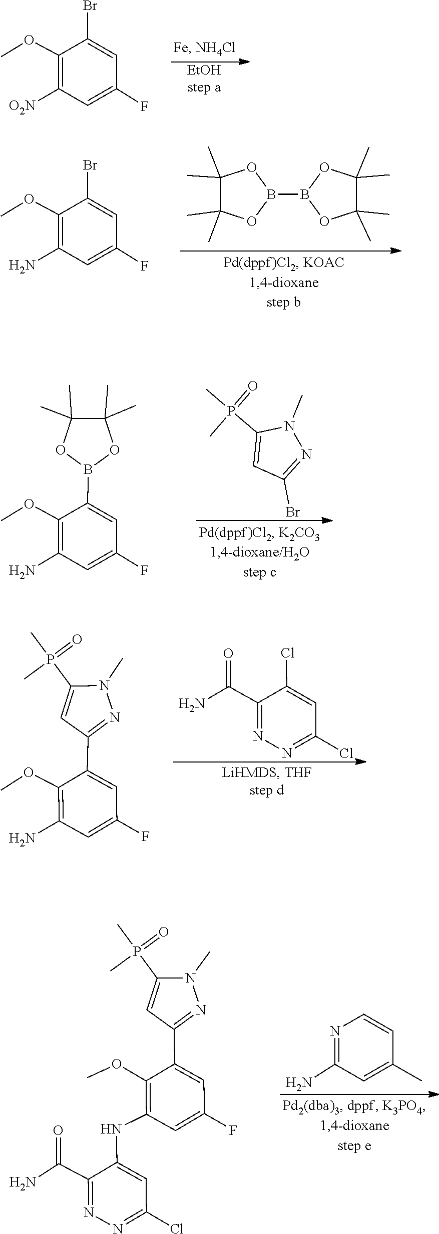

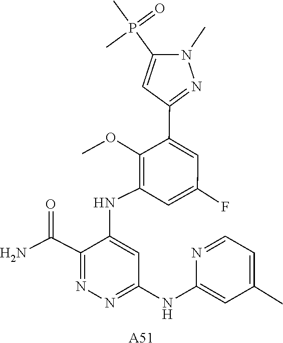

Step a. 3-Bromo-5-fluoro-2-methoxyaniline: To a solution of 1-bromo-5-fluoro-2-methoxy-3-nitrobenzene (650 mg, 1.9 mmol) in a mixed solvent of EtOH and saturated NH₄Cl aqueous solution (25 mL/5 mL) was added iron powder (3.6 g, 66 mmol). The mixture was stirred at 70° C. for 2 hours, and then filtered. The filtrate was concentrated and the residue was purified by silica gel chromatography column (PE/EA=4/1) to give the desired product (1.3 g, 92%) as a brown oil. ¹H-NMR (300 MHz, CDCl₃): δ 6.62 (dd, J=8.4, 2.7 Hz, 1H), 7.40 (dd, J=8.7, 3.9 Hz, 1H), 3.98 (s, 2H), 3.79 (s, 3H). LC-MS: m/z 220.0 [M+H]⁺.

Step b. 5-Fluoro-2-methoxy-3-(4,4,5,5-tetramethyl-1,3,2-dioxaborolan-2-yl)aniline: To a solution of 3-bromo-5-fluoro-2-methoxyaniline (704 mg, 3.2 mmol) in 1,4-dioxane (60 mL) was added (Bpin)₂ (1.6 g 6.4 mmol), KOAc (940 mg 9.6 mmol) and Pd(dppf)Cl₂ (243 mg 0.33 mmol). The mixture was stirred at 105° C. under N₂ atmosphere overnight. The solvent was removed and the residue was purified by silica gel chromatography column (PE/EA=4/1) to give the final compound (560 mg, 50%) as a yellow oil. ¹H-NMR (300 MHz, CDCl₃): δ 6.95-6.87 (m, 2H), 6.47-6.38 (m, 1H), 4.26 (s, 3H), 3.99 (s, 2H), 3.60 (s, 3H), 1.85 (d, J=13.5 Hz, 6H).

Step c. (3-(3-Amino-5-fluoro-2-methoxyphenyl)-1-methyl-1H-pyrazol-5-yl)dimethylphosphine oxide: To a solution of 5-fluoro-2-methoxy-3-(4,4,5,5-tetramethyl-1,3,2-dioxaborolan-2-yl)aniline (243 mg, 0.42 mmol) and (3-bromo-1-methyl-1H-pyrazol-5-yl)dimethylphosphine oxide (150 mg, 0.63 mmol) in 1,4-dioxane/water (5 mL/0.5 mL) was added Pd(dppf)Cl₂ (43 mg 0.063 mmol) and K₂CO₃ (174 mg 1.3 mmol). The mixture was stirred at 110° C. overnight under N₂ atmosphere. The solvent was removed and the residue was purified by silica gel chromatography column (DCM/MeOH=30/1) to give the final compound (150 mg, 50%) as a yellow oil. ¹H-NMR (300 MHz, CDCl₃): δ 6.95-6.87 (m, 2H), 6.46-6.39 (m, 1H), 4.27 (s, 3H), 3.98 (s, 2H), 3.60 (s, 3H), 1.80 (d, J=13.6 Hz, 6H). LC-MS: m/z 298.1 [M+H]⁺.

Step d. 6-Chloro-4-((3-(5-(dimethylphosphoryl)-1-methyl-1H-pyrazol-3-yl)-5-fluoro-2-methoxyphenyl)amino)pyridazine-3-carboxamide: To a solution of 4,6-dichloropyridazine-3-carboxamide (423 mg, 2.3 mmol) and (3-(3-amino-5-fluoro-2-methoxyphenyl)-1-methyl-1H-pyrazol-5-yl)dimethylphosphine oxide (450 mg, 1.5 mmol) in anhydrous tetrahydrofuran (6 mL) was added 1N LiHMDS (4.7 mL, 4.7 mmol) under N₂ atmosphere. The mixture was stirred at room temperature overnight. Saturated NH₄Cl (5 mL) aqueous solution was added and the aqueous layer was extracted with ethyl acetate (10 mL×3). The combined organic layer was dried by Na₂SO₄ and concentrated. The residue was purified by silica gel chromatography column (DCM/MeOH=20/1) to give the final compound (190 mg, 25%) as a yellow solid. ¹H-NMR (300 MHz, DMSO-d₆): δ 11.31 (s, 1H), 8.81 (s, 1H), 8.15 (s, 1H), 7.49 (d, J=11.4 Hz, 1H), 7.46-7.34 (m, 2H), 7.12 (s, 1H), 4.17 (s, 3H), 3.62 (s, 3H), 1.82 (d, J=13.8 Hz, 6H). LC-MS: m/z 452.7 [M+H]⁺.

Step e. 4-((3-(5-(Dimethylphosphoryl)-1-methyl-1H-pyrazol-3-yl)-5-fluoro-2-methoxyphenyl)amino)-6-((4-methylpyridin-2-yl)amino)pyridazine-3-carboxamide (A51): To a solution of 6-chloro-4-((3-(5-(dimethylphosphoryl)-1-methyl-1H-pyrazol-3-yl)-5-fluoro-2-methoxyphenyl)amino)pyridazine-3-carboxamide (170 mg, 0.34 mmol) in 1,4-dioxane (2 mL) was added 4-methylpyridin-2-amine (122 mg, 1.1 mmol), Pd₂(dba)₃ (34 mg, 0.037 mmol), dppf (42 mg, 0.074 mmol) and K₃PO₄ (166 mg, 0.78 mmol). The mixture was stirred at 110° C. under N₂ atmosphere in a microwave apparatus for 2.5 hours. The solvent was filtered and the filtrate was concentrated. The residue was purified by silica gel chromatography column (DCM/MeOH=12/1) to give the crude product, which was rinsed by Et₂O to give the desired product (83 mg, 46%) as a yellow solid.

The similar procedure described in Example 16 was carried out to get the Compound A56.

### Example 17, Methods AA, AE, AG, AI, AN

### Preparation of 4-((4-(5-(dimethylphosphoryl)-1-methyl-1H-pyrazol-3-yl)-3-methoxypyridin-2-yl)amino)-6-((4-methylpyridin-2-yl)amino)pyridazine-3-carboxamide (A55)

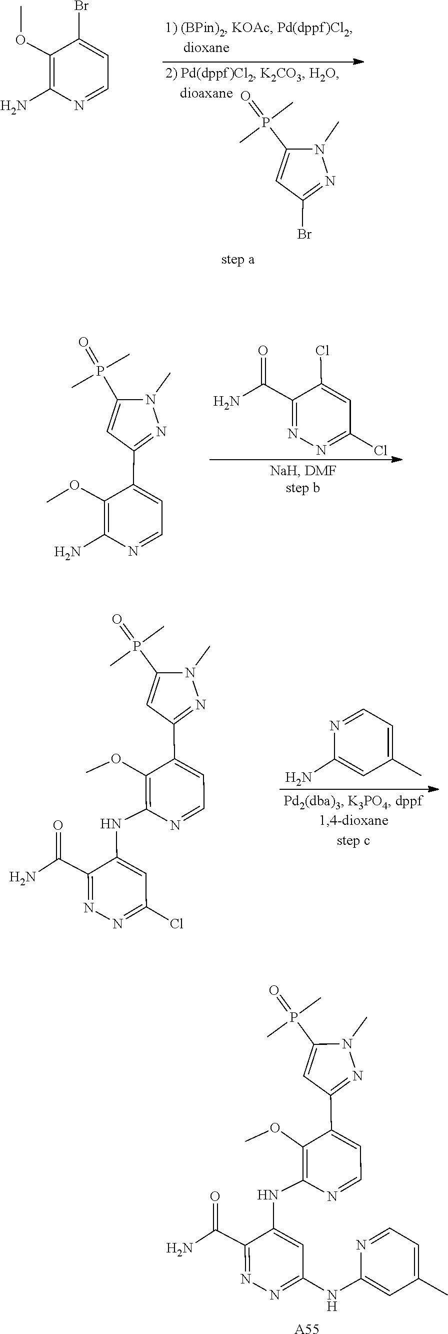

Step a. (3-(2-Amino-3-methoxypyridin-4-yl)-1-methyl-1H-pyrazol-5-yl)dimethylphosphine oxide: To a solution of 4-bromo-3-methoxypyridin-2-amine (185 mg, 0.92 mmol) in 1,4-dioxane (20 mL) was added (Bpin)₂ (460 mg, 1.8 mmol), KOAc (230 mg, 2.3 mmol), Pd(dppf)Cl₂ (66 mg, 0.090 mmol). The mixture was stirred at 100° C. under N₂ atmosphere overnight. To above mixture was added water (1 mL), K₂CO₃ (230 mg, 1.7 mmol), Pd(dppf)Cl₂ (48 mg, 0.070 mmol) and (3-bromo-1-methyl-1H-pyrazol-5-yl)dimethylphosphine oxide (156 mg, 0.66 mmol). The mixture was stirred at 100° C. for 6 hours. The solvent was removed and the residue was purified by silica gel chromatography column (DCM/MeOH=50/1) to give the desired product (60 mg, 32%) as a yellow solid. LC-MS: m/z 281.1 [M+H]⁺.

Step b. 6-Chloro-4-((4-(5-(dimethylphosphoryl)-1-methyl-1H-pyrazol-3-yl)-3-methoxypyridin-2-yl)amino)pyridazine-3-carboxamide: To a solution of 4,6-dichloropyridazine-3-carboxamide (357 mg, 1.9 mmol) and (3-(2-amino-3-methoxypyridin-4-yl)-1-methyl-1H-pyrazol-5-yl)dimethylphosphine oxide (350 mg, 1.2 mmol) in anhydrous tetrahydrofuran (8 mL) was added NaH (250 mg, 6.2 mmol) under N₂ atmosphere. The mixture was stirred at room temperature for 24 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=20/1) to give the final compound (60 mg, 11%) as a yellow solid. ¹H-NMR (300 MHz, CDCl₃): δ 12.26 (s, 1H), 9.39 (s, 1H), 8.21 (s, 1H), 8.16 (d, J=5.1 Hz, 1H), 7.51 (d, J=5.1 Hz, 1H), 7.11 (s, 1H), 5.77 (s, 1H), 4.32 (s, 3H), 3.81 (s, 3H), 1.88 (d, J=13.5 Hz, 6H). LC-MS: m/z 435.7 [M+H]⁺

Step c. 4-((4-(5-(Dimethylphosphoryl)-1-methyl-1H-pyrazol-3-yl)-3-methoxypyridin-2-yl)amino)-6-((4-methylpyridin-2-yl)amino)pyridazine-3-carboxamide (A55): To a solution of 6-chloro-4-((4-(5-(dimethylphosphoryl)-1-methyl-1H-pyrazol-3-yl)-3-methoxypyridin-2-yl)amino)pyridazine-3-carboxamide (50 mg, 0.11 mmol) in 1,4-dioxane (2 mL) was added 4-methylpyridin-2-amine (36 mg, 0.33 mmol), Pd₂(dba)₃ (10 mg, 0.011 mmol), dppf (12 mg, 0.022 mmol) and K₃PO₄ (70 mg, 0.33 mmol). The mixture was stirred at 110° C. under N₂ atmosphere in a microwave apparatus for 2 hours. The solvent was filtered and the filtrate was concentrated. The residue was purified by silica gel chromatography column (DCM/MeOH=15/1) to give the crude product, which was rinsed by Et₂O to give the desired product (27 mg, 49%) as a white solid.

The similar procedure described in Example 17 was carried out to get the Compound A60.

### Example 18, Methods AA, AF, AI, AN

### Preparation of 4-((5-(5-(dimethylphosphoryl)-1-methyl-1H-pyrazol-3-yl)-4-methoxypyridin-3-yl)amino)-6-((4-methylpyridin-2-yl)amino)pyridazine-3-carboxamide (A57)

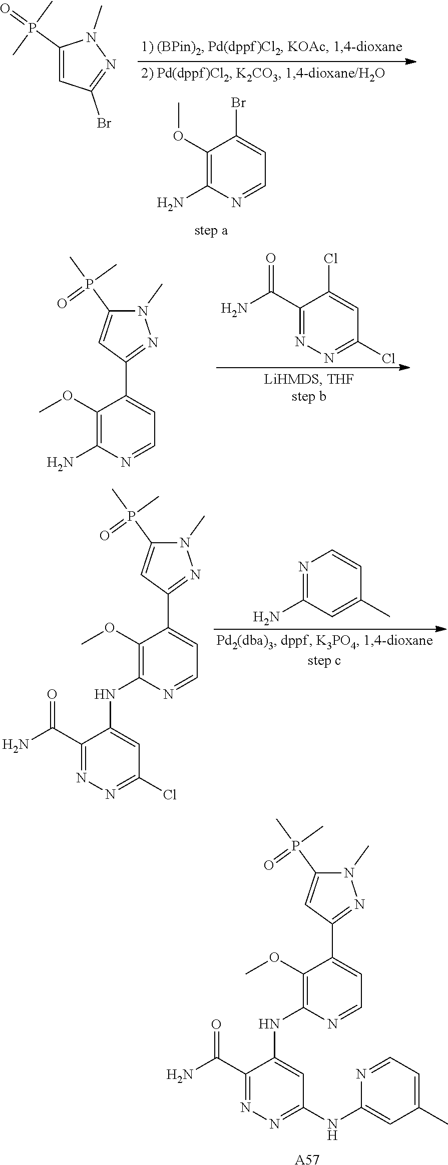

Step a. (3-(5-Amino-4-methoxypyridin-3-yl)-1-methyl-1H-pyrazol-5-yl)dimethylphosphine oxide: To a solution of (3-bromo-1-methyl-1H-pyrazol-5-yl)dimethylphosphine oxide (1.0 g, 4.2 mmol) in 1,4-dioxane (20 mL) was added (Bpin)₂ (1.6 g, 6.3 mmol), KOAc (1.2 g, 13 mmol), Pd(dppf)Cl₂ (307 mg, 0.42 mmol). The mixture was stirred at 100° C. under N₂ atmosphere overnight. Water (2 mL), K₂CO₃ (1.8 g, 13 mmol), Pd(dppf)Cl₂ (154 mg, 0.21 mmol) and 5-bromo-4-methoxypyridin-3-amine (685 mg, 3.4 mmol) was added to the solution. The mixture was stirred at 100° C. for 8 hours. The solvent was removed and the residue was purified by silica gel chromatography column (DCM/MeOH=12/1) to give the desired product (1.1 g, 92%) as a black oil. ¹H-NMR (300 MHz, CDCl₃): δ 8.37 (s, 1H), 8.10 (s, 1H), 6.84 (s, 1H), 4.29 (s, 3H), 3.69 (s, 3H), 1.87 (d, J=13.8 Hz, 6H). LC-MS: m/z 280.9 [M+H]⁺.

Step b. 6-Chloro-4-((5-(5-(dimethylphosphoryl)-1-methyl-1H-pyrazol-3-yl)-4-methoxypyridin-3-yl)amino)pyridazine-3-carboxamide: To a solution of 4,6-dichloropyridazine-3-carboxamide (165 mg, 0.43 mmol) and (3-(5-amino-4-methoxypyridin-3-yl)-1-methyl-1H-pyrazol-5-yl)dimethylphosphine oxide (200 mg, 0.36 mmol) in anhydrous tetrahydrofuran (8 mL) was added 1N LiHMDS (2.1 mL, 2.1 mmol) under N₂ atmosphere. The mixture was stirred at room temperature for 24 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=20/1) to give the final compound (140 mg, 89%) as a yellow oil. ¹H-NMR (300 MHz, DMSO-d₆): δ 10.91 (s, 1H), 8.86 (s, 1H), 8.78 (s, 1H), 8.60 (s, 1H), 8.13 (s, 1H), 7.08 (s, 1H), 7.06 (s, 1H), 4.18 (s, 3H), 3.75 (s, 3H), 1.83 (d, J=13.8 Hz, 6H). LC-MS: m/z 435.7 [M+H]⁺.

Step c. 4-((5-(5-(Dimethylphosphoryl)-1-methyl-1H-pyrazol-3-yl)-4-methoxypyridin-3-yl)amino)-6-((4-methylpyridin-2-yl)amino)pyridazine-3-carboxamide (A57): To a solution of 6-chloro-4-((5-(5-(dimethylphosphoryl)-1-methyl-1H-pyrazol-3-yl)-4-methoxypyridin-3-yl)amino)pyridazine-3-carboxamide (135 mg, 0.31 mmol) in 1,4-dioxane (2 mL) was added 4-methylpyridin-2-amine (101 mg, 0.93 mmol), Pd₂(dba)₃ (29 mg, 0.030 mmol), dppf (34 mg, 0.060 mmol) and K₃PO₄ (198 mg, 0.93 mmol). The mixture was stirred at 115° C. under N₂ atmosphere in a microwave apparatus for 2.5 hours. The solvent was filtered and the filtrate was concentrated. The residue was purified by silica gel chromatography column (DCM/MeOH=30/1) to give the crude product, which was rinsed by Et₂O to give the desired product (60 mg, 38%) as a gray solid.

The similar procedure described in Example 18 was carried out to get the Compound A58.

### Example 19, Method AB, AC, AJ, AN

### Preparation of 6-(cyclopropanecarboxamido)-4-((4′-(dimethylphosphoryl)-2-methoxy-[1,1′-biphenyl]-3-yl)amino)nicotinamide (A61)

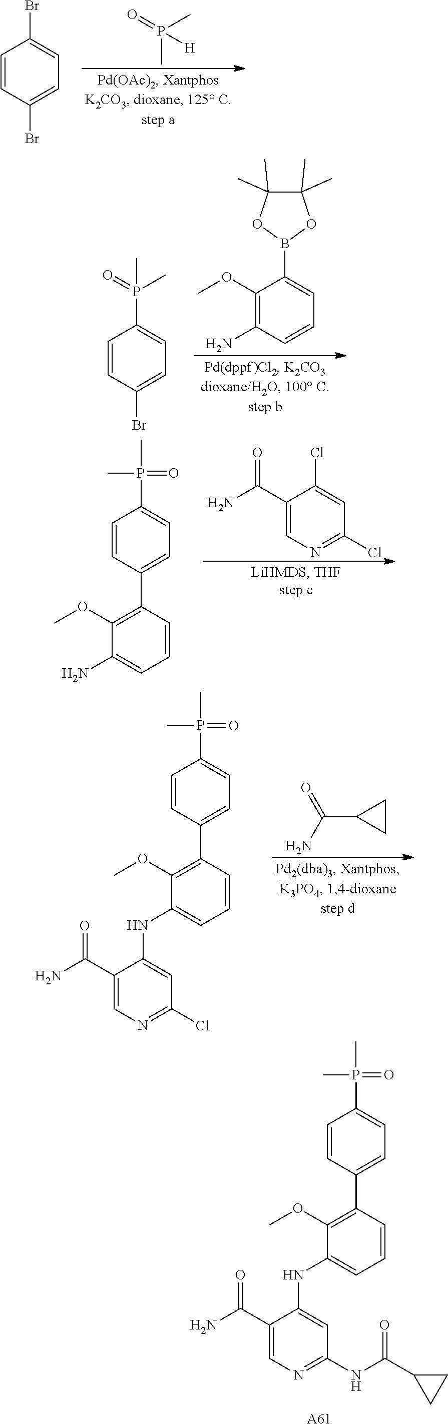

Step a. (4-Bromophenyl)dimethylphosphine oxide: To a solution of 1,4-dibromobenzene (585 mg, 2.5 mmol) in 1,4-dioxane (10 mL) was added K₂CO₃ (518 mg, 3.8 mmol), dimethylphosphine oxide (234 mg, 3.0 mmol), Pd(OAc)₂ (56 mg, 0.25 mmol) and Xantphos (115 mg, 0.20 mmol). The mixture was stirred at 125° C. under N₂ atmosphere for 2 hours. The solvent was concentrated and the residue was purified by silica gel chromatography column (DCM/MeOH=100/3) to give the final compound (120 mg, 21%) as a yellow oil. LC-MS: m/z 233.0 [M+H]⁺.

Step b. (3′-Amino-2′-methoxy-[1,1′-biphenyl]-4-yl)dimethylphosphine oxide: To a solution of 2-methoxy-3-(4,4,5,5-tetramethyl-1,3,2-dioxaborolan-2-yl)aniline (107 mg, 0.43 mmol) and (4-bromophenyl)dimethylphosphine oxide (90 mg, 0.39 mmol) in 1,4-dioxane/water (10 mL/1 mL) was added Pd(dppf)Cl₂ (28 mg, 0.039 mmol) and K₂CO₃ (107 mg, 0.78 mmol). The mixture was stirred at 100° C. overnight under N₂ atmosphere. The solvent was removed and the residue was purified by silica gel chromatography column (DCM/MeOH=100/3) to give the compound (65 mg, 61%) as a brown solid. ¹H-NMR (300 MHz, CDCl₃): δ 7.88-7.62 (m, 4H), 7.07-6.92 (m, 1H), 6.85-6.66 (m, 2H), 3.95 (br, s, 2H), 3.40 (s, 3H), 1.87 (d, J=13.8 Hz, 6H). LC-MS: m/z 276.1 [M+H]⁺.

Step c. 6-Chloro-4-((4′-(dimethylphosphoryl)-2-methoxy-[1,1′-biphenyl]-3-yl)amino)nicotinamide: To a solution of 4,6-dichloronicotinamide (104 mg, 0.54 mmol) and (3′-amino-2′-methoxy-[1,1′-biphenyl]-4-yl)dimethylphosphine oxide (100 mg, 0.36 mmol) in anhydrous tetrahydrofuran (3 mL) was added 1N LiHMDS (1.4 mL, 1.4 mmol) under N₂ atmosphere. The mixture was stirred at room temperature overnight. Saturated NH₄Cl (5 mL) aqueous solution was added and the aqueous layer was extracted with ethyl acetate (10 mL×3). The combined organic layer was dried by Na₂SO₄ and concentrated. The residue was purified by silica gel chromatography column (DCM/MeOH=15/1) to give the final compound (94 mg, 61%) as a yellow oil.

Step d. 6-(Cyclopropanecarboxamido)-4-((4′-(dimethylphosphoryl)-2-methoxy-[1,1′-biphenyl]-3-yl)amino)nicotinamide (A61): To a solution of 6-chloro-4-((4′-(dimethylphosphoryl)-2-methoxy-[1,1′-biphenyl]-3-yl)amino)nicotinamide (94 mg, 0.22 mmol) in 1,4-dioxane (2 mL) was added cyclopropanecarboxamide (56 mg, 0.66 mmol), Pd₂(dba)₃ (21 mg, 0.020 mmol), Xantphos (13 mg, 0.020 mmol) and K₃PO₄ (92 mg, 0.43 mmol). The mixture was stirred at 135° C. under N₂ atmosphere in a microwave apparatus for 1.5 hours. The solvent was filtered and the filtrate was concentrated. The residue was purified by silica gel chromatography column (DCM/MeOH=15/1) to give the crude product, which was rinsed by Et₂O to give the desired product (17 mg, 18%) as a yellow solid.

The similar procedure described in Example 19 was carried out to get the Compound A62.

### Example 20, Method AB, AK, AN

### Preparation of 6-(cyclopropanecarboxamido)-4-((3-(5-(dimethylphosphoryl)pyrazin-2-yl)-2-methoxyphenyl)amino)nicotinamide (A63)

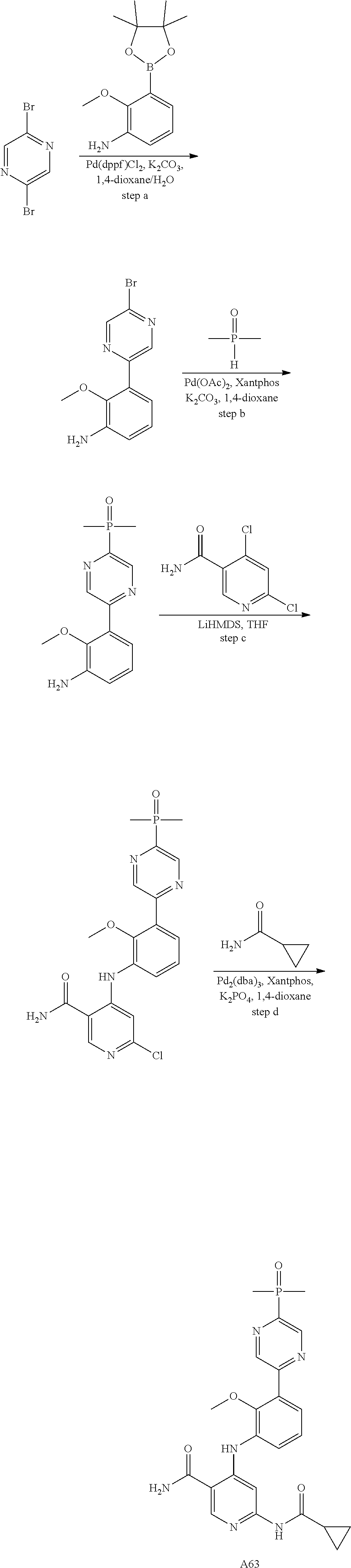

Step a. 3-(5-Bromopyrazin-2-yl)-2-methoxyaniline: To a solution of 2,5-dibromopyrazine (1.0 g, 4.2 mmol) and 2-methoxy-3-(4,4,5,5-tetramethyl-1,3,2-dioxaborolan-2-yl)aniline (1.1 g, 4.2 mmol) in 1,4-dioxane/water (100 mL/5 mL) was added Pd(dppf)Cl₂ (0.31 g, 0.42 mmol) and K₂CO₃ (1.2 g, 8.4 mmol). The mixture was stirred at 90° C. overnight under N₂ atmosphere. The solvent was removed and the residue was purified by silica gel chromatography column (PE/EA=2/1) to give the compound (470 mg, 40%) as a yellow oil. ¹H-NMR (300 MHz, DMSO-d₆): δ 8.91 (s, 1H), 8.84 (s, 1H), 6.97 (t, J=7.5 Hz, 1H), 6.88 (d, J=7.8 Hz, 1H), 6.85 (s, 1H), 5.14 (s, 2H), 3.45 (s, 3H). LC-MS: m/z 280.0 [M+H]⁺.

Step b. (5-(3-Amino-2-methoxyphenyl)pyrazin-2-yl)dimethylphosphine oxide: To a solution of 3-(5-bromopyrazin-2-yl)-2-methoxyaniline (200 mg, 0.71 mmol) in 1,4-dioxane (10 mL) was added K₂CO₃ (160 mg, 1.1 mmol), dimethylphosphine oxide (84 mg, 1.1 mmol), Pd(OAc)₂ (20 mg, 0.072 mmol) and Xantphos (42 mg, 0.072 mmol). The mixture was stirred at 125° C. under N₂ atmosphere for 1 hour. The solvent was concentrated and the residue was purified by silica gel chromatography column (DCM/MeOH=100/3) to give the final compound (194 mg, 97%) as a cyan solid. ¹H-NMR (300 MHz, DMSO-d₆): δ 9.17 (s, 1H), 9.12 (s, 1H), 7.04-6.96 (m, 1H), 6.92 (d, J=6.9 Hz, 1H), 6.87 (d, J=7.5 Hz, 1H), 5.18 (s, 2H), 3.57 (s, 3H), 1.77 (d, J=13.8 Hz, 6H). LC-MS: m/z 278.1 [M+H]⁺.

Step c. 6-Chloro-4-((3-(5-(dimethylphosphoryl)pyrazin-2-yl)-2-methoxyphenyl)amino)nicotinamide: To a solution of 4,6-dichloronicotinamide (176 mg, 0.92 mmol) and (5-(3-amino-2-methoxyphenyl)pyrazin-2-yl)dimethylphosphine oxide (170 mg, 0.61 mmol) in anhydrous tetrahydrofuran (7 mL) was added 1N LiHMDS (2.4 mL, 2.4 mmol) under N₂ atmosphere. The mixture was stirred at room temperature for 2 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=15/1) to give the final compound (50 mg, 19%) as a yellow solid. ¹H-NMR (300 MHz, DMSO-d₆): δ 10.87 (s, 1H), 9.22 (s, 1H), 9.18 (s, 1H), 8.60 (s, 1H), 8.42-8.33 (m, 1H), 7.85-7.78 (m, 1H), 7.64-7.60 (m, 2H), 7.39 (t, J=8.4 Hz, 1H), 6.94 (s, 1H), 3.54 (s, 3H), 1.79 (d, J=13.8 Hz, 6H). LC-MS: m/z 431.7 [M+H]⁺.

Step d. 6-(Cyclopropanecarboxamido)-4-((3-(5-(dimethylphosphoryl)pyrazin-2-yl)-2-methoxyphenyl)amino)nicotinamide (A63): To a solution of 6-chloro-4-((3-(5-(dimethylphosphoryl)pyrazin-2-yl)-2-methoxyphenyl)amino)nicotinamide (48 mg, 0.11 mmol) in 1,4-dioxane (2 mL) was added cyclopropanecarboxamide (28 mg, 0.33 mmol), Pd₂(dba)₃ (6.0 mg, 0.010 mmol), Xantphos (10 mg, 0.010 mmol) and K₃PO₄ (47 mg, 0.22 mmol). The mixture was stirred at 120° C. under N₂ atmosphere in a microwave apparatus for 2 hours. The solvent was filtered and the filtrate was concentrated. The residue was purified by silica gel chromatography column (DCM/MeOH=30/1) to give the crude product, which was rinsed by Et₂O to give the desired product (10 mg, 19%) as a yellow solid.

The similar procedure described in Example 20 was carried out to get the Compound A64.

### Example 21, Method AQ, AN

### Preparation of 4-((4-(dicyclopropylphosphoryl)-2-methoxyphenyl)amino)-6-((4-methylpyridin-2-yl)amino)pyridazine-3-carboxamide (B1)

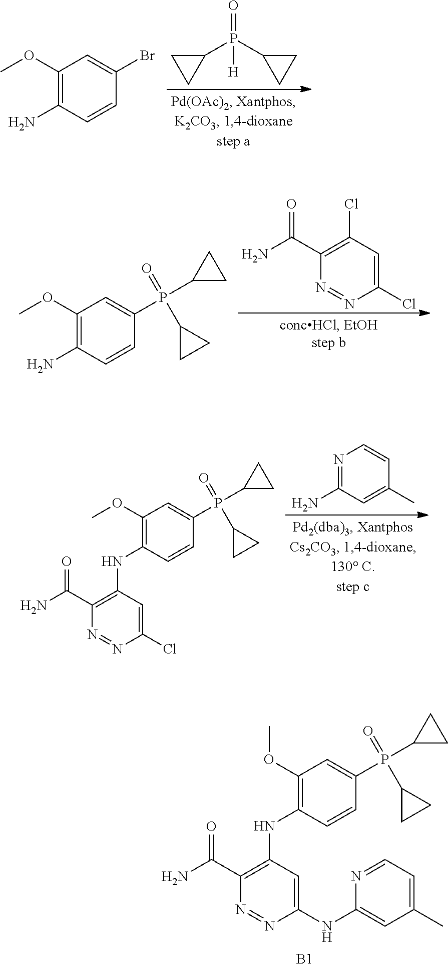

Step a. (4-Amino-3-methoxyphenyl)dicyclopropylphosphine oxide: To a solution of 4-bromo-2-methoxyaniline (3.3 g, 16 mmol) in 1,4-dioxane (10 mL) was added K₂CO₃ (3.4 g, 25 mmol), dicyclopropylphosphine oxide (4.3 g, 33 mmol), Pd(OAc)₂ (370 mg, 1.7 mmol) and Xantphos (953 mg, 1.7 mmol). The mixture was stirred at 120° C. under N₂ atmosphere for 2 hours. The solvent was concentrated and the residue was purified by silica gel chromatography column (DCM/MeOH=100/3) to give the final compound (2.2 g, 54%) as a brown solid. ¹H-NMR (300 MHz, CDCl₃): δ 7.22 (d, J=11.4 Hz, 1H), 7.16 (d, J=10.5 Hz, 1H), 6.74 (s, 1H), 4.10 (s, 2H), 3.90 (s, 3H), 1.04-0.72 (m, 10H). LC-MS: m/z 251.9 [M+H]⁺.

Step b. 6-Chloro-4-((4-(dicyclopropylphosphoryl)-2-methoxyphenyl)amino)pyridazine-3-carboxamide: To a solution of 4,6-dichloropyridazine-3-carboxamide (1.4 g, 7.2 mmol) and (4-amino-3-methoxyphenyl)dicyclopropylphosphine oxide (1.5 g, 6.0 mmol) in EtOH (40 mL) was added a catalytic amount of conc. HCl (one drop). The mixture was stirred at 120° C. for 2.5 hours in a microwave reactor. The solvent was concentrated in vacuum and the residue was purified by silica gel chromatography column (DCM/MeOH=20/1) to give the desired product (770 mg, 32%) as a white solid. ¹H-NMR (300 MHz, CDCl₃): δ 10.75 (s, 1H), 8.07 (s, 1H), 7.54-7.37 (m, 3H), 7.10 (s, 1H), 5.70 (s, 1H), 3.95 (s, 3H), 1.12-0.81 (m, 10H). LC-MS: m/z 406.8 [M+H]⁺.

Step c. 4-((4-(Dicyclopropylphosphoryl)-2-methoxyphenyl)amino)-6-((4-methylpyridin-2-yl)amino)pyridazine-3-carboxamide (B1): To a solution of 6-chloro-4-((4-(dicyclopropylphosphoryl)-2-methoxyphenyl)amino)pyridazine-3-carboxamide (70 mg, 0.14 mmol) in 1,4-dioxane (2 mL) was added 4-methylpyridin-2-amine (49 mg, 0.0.42 mmol), Pd₂(dba)₃ (15 mg, 0.014 mmol), Xantphos (9 mg, 0.014 mmol) and Cs₂CO₃ (91 mg, 0.28 mmol). The mixture was stirred at 130° C. under N₂ atmosphere in a microwave apparatus for 1.5 hours. The solvent was filtered and the filtrate was concentrated. The residue was purified by silica gel chromatography column (DCM/MeOH=10/1) to give the crude product, which was rinsed by Et₂O to give the desired product (10 mg, 24%) as a yellow solid.

The similar procedure described in Example 21 was carried out to get the Compound B2, B4, B5, B6, B7, and B8.

### Example 22, Method AQ, AN

### Preparation of 6-(cyclopropanecarboxamido)-4-((4-(diethylphosphoryl)-2-methoxyphenyl)amino)nicotinamide (B3)

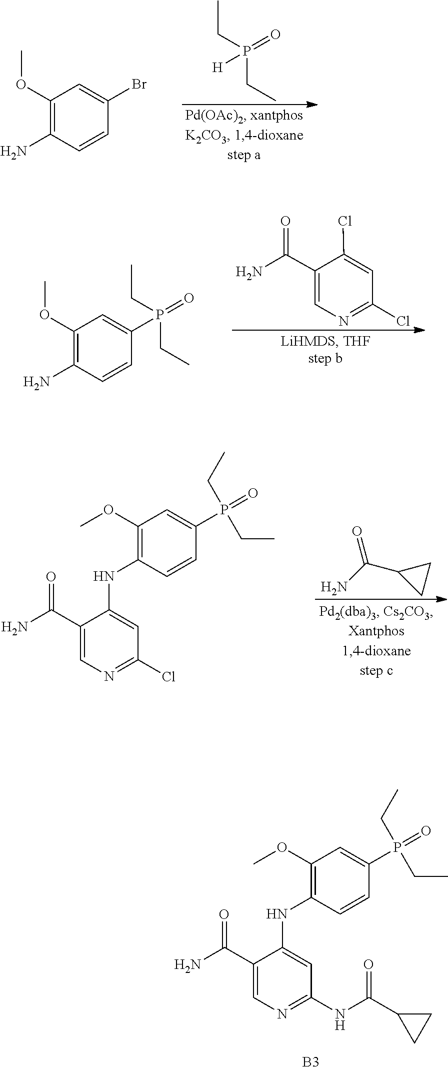

Step a. (4-Amino-3-methoxyphenyl)diethylphosphine oxide: To a solution of 4-bromo-2-methoxyaniline (700 mg, 3.5 mmol) in 1,4-dioxane (20 mL) was added K₂CO₃ (731 mg, 5.3 mmol), diethylphosphine oxide (742 mg, 7.0 mmol), Pd(OAc)₂ (79 mg, 0.35 mmol) and Xantphos (203 mg, 0.35 mmol). The mixture was stirred at 115° C. under N₂ atmosphere for 12 hours. The solvent was concentrated and the residue was purified by silica gel chromatography column (DCM/MeOH=50/1) to give the final compound (400 mg, 51%) as a yellow oil. ¹H-NMR (300 MHz, CDCl₃): δ 7.19 (d, J=11.1 Hz, 1H), 6.99-6.88 (m, 1H), 6.78-6.69 (m, 1H), 4.11 (s, 2H), 3.91 (s, 3H), 2.00-1.81 (m, 4H), 1.24-1.02 (m, 6H). LC-MS: m/z 227.9 [M+H]⁺.

Step b. 6-Chloro-4-((4-(diethylphosphoryl)-2-methoxyphenyl)amino)nicotinamide: To a solution of 4,6-dichloronicotinamide (189 mg, 0.99 mmol) and (4-amino-3-methoxyphenyl)diethylphosphine oxide (150 mg, 0.66 mmol) in anhydrous tetrahydrofuran (7 mL) was added 1N LiHMDS (2.7 mL, 2.7 mmol) under N₂ atmosphere. The mixture was stirred at room temperature for 2 hours. Saturated NH₄Cl (30 mL) aqueous solution was added and the aqueous layer was extracted with ethyl acetate (30 mL×3). The combined organic layer was dried by Na₂SO₄ and concentrated. The residue was purified by silica gel chromatography column (DCM/MeOH=20/1) to give the final compound (150 mg, 60%) as a yellow solid. ¹H-NMR (300 MHz, DMSO-d₆): δ 10.79 (s, 1H), 8.59 (s, 1H), 8.32 (s, 1H), 7.74 (s, 1H), 7.62-7.54 (m, 1H), 7.40-7.29 (m, 2H), 7.03 (s, 1H), 3.89 (s, 3H), 2.02-1.83 (m, 4H), 1.05-0.85 (m, 6H). LC-MS: m/z 381.8 [M+H]⁺.

Step c. 6-(Cyclopropanecarboxamido)-4-((4-(diethylphosphoryl)-2-methoxyphenyl)amino)nicotinamide (B3): To a solution of 6-chloro-4-((4-(diethylphosphoryl)-2-methoxyphenyl)amino)nicotinamide (100 mg, 0.26 mmol) in 1,4-dioxane (2 mL) was added cyclopropanecarboxamide (67 mg, 0.79 mmol), Pd₂(dba)₃ (24 mg, 0.026 mmol), Xantphos (15 mg, 0.026 mmol) and Cs₂CO₃ (170 mg, 0.52 mmol). The mixture was stirred at 130° C. under N₂ atmosphere in a microwave apparatus for 3 hours. The solvent was filtered and the filtrate was concentrated. The residue was purified by silica gel chromatography column (DCM/MeOH=30/1) to give the crude product, which was rinsed by Et₂O to give the desired product (14 mg, 12%) as a yellow solid.

The similar procedure described in Example 22 was carried out to get the Compound B9.

### Example 23, Method AR

### Preparation of 6-(cyclopropanecarboxamido)-4-((3-(dicyclopropylphosphoryl)-2-methoxyphenyl)amino)pyridazine-3-carboxamide (B10)

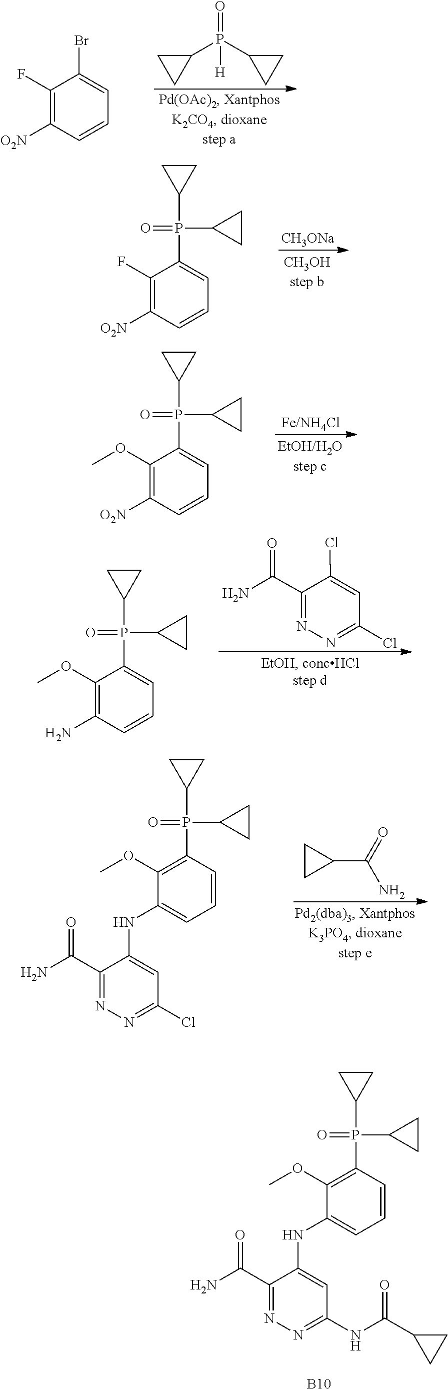

Step a. Dicyclopropyl(2-fluoro-3-nitrophenyl)phosphine oxide: To a solution of 1-bromo-2-fluoro-3-nitrobenzene (1.8 g, 8.2 mmol) in 1,4-dioxane (20 mL) was added K₃PO₄ (2.6 g, 12 mmol), dicyclopropylphosphine oxide (2.1 g, 16 mmol), Pd(OAc)₂ (184 mg, 0.80 mmol) and Xantphos (475 mg, 0.80 mmol). The mixture was stirred at 120° C. under N₂ atmosphere for 0.5 hour in a microwave reactor. The solvent was concentrated and the residue was purified by silica gel chromatography column (DCM/MeOH=30/1) to give the final compound (600 mg, 27%) as a yellow oil. ¹H-NMR (300 MHz, CDCl₃): δ 8.28-8.04 (m, 2H), 7.44 (t, J=7.5 Hz, 1H), 1.30-0.70 (m, 10H). LC-MS: m/z 269.9 [M+H]⁺.

Step b. Dicyclopropyl(2-methoxy-3-nitrophenyl)phosphine oxide: To a solution of dicyclopropyl(2-fluoro-3-nitrophenyl)phosphine oxide (600 mg, 2.2 mmol) in MeOH (10 mL) was added NaOCH₃ (181 mg, 3.3 mmol). The mixture was stirred at room temperature for 4 hours. The solvent was concentrated in vacuum and the residue was purified by silica gel chromatography column (DCM/MeOH=30/1) to give the final compound (370 mg, 59%) as a yellow oil. ¹H-NMR (300 MHz, CDCl₃): δ 8.09-8.07 (m, 1H), 8.01 (d, J=7.8 Hz, 1H), 7.31 (t, J=7.5 Hz, 1H), 3.96 (s, 3H), 1.30-0.70 (m, 10H). LC-MS: m/z 281.9 [M+H]⁺.

Step c. (3-Amino-2-methoxyphenyl)dicyclopropylphosphine oxide: To a solution of dicyclopropyl(2-methoxy-3-nitrophenyl)phosphine oxide (300 mg, 1.1 mmol) in a mixed solvent of EtOH and water (15 mL/5 mL) was added iron powder (240 mg, 4.4 mmol) and NH₄Cl (115 mg, 2.2 mmol). The mixture was stirred at 80° C. for 3 hours, and then filtered. The filtrate was concentrated and the residue was purified by silica gel chromatography column (DCM/MeOH=20/1) to give the desired product (160 mg, 60%) as a brown oil. ¹H-NMR (300 MHz, CDCl₃): δ 7.19 (d, J=12.0 Hz, 1H), 7.02 (t, J=6.9 Hz, 1H), 6.92 (d, J=7.8 Hz, 1H), 3.91 (s, 3H), 3.81 (s, 2H), 1.26-0.74 (m, 10H). LC-MS: m/z 251.9 [M+H]⁺.

Step d. 6-Chloro-4-((3-(dicyclopropylphosphoryl)-2-methoxyphenyl)amino)pyridazine-3-carboxamide: To a solution of 4,6-dichloropyridazine-3-carboxamide (147 mg, 0.80 mmol) and (3-amino-2-methoxyphenyl)dicyclopropylphosphine oxide (160 mg, 0.60 mmol) in EtOH (2 mL) was added a catalytic amount of conc. HCl (one drop). The mixture was stirred at 120° C. for 2.5 hours in a microwave reactor. The solvent was concentrated in vacuum and the residue was purified by silica gel chromatography column (DCM/MeOH=20/1) to give the desired product (120 mg, 46%) as a yellow solid. ¹H-NMR (300 MHz, DMSO-d₆): δ 10.94 (s, 1H), 8.78 (s, 1H), 8.13 (s, 1H), 7.70 (d, J=7.5 Hz, 1H), 7.56 (d, J=10.2 Hz, 1H), 7.34 (t, J=6.9 Hz, 1H), 7.01 (s, 1H), 3.76 (s, 3H), 1.38-1.25 (m, 2H), 0.92-0.49 (m, 8H). LC-MS: m/z 406.8 [M+H]⁺.

Step e. 6-(Cyclopropanecarboxamido)-4-((3-(dicyclopropylphosphoryl)-2-methoxyphenyl)amino)pyridazine-3-carboxamide (B10): To a solution of 6-chloro-4-((3-(dicyclopropylphosphoryl)-2-methoxyphenyl)amino)pyridazine-3-carboxamide (60 mg, 0.15 mmol) in 1,4-dioxane (2 mL) was added cyclopropanecarboxamide (38 mg, 0.44 mmol), Pd₂(dba)₃ (14 mg, 0.010 mmol), Xantphos (9 mg, 0.010 mmol) and K₃PO₄ (63 mg, 0.30 mmol). The mixture was stirred at 115° C. under N₂ atmosphere in a microwave apparatus for 2 hours. The solvent was filtered and the filtrate was concentrated. The residue was purified by silica gel chromatography column (DCM/MeOH=10/1) to give the crude product, which was rinsed by Et₂O to give the desired product (1.3 mg, 2%) as a yellow solid.

Table 1 shows a selection of the compounds prepared according to the methods described above and the method numbers are indicated in the third column of the table.

TABLE 1
Selected compounds (A1-A67, B1-B10) of the present invention

| NO. | Structure | Method | ¹HNMR & LCMS-MS |
|---|---|---|---|
| A1 | 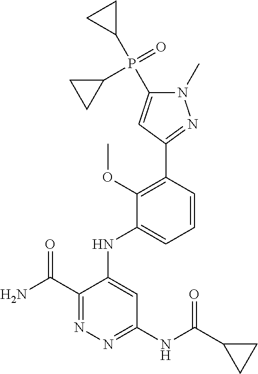 [SMILES: COc1c(Nc2cc(NC(=O)C3CC3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.32 (s, 1H), 11.02 (s, 1H), 8.55 (s, 1H), 8.16 (s, 1H), 7.88 (s, 1H), 7.69 (d, J = 7.5 Hz, 1H), 7.43 (d, J = 7.5 Hz, 1H), 7.26 (d, J = 7.8 Hz, 1H), 7.23 (s, 1H), 4.13 (s, 3H), 3.61 (s, 3H), 2.16-1.98 (m, 1H), 1.45-1.24 (m, 2H), 0.99-0.68 (m, 12H). LC-MS: m/z 536.3 [M + H]⁺. |
| A2 | 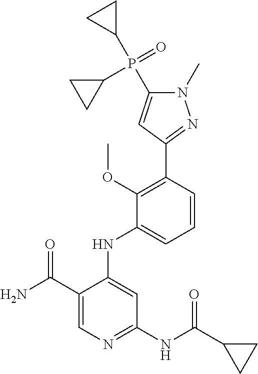 [SMILES: COc1c(Nc2cc(NC(=O)C3CC3)ncc2C(N)=O)cccc1-c1cc(P(=O)(C2CC2)C2CC2)n(C)n1] | AA, AB, AC, AN | ¹H-NMR (300 MHz, DMSO-d₆): δ 10.94 (s, 1H), 10.79 (s, 1H), 8.60 (s, 1H), 8.18 (s, 1H), 8.01 (s, 1H), 7.61 (s, 1H), 7.50 (s, 1H), 7.40 (s, 1H), 7.30-7.18 (m, 1H), 7.07 (s, 1H), 4.12 (s, 3H), 3.61 (s, 3H), 2.06- 1.94 (m, 1H), 1.42-1.29 (m, 2H), 1.00-0.71 (m, 12H). LC-MS: m/z 534.8 [M + H]⁺. |
| A3 | 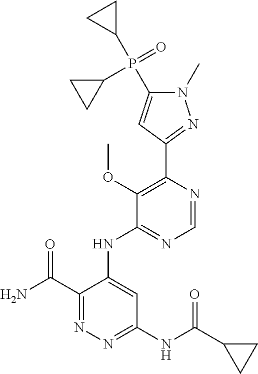 [SMILES: COc1c(Nc2cc(NC(=O)C3CC3)nnc2C(N)=O)ncnc1-c1cc(P(=O)(C2CC2)C2CC2)n(C)n1] | AA, AD, AF, AI, AN | ¹H-NMR (300 MHz, DMSO-d₆): δ 12.86 (s, 1H), 11.49 (s, 1H), 9.94 (s, 1H), 8.76 (s, 1H), 6.85 (s, 1H), 8.12 (s, 1H), 7.44 (s, 1H), 4.19 (s, 3H), 3.91 (s, 3H), 2.20-2.09 (m, 1H), 1.50-1.30 (m, 2H), 0.99-0.74 (m, 12H). LC-MS: m/z 537.7 [M + H]⁺. |
| A4 | 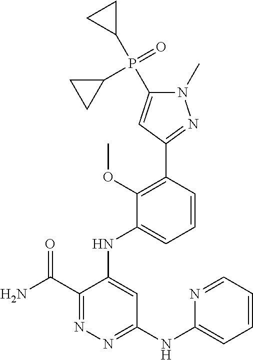 [SMILES: COc1c(Nc2cc(Nc3ccccn3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.04 (s, 1H), 10.17 (s, 1H), 8.49 (s, 1H), 8.24- 8.13 (m, 2H), 7.79-7.75 (m, 1H), 7.71-7.68 (m, 2H), 7.61-7.53 (m, 2H), 7.34-7.28 (m, 1H), 7.26-7.21 (m, 1H), 6.96-6.86 (m, 1H), 4.13 (s, 3H), 3.64 (s, 3H), 1.40-1.29 (m, 2H), 0.96-0.76 (m, 8H). LC-MS: m/z 544.8 [M + H]⁺. |
| A5 | 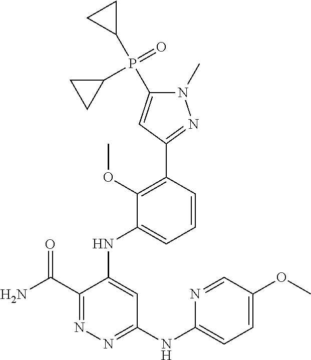 [SMILES: COc1ccc(Nc2cc(Nc3cccc(-c4cc(P(=O)(C5CC5)C5CC5)n(C)n4)c3OC)c(C(N)=O)nn2)nc1] | AA, AB, AC, AI, AN | ¹H-NMR (300 MHz, DMSO-d₆): δ 11.02 (s, 1H), 9.97 (s, 1H), 8.47 (s, 1H), 7.98 (s, 1H), 7.91 (s, 1H), 7.73 (s, 1H), 7.68-7.59 (m, 2H), 7.54 (d, J = 7.2 Hz, 1H), 7.42 (d, J = 8.1 Hz, 1H), 7.35-7.28 (m, 1H), 7.24 (s, 1H), 4.13 (s, 3H), 3.78 (s, 3H), 3.63 (s, 3H), 1.44-1.27 (m, 2H), 0.98-0.71 (m, 8H). LC-MS: m/z 574.8 [M + H]⁺. |
| A6 | 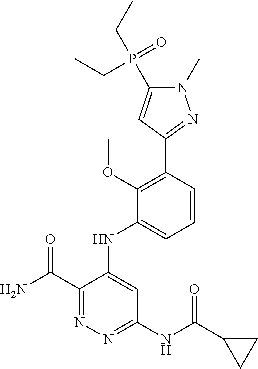 [SMILES: CCP(=O)(CC)c1cc(-c2cccc(Nc3cc(NC(=O)C4CC4)nnc3C(N)=O)c2OC)nn1C] | AA, AB, AC, AI, AN | ¹H-NMR (300 MHz, DMSO-d₆): δ 11.32 (s, 1H), 11.02 (s, 1H), 8.54 (s, 1H), 8.15 (s, 1H), 7.88 (s, 1H), 7.67 (d, J = 6.9 Hz, 1H), 7.42 (d, J = 6.6 Hz, 1H), 7.36-7.17 (m, 1H), 7.06 (s, 1H), 4.17 (s, 3H), 3.58 (s, 3H), 2.17-1.95 (m, 5H), 1.17-0.95 (m, 6H), 0.84-0.75 (m, 4H). LC-MS: m/z 511.8 [M + H]⁺. |
| A7 | 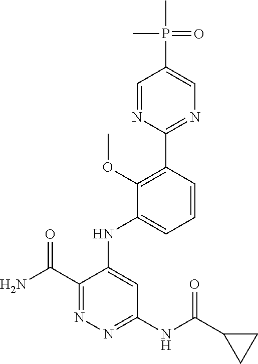 [SMILES: COc1c(Nc2cc(NC(=O)C3CC3)nnc2C(N)=O)cccc1-c1ncc(P(C)(C)=O)cn1] | AB, AK, AI, AN | ¹H-NMR (300 MHz, DMSO-d₆): δ 11.34 (s, 1H), 11.02 (s, 1H), 9.24 (s, 2H), 8.55 (s, 1H), 8.15 (s, 1H), 7.89 (s, 1H), 7.67-7.53 (m, 2H), 7.34 (s, 1H), 3.70 (s, 3H), 2.15- 2.02 (m, 1H), 1.83 (d, J = 13.8 Hz, 6H), 0.90-0.75 (m, 4H). LC-MS: m/z 481.8 [M + H]⁺. |
| A8 | 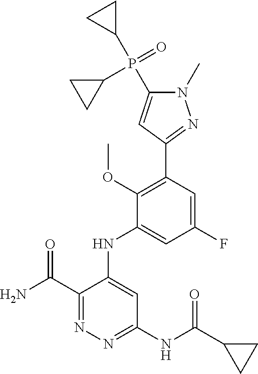 [SMILES: COc1c(Nc2cc(NC(=O)C3CC3)nnc2C(N)=O)cc(F)cc1-c1cc(P(=O)(C2CC2)C2CC2)n(C)n1] | AA, AB, AC, AI, AN | ¹H-NMR (300 MHz, DMSO-d₆): δ 11.40 (s, 1H), 11.23 (s, 1H), 8.59 (s, 1H), 8.26 (s, 1H), 7.93 (s, 1H), 7.58-7.16 (m, 3H), 4.13 (s, 3H), 3.62 (s, 3H), 2.16-2.05 (m, 1H), 1.41-1.27 (m, 2H), 1.04-0.65 (m, 12H). LC-MS: m/z 553.8 [M + H]⁺. |
| A9 | 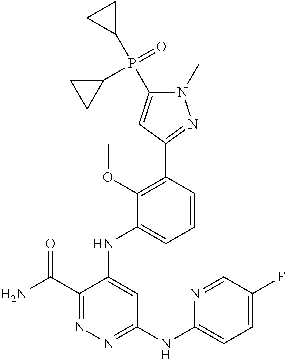 [SMILES: COc1c(Nc2cc(Nc3ccc(F)cn3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.04 (s, 1H), 10.21 (s, 1H), 8.50 (s, 1H), 8.18 (s, 1H), 7.97 (s, 1H), 7.77 (s, 1H), 7.75-7.63 (m, 3H), 7.54 (d, J = 7.5 Hz, 1H), 7.32 (t, J = 7.8 Hz, 1H), 7.24 (s, 1H), 4.13 (s, 3H), 3.63 (s, 3H), 1.43-1.26 (m, 2H), 1.01-0.63 (m, 8H). LC-MS: m/z 562.8 [M + H]⁺. |
| A10 | 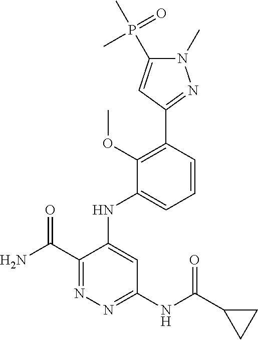 [SMILES: COc1c(Nc2cc(NC(=O)C3CC3)nnc2C(N)=O)cccc1-c1cc(P(C)(C)=O)n(C)n1] | AA, AB, AC, AI, AN | ¹H-NMR (300 MHz, DMSO-d₆): δ 11.31 (s, 1H), 11.01 (s, 1H), 8.54 (s, 1H), 8.14 (s, 1H), 7.88 (s, 1H), 7.67 (d, J = 7.5 Hz, 1H), 7.42 (d, J = 7.8 Hz, 1H), 7.28-7.20 (m, 1H), 7.07 (s, 1H), 4.16 (s, 3H), 3.61 (s, 3H), 2.14-2.02 (m, 1H), 1.82 (d, J = 13.8 Hz, 6H), 0.91-0.76 (m, 4H). LC-MS: m/z 483.8 [M + H]⁺. |
| A11 | 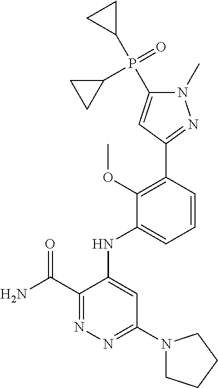 [SMILES: COc1c(Nc2cc(N3CCCC3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 10.92 (s, 1H), 8.32 (s, 1H), 7.62 (d, J = 7.5 Hz, 1H), 7.59-7.52 (m, 2H), 7.29-7.17 (m, 2H), 6.23 (s, 1H), 4.12 (s, 3H), 3.62 (s, 3H), 3.53-3.42 (m, 4H), 1.99-1.89 (m, 4H), 1.47-1.28 (m, 2H), 1.00-0.72 (m, 8H). LC-MS: m/z 521.8 [M + H]⁺. |
| A12 | 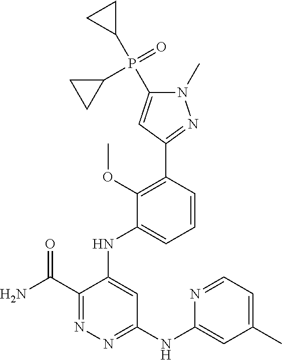 [SMILES: COc1c(Nc2cc(Nc3cc(C)ccn3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.05 (s, 1H), 10.09 (s, 1H), 8.52 (s, 1H), 8.15 (s, 1H), 8.07 (s, 1H), 7.77 (s, 1H), 7.68 (d, J = 7.2 Hz, 1H), 7.57 (d, J = 7.2 Hz, 1H), 7.48 (s, 1H), 7.32 (d, J = 7.5 Hz, 1H), 7.25 (s, 1H), 6.80 (s, 1H), 4.14 (s, 3H), 3.65 (s, 3H), 2.29 (s, 3H), 1.49-1.25 (m, 2H), 0.98- 0.74 (m, 8H). LC-MS: m/z 558.8 [M + H]⁺. |
| A13 | 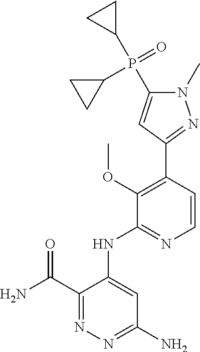 [SMILES: COc1c(-c2cc(P(=O)(C3CC3)C3CC3)n(C)n2)ccnc1Nc1cc(N)nnc1C(N)=O] | AA, AE, AG, AI, AL | ¹H-NMR (300 MHz, DMSO-d₆): δ 12.27 (s, 1H), 8.37 (s, 1H), 8.22 (s, 1H), 8.09 (s, 1H), 7.62 (s, 1H), 7.43 (s, 1H), 7.42 (d, J = 3.6 Hz, 1H), 6.78 (s, 2H), 4.15 (s, 3H), 3.75 (s, 3H), 1.50-1.29 (m, 2H), 1.03-0.65 (m, 8H). LC-MS: m/z 468.8 [M + H]⁺. |
| A14 | 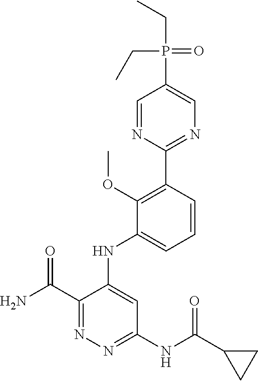 [SMILES: CCP(=O)(CC)c1cnc(-c2cccc(Nc3cc(NC(=O)C4CC4)nnc3C(N)=O)c2OC)nc1] | AA, AB, AK, AI, AN | ¹H-NMR (300 MHz, DMSO-d₆): δ 11.35 (s 1H), 11.03 (s, 1H), 9.19 (d, J = 4.5 Hz, 2H), 8.55 (s, 1H), 8.15 (s, 1H), 7.89 (s, 1H), 7.63 (d, J = 7.8 Hz, 2H), 7.41-7.29 (m, 1H), 3.69 (s, 3H), 2.20-1.97 (m, 4H), 1.27-1.19 (m, 1H), 1.09-0.94 (m, 6H), 0.81-0.69 (m, 4H). LC-MS: m/z 509.8 [M + H]⁺. |
| A15 | 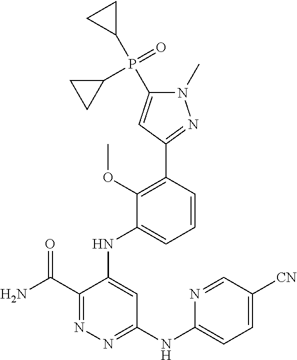 [SMILES: COc1c(Nc2cc(Nc3ccc(C#N)cn3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.08 (s, 1H), 10.74 (s, 1H), 8.63 (s, 1H), 8.55 (s, 1H), 8.12 (d, J = 7.5 Hz, 1H), 8.06 (s, 1H), 7.84 (s, 1H), 7.78 (d, J = 8.7 Hz, 1H), 7.69 (d, J = 7.5 Hz, 1H), 7.55 (d, J = 7.8 Hz, 1H), 7.39-7.30 (m, 1H), 7.23 (s, 1H), 4.13 (s, 3H), 3.63 (s, 3H), 1.50-1.22 (m, 2H), 1.02-0.55 (m, 8H). LC-MS: m/z 570.1 [M + H]⁺. |
| A16 | 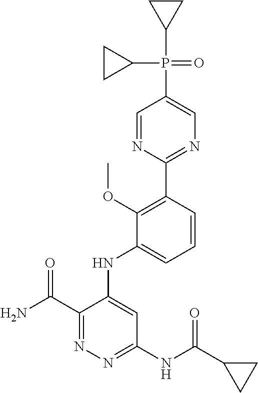 [SMILES: COc1c(Nc2cc(NC(=O)C3CC3)nnc2C(N)=O)cccc1-c1ncc(P(=O)(C2CC2)C2CC2)cn1] | AA, AB, AK, AI, AN | ¹H-NMR (300 MHz, DMSO-d₆): δ 11.33 (s, 1H), 11.08 (s, 1H), 9.26 (s, 2H), 8.54 (s, 1H), 8.15 (s, 1H), 7.88 (s, 1H), 7.62-7.57 (m, 2H), 7.35 (s, 1H), 3.72 (s, 3H), 2.15- 2.04 (m, 1H), 1.52-1.37 (m, 2H), 1.05-0.72 (m, 12H). LC-MS: m/z 533.8 [M + H]⁺. |
| A17 | 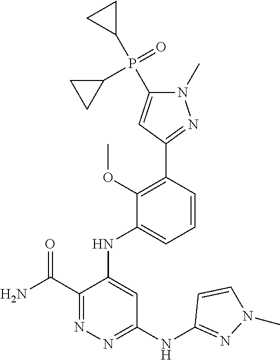 [SMILES: COc1c(Nc2cc(Nc3ccn(C)n3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 10.99 (s, 1H), 9.78 (s, 1H), 8.41 (s, 1H), 7.73 (s, 1H), 7.69-7.61 (m, 2H), 7.59-7.49 (m, 2H), 7.30 (d, J = 7.5 Hz, 1H), 7.27-7.18 (m, 1H), 6.15 (s, 1H), 4.13 (s, 3H), 3.71 (s, 3H), 3.63 (s, 3H), 1.41-1.28 (m, 2H), 0.99- 0.64 (m, 8H). LC-MS: m/z 547.8 [M + H]⁺. |
| A18 | 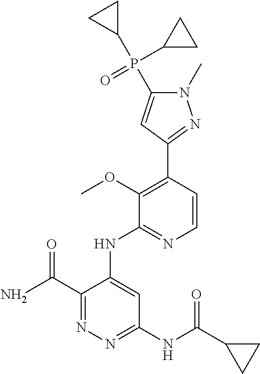 [SMILES: COc1c(-c2cc(P(=O)(C3CC3)C3CC3)n(C)n2)ccnc1Nc1cc(NC(=O)C2CC2)nnc1C(N)=O] | AA, AE, AG, AI, AN | ¹H-NMR (300 MHz, DMSO-d₆): δ 12.43 (s, 1H), 11.33 (s, 1H), 9.86 (s, 1H), 8.64 (s, 1H), 8.11 (d, J = 4.5 Hz, 1H), 7.97 (s, 1H), 7.47 (d, J = 4.5 Hz, 1H), 7.37 (s, 1H), 4.16 (s, 3H), 3.77 (s, 3H), 2.15-2.06 (m, 1H), 1.48-1.29 (m, 2H), 1.06-0.58 (m, 12H). LC-MS: m/z 536.8 [M + H]⁺. |
| A19 | 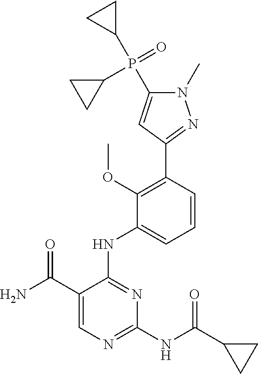 [SMILES: COc1c(Nc2nc(NC(=O)C3CC3)ncc2C(N)=O)cccc1-c1cc(P(=O)(C2CC2)C2CC2)n(C)n1] | AA, AB, AC, AN | ¹H-NMR (300 MHz, DMSO-d₆): δ 12.07 (s, 1H), 10.91 (s, 1H), 9.24-9.17 (m, 1H), 8.83 (s, 1H), 8.28-8.17 (m, 1H), 7.61 (s, 1H), 7.48 (s, 1H), 7.23 (s, 1H), 7.16-7.08 (m, 1H), 4.11 (s, 3H), 3.66 (s, 3H), 2.21- 2.10 (m, 1H), 1.44-1.33 (m, 2H), 0.99-0.66 (m, 12H). LC-MS: m/z 535.8 [M + H]⁺. |
| A20 | 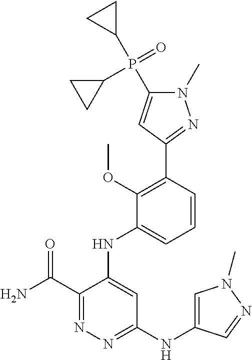 [SMILES: COc1c(Nc2cc(Nc3cnn(C)c3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 10.68 (s, 1H), 9.04 (s, 1H), 8.30 (s, 1H), 7.80 (s, 1H), 7.60-7.50 (m, 2H), 7.38-7.30 (m, 2H), 7.17-7.07 (m, 2H), 6.44 (s, 1H), 4.03 (s, 3H), 3.70 (s, 3H), 3.53 (s, 3H), 1.31-1.19 (m, 2H), 0.89-0.60 (m, 8H). LC-MS: m/z 547.8 [M + H]⁺. |
| A21 | 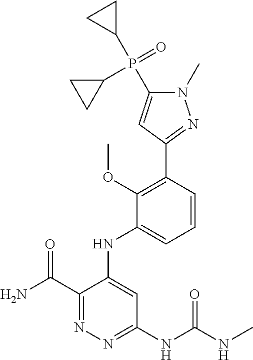 [SMILES: CNC(=O)Nc1cc(Nc2cccc(-c3cc(P(=O)(C4CC4)C4CC4)n(C)n3)c2OC)c(C(N)=O)nn1] | AA, AB, AC, AI, AN | ¹H-NMR (300 MHz, DMSO-d₆): δ 10.94 (s, 1H), 9.49 (s, 1H), 8.45 (s, 1H), 7.77 (s, 1H), 7.69-7.61 (m, 2H), 7.40 (d, J = 7.8 Hz, 1H), 7.32 (s, 1H), 7.23 (d, J = 8.1 Hz, 1H), 7.19 (s, 1H), 4.08 (s, 3H), 3.57 (s, 3H), 2.64 (d, J = 3.3 Hz, 3H), 1.37-1.23 (m, 2H), 0.93-0.82 (m, 4H), 0.81-0.68 (m, 4H). LC-MS: m/z 524.8 [M + H]⁺. |
| A22 | 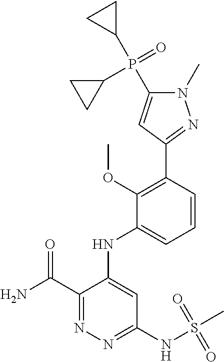 [SMILES: COc1c(Nc2cc(NS(C)(=O)=O)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.02 (s, 1H), 8.34 (s, 1H), 7.94 (s, 1H), 7.72 (d, J = 7.2 Hz, 1H), 7.43 (d, J = 7.8 Hz, 1H), 7.28 (d, J = 7.5 Hz, 1H), 7.22 (s, 1H), 6.99 (s, 1H), 4.12 (s, 3H), 3.62 (s, 3H), 2.94 (s, 3H), 1.41-1.27 (m, 2H), 0.96-0.75 (m, 8H). LC-MS: m/z 545.7 [M + H]⁺. |
| A23 | 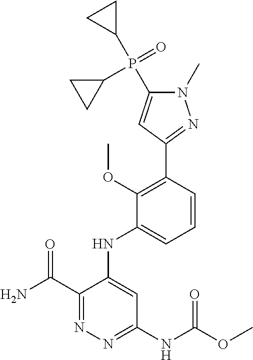 [SMILES: COC(=O)Nc1cc(Nc2cccc(-c3cc(P(=O)(C4CC4)C4CC4)n(C)n3)c2OC)c(C(N)=O)nn1] | AA, AB, AC, AI, AN | ¹H-NMR (300 MHz, DMSO-d₆): δ 11.01 (s, 1H), 10.82 (s, 1H), 8.51 (s, 1H), 7.88- 7.80 (m, 2H), 7.70 (d, J = 5.4 Hz, 1H), 7.46 (d, J = 7.8 Hz, 1H), 7.34-7.21 (m, 1H), 7.24 (s, 1H), 4.13 (s, 3H), 3.67 (s, 3H), 3.62 (s, 3H), 1.42-1.28 (m, 2H), 1.03- 0.66 (m, 8H). LC-MS: m/z 525.8 [M + H]⁺. |
| A24 | 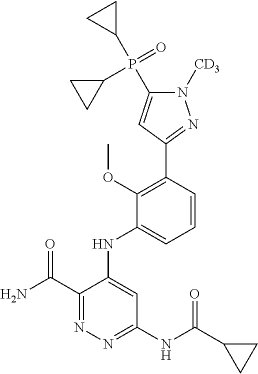 [SMILES: [2H]C([2H])([2H])n1nc(-c2cccc(Nc3cc(NC(=O)C4CC4)nnc3C(N)=O)c2OC)cc1P(=O)(C1CC1)C1CC1] | AA, AB, AC, AI, AN | ¹H-NMR (300 MHz, DMSO-d₆): δ 11.31 (s, 1H), 11.02 (s, 1H), 8.54 (s, 1H), 8.15 (s, 1H), 7.88 (s, 1H), 7.69 (d, J = 7.5 Hz, 1H), 7.43 (d, J = 8.1 Hz, 1H), 7.29-7.20 (m, 2H), 3.61 (s, 3H), 2.12-2.05 (m, 1H), 1.39- 1.29 (m, 2H), 0.99-0.72 (m, 12H). LC-MS: m/z 538.9 [M + H]⁺. |
| A25 | 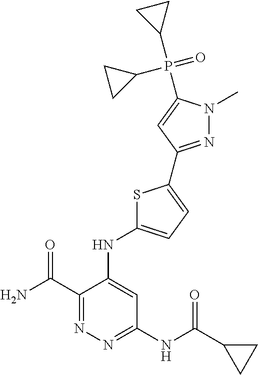 [SMILES: Cn1nc(-c2ccc(Nc3cc(NC(=O)C4CC4)nnc3C(N)=O)s2)cc1P(=O)(C1CC1)C1CC1] | AA, AM, AI, AN | ¹H-NMR (300 MHz, DMSO-d₆): δ 11.36 (s, 1H), 10.81 (s, 1H), 8.56 (s, 1H), 8.05 (s, 1H), 7.93 (s, 1H), 7.34 (d, J = 3.6 Hz, 1H), 7.17 (s, 1H), 6.93 (d, J = 3.6 Hz, 1H), 4.05 (s, 3H), 2.11-2.02 (m, 1H), 1.39-1.21 (m, 2H), 1.03-0.70 (m, 12H). LC-MS: m/z 511.7 [M + H]⁺. |
| A26 | 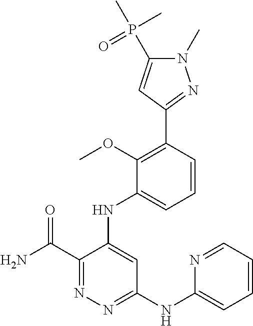 [SMILES: COc1c(Nc2cc(Nc3ccccn3)nnc2C(N)=O)cccc1-c1cc(P(C)(C)=O)n(C)n1] | AA, AB, AC, AI, AN | ¹H-NMR (300 MHz, DMSO-d₆): δ 11.01 (s, 1H), 10.16 (s, 1H), 8.47 (s, 1H), 8.23- 8.13 (m, 2H), 7.78-7.61 (m, 3H), 7.56 (d, J = 8.4 Hz, 2H), 7.33-7.24 (m, 1H), 7.08 (s, 1H), 6.96-6.88 (m, 1H), 4.16 (s, 3H), 3.62 (s, 3H), 1.81 (d, J = 13.8 Hz, 6H). LC-MS: m/z 492.8 [M + H]⁺. |
| A27 | 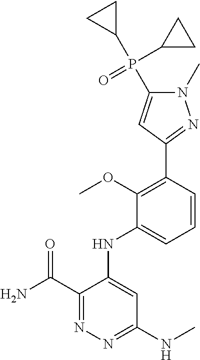 [SMILES: CNc1cc(Nc2cccc(-c3cc(P(=O)(C4CC4)C4CC4)n(C)n3)c2OC)c(C(N)=O)nn1] | AA, AB, AC, AI, AN | ¹H-NMR (300 MHz, DMSO-d₆): δ 10.82 (s, 1H), 8.29 (s, 1H), 7.63 (d, J = 8.7 Hz, 1H), 7.56 (s, 1H), 7.45 (d, J = 6.9 Hz, 1H), 7.28-7.17 (m, 2H), 7.04-6.96 (m, 1H), 6.33 (s, 1H), 4.12 (s, 3H), 3.61 (s, 3H), 2.86 (d, J = 3.6 Hz, 3H), 1.45-1.29 (m, 2H), 0.87- 0.66 (m, 8H). LC-MS: m/z 481.8 [M + H]⁺. |
| A28 | 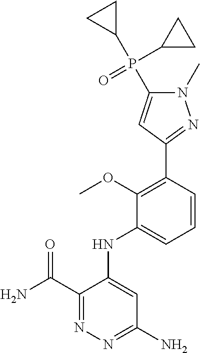 [SMILES: COc1c(Nc2cc(N)nnc2C(N)=O)cccc1-c1cc(P(=O)(C2CC2)C2CC2)n(C)n1] | AA, AB, AC, AI, AL | ¹H-NMR (300 MHz, DMSO-d₆): δ 10.78 (s, 1H), 8.28 (s, 1H), 7.66 (d, J = 7.8 Hz, 1H), 7.55 (s, 1H), 7.41 (d, J = 8.7 Hz, 1H), 7.26-7.16 (m, 2H), 6.57 (s, 2H), 6.38 (s, 1H), 4.12 (s, 3H), 3.61 (s, 3H), 1.48-1.28 (m, 2H), 0.97-0.88 (m, 8H). LC-MS: m/z 467.8 [M + H]⁺. |
| A29 | 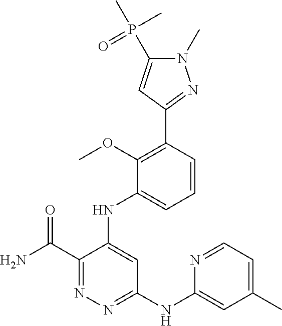 [SMILES: COc1c(Nc2cc(Nc3cc(C)ccn3)nnc2C(N)=O)cccc1-c1cc(P(C)(C)=O)n(C)n1] | AA, AB, AC, AI, AN | ¹H-NMR (300 MHz, DMSO-d₆): δ 11.03 (s, 1H), 10.06 (s, 1H), 8.49 (s, 1H), 8.13 (s, 1H), 8.05 (d, J = 5.1 Hz, 1H), 7.75 (s, 1H), 7.64 (d, J = 7.8 Hz, 1H), 7.55 (d, J = 7.8 Hz, 1H), 7.47 (s, 1H), 7.28 (t, J = 7.8 Hz, 1H), 7.08 (s, 1H), 6.78 (d, J = 4.8 Hz, 1H), 4.17 (s, 3H), 3.63 (s, 3H), 2.28 (s, 3H), 1.81 (d, J = 13.8 Hz, 6H). LC-MS: m/z 506.8 [M + H]⁺. |
| A30 | 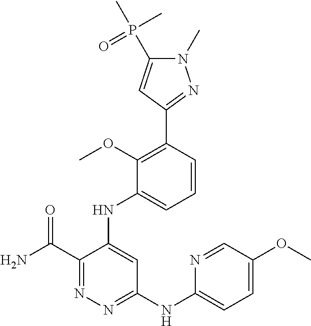 [SMILES: COc1ccc(Nc2cc(Nc3cccc(-c4cc(P(C)(C)=O)n(C)n4)c3OC)c(C(N)=O)nn2)nc1] | AA, AB, AC, AI, AN | ¹H-NMR (300 MHz, DMSO-d₆): δ 11.02 (s, 1H), 9.97 (s, 1H), 8.47 (s, 1H), 7.97 (s, 1H), 7.92 (s, 1H), 7.73 (s, 1H), 7.68-7.59 (m, 2H), 7.54 (d, J = 6.9 Hz, 1H), 7.46- 7.38 (m, 1H), 7.30 (t, J = 7.8 Hz, 1H), 7.09 (s, 1H), 4.17 (s, 3H), 3.79 (s, 3H), 3.63 (s, 3H), 1.81 (d, J = 13.8 Hz, 6H). LC-MS: m/z 522.8 [M + H]⁺. |
| A31 | 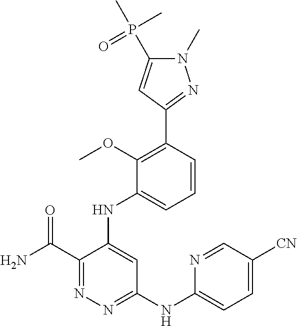 [SMILES: COc1c(Nc2cc(Nc3ccc(C#N)cn3)nnc2C(N)=O)cccc1-c1cc(P(C)(C)=O)n(C)n1] | AA, AB, AC, AI, AN | ¹H-NMR (300 MHz, DMSO-d₆): δ 11.09 (s, 1H), 10.74 (s, 1H), 8.64 (s, 1H), 8.56 (s, 1H), 8.16-8.10 (m, 1H), 8.06 (s, 1H), 7.86 (s, 1H), 7.82-7.76 (m, 1H), 7.70-7.64 (m, 1H), 7.59-7.52 (m, 1H), 7.39-7.29 (m, 1H), 7.09 (s, 1H), 4.17 (s, 3H), 3.63 (s, 3H), 1.82 (d, J = 13.5 Hz, 6H). LC-MS: m/z 517.8 [M + H]⁺. |
| A32 | 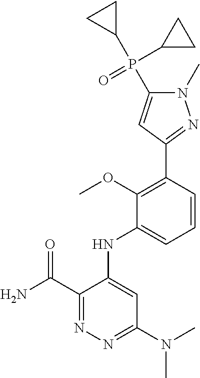 [SMILES: COc1c(Nc2cc(N(C)C)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 10.89 (s, 1H), 8.33 (s, 1H), 7.72-7.51 (m, 3H), 7.31-7.12 (m, 2H), 6.38 (s, 1H), 4.11 (s, 3H), 3.59 (s, 3H), 3.10 (s, 6H), 1.41-1.25 (m, 2H), 0.96-0.72 (m, 8H). |
| A33 | 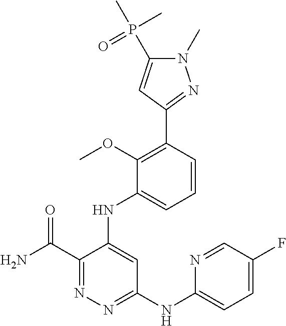 [SMILES: COc1c(Nc2cc(Nc3ccc(F)cn3)nnc2C(N)=O)cccc1-c1cc(P(C)(C)=O)n(C)n1] | AA, AB, AC, AI, AN | ¹H-NMR (300 MHz, DMSO-d₆): δ 11.03 (s, 1H), 10.20 (s, 1H), 8.49 (s, 1H), 8.18 (s, 1H), 7.96 (s, 1H), 7.77 (s, 1H), 7.74-7.69 (m, 2H), 7.67-7.62 (m, 1H), 7.56-7.49 (m, 1H), 7.35-7.27 (m, 1H), 7.08 (s, 1H), 4.17 (s, 3H), 3.63 (s, 3H), 1.82 (d, J = 14.1 Hz, 6H). LC-MS: m/z 510.7 [M + H]⁺. |
| A34 | 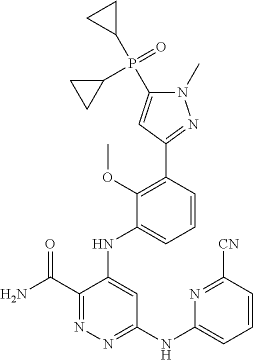 [SMILES: COc1c(Nc2cc(Nc3cccc(C#N)n3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.24 (s, 1H), 10.68 (s, 1H), 8.52 (s, 1H), 8.27 (s, 1H), 7.91 (s, 1H), 7.82 (s, 1H), 7.76 (s, 1H), 7.64-7.60 (m, 2H), 7.57 (s, 1H), 7.36 (s, 1H), 7.24 (s, 1H), 4.12 (s, 3H), 3.64 (s, 3H), 1.34-1.30 (m, 2H), 0.92-0.90 (m, 4H), 0.80-0.70 (m, 4H). LC-MS: m/z 569.8 [M + H]⁺. |
| A35 | 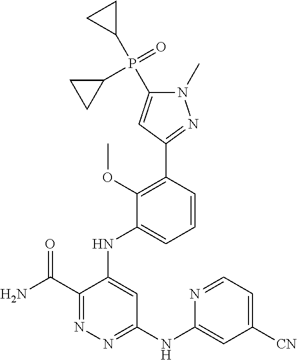 [SMILES: COc1c(Nc2cc(Nc3cc(C#N)ccn3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.06 (s, 1H), 10.50 (s, 1H), 8.60 (s, 1H), 8.46- 8.41 (m, 1H), 8.23 (s, 1H), 7.86-7.80 (m, 2H), 7.72-7.66 (m, 1H), 7.57-7.51 (m, 1H), 7.37-7.28 (m, 2H), 7.24 (s, 1H), 4.13 (s, 3H), 3.64 (s, 3H), 1.41-1.29 (m, 2H), 0.96- 0.87 (m, 4H), 0.83-0.74 (m, 4H). LC-MS: m/z 569.7 [M + H]⁺. |
| A36 | 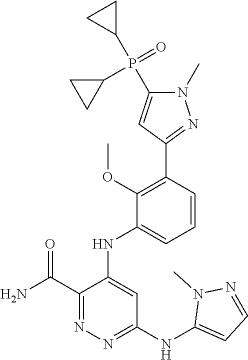 [SMILES: COc1c(Nc2cc(Nc3ccnn3C)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 10.85 (s, 1H), 9.19 (s, 1H), 8.45 (s, 1H), 7.74- 7.64 (m, 2H), 7.43 (d, J = 8.0 Hz, 1H), 7.32 (s, 1H), 7.27-7.19 (m, 2H), 6.64 (s, 1H), 6.28 (s, 1H), 4.12 (s, 3H), 3.67 (s, 3H), 3.61 (s, 3H), 1.40-1.31 (m, 2H), 0.96- 0.75 (m, 8H). LC-MS: m/z 547.8 [M + H]⁺. |
| A37 | 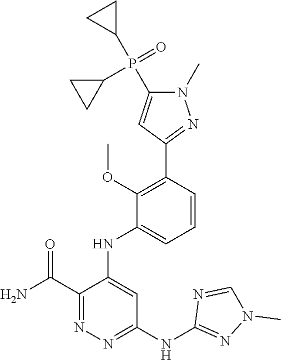 [SMILES: COc1c(Nc2cc(Nc3ncn(C)n3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.05 (s, 1H), 10.36 (s, 1H), 8.40 (s, 1H), 8.27 (s, 1H), 8.11 (s, 1H), 7.72 (s, 1H), 7.66 (d, J = 7.5 Hz, 1H), 7.56 (d, J = 7.5 Hz, 1H), 7.29 (t, J = 7.5 Hz, 1H), 7.23 (s, 1H), 4.12 (s, 3H), 3.75-3.72 (m, 3H), 3.63 (s, 3H), 1.41- 1.27 (m, 2H), 0.96-0.76 (m, 8H). LC-MS: 548.8 [M + H]⁺. |
| A38 | 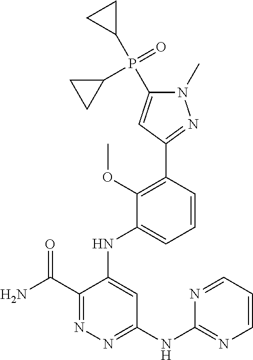 [SMILES: COc1c(Nc2cc(Nc3ncccn3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.06 (s, 1H), 10.52 (s, 1H), 8.66-8.31 (m, 4H), 7.81 (s, 1H), 7.72-7.64 (m, 1H), 7.63-7.56 (m, 1H), 7.39-7.30 (m, 1H), 7.24 (s, 1H), 7.02 (s, 1H), 4.13 (s, 3H), 3.64 (s, 3H), 1.43-1.26 (m, 2H), 1.04-0.70 (m, 8H). LC-MS: m/z 445.8 [M + H]⁺. |
| A39 | 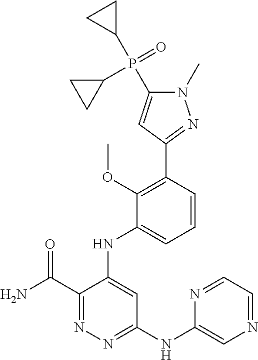 [SMILES: COc1c(Nc2cc(Nc3cnccn3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.06 (s, 1H), 10.48 (s, 1H), 8.98 (s, 1H), 8.55 (s, 1H), 8.20 (s, 1H), 8.14 (s, 1H), 7.99 (s, 1H), 7.82 (s, 1H), 7.69 (d, J = 8.4 Hz, 1H), 7.55 (d, J = 8.4 Hz, 1H), 7.36-7.27 (m, 1H), 7.24 (s, 1H), 4.13 (s, 3H), 3.63 (s, 3H), 1.53-1.20 (m, 2H), 1.02-0.68 (m, 8H). LC-MS: m/z 545.8 [M + H]⁺. |
| A40 | 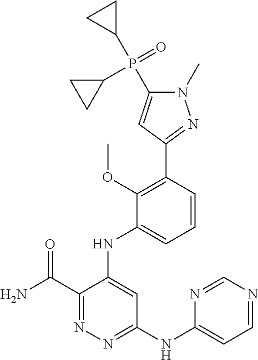 [SMILES: COc1c(Nc2cc(Nc3ccncn3)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.09 (s, 1H), 10.60 (s, 1H), 8.71 (s, 1H), 8.58 (s, 1H), 8.49 (d, J = 5.4 Hz, 1H), 8.06 (s, 1H), 7.87 (s, 1H), 7.75-7.66 (m, 2H), 7.56 (d, J = 8.1 Hz, 1H), 7.32 (t, J = 6.9 Hz, 1H), 7.24 (s, 1H), 4.13 (s, 3H), 3.64 (s, 3H), 1.40-1.28 (m, 2H), 0.99-0.72 (m, 8H). LC-MS: m/z 545.7 [M + H]⁺. |
| A41 | 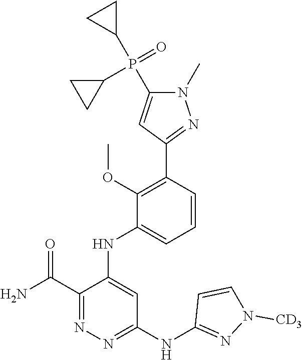 [SMILES: [2H]C([2H])([2H])n1ccc(Nc2cc(Nc3cccc(-c4cc(P(=O)(C5CC5)C5CC5)n(C)n4)c3OC)c(C(N)=O)nn2)n1] | AA, AB, AC, AI, AN | ¹H-NMR (300 MHz, DMSO-d₆): δ 11.00 (s, 1H), 9.79 (s, 1H), 8.41 (s, 1H), 7.73 (s, 1H), 7.68-7.61 (m, 2H), 7.57-7.51 (m, 2H), 7.32-7.25 (m, 1H), 7.24 (s, 1H), 6.15 (s, 1H), 4.13 (s, 3H), 3.63 (s, 3H), 1.40-1.29 (m, 2H), 0.98-0.73 (m, 8H). LC-MS: m/z 550.8 [M + H]⁺. |
| A42 | 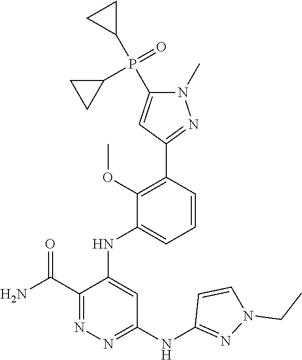 [SMILES: CCn1ccc(Nc2cc(Nc3cccc(-c4cc(P(=O)(C5CC5)C5CC5)n(C)n4)c3OC)c(C(N)=O)nn2)n1] | AA, AB, AC, AI, AN | ¹H-NMR (300 MHz, DMSO-d₆): δ 11.05 (s, 1H), 9.81 (s, 1H), 8.40 (s, 1H), 7.77 (s, 1H), 7.69-7.53 (m, 2H), 7.57 (s, 1H), 7.54 (d, J = 8.7 Hz, 1H), 7.28-7.20 (m, 2H), 6.10 (s, 1H), 4.12 (s, 3H), 3.98 (q, J = 7.2 Hz, 2H), 3.63 (s, 3H), 1.41-1.19 (m, 5H), 0.98-0.69 (m, 8H). LC-MS: m/z 561.8 [M + H]⁺. |
| A43 | 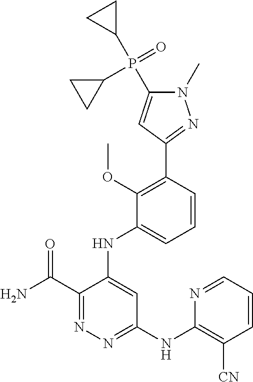 [SMILES: COc1c(Nc2cc(Nc3ncccc3C#N)nnc2C(N)=O)cccc1-c1cc(P(=O)(C2CC2)C2CC2)n(C)n1] | AA, AB, AC, AI, AN | ¹H-NMR (300 MHz, DMSO-d₆): δ 11.05 (s, 1H), 9.75 (s, 1H), 9.18 (s, 1H), 8.67 (s, 1H), 8.60-8.53 (m, 1H), 8.30 (s, 1H), 7.74- 7.69 (m, 1H), 7.58-7.55 (m, 1H), 7.33-7.28 (m, 1H), 7.25-7.20 (m, 2H), 6.79 (s, 1H), 4.13 (s, 3H), 3.68 (s, 3H), 1.35-1.30 (m, 2H), 0.96-0.90 (m, 4H), 0.84-0.78 (m, 4H). LC-MS: m/z 569.8 [M + H]⁺. |
| A44 | 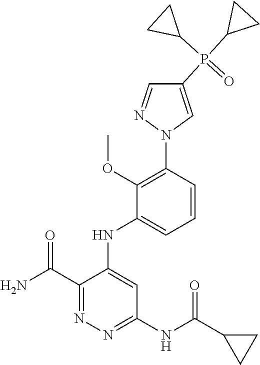 [SMILES: COc1c(Nc2cc(NC(=O)C3CC3)nnc2C(N)=O)cccc1-n1cc(P(=O)(C2CC2)C2CC2)cn1] | AO | ¹H-NMR (300 MHz, DMSO-d₆): δ 11.37 (s, 1H), 11.08 (s, 1H), 8.57 (s, 1H), 8.50 (s, 1H), 8.17 (s, 1H), 8.05 (s, 1H), 7.92 (s, 1H), 7.57-7.44 (m, 2H), 7.36 (t, J = 7.8 Hz, 1H), 3.46 (s, 3H), 2.15-2.04 (m, 1H), 1.26-1.15 (m, 2H), 1.00-0.60 (m, 12H). LC-MS: m/z 521.8 [M + H]⁺. |
| A45 | 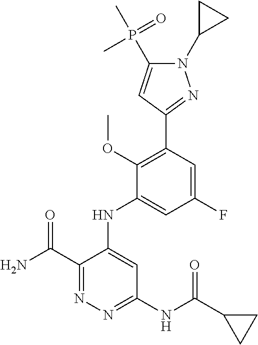 [SMILES: COc1c(Nc2cc(NC(=O)C3CC3)nnc2C(N)=O)cc(F)cc1-c1cc(P(C)(C)=O)n(C2CC2)n1] | AA, AB, AC, AI, AN | ¹H-NMR (300 MHz, DMSO-d₆): δ 11.32 (s, 1H), 10.98 (s, 1H), 8.53 (s, 1H), 8.11 (s, 1H), 7.88 (s, 1H), 7.66-7.60 (m, 1H), 7.44- 7.38 (m, 1H), 7.27-7.20 (m, 1H), 7.07 (s, 1H), 4.32-4.26 (m, 1H), 3.60 (s, 3H), 2.09- 2.03 (m, 1H), 1.86 (d, J = 13.5 Hz, 6H), 1.34-1.29 (m, 2H), 1.10-1.05 (m, 2H), 0.84-0.78 (m, 4H). LC-MS: m/z 509.8 [M + H]⁺. |
| A46 | 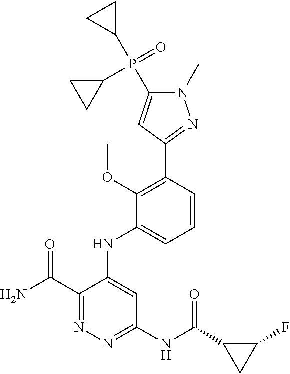 | AB, AC, AH, AI, AN | ¹H-NMR (300 MHz, DMSO-d₆): δ 11.39 (s, 1H), 11.19 (s, 1H), 8.58 (s, 1H), 8.22 (s, 1H), 7.92 (s, 1H), 7.41-7.34 (m, 1H), 7.34- 7.27 (m, 1H), 7.13 (s, 1H), 4.30-4.21 (m, 1H), 3.61 (s, 3H), 2.14-2.02 (m, 1H), 1.86 (d, J = 13.5 Hz, 6H), 1.37-1.28 (m, 2H), 1.11-1.03 (m, 2H), 0.87-0.79 (m, 4H). LC-MS: m/z 527.8 [M + H]⁺. |
| A47 | 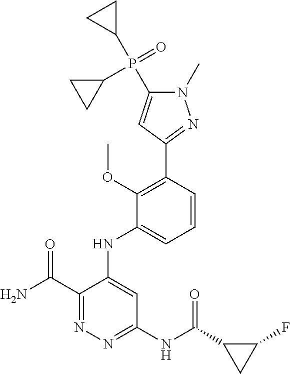 | AA, AB, AC, AI, AN | ¹H-NMR (300 MHz, DMSO-d₆): δ 11.36 (s, 1H), 11.04 (s, 1H), 8.55 (s, 1H), 8.14 (s, 1H), 7.89 (s, 1H), 7.70 (d, J = 7.5 Hz, 1H), 7.44 (d, J = 8.4 Hz, 1H), 7.31-7.24 (m, 1H), 7.23 (s, 1H), 5.22-4.68 (m, 1H), 4.13 (s, 3H), 3.62 (s, 3H), 2.30-2.23 (m, 1H), 1.75-1.51 (m, 1H), 1.43-1.30 (m, 2H), 1.27-1.18 (m, 1H), 1.02-0.63 (m, 8H). LC-MS: m/z 553.8 [M + H]⁺. |
| A48 | 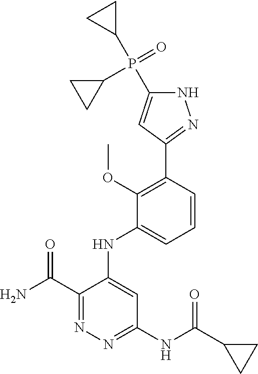 [SMILES: COc1c(Nc2cc(NC(=O)C3CC3)nnc2C(N)=O)cccc1-c1cc(P(=O)(C2CC2)C2CC2)[nH]n1] | AB, AP | ¹H-NMR (300 MHz, DMSO-d₆): δ 13.79 (s, 1H), 11.33 (s, 1H), 11.01 (s, 1H), 8.55 (s, 1H), 8.14 (s, 1H), 7.89 (s, 1H), 7.78- 7.58 (m, 1H), 7.51-7.39 (m, 1H), 7.29 (s, 1H), 7.12 (d, J = 19.8 Hz, 1H), 3.59 (s, 3H), 2.14-1.99 (m, 1H), 1.40-1.06 (m, 2H), 0.91-0.72 (m, 12H). LC-MS: m/z 521.8 [M + H]⁺. |
| A49 | 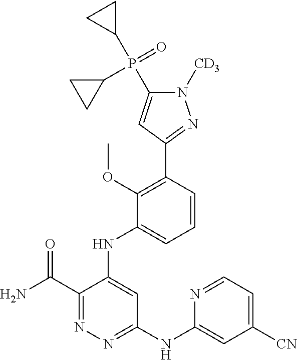 [SMILES: [2H]C([2H])([2H])n1nc(-c2cccc(Nc3cc(Nc4cc(C#N)ccn4)nnc3C(N)=O)c2OC)cc1P(=O)(C1CC1)C1CC1] | AA, AB, AC, AI, AN | ¹H-NMR (300 MHz, DMSO-d₆): δ 11.06 (s, 1H), 10.50 (s, 1H), 8.61 (s, 1H), 8.43 (d, J = 4.5 Hz, 1H), 8.23 (s, 1H), 7.89-7.77 (m, 2H), 7.69 (d, J = 6.9 Hz, 1H), 7.54 (d, J = 7.2 Hz, 1H), 7.37-7.20 (m, 3H), 3.64 (s, 3H), 1.42-1.27 (m, 2H), 1.00-0.72 (m, 8H). LC-MS: m/z 573.0 [M + H]⁺. |
| A50 | 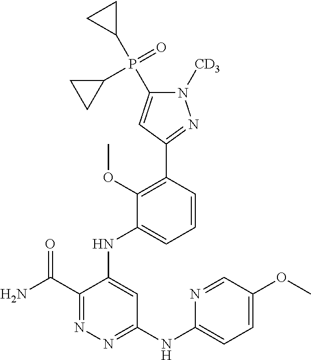 [SMILES: [2H]C([2H])([2H])n1nc(-c2cccc(Nc3cc(Nc4ccc(OC)cn4)nnc3C(N)=O)c2OC)cc1P(=O)(C1CC1)C1CC1] | AA, AB, AC, AI, AN | ¹H-NMR (300 MHz, DMSO-d₆): δ 11.02 (s, 1H), 9.98 (s, 1H), 8.47 (s, 1H), 7.98 (s, 1H), 7.91 (s, 1H), 7.73 (s, 1H), 7.69-7.59 (m, 2H), 7.54 (d, J = 8.4 Hz, 1H), 7.42 (d, J = 9.3 Hz, 1H), 7.31 (t, J = 7.8 Hz, 1H), 7.24 (s, 1H), 3.78 (s, 3H), 3.63 (s, 3H), 1.40-1.27 (m, 2H), 0.96-0.72 (m, 8H). LC-MS: m/z 577.8 [M + H]⁺. |
| A51 | 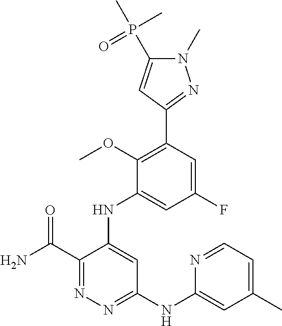 [SMILES: COc1c(Nc2cc(Nc3cc(C)ccn3)nnc2C(N)=O)cc(F)cc1-c1cc(P(C)(C)=O)n(C)n1] | AA, AB, AC, AI, AN | ¹H-NMR (300 MHz, DMSO-d₆): δ 11.29 (s, 1H), 10.16 (s, 1H), 8.55 (s, 1H), 8.23 (s, 1H), 8.06 (s, 1H), 7.81 (s, 1H), 7.53 (s, 1H), 7.47 (d, J = 9.0 Hz, 1H), 7.35 (d, J = 8.1 Hz, 1H), 7.14 (s, 1H), 6.82 (s, 1H), 4.17 (s, 3H), 3.64 (s, 3H), 2.29 (s, 3H), 1.83 (d, J = 13.8 Hz, 6H). LC-MS: m/z 524.8 [M + H]⁺. |
| A52 | 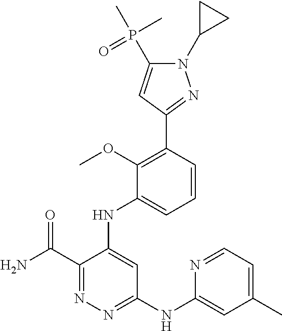 [SMILES: COc1c(Nc2cc(Nc3cc(C)ccn3)nnc2C(N)=O)cccc1-c1cc(P(C)(C)=O)n(C2CC2)n1] | AB, AC, AH, AI, AN | ¹H-NMR (300 MHz, DMSO-d₆): δ 11.01 (s, 1H), 10.07 (s, 1H), 8.50 (s, 1H), 8.12 (s, 1H), 8.05 (d, J = 4.8 Hz, 1H), 7.76 (s, 1H), 7.60 (d, J = 8.1 Hz, 1H), 7.55 (d, J = 7.8 Hz, 1H), 7.47 (s, 1H), 7.33-7.23 (m, 1H), 7.09 (s, 1H), 6.78 (d, J = 3.6 Hz, 1H), 4.41-4.25 (m, 1H), 3.63 (s, 3H), 2.28 (s, 3H), 1.86 (d, J = 13.8 Hz, 6H), 1.39-1.23 (m, 2H), 1.15-1.01 (m, 2H). LC-MS: m/z 532.8 [M + H]⁺. |
| A53 | 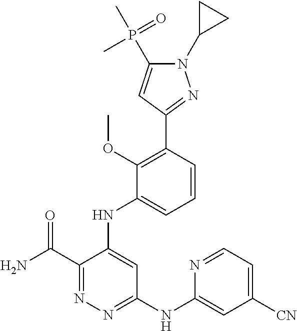 [SMILES: COc1c(Nc2cc(Nc3cc(C#N)ccn3)nnc2C(N)=O)cccc1-c1cc(P(C)(C)=O)n(C2CC2)n1] | AB, AC, AH, AI, AN | ¹H-NMR (300 MHz, DMSO-d₆): δ 11.04 (s, 1H), 10.50 (s, 1H), 8.61 (s, 1H), 8.45- 8.40 (m, 1H), 8.23 (s, 1H), 7.84 (s, 1H), 7.79 (s, 1H), 7.66-7.61 (m, 1H), 7.54-7.50 (m, 1H), 7.36-7.32 (m, 1H), 7.30-7.25 (m, 1H), 7.09 (s, 1H), 4.35-4.28 (m, 1H), 3.63 (s, 3H), 1.86 (d, J = 14.4 Hz, 6H), 1.35- 1.29 (m, 2H), 1.11-1.04 (m, 2H). LC-MS: m/z 543.8 [M + H]⁺. |
| A54 | 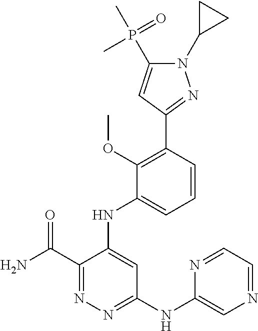 [SMILES: COc1c(Nc2cc(Nc3cnccn3)nnc2C(N)=O)cccc1-c1cc(P(C)(C)=O)n(C2CC2)n1] | AB, AC, AH, AI, AN | ¹H-NMR (300 MHz, DMSO-d₆): δ 11.05 (s, 1H), 10.49 (s, 1H), 8.98 (s, 1H), 8.56 (s, 1H), 8.20 (s, 1H), 8.14 (s, 1H), 7.98 (s, 1H), 7.83 (s, 1H), 7.65-7.60 (m, 1H), 7.56- 7.51 (m, 1H), 7.32-7.26 (m, 1H), 7.09 (s, 1H), 4.35-4.27 (m, 1H), 3.63 (s, 3H), 1.86 (d, J = 14.4 Hz, 6H), 1.34-1.28 (m, 2H), 1.11-1.05 (m, 2H). LC-MS: m/z 519.8 [M + H]⁺. |
| A55 | 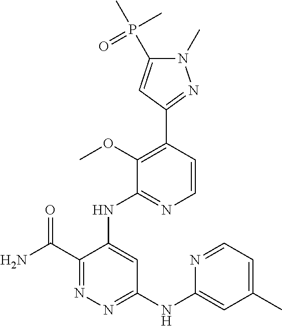 [SMILES: COc1c(-c2cc(P(C)(C)=O)n(C)n2)ccnc1Nc1cc(Nc2cc(C)ccn2)nnc1C(N)=O] | AA, AE, AG, AI, AN | ¹H-NMR (300 MHz, DMSO-d₆): δ 12.35 (s, 1H), 10.19 (s, 1H), 9.69 (s, 1H), 8.61 (s, 1H), 8.24-8.06 (m, 2H), 7.85 (s, 1H), 7.56 (s, 1H), 7.46 (s, 1H), 7.24 (s, 1H), 6.83 (s, 1H), 4.20 (s, 3H), 3.78 (s, 3H), 2.31 (s, 3H), 1.85 (d, J = 13.8 Hz, 6H). LC-MS: m/z 507.8 [M + H]⁺. |
| A56 | 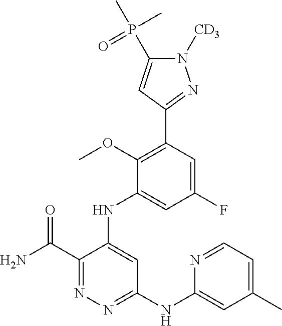 [SMILES: [2H]C([2H])([2H])n1nc(-c2cc(F)cc(Nc3cc(Nc4cc(C)ccn4)nnc3C(N)=O)c2OC)cc1P(C)(C)=O] | AA, AB, AC, AI, AN | ¹H-NMR (300 MHz, DMSO-d₆): δ 11.28 (s, 1H), 10.15 (s, 1H), 8.55 (s, 1H), 8.23 (s, 1H), 8.07 (d, J = 4.8 Hz, 1H), 7.81 (s, 1H), 7.52 (s, 1H), 7.49-7.42 (m, 1H), 7.38-7.30 (m, 1H), 7.14 (s, 1H), 6.82 (d, J = 4.6 Hz, 1H), 3.64 (s, 3H), 2.29 (s, 3H), 1.83 (d, J = 13.8 Hz, 6H). LC-MS: m/z 527.8 [M + H]⁺. |
| A57 | 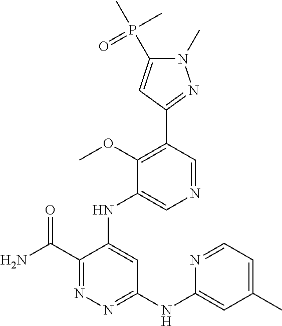 [SMILES: COc1c(Nc2cc(Nc3cc(C)ccn3)nnc2C(N)=O)cncc1-c1cc(P(C)(C)=O)n(C)n1] | AA, AF, AI, AN | ¹H-NMR (300 MHz, DMSO-d₆): δ 10.87 (s, 1H), 10.05 (s, 1H), 8.78 (s, 1H), 8.68 (s, 1H), 8.54 (s, 1H), 7.96 (d, J = 4.8 Hz, 1H), 7.83 (s, 1H), 7.80 (s, 1H), 7.53 (s, 1H), 7.08 (s, 1H), 6.77 (d, J = 5.1 Hz, 1H), 4.19 (s, 3H), 3.75 (s, 3H), 2.27 (s, 3H), 1.82 (d, J = 13.8 Hz, 6H). LC-MS: m/z 507.8 [M + H]⁺. |
| A58 | 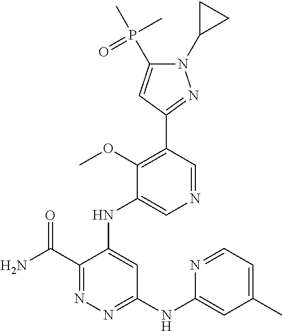 [SMILES: COc1c(Nc2cc(Nc3cc(C)ccn3)nnc2C(N)=O)cncc1-c1cc(P(C)(C)=O)n(C2CC2)n1] | AH, AF, AI, AN | ¹H-NMR (300 MHz, DMSO-d₆): δ 10.85 (s, 1H), 10.05 (s, 1H), 8.75 (s, 1H), 8.67 (s, 1H), 8.54 (s, 1H), 7.96 (d, J = 5.1 Hz, 1H), 7.81-7.70 (m, 2H), 7.54 (s, 1H), 7.01 (s, 1H), 6.76 (d, J = 5.1 Hz, 1H), 4.53-4.21 (m, 1H), 3.74 (s, 3H), 2.27 (s, 3H), 1.86 (d, J = 13.8 Hz, 6H), 1.40-1.25 (m, 2H), 1.15- 1.05 (m, 2H). LC-MS: m/z 533.8 [M + H]⁺. |
| A59 | 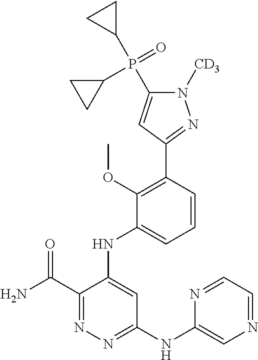 [SMILES: [2H]C([2H])([2H])n1nc(-c2cccc(Nc3cc(Nc4cnccn4)nnc3C(N)=O)c2OC)cc1P(=O)(C1CC1)C1CC1] | AA, AB, AC, AI, AN | ¹H-NMR (300 MHz, DMSO-d₆): δ 11.07 (s, 1H), 10.50 (s, 1H), 8.99 (s, 1H), 8.56 (s, 1H), 8.22-8.11 (m, 2H), 8.00 (s, 1H), 7.82 (s, 1H), 7.69 (d, J = 7.5 Hz, 1H), 7.55 (d, J = 7.5 Hz, 1H), 7.30 (t, J = 8.1 Hz, 1H), 7.24 (s, 1H), 3.63 (s, 3H), 1.41-1.29 (m, 2H), 0.99-0.72 (m, 8H). LC-MS: m/z 548.8 [M + H]⁺. |
| A60 | 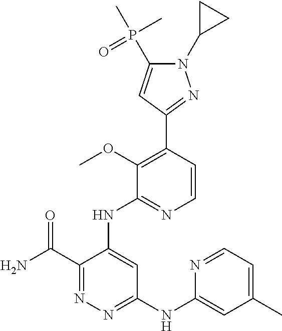 [SMILES: COc1c(-c2cc(P(C)(C)=O)n(C3CC3)n2)ccnc1Nc1cc(Nc2cc(C)ccn2)nnc1C(N)=O] | AE, AG, AH, AI, AN | ¹H-NMR (300 MHz, DMSO-d₆): δ 12.35 (s, 1H), 10.19 (s, 1H), 9.69 (s, 1H), 8.60 (s, 1H), 8.19 (d, J = 4.8 Hz, 1H), 8.13 (d, J = 5.1 Hz, 1H), 7.85 (s, 1H), 7.56 (s, 1H), 7.42 (d, J = 5.1 Hz, 1H), 7.25 (s, 1H), 6.83 (d, J = 4.5 Hz, 1H), 4.41-4.25 (m, 1H), 3.78 (s, 3H), 2.31 (s, 3H), 1.89 (d, J = 13.8 Hz, 6H), 1.43-1.30 (m, 2H), 1.19-1.07 (m, 2H). LC-MS: m/z 533.8 [M + H]⁺. |
| A61 | 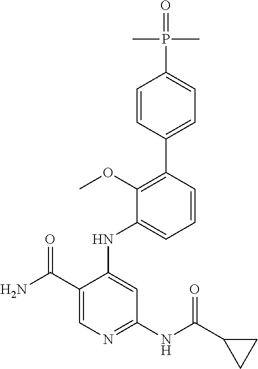 [SMILES: COc1c(Nc2cc(NC(=O)C3CC3)ncc2C(N)=O)cccc1-c1ccc(P(C)(C)=O)cc1] | AB, AC, AJ, AN | ¹H-NMR (300 MHz, DMSO-d₆): δ 10.94 (s, 1H), 10.78 (s, 1H), 8.60 (s, 1H), 8.15 (s, 1H), 8.08 (s, 1H), 7.90-7.78 (m, 2H), 7.76- 7.63 (m, 2H), 7.54-7.41 (m, 2H), 7.26 (t, J = 8.0 Hz, 1H), 7.15 (d, J = 7.6 Hz, 1H), 3.37 (s, 3H), 2.03-1.95 (m, 1H), 1.69 (d, J = 13.2 Hz, 6H), 0.85-0.66 (m, 4H). LC-MS: m/z 478.8 [M + H]⁺. |
| A62 | 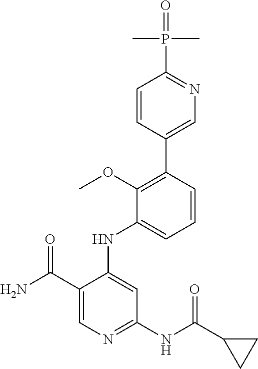 [SMILES: COc1c(Nc2cc(NC(=O)C3CC3)ncc2C(N)=O)cccc1-c1ccc(P(C)(C)=O)nc1] | AB, AC, AJ, AN | ¹H-NMR (300 MHz, DMSO-d₆): δ 10.95 (s, 1H), 10.79 (s, 1H), 8.93 (s, 1H), 8.61 (s, 1H), 8.20-8.13 (m, 2H), 8.07-8.01 (m, 2H), 7.55-7.49 (m, 2H), 7.33-7.28 (m, 1H), 7.25-7.20 (m, 1H), 3.41 (s, 3H), 2.02-1.97 (m, 1H), 1.71 (d, J = 12.9 Hz, 6H), 0.80- 0.76 (m, 4H). LC-MS: m/z 479.8 [M + H]⁺. |
| A63 | 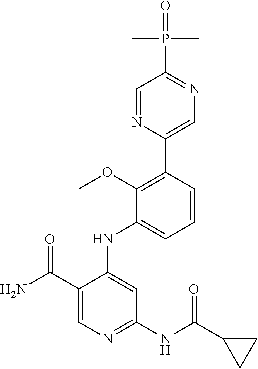 [SMILES: COc1c(Nc2cc(NC(=O)C3CC3)ncc2C(N)=O)cccc1-c1cnc(P(C)(C)=O)cn1] | AB, AK, AN | ¹H-NMR (300 MHz, DMSO-d₆): δ 10.94 (s, 1H), 10.78 (s, 1H), 9.23 (s, 1H), 9.18 (s, 1H), 8.61 (s, 1H), 8.18 (s, 1H), 8.04 (s, 1H), 7.60 (d, J = 7.5 Hz, 1H), 7.54-7.50 (m, 2H), 7.40-7.29 (m, 1H), 3.54 (s, 3H), 1.79 (d, J = 13.8 Hz, 6H), 1.16-1.02 (m, 1H), 0.85-0.72 (m, 4H). LC-MS: m/z 480.8 [M + H]⁺. |
| A64 | 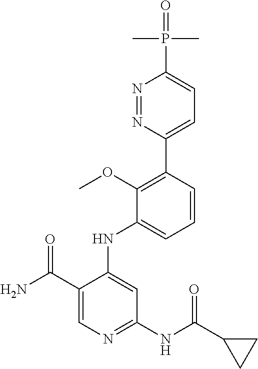 [SMILES: COc1c(Nc2cc(NC(=O)C3CC3)ncc2C(N)=O)cccc1-c1ccc(P(C)(C)=O)nn1] | AB, AK, AN | ¹H-NMR (300 MHz, DMSO-d₆): δ 10.97 (s, 1H), 10.80 (s, 1H), 8.62 (s, 1H), 8.28- 8.17 (m, 3H), 8.06 (s, 1H), 7.63-7.50 (m, 3H), 7.42-7.28 (m, 1H), 3.50 (s, 3H), 2.02- 1.96 (m, 1H), 1.85 (d, J = 14.1 Hz, 6H), 0.82-0.74 (m, 4H). LC-MS: m/z: 480.8 [M + H]⁺. |
| A65 | 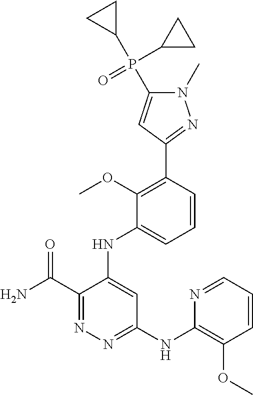 [SMILES: COc1cccnc1Nc1cc(Nc2cccc(-c3cc(P(=O)(C4CC4)C4CC4)n(C)n3)c2OC)c(C(N)=O)nn1] | AA, AB, AC, AI, AN | ¹H-NMR (300 MHz, DMSO-d₆): δ 11.10 (s, 1H), 8.60 (s, 1H), 8.55-8.45 (m, 2H), 7.81-7.73 (m, 2H), 7.71-7.66 (m, 1H), 7.60-7.54 (m, 1H), 7.42-7.36 (m, 1H), 7.35-7.29 (m, 1H), 7.24 (s, 1H), 7.01-6.93 (m, 1H), 4.13 (s, 3H), 3.92 (s, 3H), 3.63 (s, 3H), 1.40-1.28 (m, 2H), 0.96-0.87 (m, 4H), 0.84-0.75 (m, 4H). LC-MS: m/z 574.8 [M + H]⁺. |
| A66 | 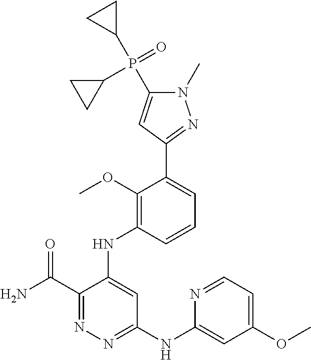 [SMILES: COc1ccnc(Nc2cc(Nc3cccc(-c4cc(P(=O)(C5CC5)C5CC5)n(C)n4)c3OC)c(C(N)=O)nn2)c1] | AA, AB, AC, AI, AN | ¹H-NMR (300 MHz, DMSO-d₆): δ 11.04 (s, 1H), 10.05 (s, 1H), 8.50 (s, 1H), 8.19 (s, 1H), 8.01 (d, J = 5.7 Hz, 1H), 7.76 (s, 1H), 7.66 (d, J = 7.5 Hz, 1H), 7.55 (d, J = 8.1 Hz, 1H), 7.29 (t, J = 7.8 Hz, 1H), 7.23- 7.20 (m, 2H), 6.57 (d, J = 4.8 Hz, 1H), 4.13 (s, 3H), 3.80 (s, 3H), 3.63 (s, 3H), 1.40-1.27 (m, 2H), 1.04-0.69 (m, 8H). LC-MS: m/z 574.8 [M + H]⁺. |
| A67 | 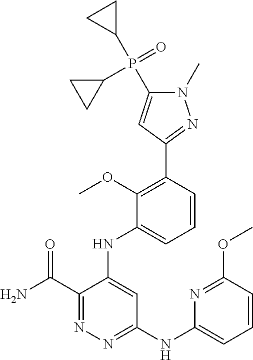 [SMILES: COc1cccc(Nc2cc(Nc3cccc(-c4cc(P(=O)(C5CC5)C5CC5)n(C)n4)c3OC)c(C(N)=O)nn2)n1] | AA, AB, AC, AI, AN | ¹H-NMR (300 MHz, DMSO-d₆): δ 10.78 (s, 1H), 10.15 (s, 1H), 8.46 (s, 1H), 7.94 (s, 1H), 7.75 (s, 1H), 7.75-7.71 (m, 1H), 7.57 (t, J = 7.5 Hz, 1H), 7.47 (d, J = 7.2 Hz, 1H), 7.29-7.22 (m, 1H), 7.21 (s, 1H), 6.99 (d, J = 7.8 Hz, 1H), 6.27 (d, J = 8.1 Hz, 1H), 4.13 (s, 3H), 3.62 (s, 3H), 3.43 (s, 3H), 1.43-1.24 (m, 2H), 0.98-0.69 (m, 8H). LC-MS: m/z 574.8 [M + H]⁺. |
| B1 | 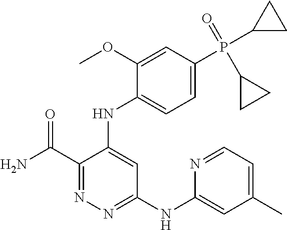 [SMILES: COc1cc(P(=O)(C2CC2)C2CC2)ccc1Nc1cc(Nc2cc(C)ccn2)nnc1C(N)=O] | AQ, AN | ¹H-NMR (300 MHz, DMSO-d₆): δ 10.93 (s, 1H), 10.09 (s, 1H), 8.47 (s, 1H), 8.14 (s, 1H), 8.07 (d, J = 4.8 Hz, 1H), 7.72 (s, 1H), 7.69-7.63 (m, 1H), 7.51-7.45 (m, 2H), 7.42 (s, 1H), 6.80 (d, J = 4.5 Hz, 1H), 3.91 (s, 3H), 2.28 (s, 3H), 1.37-1.22 (m, 2H), 0.91- 0.57 (m, 8H). LC-MS: m/z 478.8 [M + H]⁺. |
| B2 | 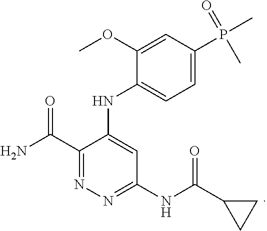 [SMILES: COc1cc(P(C)(C)=O)ccc1Nc1cc(NC(=O)C2CC2)nnc1C(N)=O] | AQ, AN | ¹H-NMR (300 MHz, DMSO-d₆): δ 11.34 (s, 1H), 10.97 (s, 1H), 8.51 (s, 1H), 8.19 (s, 1H), 7.84 (s, 1H), 7.57-7.52 (m, 1H), 7.46- 7.42 (m, 1H), 7.39-7.35 (m, 1H), 3.90 (s, 3H), 2.11-2.06 (m, 1H), 1.68 (d, J = 13.2 Hz, 6H), 0.87-0.81 (m, 4H). LC-MS: m/z 404.0 [M + H]⁺. |
| B3 | 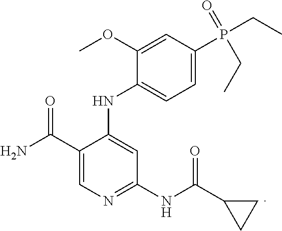 [SMILES: CCP(=O)(CC)c1ccc(Nc2cc(NC(=O)C3CC3)ncc2C(N)=O)c(OC)c1] | AQ, AN | ¹H-NMR (300 MHz, DMSO-d₆): δ 10.89 (s, 1H), 10.78 (s, 1H), 8.58 (s, 1H), 8.12 (s, 1H), 7.56-7.51 (m, 1H), 7.46 (s, 1H), 7.37- 7.21 (m, 3H), 3.88 (s, 3H), 2.01-1.86 (m, 5H), 1.01-0.90 (m, 6H), 0.85-0.74 (m, 4H). LC-MS: m/z 430.9 [M + H]⁺. |
| B4 | 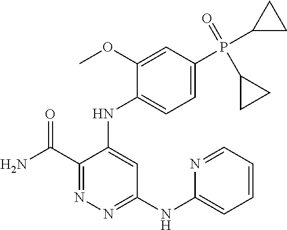 [SMILES: COc1cc(P(=O)(C2CC2)C2CC2)ccc1Nc1cc(Nc2ccccn2)nnc1C(N)=O] | AQ, AN | ¹H-NMR (300 MHz, DMSO-d₆): δ 10.93 (s, 1H), 10.18 (s, 1H), 8.45 (s, 1H), 8.23- 8.14 (m, 2H), 7.77-7.63 (m, 3H), 7.58 (d, J = 8.1 Hz, 1H), 7.51-7.40 (m, 2H), 6.97- 6.90 (m, 1H), 3.91 (s, 3H), 1.38-1.25 (m, 2H), 0.93-0.58 (m, 8H). LC-MS: m/z 464.8 [M + H]⁺. |
| B5 | 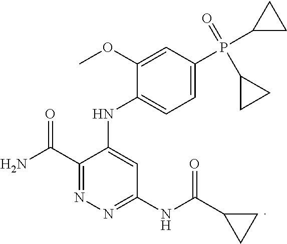 [SMILES: COc1cc(P(=O)(C2CC2)C2CC2)ccc1Nc1cc(NC(=O)C2CC2)nnc1C(N)=O] | AQ, AN | ¹H-NMR (300 MHz, DMSO-d₆): δ 11.35 (s, 1H), 11.01 (s, 1H), 8.51 (s, 1H), 8.21 (s, 1H), 7.84 (s, 1H), 7.57-7.52 (m, 1H), 7.48- 7.38 (m, 2H), 3.90 (s, 3H), 2.12-2.05 (m, 1H), 1.36-1.34 (m, 2H), 0.95-0.34 (m, 12H). LC-MS: m/z 455.8 [M + H]⁺. |
| B6 | 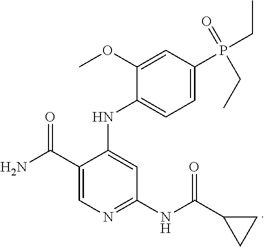 [SMILES: CCP(=O)(CC)c1ccc(Nc2cc(NC(=O)C3CC3)ncc2C(N)=O)c(OC)c1] | AQ, AN | ¹H-NMR (300 MHz, DMSO-d₆) 11.34 (s, 1H), 11.01 (s, 1H), 8.51 (s, 1H), 8.21 (s, 1H), 7.84 (s, 1H), 7.54 (s, 1H), 7.41-7.30 (m, 2H), 3.89 (s, 3H), 2.11-2.05 (m, 1H), 2.01-1.85 (m, 4H), 1.04-0.90 (m, 6H), 0.90-0.79 (m, 4H). LC-MS: m/z 432.1 [M + H]⁺. |
| B7 | 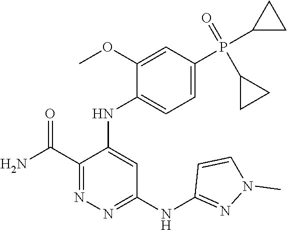 [SMILES: COc1cc(P(=O)(C2CC2)C2CC2)ccc1Nc1cc(Nc2ccn(C)n2)nnc1C(N)=O] | AQ, AN | ¹H-NMR (300 MHz, DMSO-d₆): δ 10.86 (s, 1H), 9.82 (s, 1H), 8.37 (s, 1H), 7.81 (s, 1H), 7.68-7.60 (m, 2H), 7.55 (s, 1H), 7.49- 7.37 (m, 2H), 6.13 (s, 1H), 3.91 (s, 3H), 3.72 (s, 3H), 1.39-1.22 (m, 2H), 0.88-0.59 (m, 8H). LC-MS: m/z 467.8 [M + H]⁺. |
| B8 | 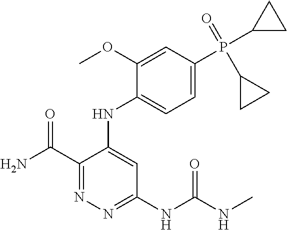 [SMILES: CNC(=O)Nc1cc(Nc2ccc(P(=O)(C3CC3)C3CC3)cc2OC)c(C(N)=O)nn1] | AQ, AN | ¹H-NMR (300 MHz, DMSO-d₆): δ 10.96 (s, 1H), 9.55 (s, 1H), 8.46 (s, 1H), 7.77- 7.70 (m, 2H), 7.56 (d, J = 7.8 Hz, 1H), 7.46 (s, 1H), 7.43-7.34 (m, 2H), 3.91 (s, 3H), 2.69 (s, 3H), 1.35-1.23 (m, 2H), 0.92- 0.61 (m, 8H). LC-MS: m/z 444.8 [M + H]⁺. |
| B9 | 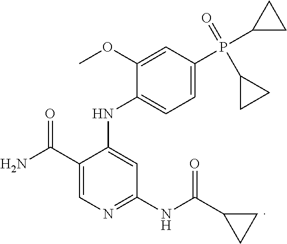 [SMILES: COc1cc(P(=O)(C2CC2)C2CC2)ccc1Nc1cc(NC(=O)C2CC2)ncc1C(N)=O] | AQ, AN | ¹H-NMR (300 MHz, DMSO-d₆): δ 10.89 (s, 1H), 10.79 (s, 1H), 8.59 (s, 1H), 8.23- 8.06 (m, 2H), 7.53 (s, 1H), 7.49-7.26 (m, 3H), 3.89 (s, 3H), 2.02-1.95 (m, 1H), 1.35- 1.23 (m, 2H), 0.95-0.50 (m, 12H). LC-MS: m/z 454.8 [M + H]⁺. |
| B10 | 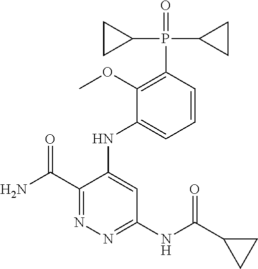 [SMILES: COc1c(Nc2cc(NC(=O)C3CC3)nnc2C(N)=O)cccc1P(=O)(C1CC1)C1CC1] | AR | ¹H-NMR (300 MHz, DMSO-d₆): δ 11.31 (s, 1H), 10.79 (s, 1H), 8.56 (s, 1H), 7.90- 7.80 (m, 2H), 7.63-7.46 (m, 2H), 7.31 (t, J = 6.3 Hz, 1H), 3.72 (s, 3H), 2.10-2.01 (m, 1H), 1.38-1.28 (m, 2H), 0.88-0.50 (m, 12H). LC-MS: m/z 455.8 [M + H]⁺. |

### Example 24, Luciferase Assay

### Experimental Procedure:

1. L929 ISRE cells (5000 cells/well) were seeded on 96-well plates and incubated overnight at room temperature-;
2. The cells were pretreated by adding different concentrations (10 nM, 50 nM, 100 nM, 200 nM or 1000 nM) of the test compounds for 2 hours;
3. Added IFN-α (100 ng/mL) to stimulate cells for 6 hours;
4. Removed the upper medium, add PBS (100 μL) and washed, then removed PBS;
5. Added PLB lysis solution (50 μL) to each well, shook the well for 15 minutes;
6. Transferred PLB lysis solution (30 μL) to a new 96-well whiteboard, added LAR II reagent (LAR II), then quickly read the 450 nm OD value with a plate washer;
7. Added Stop & Glo reagent (30 μL), then read the 450 nm OD value using the plate washer.
8. The final result was the OD value in step 6/OD value in step 7.

TABLE 2
Inhibitory activity of compounds on IFNα
induced TYK2/JAK1 mediated STAT activation

| | Inhibitory | Inhibitory | Inhibitory |
|---|---|---|---|
| | activity % | activity % | activity % |
| NO. | (50 nM) | (200 nM) | (1000 nM) |
| A1 | 34 | 87 | 98 |
| A2 | 26 | 57 | 94 |
| A3 | 34 | 39 | 71 |
| A4 | 61 | 93 | 99 |
| A5 | 91 | 97 | 99 |
| A6 | 23 | 28 | 96 |
| A7 | 18 | 25 | 64 |
| A8 | 84 | 88 | 96 |
| A11 | 6 | 86 | 95 |
| A12 | 25 | 33 | 93 |
| A13 | 9 | 71 | 95 |
| A14 | / | 23 | 76 |
| A17 | / | 42 | 82 |
| A18 | 62 | 79 | 79 |
| A19 | / | 28 | 71 |
| A21 | 16 | 39 | 74 |
| A23 | / | 38 | 85 |
| A24 | 39 | 90 | 95 |
| A26 | / | / | 80 |
| A29 | / | / | 93 |
| A30 | 14 | 58 | 90 |
| A31 | 9 | 54 | 75 |
| A33 | 45 | 62 | 81 |
| A34 | 17 | 67 | 95 |
| A35 | 54 | 84 | 98 |
| A38 | 36 | 66 | 89 |
| A39 | 59 | 78 | 97 |
| A40 | 61 | 80 | 96 |
| A41 | 55 | 90 | 95 |
| A42 | 76 | 90 | 92 |
| A43 | 0 | 18 | 64 |
| A47 | 8 | 19 | 62 |
| A48 | 17 | 56 | 76 |
| A49 | 33 | 86 | 87 |
| A50 | / | / | 77 |
| A51 | 55 | 82 | 86 |
| A52 | / | 9 | 63 |
| A55 | 34 | 63 | 90 |
| A56 | 31 | 70 | 95 |
| A58 | / | / | 86 |
| A59 | / | / | 86 |
| A60 | / | / | 66 |
| A61 | 8 | 23 | 59 |
| A62 | 3 | 5 | 56 |
| A63 | 33 | 43 | 75 |
| A65 | 36 | 43 | 93 |
| A66 | 59 | 82 | 96 |
| A67 | 88 | 95 | 95 |
| B1 | / | / | 0 |
| B3 | / | / | 19 |
| B4 | / | / | 8 |
| B5 | / | / | 0 |
| B6 | / | / | 0 |
| B7 | / | / | 0 |
| B10 | / | / | 28 |
| BMS986165 | 62 | 94 | 99 |

Symbol “/” indicated that inhibition was not detected; BMS986165 is the reference for comparison.

According to Table 2, Compounds A1-A8, A11-A14, A17-A19, A21, A23-A24, A26, A29-A31, A33-A35, A38-A42, A48-A51, A55-A56, A58-A59, A63, and A65-A67 displayed inhibitory activities on IFNα, induced TYK2/JAK1 mediated STAT activation. Furthermore, by comparing the inhibitory effects of the five groups of Compounds A1, B5 and B10, A4 and B4, A6 and B6, A12 and B1, A17 and B7, respectively, it can be tentatively summarized that the compounds of the A series containing the B ring (e.g., pyrazole ring) have higher inhibitory activity compared with the compounds of the B series that do not contain the B ring in the general structure (see, e.g., Formulas (I)-(IV)).

### Example 25, Enzyme-Linked Immunosorbent Assays (ELISA)

### Experimental Procedure:

1. Soak plate: Add 300 μL 1× washing solution and let stand for 30 seconds. After discarding the washing solution, anhydrous the microporous plate on absorbent paper.
2. Add standard: Add 100 μL of 2-fold standard to standard well. Add 100 μL standard dilution (serum/plasma sample) or culture medium (cell culture supernatant sample) to blank well.
3. Add sample: Serum/plasma: Add 50 μL 1× assay buffer and 50 μL sample to sample well. Cell culture supernatant: Add 100 μL cell culture supernatant to sample well.
4. Add detection antibody: Add 50 μL diluted detection antibody (1:100 diluted) to each well. Make sure steps 4, 5 and 6 are added continuously without interruption. The sampling process was completed within 15 minutes.
5. Incubation: Use sealing plate film to seal the plate. Shake at 300 rpm and incubate for 2 hours at room temperature.
6. Washing: Discard the liquid, add 300 μL of washing solution and wash the plate 6 times. Pat anhydrous on blotting paper after each wash. To obtain the desired experimental performance, the residual liquid must be completely removed.
7. Enzyme addition: Add 100 μL diluted horseradish peroxidase labeled streptavidin to each well (1:100 diluted).
8. Incubation: Seal the plate with a new sealing film. Shake at 300 rpm and incubate at room temperature for 45 minutes.
9. Washing: Repeat step 8.
10. Add substrate for color development: Add 100 μL of color development substrate TMB to each well, keep away from light, and incubate at room temperature for 5-30 minutes.
11. Add Stop Solution: Add 100 μL stop solution to each well. The color changed from blue to yellow. If the color appears green or the color changes unevenly, gently tap the frame of the plate to mix well.
12. Assay readout: Within 30 minutes, perform dual wavelength detection using a microplate reader to measure the OD at the absorption maximum at 450 nm and reference wavelengths at 570 nm or 630 nm. The calibrated OD value is the measured value at 450 nm minus the measured value at 570 nm or 630 nm.

TABLE 3
ELISA results of test compounds.

| NO. | IC₅₀ (nM) | NO. | IC₅₀ (nM) |
|---|---|---|---|
| A1 | 6.3 | BMS986165 | 16.6 |

As shown in table 3. Compound A1 effectively inhibited IFNα induced CXCL-10 in PBMC, thereby indicating the ability of Compound A1 to inhibit the JAK1/TYK2 signaling pathway. The inhibitory effect of A1 was better than that of BMS986165.

### Example 26, Competitive Binding Assay

### Experimental Procedure:

For most assays, kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111X stocks in 100% DMSO. Dissociation constants (Kds) were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

An 11-point 3-fold serial dilution of each test compound was prepared in 100% DMSO at 100× final test concentration and subsequently diluted to 1× in the assay (final DMSO concentration=1%). Most Kds were determined using a compound top concentration=30,000 nM. If the initial Kd determined was <0.5 nM (the lowest concentration tested), the measurement was repeated with a serial dilution starting at a lower top concentration. A Kd value reported as 40,000 nM indicates that the Kd was determined to be >30,000 nM.

Binding constants (Kds) were calculated with a standard dose-response curve using the Hill equation:

$$Response = Background + \frac{Signal - Background}{1 + ( {Kd}^{Hill Slope} / {Dose}^{Hill Slope} )}$$

The Hill Slope was set to −1.

Curves were fitted using a non-linear least square fit with the Levenberg-Marquardt algorithm.

TABLE 4
Competitive binding affinity of compounds to TYK2 JH2,
JAK1 JH2, JAK1 JH1 and JAK2 JH1, JAK3 JH1 and TYK2 JH1

| | TYK2 JH2 | JAK1 JH2 | JAK1 JH1 | JAK2 JH1 | JAK3 JH1 | TYK2 JH1 |
|---|---|---|---|---|---|---|
| NO. | Kd (nM) | Kd (nM) | Kd (nM) | Kd (nM) | Kd (nM) | Kd (nM) |
| A1 | 0.0044 ± 0.003 | 0.02 ± 0.002 | 6900 ± 900 | 790 ± 40 | 210 ± 15 | 550 ± 50 |

As shown in Table 4, Compound A1 displayed strong affinity to TYK2 JH2 and JAK1 JH2, and weak affinity to JAK1 JH1, JAK2 JH1, JAK3 JH1 and TYK2 JH1. Compound A1 is more than 10,000 times more selective to bind JAK1 JH2 and TYK2 JH2 than to bind JAK1 JH1, JAK2 JH1, JAK3 JH1 and TYK2 JH1.

### Example 27, Selectivity Evaluation

### Purpose 1: To Test the Inhibitory Activity on JAK2/JAK2 Pathway

### Experimental Procedure:

HEL cells were digested by trypsin, re-suspended in 1640 medium and counted. The compounds to be measured were added to 6 well plates (1 million/per well) for 2 hours, and then thrombopoietin (TPO)(final concentration: 100 ng/mL) was added to stimulate cells for 30 minutes. The cells were collected and lysed with protein lysate. The protein was quantitatively tested by bicinchoninic acid (BCA) kit. Finally, p-STAT3/STAT3 and internal reference protein β-actin were detected by Western blot.

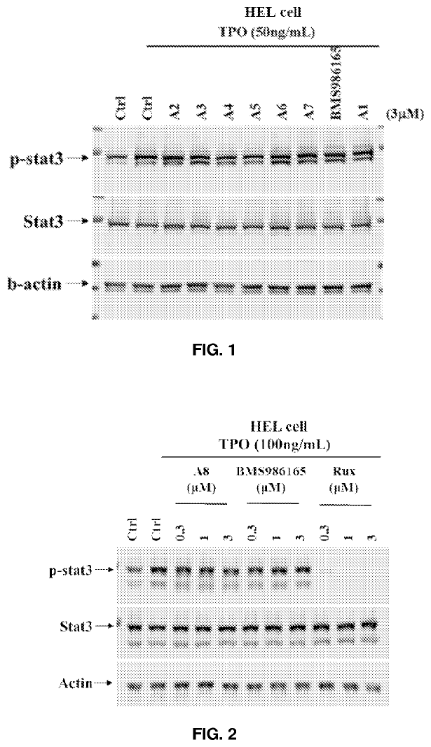

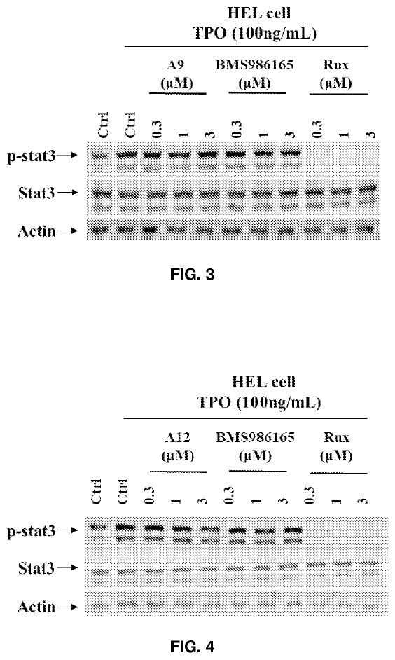

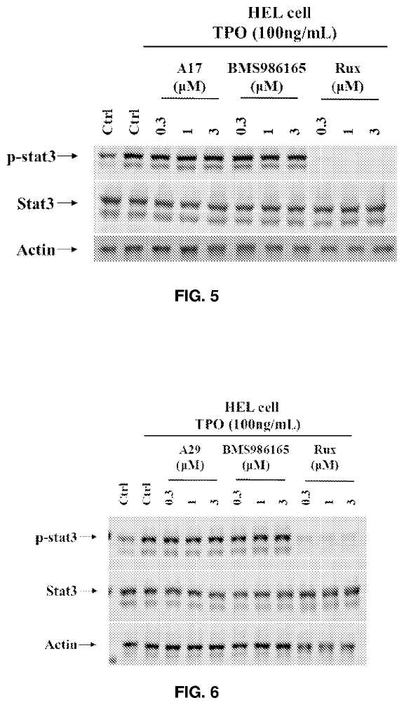

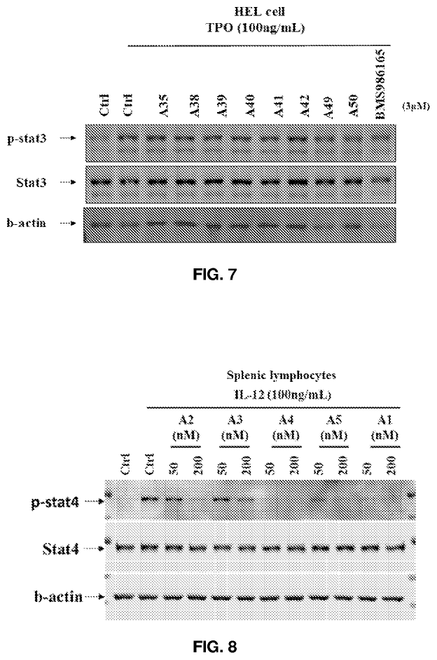

As shown in FIGS. 1-7, Compounds A1-A9, A12, A17, A29, A35, A38-A42, and A49-A50 were similar to BMS986165 and had no effect on TPO-induced downstream STAT3 phosphorylation. This observation indicated that these compounds have no inhibitory activity on the JAK2/JAK2 pathway. Therefore, Compounds A1-A9, A12, A17, A29, A35, A38-A42, A49-A50 had no inhibitory activity on JAK2. Pan-JAK inhibitor Ruxolitinib (Rux in FIGS. 2-6) can inhibit the JAK2/JAK2 pathway. JAK2 can pair with itself and is essential in platelet production, erythropoiesis, myelopoiesis, and keratinocyte production. Hence, inhibition of the JAK2 activity can lead to serious adverse effects such as thrombocytopenia and anemia. The compounds of the present invention have no JAK2 inhibitory activity and can avoid these adverse effects caused by the inhibition of JAK2.

### Purpose 2: To Test the Inhibitory Activity on the TYK2/JAK2 Pathway

### Experimental Procedure:

The spleen of mice was isolated and ground into single cells with bent tweezers. The single cells of the spleen were centrifuged for 5 minutes (400 g), and erythrocytes were lysed with Ammonium-Chloride-Potassium (ACK) solution and neutralized in 1640 medium, centrifuged for 5 minutes (400 g), and then re-suspended in 1640 medium. The spleen single cell suspension was counted, transferred to and incubated on a 6-well plate (5 million/well). The compounds to be tested were added. After 2 hours, IL-12 (50 ng/mL) was added to stimulate the cells for 1 h. The cells were collected and lysed with protein lysate. The protein was quantitatively tested by BCA kit. Finally, p-STAT4/STAT4 and internal reference protein S-actin were detected by Western blot.

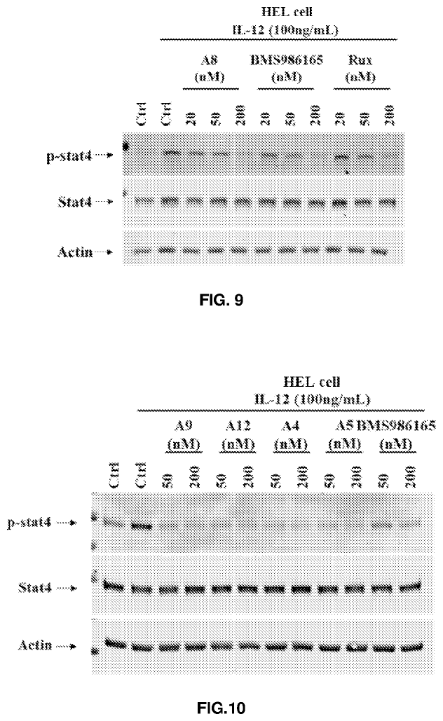

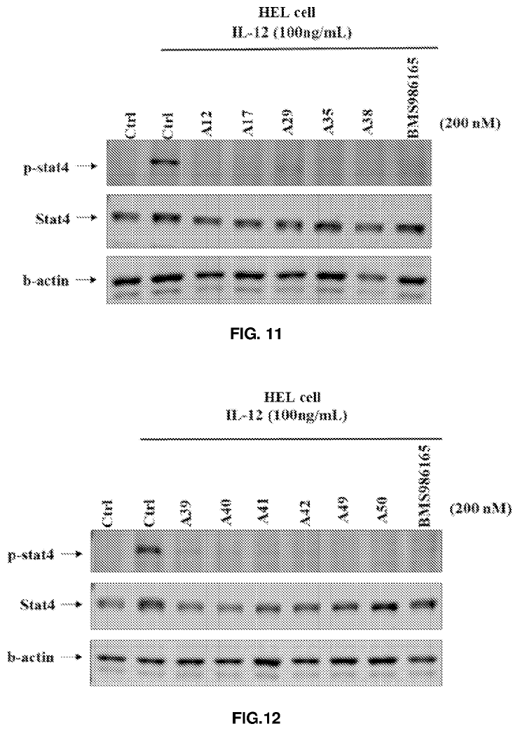

As shown in FIGS. 8-12, Compounds A1-A5, A8-A9, A12, A17, A29, A35, A38-A42, and A49-A50 effectively inhibited the downstream STAT4 phosphorylation induced by IL-12, indicating that the compounds can inhibit the TYK2/JAK2 pathway, and the inhibitory activity was better than that of BMS986165. Combined with the results in FIGS. 1-7 showing that Compounds A1-A5, A8, A9, A12, A17, A29, A35, A38-A42, A49, and A50 did not inhibit JAK2, their inhibitory activities on the TYK2/JAK2 pathway mainly came from their selective inhibition of TYK2. Overall, the activity of A1-A5, A8-A9, A12, A17, A29, A35, A38-A42, and A49-A50 on TYK2 was superior to BMS986165.

### Purpose 3: To Test the Inhibitory Activity of JAK1/JAK2 Pathway

### Experimental Procedure:

HT-29 cells were digested by trypsin and suspended in Dulbecco's Modified Eagle Medium (DMEM) and counted. The tested compounds were added into the 6 well plates (400 000/per well). After 2 hours, IFN-γ (100 ng/mL) was added to stimulate cells for 30 minutes. The cells were collected and lysed with protein lysate. The protein was quantitatively tested by BCA kit. Finally, p-STAT1/STAT1 and internal reference protein β-actin were detected by Western blot.

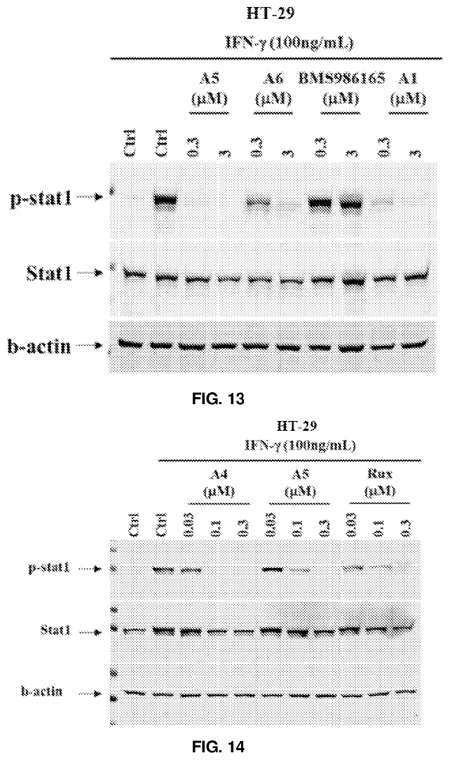

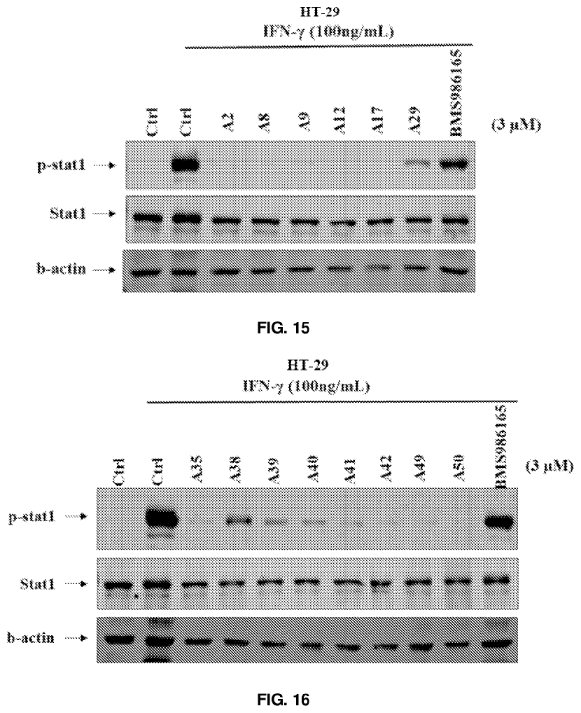

As shown in FIGS. 13-16, Compound BMS986165 had no effect on downstream STAT1 phosphorylation induced by IFN-γ, indicating that it had no inhibitory effect on the JAK1/JAK2 pathway, while Compounds A1-A2, A4-A6, A8-A9, A12, A17, A29, A35, A38-A42, A49-A50 and Ruxolitinib inhibited JAK1/JAK2 pathway in a concentration-dependent manner. Combined with the results in FIGS. 1-7 showing that Compounds A1-A2, A4-A6, A8-A9, A12, A17, A29, A35, A38-A42, A49-A50 had no inhibitory activity on JAK2, the inhibitory effects of A1-A2, A4-A6, A8-A9, A12, A17, A29, A35, A38-A42, and A49-A50 on the JAK1/JAK2 pathway came from their inhibition of JAK1. As a consequence, A1-A2, A4-A6, A8-A9, A12, A17, A29, A35, A38-A42, and A49-A50 had additional biological functions than BMS986165. With these unique selective inhibition profiles, the compounds of the present disclosure may become more efficiently treatment for autoimmune diseases.

1. A compound of Formula (I): 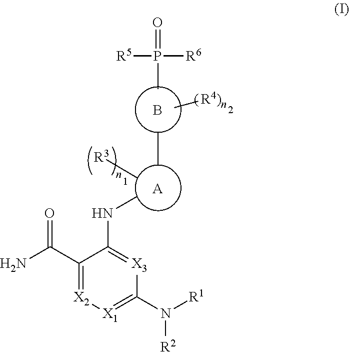
or a pharmaceutically acceptable salt, ester, solvate, prodrug, isotope-labeled derivative, or isomer thereof, wherein:
n₁ is 0, 1, 2, 3 or 4;
n₂ is 0, 1, 2, 3 or 4;
X₁ is N or CH;
X₂ is N or CH;
X₃ is N or CR⁷;
ring A is C₆₋₁₀ aryl or 5-10 membered heteroaryl;
ring B is C₆₋₁₀ aryl or 5-10 membered heteroaryl, or ring B is absent, wherein when ring
B is absent, 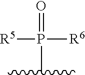
directly connects with ring A;
R¹ is hydrogen, C₁₋₆ alkyl, C₁₋₆ deuterated alkyl, C₂₋₆ alkenyl, C₂₋₆ alkynyl, C₃₋₆ cycloalkyl, 3-6 membered heterocycloalkyl, aryl, heteroaryl, —(CH₂)ₚOR^{b}, —(CH₂)ₚSR^{b}, —(CH₂)ₚC(O)R^{b}, —(CH₂)ₚC(O)OR^{b}, —(CH₂)ₚOC(O)R^{b}, —(CH₂)ₚNR^{c}R^{d}, —(CH₂)ₚC(O)NR^{c}R^{d}, —(CH₂)ₚNR^{b}C(O)R^{e}, —(CH₂)ₚNR^{b}C(O)OR^{e}, —S(O)_{q}NR^{c}R^{d} or —S(O)_{q}R^{e}, wherein C₁₋₆ alkyl, C₁₋₆ deuterated alkyl, C₂₋₆ alkenyl, C₂₋₆ alkynyl, C₃₋₆ cycloalkyl, 3-6 membered heterocycloalkyl, aryl, and heteroaryl, are unsubstituted or substituted with one or more groups independently selected from the group consisting of hydrogen, deuterium, halide, amino, —NO₂, —CN, —OH, C₁₋₆ alkyl, deuterated C₁₋₆ alky, C₁₋₆ halo-alkyl, C₁₋₆ alkoxide, C₁₋₃ halo-alkoxide, C₂₋₆ alkenyl, C₂₋₆ alkynyl, substituted or unsubstituted C₃₋₆ cycloalkyl, substituted or unsubstituted 3-6 membered heterocycloalkyl, substituted or unsubstituted aryl, and substituted or unsubstituted heteroaryl;
R² is hydrogen, C₁₋₄ alkyl, —(CH₂)ₚ-phenyl, or —(CH₂)ₚ-5-7 membered heterocycloalkyl, wherein C₁₋₄ alkyl is substituted with 0-1 R^{a}, wherein phenyl is substituted with 0-3 R^{a}, wherein 5-7 membered heterocycloalkyl comprises 1-4 hetero atoms or hetero atom groups in the ring, wherein hetero atom or hetero atom group is independently NH, N, O, S(O)_{q}, PH(O)ᵣ, or P(O)ᵣ, wherein heterocycloalkyl is substituted with 0-3 R^{a};
or R¹ and R², together with nitrogen attached thereto, form a 3-14 membered heterocycloalkyl, wherein 3-14 membered heterocycloalkyl is unsubstituted or substituted with one or more groups independently selected from the group consisting of deuterium, halide, amino, —NO₂, —CN, —OH, C₁₋₆ alkyl, deuterated C₁₋₆ alky, C₁₋₆ halo-alkyl, C₁₋₆ alkoxide, C₁₋₆ halo-alkoxide, C₂₋₆ alkenyl, and C₂₋₆ alkynyl;
if present, each R³ is independently hydrogen, deuterium, halide, —OH, amino, —SH, —NO₂, —CN, —P(O)R^{c}R^{d}, C₁₋₆ alkyl, —C(O)NH₂, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₃₋₆heterocycloalkyl, C₆₋₁₀ aryl or 5-10 membered heteroaryl, wherein C₁₋₆ alkyl, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₃₋₆heterocycloalkyl, C₆₋₁₀ aryl and 5-10 membered heteroaryl are unsubstituted or substituted with one or more groups independently selected from the group consisting of deuterium, halide, amino, —NO₂, —CN, —OH, and C₁₋₃ alkyl;
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from the group consisting of deuterium, halide, amino, —NO₂, —CN and —OH;
each of R⁵ and R⁶ is independently C₁₋₆ alkyl, C₁₋₆ deuterated alkyl, C₂₋₆ alkenyl, C₂₋₆ alkynyl, or C₃₋₆ cycloalkyl, wherein C₁₋₆ alkyl, C₁₋₆ deuterated alkyl, C₂₋₆ alkenyl, C₂₋₆ alkynyl, and C₃₋₆ cycloalkyl are unsubstituted or substituted with one or more groups independently selected from the group consisting of deuterium, halide, amino, —NO₂, —CN and —OH;
or R⁵ and R⁶, together with phosphorus attached thereto, form a 5-6 membered heterocycloalkyl, wherein 5-6 membered heterocycloalkyl is unsubstituted or substituted with one or more groups independently selected from the group consisting of deuterium, halide, amino, —NO₂, —CN and —OH;
if present, R⁷ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from the group consisting of deuterium, halide, amino, —NO₂, —CN and —OH;
if present, each of R^{a}, R^{b}, R^{c}, R^{d} and R^{e} is independently hydrogen, deuterium, halide, amino, —NO₂, —CN, —OH, alkyl, deuterated alkyl, halo-alkyl, alkoxy, halo-alkoxy, alkenyl, alkynyl, cycloalkyl, heterocycloalkyl, aryl, or heteroaryl, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from the group consisting of deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted cycloalkyl, substituted and unsubstituted heterocycloalkyl, substituted or unsubstituted aryl, and substituted or unsubstituted heteroaryl; or any two of adjacent or non-adjacent R^{a}, R^{b}, R^{c}, R^{d} and R^{e} form a cycloalkyl, heterocycloalkyl, aryl or heteroaryl, wherein cycloalkyl, heterocycloalkyl, aryl and heteroaryl are unsubstituted or substituted with one or more groups independently selected from the group consisting of deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted 6-10 membered aryl, and substituted or unsubstituted 5-10 membered heteroaryl;
if present, each p is independently 0, 1 or 2;
if present, each q is independently 1 or 2; and
if present, each r is 0 or 1;
with the proviso that when B is absent and 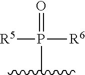
is directly connects with ring A, the compound is not

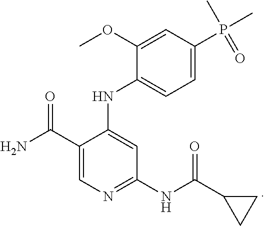 [SMILES: COc1cc(P(C)(C)=O)ccc1Nc1cc(NC(=O)C2CC2)ncc1C(N)=O]

2. The compound of claim 1, or the pharmaceutically acceptable salt, ester, solvate, prodrug, isotope-labeled derivative, or isomer thereof, wherein:
R¹ is hydrogen, C₁₋₆ alkyl, C₁₋₆ deuterated alkyl, C₂₋₆ alkenyl, C₂₋₆ alkynyl, C₃₋₆ cycloalkyl, 3-6 membered heterocycloalkyl, C₆₋₁₀ aryl, 5-10 membered heteroaryl, —C(O)R^{b}, —C(O)OR^{b}, —C(O)NR^{c}R^{d}, —S(O)_{q}NR^{c}R^{d} or —S(O)_{q}R^{e}, wherein C₁₋₆ alkyl, C₁₋₆ deuterated alkyl, C₂₋₆ alkenyl, C₂₋₆ alkynyl, C₃₋₆ cycloalkyl, 3-6 membered heterocycloalkyl, C₆₋₁₀ aryl, and 5-10 membered heteroaryl, are unsubstituted or substituted with one or more groups independently selected from the group consisting of deuterium, halide, amino, —NO₂, —CN, —OH, C₁₋₆ alkyl, deuterated C₁₋₆ alky, C₁₋₆ halo-alkyl, C₁₋₆ alkoxide, C₁₋₆ halo-alkoxide, C₂₋₆ alkenyl, C₂₋₆ alkynyl, substituted or unsubstituted C₃₋₆ cycloalkyl, substituted or unsubstituted 3-6 membered heterocycloalkyl, substituted or unsubstituted C₆₋₁₀ aryl, and substituted or unsubstituted 5-10 membered heteroaryl;
if present, each of R^{a}, R^{b}, R^{c}, R^{d} and R^{e} is independently hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, and 5-10 membered heteroaryl are unsubstituted or substituted with one or more groups independently selected from the group consisting of deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl; or any two of adjacent or non-adjacent R^{a}, R^{b}, R^{c}, R^{d} and R^{e} form a C₃₋₆ cycloalkyl, C₃₋₆heterocycloalkyl, C₆₋₁₀ aryl or 5-10 membered heteroaryl, wherein C₃₋₆ cycloalkyl, C₃₋₆heterocycloalkyl, C₆₋₁₀ aryl and 5-10 membered heteroaryl are unsubstituted or substituted with one or more groups independently selected from the group consisting of deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted or unsubstituted 3-6 membered heterocycloalkyl, substituted or unsubstituted C₆₋₁₀ aryl, and substituted or unsubstituted 5-10 membered heteroaryl; and
if present, q is independently 1 or 2.

3. The compound of claim 1, or the pharmaceutically acceptable salt, ester, solvate, prodrug, isotope-labeled derivative, or isomer thereof, wherein:
ring A is phenyl or 5-6 membered heteroaryl;
ring B is C₆₋₁₀ aryl or 5-10 membered heteroaryl, preferably phenyl or 5-6 membered heteroaryl;
if present, each R³ is independently halide, —CN, C₁₋₆ alkyl, C₁₋₆ deuterated alkyl, —O(C₁₋₆ alkyl), —O(C₁₋₆ deuterated alkyl), C₃₋₆ cycloalkyl, or —C(O)NH₂, preferably halide or —O(C₁₋₆ alkyl);
R⁵ is C₁₋₆ alkyl or C₃₋₆ cycloalkyl; preferably C₁₋₃ alkyl or cyclopropyl; and
R⁶ is C₁₋₆ alkyl or C₃₋₆ cycloalkyl; preferably C₁₋₃ alkyl or cyclopropyl.

4. The compound of claim 1, wherein the compound is of Formula (II): 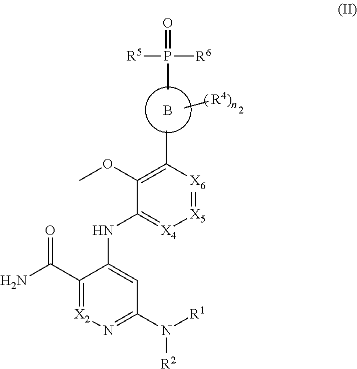
or a pharmaceutically acceptable salt, ester, solvate, prodrug, isotope-labeled derivative, or isomer thereof, wherein:
X₄ is N or CR⁸;
X₅ is N or CR⁸;
X₆ is N or CR⁸;
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from the group consisting of deuterium, halide, amino, —NO₂, —CN and —OH; and
n₂, X₂, ring B, R¹, R², R⁴, R⁵ and R⁶ are according to claim 1.

5. The compound of claim 1, or the pharmaceutically acceptable salt, ester, solvate, prodrug, isotope-labeled derivative, or isomer thereof, wherein:
R² is according to claim 1;
R¹ is selected from the group consisting of: 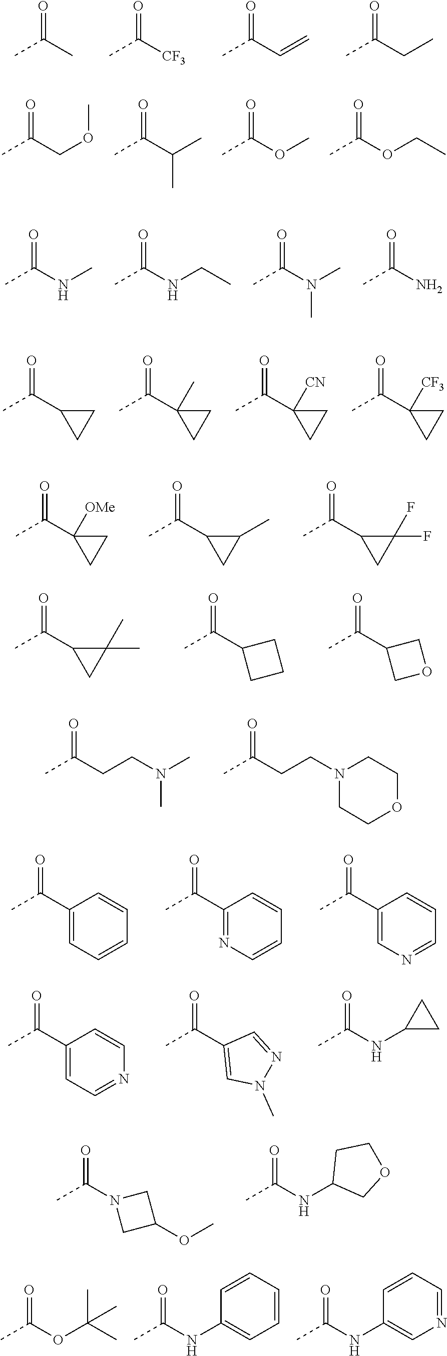 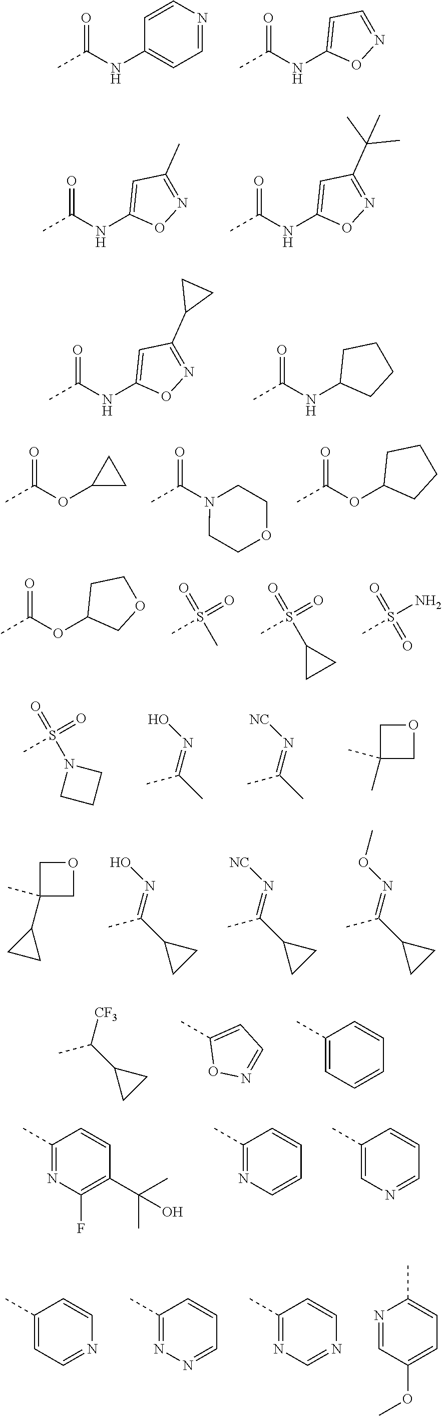 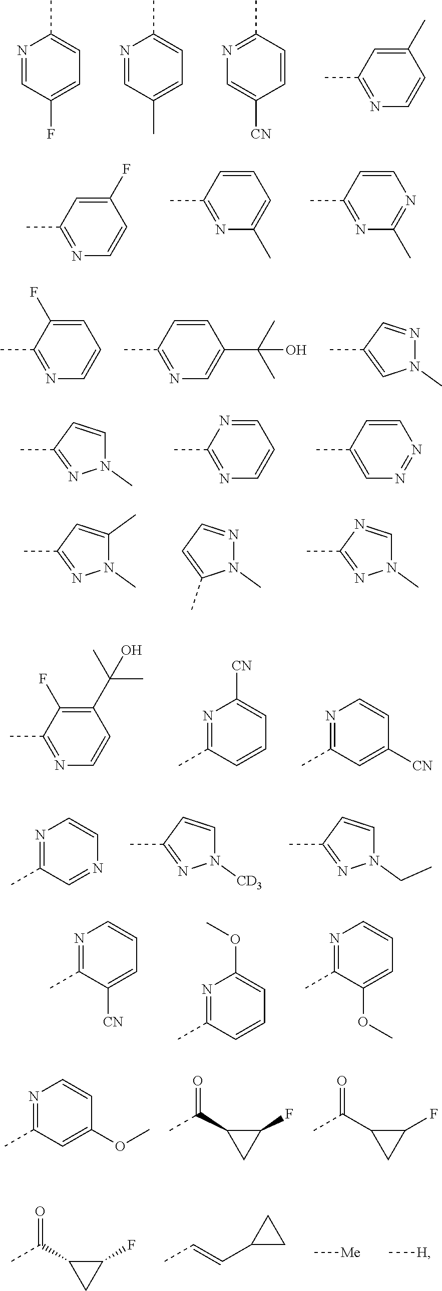
each substituted with 0-3 R⁹, with the proviso that when R¹ is H, R¹ is unsubstituted;
if present, each R⁹ is independently deuterium, halide, amino, —NO₂, —CN, —OH, C₁₋₃ alkyl, or C₁₋₃ alkoxide.

6. The compound claim 5, or the pharmaceutically acceptable salt, ester, solvate, prodrug, isotope-labeled derivative, or isomer thereof, wherein:
R² is according to claim 1;
R¹ is selected from the group consisting of: 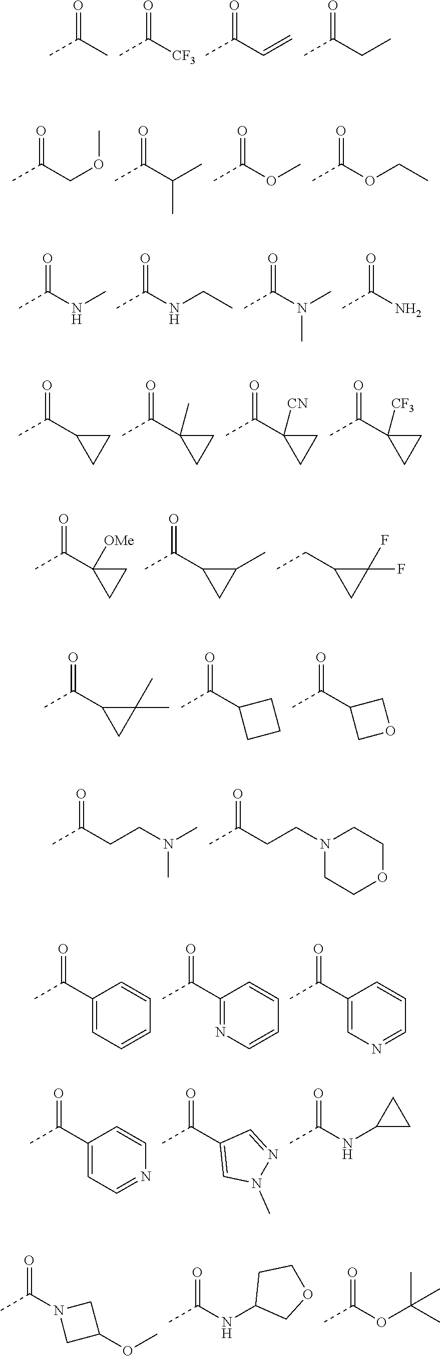 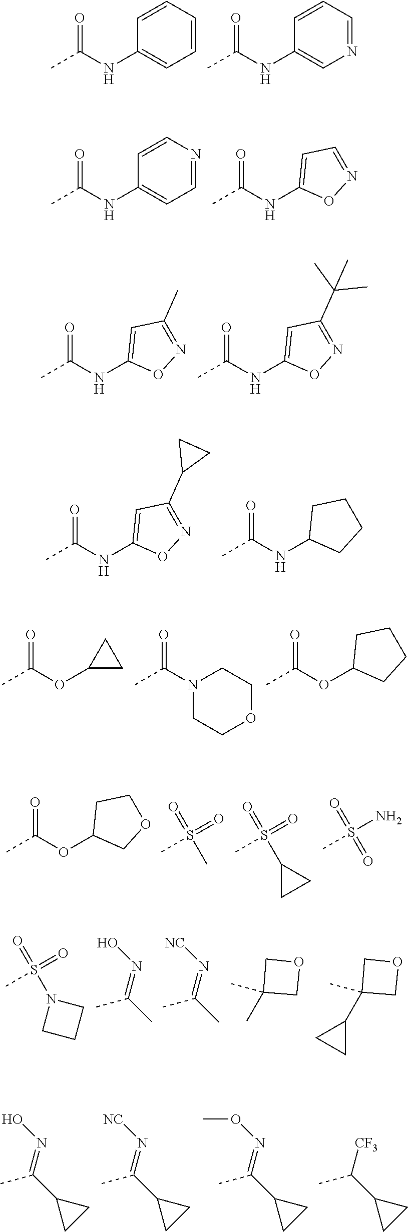 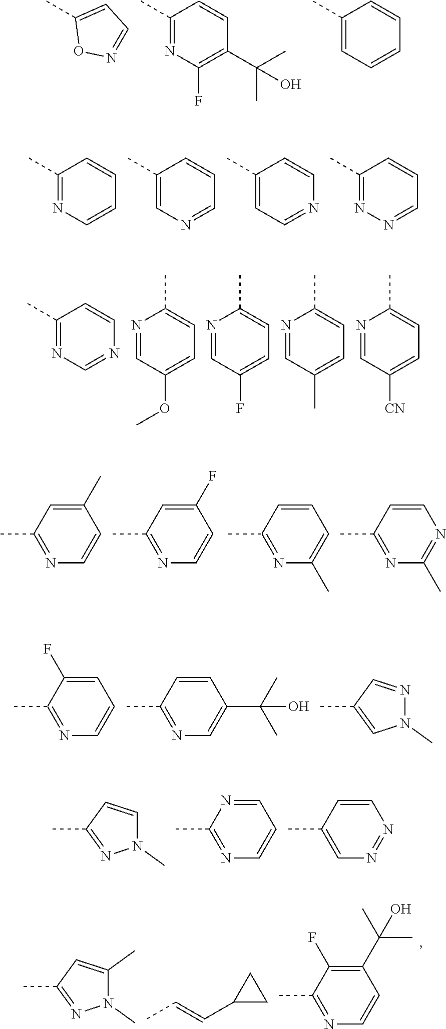
each substituted with 0-3 R⁹.

7. (canceled)

8. (canceled)

9. The compound of claim 4, wherein the compound is of Formula (III): 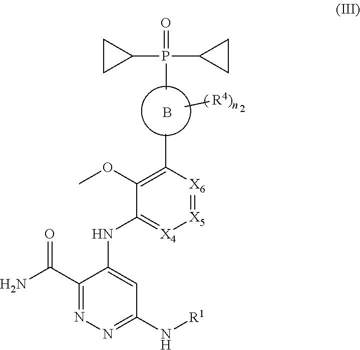
or a pharmaceutically acceptable salt, ester, solvate, prodrug, isotope-labeled derivative, or isomer thereof, wherein n₂, ring B, R¹, R⁴, X₄, X₅ and X₆ are according to claim 4.

10. The compound of claim 9, or the pharmaceutically acceptable salt, ester, solvate, prodrug, isotope-labeled derivative, or isomer thereof, wherein: 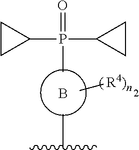
is selected from the group consisting of: 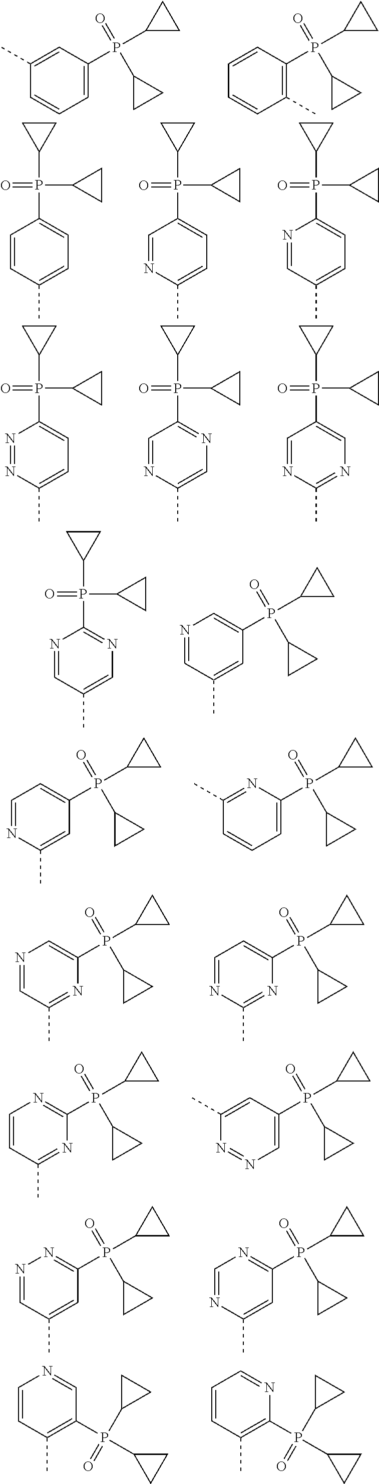 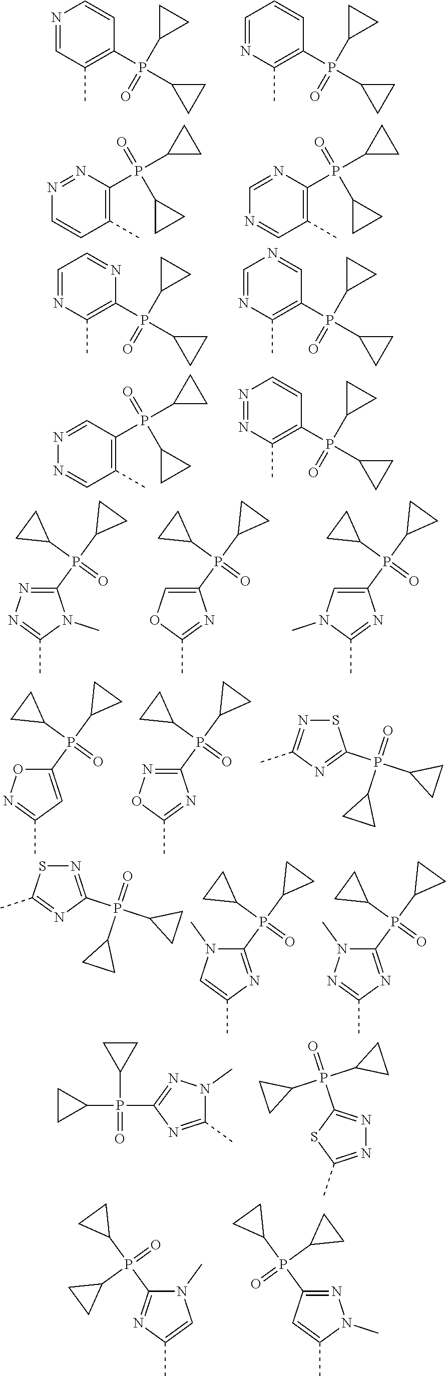 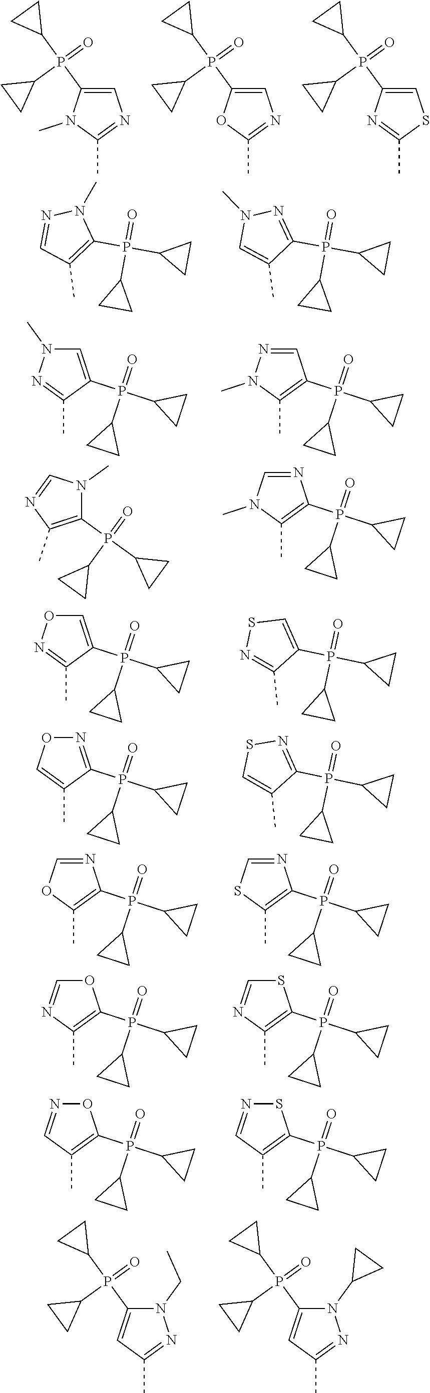 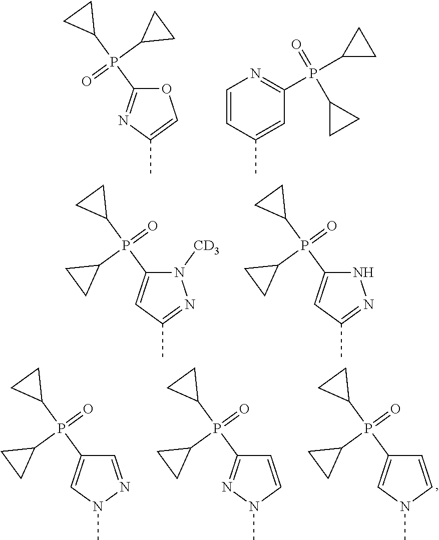
each substituted with 0 to 3 R¹¹;
if present, each R¹¹ is independently deuterium, halide, amino, —CN, —OH, C₁₋₃ alkyl or C₁₋₃ alkoxy.

11. The compound of claim 10, or the pharmaceutically acceptable salt, ester, solvate, prodrug, isotope-labeled derivative, or isomer thereof, wherein: 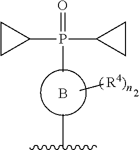
is selected from the group consisting of: 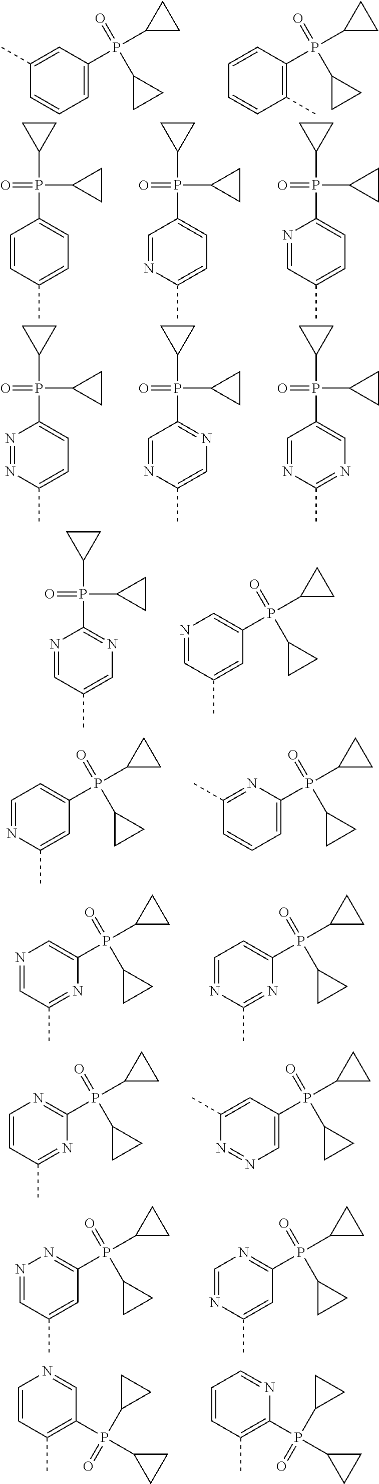 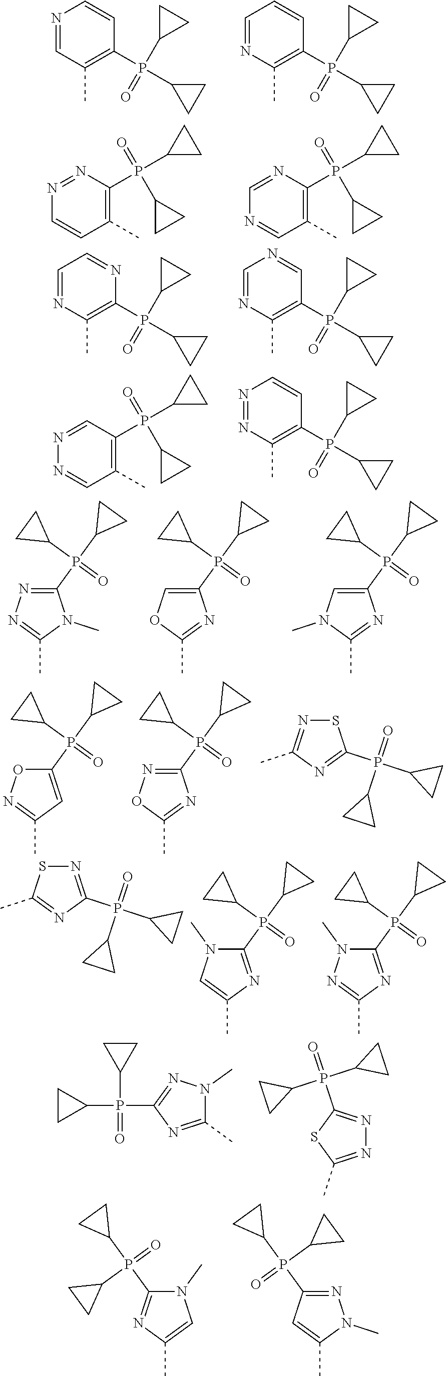 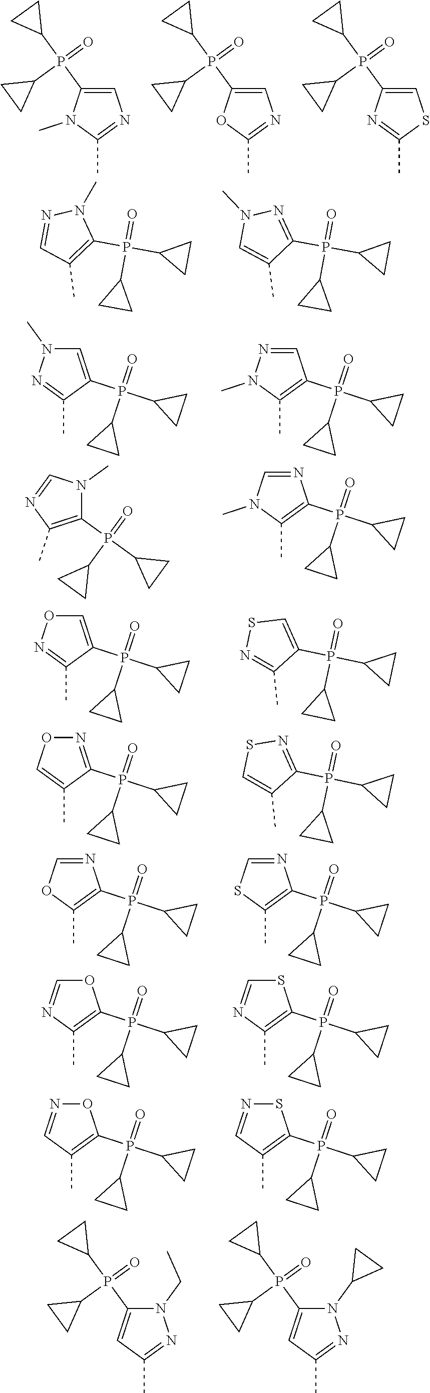 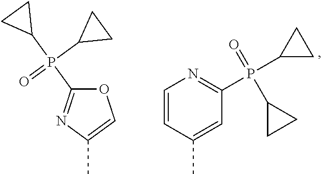
each substituted with 0 to 3 R¹¹.

12. The compound of claim 9, or the pharmaceutically acceptable salt, ester, solvate, prodrug, isotope-labeled derivative, or isomer thereof, wherein:
R¹ is independently selected from the group consisting of: 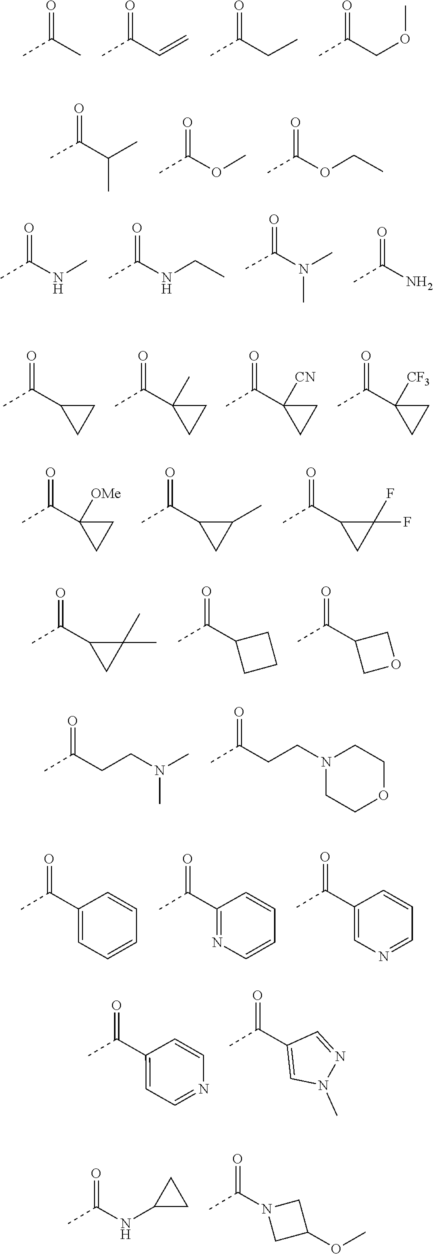 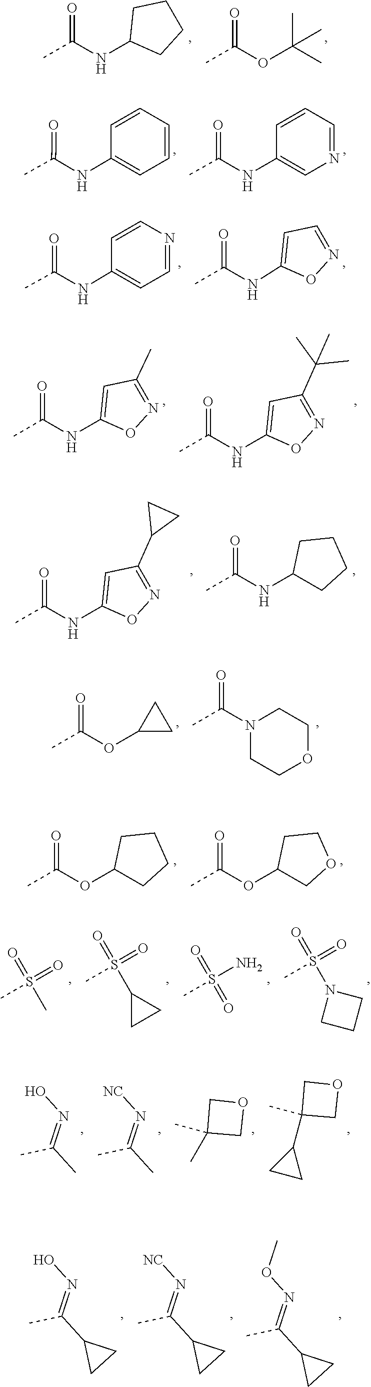 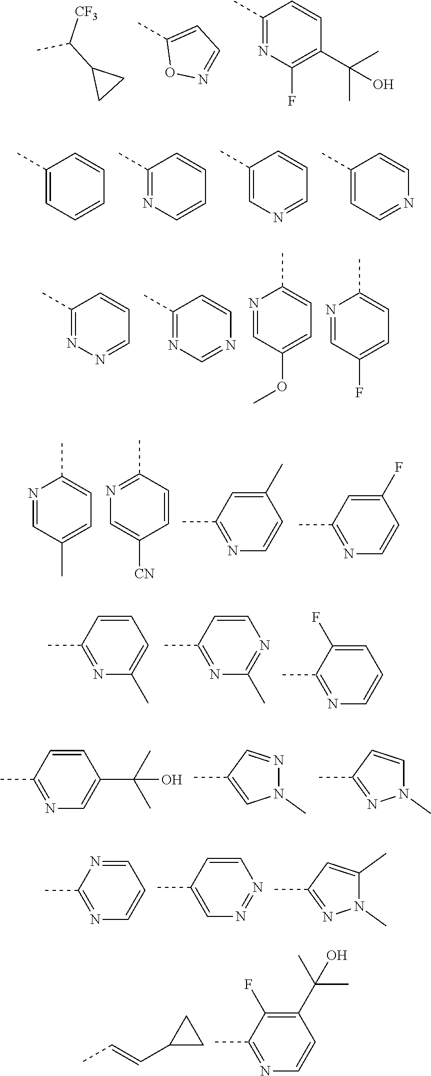 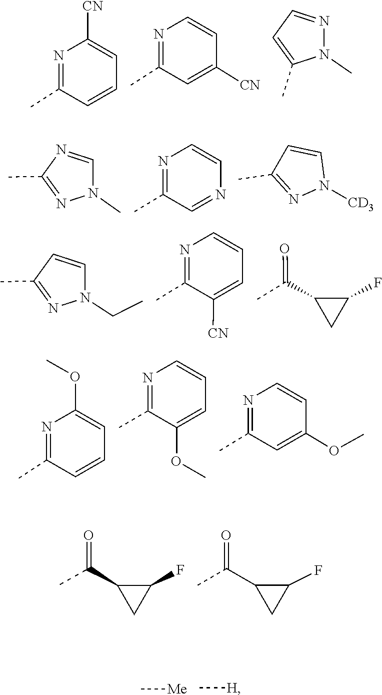
each substituted with 0 to 3 R⁹, with the proviso that when R¹ is H, R¹ is unsubstituted;
if present, R⁹ is independently deuterium, halide, amino, —NO₂, —CN, —OH, C₁₋₃ alkyl, or C₁₋₃ alkoxide.

13. The compound of claim 12, or the pharmaceutically acceptable salt, ester, solvate, prodrug, isotope-labeled derivative, or isomer thereof, wherein R¹ is independently selected from the group consisting of: 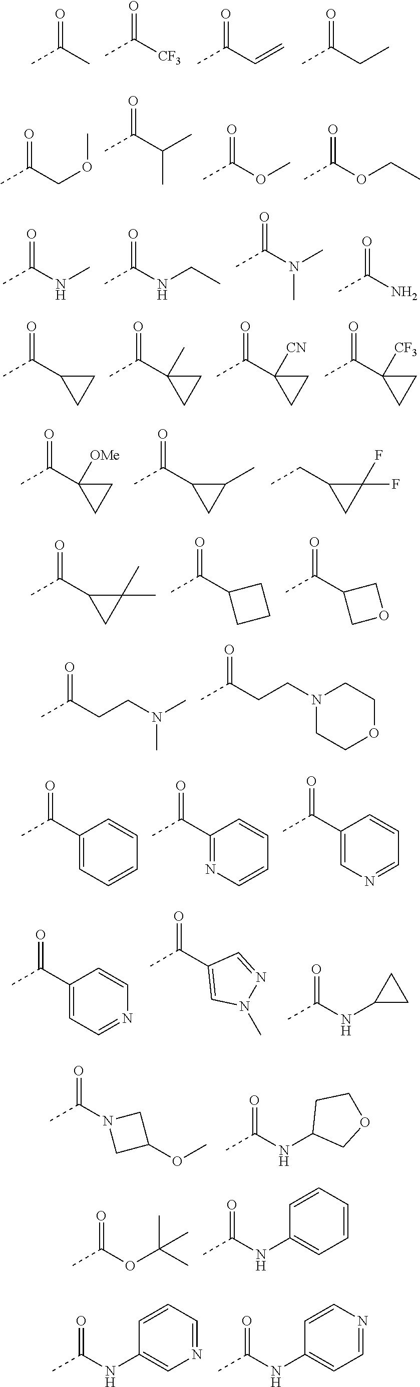 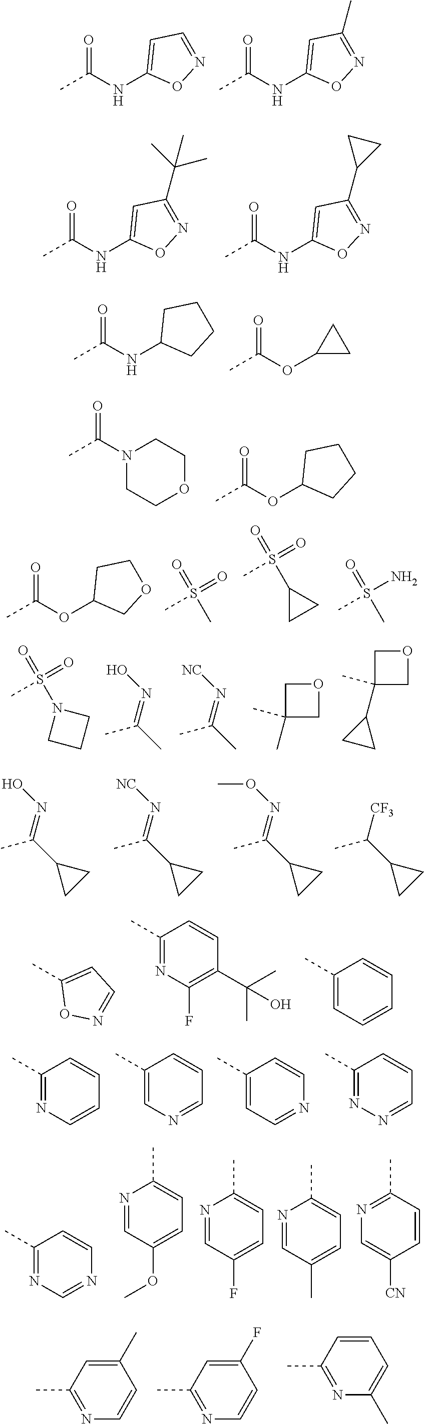 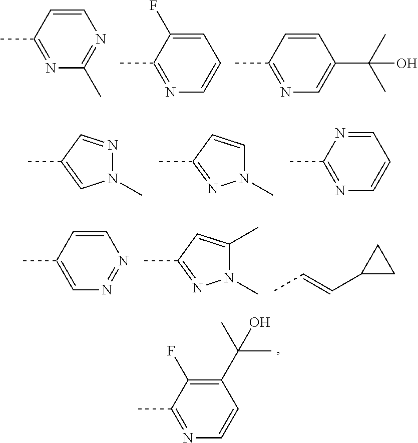
each substituted with 0 to 3 R⁹.

14. The compound of claim 1, wherein the compound is of Formula (IV): 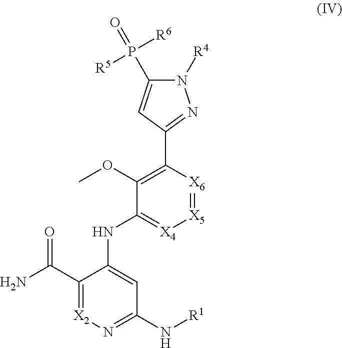
or a pharmaceutically acceptable salt, ester, solvate, prodrug, isotope-labeled derivative, or isomer thereof, wherein:
X₂ is N or CH;
X₄ is N or CR⁸;
X₅ is N or CR⁸;
X₆ is N or CR⁸;
if present, each R⁸ is independently hydrogen, deuterium, halide, —OH, amino, or —CN;
R¹ is hydrogen, C₁₋₆ alkyl, C₃₋₆ cycloalkyl, 3-6 membered heterocycloalkyl, C₆₋₁₀ aryl, 5-10 membered heteroaryl, —C(O)R^{b}, —C(O)OR^{b}, —C(O)NR^{c}R^{d} or —S(O)_{q}R^{e}, wherein C₁₋₆ alkyl, C₃₋₆ cycloalkyl, 3-6 membered heterocycloalkyl, C₆₋₁₀ aryl, and 5-10 membered heteroaryl, are unsubstituted or substituted with one or more groups independently selected from the group consisting of deuterium, halide, amino, —CN, —OH, C₁₋₆ alkyl, deuterated C₁₋₆ alkyl and C₁₋₆ alkoxide;
R⁴ is hydrogen, C₁₋₆ alkyl, deuterated C₁₋₆ alkyl, and C₃₋₆ cycloalkyl, wherein C₁₋₆ alkyl and C₃₋₆ cycloalkyl are unsubstituted or substituted with one or more groups independently selected from the group consisting of halide, amino, —CN and —OH;
each of R⁵ and R⁶ is independently C₁₋₆ alkyl or C₃₋₆ cycloalkyl;
if present, each of R^{b}, R^{c}, R^{d} and R^{e} is independently hydrogen, halide, amino, —CN, —OH, C₁₋₆ alkyl or C₃₋₆ cycloalkyl, wherein C₁₋₆ alkyl and C₃₋₆ cycloalkyl are unsubstituted or substituted with one or more groups independently selected from the group consisting of halide, amino, —CN and —OH; and
if present, q is 1 or 2.

15. The compound of claim 14, or the pharmaceutically acceptable salt, ester, solvate, prodrug, isotope-labeled derivative, or isomer thereof, wherein:
X₂ is N;
X₄ is N or CR⁸;
X₅ is N or CR⁸;
X₆ is N or CR⁸;
if present, each R⁸ is independently hydrogen or halide, preferably hydrogen;
R¹ is hydrogen, C₁₋₆ alkyl, 5-10 membered heteroaryl, —C(O)R^{b}, —C(O)OR^{b}, —C(O)NR^{c}R^{d} or —S(O)_{q}R^{e}, wherein C₁₋₆ alkyl and 5-10 membered heteroaryl, are unsubstituted or substituted with one or more groups independently selected from the group consisting of halide, —CN, C₁₋₆ alkyl, deuterated C₁₋₆ alkyl and C₁₋₆ alkoxide;
R⁴ is hydrogen, C₁₋₆ alkyl, deuterated C₁₋₆ alkyl, or C₃₋₆ cycloalkyl;
each of R⁵ and R⁶ is independently C₁₋₆ alkyl or C₃₋₆ cycloalkyl;
if present, each of R^{b}, R^{c}, R^{d} and R^{e} is independently hydrogen, C₁₋₆ alkyl or C₃₋₆ cycloalkyl, wherein C₁₋₆ alkyl and C₃₋₆ cycloalkyl are unsubstituted or substituted with one or more halides; and
if present, q is 2.

16. The compound of claim 1, wherein the compound is of Formula (V): 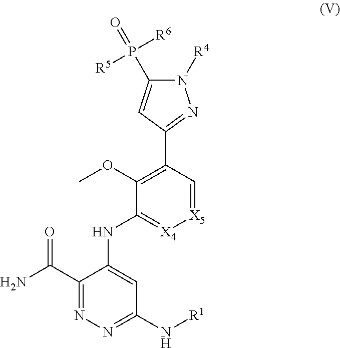
or a pharmaceutically acceptable salt, ester, solvate, prodrug, isotope-labeled derivative, or isomer thereof, wherein:
X₄ is N or CH;
X₅ is N or CR⁸;
if present, each R⁸ is independently hydrogen or fluorine, preferable hydrogen;
R¹ is 5-10 membered heteroaryl or —C(O)R^{b}, wherein 5-10 membered heteroaryl is unsubstituted or substituted with one or more groups independently selected from the group consisting of halide, —CN, C₁₋₃ alkyl, deuterated C₁₋₃ alkyl and C₁₋₃ alkoxide;
R⁴ is independently hydrogen, C₁₋₃ alkyl, deuterated C₁₋₃ alkyl and C₃₋₆ cycloalkyl;
each of R⁵ and R⁶ is independently C₁₋₃ alkyl or C₃₋₆ cycloalkyl; and
if present, R^{b} is independently C₃₋₆ cycloalkyl, wherein C₃₋₆ cycloalkyl is unsubstituted or substituted with one or more fluorine.

17. A compound or a pharmaceutically acceptable salt, ester, solvate, prodrug, isotope-labeled derivative, or isomer thereof, wherein the compound is selected from the group consisting of:

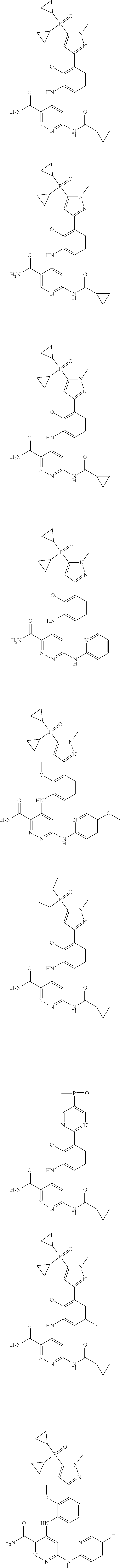

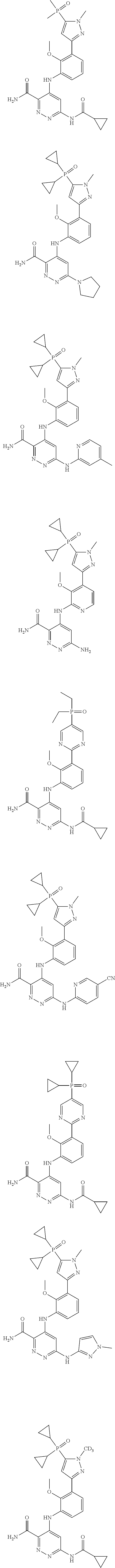

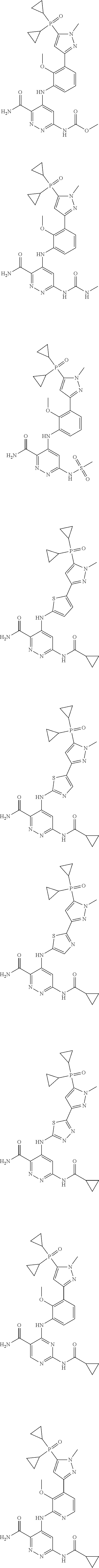

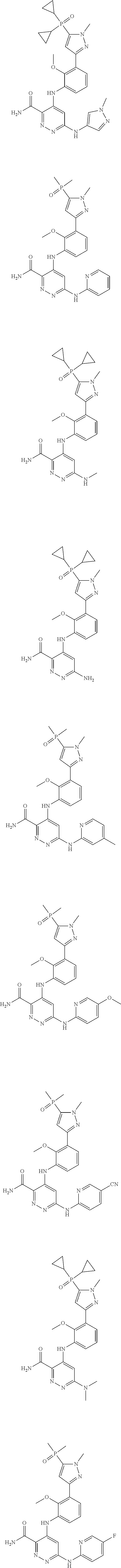

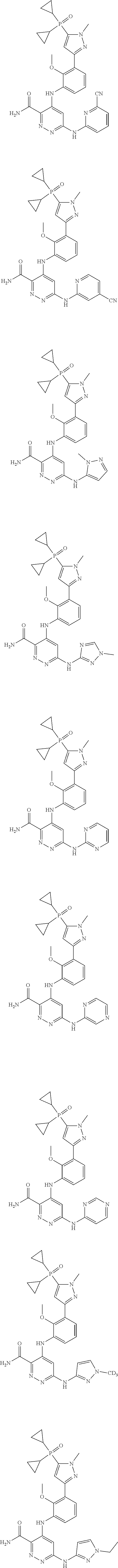

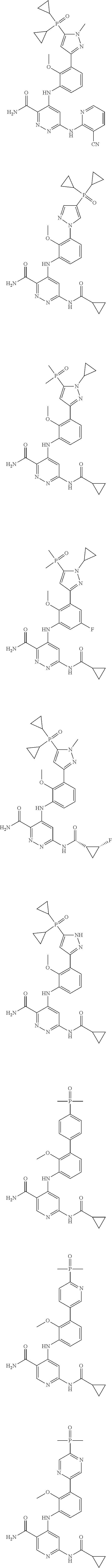

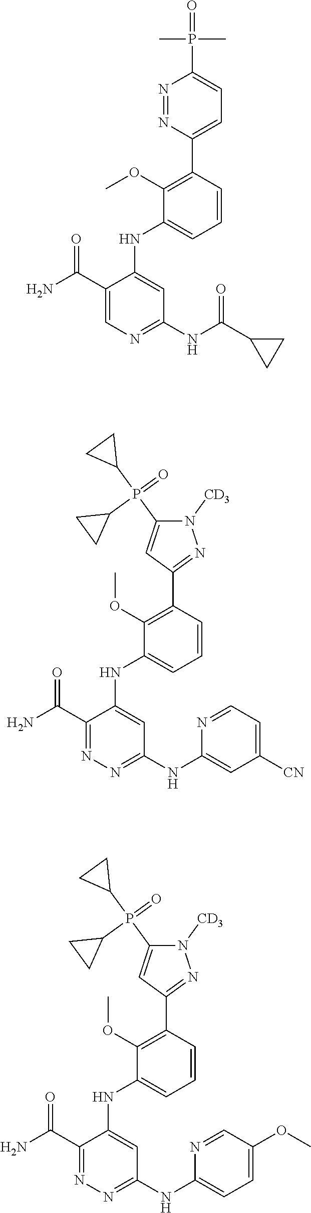 [SMILES: COc1c(Nc2cc(NC(=O)C3CC3)ncc2C(N)=O)cccc1-c1ccc(P(C)(C)=O)nn1 ; [2H]C([2H])([2H])n1nc(-c2cccc(Nc3cc(Nc4cc(C#N)ccn4)nnc3C(N)=O)c2OC)cc1P(=O)(C1CC1)C1CC1 ; [2H]C([2H])([2H])n1nc(-c2cccc(Nc3cc(Nc4ccc(OC)cn4)nnc3C(N)=O)c2OC)cc1P(=O)(C1CC1)C1CC1]

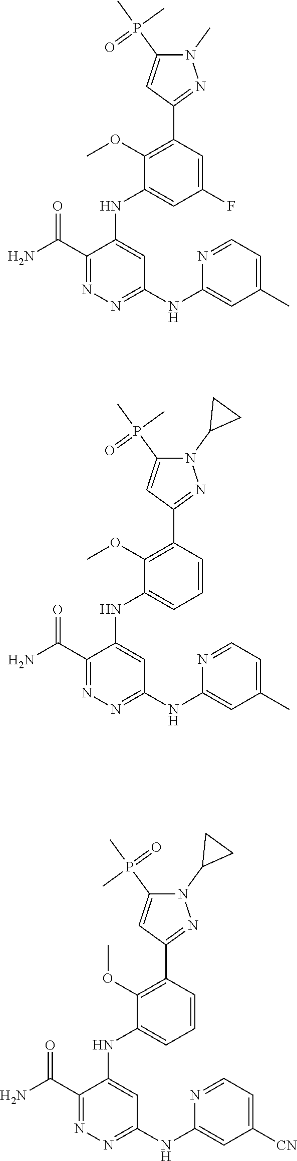

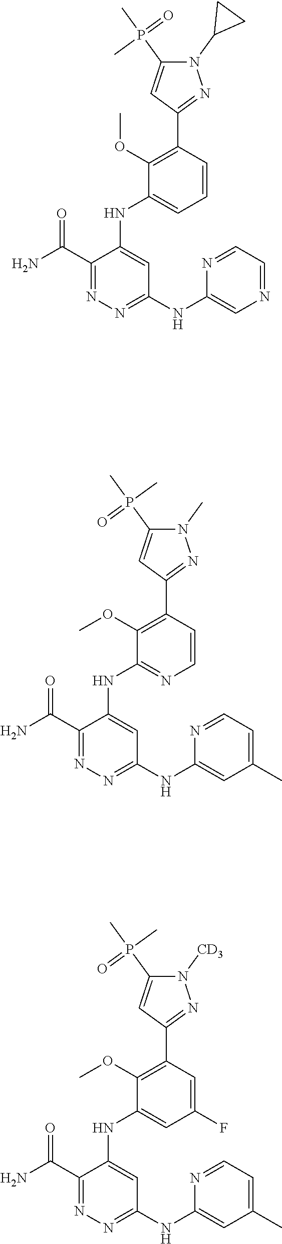

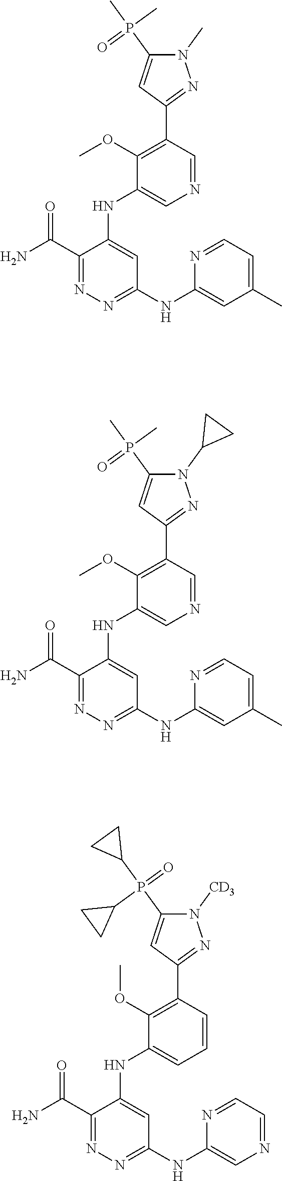

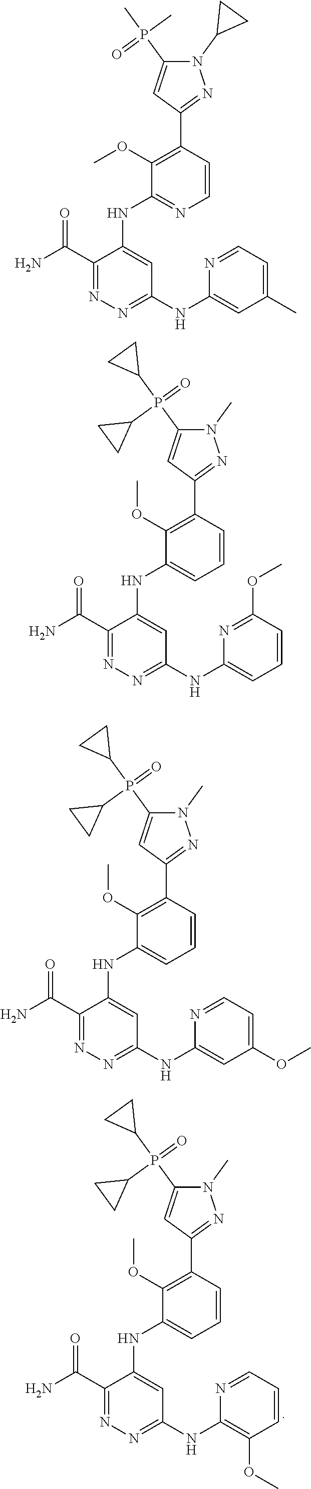

18. A pharmaceutical composition comprising a therapeutically effective amount of the compound of claim 1 or a pharmaceutically acceptable salt, ester, solvate, prodrug, isotope-labeled derivative, or isomer thereof, and a pharmaceutically acceptable carrier.

19. A composition comprising:
(i) the compound of claim 1 or a pharmaceutically acceptable salt, ester, solvate, prodrug, isotope-labeled derivative, or isomer thereof; and
(ii) one or more additional therapeutic agent selected from the group consisting of anti-autoimmune/anti-inflammatory agent, anti-tumor/anti-cancer agent, anti-allergic agent, anti-transplant rejection agent, anti-neurodegenerative agent, anti-asthma agent and other anti-obstructive airway disease agent.

20. A method for treating a disease or disorder by inhibiting TYK2 and/or JAK1 mediated signal transduction in a subject suffering therefrom, comprising administering to the subject a therapeutically effective amount of the compound of claim 1, or a pharmaceutically acceptable salt, ester, solvate, prodrug, isotope-labeled derivative, or isomer thereof, wherein the disease or disorder is autoimmune disease or inflammation disease, cancer or tumor, allergy, transplant rejection, neurodegenerative disease, asthma or other obstructive airway diseases;
wherein the autoimmune disease or inflammation disease is enteritis, skin disease, eye disease, arthritis, Hashimoto's thyroiditis, autoimmune hemolytic anemia, autoimmune atrophic gastritis, autoimmune encephalomyelitis, Goodpasture syndrome, autoimmune thrombocytopenia, sympathetic ophthalmitis, myositis, primary biliary cirrhosis, hepatitis, primary sclerosing cholangitis, chronic invasive hepatitis, nonalcoholic fatty liver disease, nonalcoholic steatohepatitis, ulcerative colitis, membranous glomerulopathy, systemic lupus erythematosus, rheumatoid arthritis, psoriatic arthritis, polyarthritis dermatomyositis, type I Interferonopathies (including Aicardi-Goutières syndrome) and other systemic sclerosis caused by overexpression of type I interferon, Mendelian disease, multiple arteritis nodosa, multiple sclerosis, relapsing multiple sclerosis, primary progressive multiple sclerosis, secondary progressive multiple sclerosis and bullous pemphigus, Cogan's syndrome, ankylosing spondylitis, Wegener's granulomatosis, autoimmune alopecia, diabetes, or thyroid inflammation;
wherein the enteritis is Crohn's disease, ulcerative colitis, inflammatory bowel disease, celiac disease, proctitis, eosinophilic gastroenteritis, or mastocytosis;
wherein the skin disease is atopic dermatitis, eczema, psoriasis, scleroderma, pruritus or other symptoms of itching, vitiligo, or alopecia;
wherein the eye disease is keratoconjunctivitis, uveitis (including uveitis associated with Behçet's disease and uveitis caused by the lens), keratitis, herpetic keratitis, keratoconus, muscular dystrophic epithelial keratitis inflammation, corneal leukopenia, anterior uveitis, scleritis, Mooren's ulcer, Graves ophthalmopathy, Vogt-Koyanagi-Harada syndrome, keratoconjunctivitis sicca, vesicular, iridocyclitis iridosarcoidosis, endocrine ophthalmopathy, sympathetic ophthalmitis, allergic conjunctivitis, or ocular neovascularization;
wherein the diabetes is type 1 diabetes or diabetic complications;
wherein the cancer or tumor is digestive/gastrointestinal cancers, colon cancers, liver cancers, skin cancers (including mast cell and squamous cell carcinomas), breast cancers, ovarian cancers, prostate cancers, lymphomas, leukemia (including acute myeloid leukemia and chronic myeloid leukemia), kidney cancer, lung cancer, muscle cancer, bone cancer, bladder cancer, brain cancer, melanoma (including oral and metastatic melanoma), Kaposi's sarcoma (including multiple myeloma), myeloproliferative disorders, proliferative diabetic retinopathy, or diseases/tumors associated with vascular hyperplasia;
wherein the neurodegenerative disease is motor neuron disease, Alzheimer's disease, Parkinson's disease, amyotrophic lateral sclerosis, Huntington's disease, cerebral ischemia, neurodegenerative diseases caused by trauma, injury, glutamate neurotoxicity or hypoxia, stroke, myocardial ischemia, renal ischemia, heart disease, cardiac hypertrophy, atherosclerosis, arteriosclerosis, ischemia/reperfusion injury of organ hypoxia or platelet aggregation;
wherein the allergy is allergic dermatitis in subjects (including allergic diseases in horses, such as allergy to bites), summer eczema, itchy horseshoes, cramps, airway inflammation, recurrent airway obstruction, airway hyperresponsiveness, and chronic obstructive pulmonary disease;
wherein the asthma or other obstructive airway diseases are chronic or excessive asthma, delayed asthma, bronchitis, bronchial asthma, allergic asthma, intrinsic asthma, extrinsic asthma or dusty asthma;
wherein the transplant rejection is islet transplant rejection, bone marrow transplant rejection, graft-versus-host disease, organ and cell transplant rejection (the organ and cell are bone marrow, cartilage, cornea, heart, intervertebral disc, islet, kidney, extremity, liver, lung, muscle, myoblasts, nerves, pancreas, skin, small intestine or trachea), or xenograft rejection.

21.-23. (canceled)

24. A method for treating a disease or disorder by inhibiting TYK2 and/or JAK1 mediated signal transduction in a subject suffering therefrom, comprising administering to the subject a therapeutically effective amount of the pharmaceutical composition of claim 18, wherein the disease or disorder is autoimmune disease or inflammation disease, cancer or tumor, allergy, transplant rejection, neurodegenerative disease, asthma or other obstructive airway diseases;
wherein the autoimmune disease or inflammation disease is enteritis, skin disease, eye disease, arthritis, Hashimoto's thyroiditis, autoimmune hemolytic anemia, autoimmune atrophic gastritis, autoimmune encephalomyelitis, Goodpasture syndrome, autoimmune thrombocytopenia, sympathetic ophthalmitis, myositis, primary biliary cirrhosis, hepatitis, primary sclerosing cholangitis, chronic invasive hepatitis, nonalcoholic fatty liver disease, nonalcoholic steatohepatitis, ulcerative colitis, membranous glomerulopathy, systemic lupus erythematosus, rheumatoid arthritis, psoriatic arthritis, polyarthritis dermatomyositis, type I Interferonopathies (including Aicardi-Goutières syndrome) and other systemic sclerosis caused by overexpression of type I interferon, Mendelian disease, multiple arteritis nodosa, multiple sclerosis, relapsing multiple sclerosis, primary progressive multiple sclerosis, secondary progressive multiple sclerosis and bullous pemphigus, Cogan's syndrome, ankylosing spondylitis, Wegener's granulomatosis, autoimmune alopecia, diabetes, or thyroid inflammation;
wherein the enteritis is Crohn's disease, ulcerative colitis, inflammatory bowel disease, celiac disease, proctitis, eosinophilic gastroenteritis, or mastocytosis;
wherein the skin disease is atopic dermatitis, eczema, psoriasis, scleroderma, pruritus or other symptoms of itching, vitiligo, or alopecia;
wherein the eye disease is keratoconjunctivitis, uveitis (including uveitis associated with Behçet's disease and uveitis caused by the lens), keratitis, herpetic keratitis, keratoconus, muscular dystrophic epithelial keratitis inflammation, corneal leukopenia, anterior uveitis, scleritis, Mooren's ulcer, Graves ophthalmopathy, Vogt-Koyanagi-Harada syndrome, keratoconjunctivitis sicca, vesicular, iridocyclitis iridosarcoidosis, endocrine ophthalmopathy, sympathetic ophthalmitis, allergic conjunctivitis, or ocular neovascularization;
wherein the diabetes is type 1 diabetes or diabetic complications;
wherein the cancer or tumor is digestive/gastrointestinal cancers, colon cancers, liver cancers, skin cancers (including mast cell and squamous cell carcinomas), breast cancers, ovarian cancers, prostate cancers, lymphomas, leukemia (including acute myeloid leukemia and chronic myeloid leukemia), kidney cancer, lung cancer, muscle cancer, bone cancer, bladder cancer, brain cancer, melanoma (including oral and metastatic melanoma), Kaposi's sarcoma (including multiple myeloma), myeloproliferative disorders, proliferative diabetic retinopathy, or diseases/tumors associated with vascular hyperplasia;
wherein the neurodegenerative disease is motor neuron disease, Alzheimer's disease, Parkinson's disease, amyotrophic lateral sclerosis, Huntington's disease, cerebral ischemia, neurodegenerative diseases caused by trauma, injury, glutamate neurotoxicity or hypoxia, stroke, myocardial ischemia, renal ischemia, heart disease, cardiac hypertrophy, atherosclerosis, arteriosclerosis, ischemia/reperfusion injury of organ hypoxia or platelet aggregation;
wherein the allergy is allergic dermatitis in subjects (including allergic diseases in horses, such as allergy to bites), summer eczema, itchy horseshoes, cramps, airway inflammation, recurrent airway obstruction, airway hyperresponsiveness, and chronic obstructive pulmonary disease;
wherein the asthma or other obstructive airway diseases are chronic or excessive asthma, delayed asthma, bronchitis, bronchial asthma, allergic asthma, intrinsic asthma, extrinsic asthma or dusty asthma;
wherein the transplant rejection is islet transplant rejection, bone marrow transplant rejection, graft-versus-host disease, organ and cell transplant rejection (the organ and cell are bone marrow, cartilage, cornea, heart, intervertebral disc, islet, kidney, extremity, liver, lung, muscle, myoblasts, nerves, pancreas, skin, small intestine or trachea), or xenograft rejection.

25. A method for treating a disease or disorder by inhibiting TYK2 and/or JAK1 mediated signal transduction in a subject suffering therefrom, comprising administering to the subject a therapeutically effective amount of the composition of claim 19, wherein the disease or disorder is autoimmune disease or inflammation disease, cancer or tumor, allergy, transplant rejection, neurodegenerative disease, asthma or other obstructive airway diseases;
wherein the autoimmune disease or inflammation disease is enteritis, skin disease, eye disease, arthritis, Hashimoto's thyroiditis, autoimmune hemolytic anemia, autoimmune atrophic gastritis, autoimmune encephalomyelitis, Goodpasture syndrome, autoimmune thrombocytopenia, sympathetic ophthalmitis, myositis, primary biliary cirrhosis, hepatitis, primary sclerosing cholangitis, chronic invasive hepatitis, nonalcoholic fatty liver disease, nonalcoholic steatohepatitis, ulcerative colitis, membranous glomerulopathy, systemic lupus erythematosus, rheumatoid arthritis, psoriatic arthritis, polyarthritis dermatomyositis, type I Interferonopathies (including Aicardi-Goutières syndrome) and other systemic sclerosis caused by overexpression of type I interferon, Mendelian disease, multiple arteritis nodosa, multiple sclerosis, relapsing multiple sclerosis, primary progressive multiple sclerosis, secondary progressive multiple sclerosis and bullous pemphigus, Cogan's syndrome, ankylosing spondylitis, Wegener's granulomatosis, autoimmune alopecia, diabetes, or thyroid inflammation;
wherein the enteritis is Crohn's disease, ulcerative colitis, inflammatory bowel disease, celiac disease, proctitis, eosinophilic gastroenteritis, or mastocytosis;
wherein the skin disease is atopic dermatitis, eczema, psoriasis, scleroderma, pruritus or other symptoms of itching, vitiligo, or alopecia;
wherein the eye disease is keratoconjunctivitis, uveitis (including uveitis associated with Behçet's disease and uveitis caused by the lens), keratitis, herpetic keratitis, keratoconus, muscular dystrophic epithelial keratitis inflammation, corneal leukopenia, anterior uveitis, scleritis, Mooren's ulcer, Graves ophthalmopathy, Vogt-Koyanagi-Harada syndrome, keratoconjunctivitis sicca, vesicular, iridocyclitis iridosarcoidosis, endocrine ophthalmopathy, sympathetic ophthalmitis, allergic conjunctivitis, or ocular neovascularization;
wherein the diabetes is type 1 diabetes or diabetic complications;
wherein the cancer or tumor is digestive/gastrointestinal cancers, colon cancers, liver cancers, skin cancers (including mast cell and squamous cell carcinomas), breast cancers, ovarian cancers, prostate cancers, lymphomas, leukemia (including acute myeloid leukemia and chronic myeloid leukemia), kidney cancer, lung cancer, muscle cancer, bone cancer, bladder cancer, brain cancer, melanoma (including oral and metastatic melanoma), Kaposi's sarcoma (including multiple myeloma), myeloproliferative disorders, proliferative diabetic retinopathy, or diseases/tumors associated with vascular hyperplasia;
wherein the neurodegenerative disease is motor neuron disease, Alzheimer's disease, Parkinson's disease, amyotrophic lateral sclerosis, Huntington's disease, cerebral ischemia, neurodegenerative diseases caused by trauma, injury, glutamate neurotoxicity or hypoxia, stroke, myocardial ischemia, renal ischemia, heart disease, cardiac hypertrophy, atherosclerosis, arteriosclerosis, ischemia/reperfusion injury of organ hypoxia or platelet aggregation;
wherein the allergy is allergic dermatitis in subjects (including allergic diseases in horses, such as allergy to bites), summer eczema, itchy horseshoes, cramps, airway inflammation, recurrent airway obstruction, airway hyperresponsiveness, and chronic obstructive pulmonary disease;
wherein the asthma or other obstructive airway diseases are chronic or excessive asthma, delayed asthma, bronchitis, bronchial asthma, allergic asthma, intrinsic asthma, extrinsic asthma or dusty asthma;
wherein the transplant rejection is islet transplant rejection, bone marrow transplant rejection, graft-versus-host disease, organ and cell transplant rejection (the organ and cell are bone marrow, cartilage, cornea, heart, intervertebral disc, islet, kidney, extremity, liver, lung, muscle, myoblasts, nerves, pancreas, skin, small intestine or trachea), or xenograft rejection.
